SEQUENCE LISTING (59 sequences)

SEQ ID NO: 1 - protein, 12 aa, Homo sapiens
  Spacer (IgG4hinge)
ESKYGPPCPPCP

SEQ ID NO: 2 - DNA, 36 bp, Homo sapiens
  Spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28
FWVLVVVGGVLACYSLLVTVAFIIFWV

SEQ ID NO: 9 - protein, 66 aa, Homo sapiens
  CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28
RSKRSRLLHSDYMNMTPRRPGPTRKHYQPYAPPRDFAAYRS

SEQ ID NO: 11 - protein, 41 aa, Homo sapiens
  CD28
RSKRSRGGHSDYMNMTPRRPGPTRKHYQPYAPPRDFAAYRS

SEQ ID NO: 12 - protein, 42 aa, Homo sapiens
  4-1BB
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 9 aa, Artificial Sequence
  Linker
  REPEAT (5)...(9): SGGGG is repeated 5 times
PGGGSGGGG

SEQ ID NO: 23 - protein, 17 aa, Artificial Sequence
  Linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  HC-CDR3
HYYYGGSYAMDY

SEQ ID NO: 55 - protein, 7 aa, Artificial Sequence
  LC-CDR2
HTSRLHS

SEQ ID NO: 56 - protein, 9 aa, Artificial Sequence
  LC-CDR3
QQGNTLPYT

SEQ ID NO: 57 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 58 - protein, 5 aa, Artificial Sequence
  Hinge
  VARIANT (1)...(1): Xaa is glycine, cysteine or arginine
  VARIANT (4)...(4): Xaa is cysteine or threonine
XPPXP

SEQ ID NO: 59 - protein, 18 aa, Artificial Sequence
  Linker
GSTSGSGKPGSGEGSTKG

(19) United States
(12) Patent Application Publication
(10) Pub. No.: US 2021/0121466 A1
(43) Pub. Date: Apr. 29, 2021

(54) COMBINATION THERAPY OF A CHIMERIC ANTIGEN RECEPTOR (CAR) T CELL THERAPY AND A KINASE INHIBITOR

(71) Applicant: Juno Therapeutics, Inc., Seattle, WA (US)

(72) Inventors: Stanley R. FRANKEL, Summit, NJ (US); Jens HASSKARL, Boudry (CH); Jason A. DUBOVSKY, Seattle, WA (US)

(73) Assignee: Juno Therapeutics, Inc., Seattle, WA (US)

(21) Appl. No.: 17/051,714

(22) PCT Filed: Apr. 30, 2019
(86) PCT No.: PCT/US2019/030084

§ 371 Date: Oct. 29, 2020

Related U.S. Application Data
(60) Provisional application No. 62/666,653, filed on May 3, 2018.

(51) Int. Cl. A61K 31/519; A61P 35/00; C07K 14/705; A61K 35/17; C07K 16/28

(52) U.S. Cl. CPC A61K 31/519; A61P 35/00; C07K 16/2818; A61K 35/17; C07K 14/70596

(57) ABSTRACT

Provided are combination therapies involving immunotherapies e.g., a chimeric antigen receptor (CAR) T cell therapy, and the use of a kinase inhibitor, e.g., a BTK/1TK inhibitor, e.g. Ibrutinib, for treating subjects having cancers, such as certain B cell malignancies, and related methods, compositions, uses and articles of manufacture. The CART cell therapy includes cells that express recombinant receptors such as anti-CD19 CARS. In some embodiments, the B-cell malignancy is a non-Hodgkin lymphoma (NHL), such as relapsed or refractory NHL or specific NHL subtype.

### CROSS-REFERENCE TO RELATED APPLICATIONS

This application claims priority from U.S. provisional application No. 62/666,653, filed May 3, 2018, entitled “COMBINATION THERAPY OF A T CELL THERAPY AND A KINASE INHIBITOR,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042017540SeqList.txt, created Apr. 30, 2019, which is 34.4 kilobytes in size. The information in the electronic format of the Sequence Listing is incorporated by reference in its entirety.

### FIELD

The present disclosure relates in some aspects to methods, compositions, uses and articles of manufacture of combination therapies involving immunotherapies, such as adoptive cell therapy, e.g., T cell therapy, and the use of a kinase inhibitor, e.g., a BTK/ITK inhibitor, for treating subjects with disease and conditions such as certain B cell malignancies, and related methods, compositions, uses and articles of manufacture. The T cell therapy includes cells that express recombinant receptors such as chimeric antigen receptors (CARs). In some embodiments, the disease or condition is a non-Hodgkin lymphoma (NHL), such as relapsed or refractory NHL or specific NHL subtype.

### BACKGROUND

Various strategies are available for immunotherapy, for example administering engineered T cells for adoptive therapy. For example, strategies are available for engineering T cells expressing genetically engineered antigen receptors, such as CARs, and administering compositions containing such cells to subjects. Improved strategies are needed to improve efficacy of the cells, for example, improving the persistence, activity and/or proliferation of the cells upon administration to subjects. Provided are methods, compositions, kits, and systems that meet such needs.

### SUMMARY

Provided herein are methods, compositions, uses, article of manufacture involving combination therapies involving administration of an immunotherapy involving a cell therapy, such as a T cell therapy, and administering to the subject a kinase inhibitor as described herein, such as ibrutinib, to a subject having a cancer, e.g., a B cell malignancy. In some aspects, the B cell malignancy is a non-Hodgkin lymphoma (NHL), such as relapsed or refractory NHL or specific NHL subtype. In some aspects, the provided methods, uses, and article of manufacture involve the administration of a T cell therapy such as CAR-expressing T cells comprises an antigen-binding domain that binds to an antigen expressed on B cells.

Provided herein is a method of treatment including administering to a subject having a cancer an effective amount of a kinase inhibitor that is or includes the structure

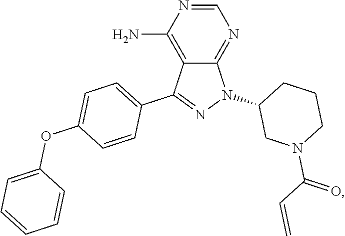 [SMILES: C=CC(=O)N1CCC[C@@H](n2nc(-c3ccc(Oc4ccccc4)cc3)c3c(N)ncnc32)C1]

or a pharmaceutically acceptable salt thereof; and administering an autologous T cell therapy to the subject, said T cell therapy containing a dose of genetically engineered T cells expressing a chimeric antigen receptor (CAR) that specifically binds to a CD19, wherein, prior to administering the T cell therapy, a biological sample has been obtained from the subject and processed, the processing includes genetically modifying T cells from the sample, optionally by introducing a nucleic acid molecule encoding the CAR into said T cells, wherein the administration of the kinase inhibitor is initiated at least at or about 3 days prior to the obtaining of the sample and is carried out in a dosing regimen comprising repeat administrations of the inhibitor at a dosing interval, over a period of time that extends at least to include administration on or after the day that the sample is obtained from the subject.

Provided herein is a method of treatment including administering to a subject having a cancer an effective amount of a kinase inhibitor that is or includes the structure

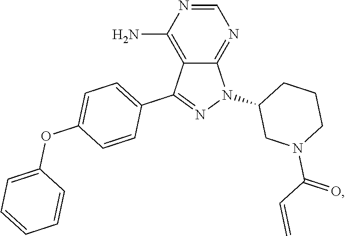 [SMILES: C=CC(=O)N1CCC[C@@H](n2nc(-c3ccc(Oc4ccccc4)cc3)c3c(N)ncnc32)C1]

or a pharmaceutically acceptable salt thereof; obtaining from the subject a biological sample and processing T cells of said sample, thereby generating a composition containing genetically engineered T cells that express a chimeric antigen receptor (CAR) that specifically binds to a CD19; and administering to the subject an autologous T cell therapy containing a dose of the genetically engineered T cells, wherein the administration of the kinase inhibitor is carried out in a dosing regimen that is initiated at least at or about 3 days prior to the obtaining of the sample and that comprises repeat administrations of the inhibitor, at a dosing interval, over a period of time and extends at least to include administration of the inhibitor on or after the day that the sample is obtained from the subject.

Provided herein is a method of treatment including administering to a subject having a cancer an effective amount of a kinase inhibitor having the structure

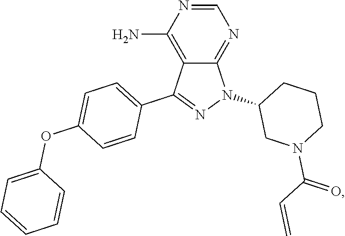 [SMILES: C=CC(=O)N1CCC[C@@H](n2nc(-c3ccc(Oc4ccccc4)cc3)c3c(N)ncnc32)C1]

or a pharmaceutically acceptable salt thereof, wherein the subject is a candidate for treatment or is to be treated with an autologous T cell therapy, said T cell therapy containing a dose of genetically engineered T cells expressing a chimeric antigen receptor (CAR) that specifically binds to a CD19, wherein prior to administering the T cell therapy, a biological sample has been obtained from the subject and processed, the processing comprising genetically modifying T cells from the sample, optionally by introducing a nucleic acid molecule encoding the CAR into said T cells; and the administration of the kinase inhibitor is initiated at least at or about 3 days prior to the obtaining of the sample and is carried out in a dosing regimen comprising repeat administrations of the inhibitor at a dosing interval for a period of time that extends at least to include administration on or after the day that the sample is obtained from the subject. In some embodiments, the method further includes administering to the subject the T cell therapy.

In some embodiments, subsequent to initiation the administration of the kinase inhibitor and prior to the administration of the T cell therapy, the subject has been preconditioned with a lymphodepleting therapy. In some aspects, subsequent to initiating the administration of the kinase inhibitor and prior to the administration of the T cell therapy, administering a lymphodepleting therapy to the subject. In some example, the administration of the kinase inhibitor is discontinued or halted during the lymphodepleting therapy.

In some embodiments, the dosing regimen includes administration of the kinase inhibitor over a period of time that extends at least to include administration up to the initiation of the lymphodepleting therapy. In some examples, the dosing regimen includes administration of the kinase inhibitor over a period of time that includes administration up to the initiation of the lymphodepleting therapy, followed by discontinuing or halting administration of the kinase inhibitor during the lymphodepleting therapy and then further administration of the kinase inhibitor for a period that extends for at least 15 days after initiation of administration of the T cell therapy.

Provided herein is a method of treatment including administering to a subject having a cancer an effective amount of a kinase inhibitor having the structure

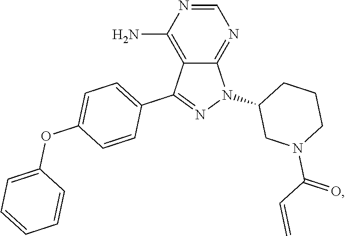 [SMILES: C=CC(=O)N1CCC[C@@H](n2nc(-c3ccc(Oc4ccccc4)cc3)c3c(N)ncnc32)C1]

or a pharmaceutically acceptable salt thereof; administering a lymphodepleting therapy to the subject; and administering an autologous T cell therapy to the subject, said T cell therapy containing a dose of genetically engineered T cells expressing a chimeric antigen receptor (CAR) that specifically binds to a CD19, wherein, prior to administering the T cell therapy containing the biological sample has been obtained from the subject and processed, the processing comprising genetically modifying T cells from the sample, optionally by introducing a nucleic acid molecule encoding the CAR into said T cells, wherein the administration of the kinase inhibitor is initiated at least at or about 3 days prior to obtaining the obtaining of the sample and is carried out in a dosing regimen comprising repeat administrations of the kinase inhibitor at a dosing interval over a period of time that includes administration up to the initiation of the lymphodepleting therapy, followed by discontinuing or halting administration of the kinase inhibitor during the lymphodepleting therapy, and then further administration of the kinase inhibitor for a period that extends for at least 15 days after initiation of administration of the T cell therapy. In some embodiments, the method further includes obtaining from the subject the biological sample and processing T cells of said sample, thereby generating a composition containing the genetically engineered T cells that express the chimeric antigen receptor (CAR) that specifically binds to a CD19.

In some of any such embodiments, the administration of the kinase inhibitor is initiated at least at or about 4 days, at least at or about 5 days, at least at or 6 days, at least at or about 7 days, at least at or about 14 days or more prior to obtaining the sample from the subject. In some examples, administration of the kinase inhibitor is initiated at least or at or about 5 days to 7 days prior to the obtaining the sample from the subject.

In some embodiments, administration of the lymphodepleting therapy is completed within 7 days prior to initiation of the administration of the T cell therapy. In some embodiments, administration of the lymphodepleting therapy is completed 2 to 7 days prior to initiation of the administration of the T cell therapy.

In some embodiments, the further administration is for a period that extends 15 days to 29 days after initiation of administration of the T cell therapy. In some embodiments, the further administration of the kinase inhibitor is for a period that extends at or about or greater than three months after initiation of administration of the T cell therapy. In some of any such embodiments, the administration of the kinase inhibitor is carried out once per day on each day it is administered during the dosing regimen.

In some of any such embodiments, the effective amount comprises from or from about 140 mg to or to about 840 mg or from or from about 140 mg to or to about 560 mg per each day the kinase inhibitor is administered.

In some examples, the effective amount includes from or from about 140 mg to or to about 560 mg per each day the kinase inhibitor is administered.

Provided herein is a method of treatment including administering to a subject having a cancer a kinase inhibitor, wherein the kinase inhibitor is or includes the structure

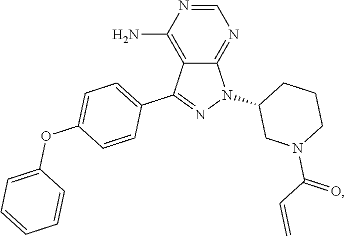 [SMILES: C=CC(=O)N1CCC[C@@H](n2nc(-c3ccc(Oc4ccccc4)cc3)c3c(N)ncnc32)C1]

or is a pharmaceutically acceptable salt thereof; and administering an autologous T cell therapy to the subject, said T cell therapy containing a dose of genetically engineered T cells expressing a chimeric antigen receptor (CAR) that specifically binds to a CD19, wherein, prior to administering the T cell therapy a biological sample has been obtained from the subject and processed, the processing comprising genetically modifying T cells from the sample, optionally by introducing a nucleic acid molecule encoding the CAR into said T cells, wherein the administration of the kinase inhibitor is initiated at least at or about 5 to 7 days prior to the obtaining of the sample and is carried out in a dosing regimen comprising repeat administration of the kinase inhibitor at a dosing interval over a period of time that extends at least to include administration on or after the day that the sample is obtained from the subject and further administration that extends for at or about or greater than three months after initiation of administration of the T cell therapy, wherein the kinase inhibitor is administered in an amount from or from about 140 mg to or to about 560 mg once per day each day it is administered during the dosing regimen. In some embodiments, subsequent to initiating administration of the kinase inhibitor and prior to the administration of the T cell therapy, the subject has been preconditioned with a lymphodepleting therapy. In some cases, the method further includes, subsequent to the administration of the kinase inhibitor and prior to the administration of the T cell therapy, administering a lymphodepleting therapy to the subject.

In some embodiments, the administration of the lymphodepleting therapy is completed within 7 days prior to initiation of the administration of the T cell therapy. In some examples, the administration of the lymphodepleting therapy is completed 2 to 7 days prior to initiation of the administration of the T cell therapy. In some cases, the dosing regimen includes discontinuing administration of the kinase inhibitor during the lymphodepleting therapy.

Provided herein is a method of treatment including administering to a subject having a cancer a kinase inhibitor, wherein the kinase inhibitor has the structure

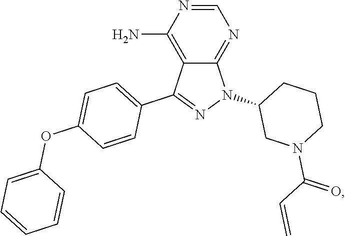 [SMILES: C=CC(=O)N1CCC[C@@H](n2nc(-c3ccc(Oc4ccccc4)cc3)c3c(N)ncnc32)C1]

or is a pharmaceutically acceptable salt thereof; and administering a lymphodepleting therapy to the subject; and administering an autologous T cell therapy to the subject within 2 to 7 days after completing the lymphodepleting therapy, said T cell therapy comprising a dose of genetically engineered T cells expressing a chimeric antigen receptor (CAR) that specifically binds to a CD19, wherein, prior to administering the T cell therapy a biological sample has been obtained from the subject and processed, the processing comprising genetically modifying T cells from the sample, optionally by introducing a nucleic acid molecule encoding the CAR into said T cells, wherein the administration of the kinase inhibitor is initiated at least at or about 5 to 7 days prior to the obtaining of the sample and is carried out in a dosing regimen comprising administration of the kinase inhibitor up to the initiation of the lymphodepleting therapy, discontinuing administration of the kinase inhibitor during the lymphodepleting therapy and further administration of the kinase inhibitor for a period that extends for at or greater than three months after initiation of administration of the T cell therapy, wherein the kinase inhibitor is administered in an amount from or from about 140 mg to or to about 560 mg once per day each day it is administered during the dosing regimen.

In some embodiments, the method further includes obtaining from the subject the biological sample and processing T cells of said sample, thereby generating a composition comprising the genetically engineered T cells that express the chimeric antigen receptor (CAR) that specifically binds to a CD19. In some of any such embodiments, the administration of the kinase inhibitor per day it is administered is from or from about 280 mg to or to about 560 mg. In some aspects, administration of the kinase inhibitor is initiated a minimum of at or about 7 days prior to obtaining the sample from the subject.

In some of any such embodiments, the administration of the kinase inhibitor is initiated from or from about 30 to or to about 40 days prior to initiating the administration of the T cell therapy; the sample is obtained from the subject from or from about 23 to or to about 38 days prior to initiating the administration of the T cell therapy; and/or the lymphodepleting therapy is completed at or about 5 to 7 days prior to initiating administration of the T cell therapy. In some embodiments, the administration of the kinase inhibitor is initiated at or about 35 days prior to initiating the administration of the T cell therapy; the sample is obtained from the subject from or from about 28 to or to about 32 days prior to initiating the administration of the T cell therapy; and/or the lymphodepleting therapy is completed at or about 5 to 7 days prior to initiating administration of the T cell therapy.

In some embodiments, the lymphodepleting therapy includes the administration of fludarabine and/or cyclophosphamide. In some embodiments, the lymphodepleting therapy includes administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days, or wherein the lymphodepleting therapy includes administration of cyclophosphamide at or about 500 mg/m². In some examples, the lymphodepleting therapy includes administration of cyclophosphamide at or about 300 mg/m² and fludarabine at or about 30 mg/m² daily for 3 days; and/or the lymphodepleting therapy includes administration of cyclophosphamide at or about 500 mg/m² and fludarabine at or about 30 mg/m² daily for 3 days.

In some embodiments, the administration of the kinase inhibitor per day it is administered is at an amount of at or about 140 mg. In some embodiments, the administration of the kinase inhibitor per day it is administered is at an amount of at or about 280 mg. In some embodiments, the administration of the kinase inhibitor per day it is administered is at an amount of at or about 420 mg. In some cases, the administration of the kinase inhibitor per day it is administered is at an amount of at or about 560 mg.

In some embodiments, the period extends for at or about or greater than four months after the initiation of the administration of the T cell therapy. In some cases, the period extends for at or about or greater than five months after the initiation of the administration of the T cell therapy. In some embodiments, the further administration is for a period that extends at or about or greater than six months.

In some of any such embodiments, the further administration of the kinase inhibitor is stopped at the end of the period, if, at the end of the period, the subject exhibits a complete response (CR) following the treatment. In some embodiments, the further administration of the kinase inhibitor is stopped at the end of the period if, at the end of the period, the cancer has progressed or relapsed following remission after the treatment. In some cases, the period extends for from or from at or about three months to at or six months. In some examples, the period extends for at or about three months after initiation of administration of the T cell therapy.

In some embodiments, the period extends for at or about 3 months after initiation of administration of the T cell therapy if the subject has, prior to at or about 3 months, achieved a complete response (CR) following the treatment or the cancer has progressed or relapsed following remission after the treatment. In some cases, the period extends for at or about 3 months after initiation of administration of the T cell therapy if the subject has at 3 months achieved a complete response (CR). In some examples, the period extends for at or about six months after initiation of administration of the T cell therapy. In some embodiments, the period extends for at or about 6 months after initiation of administration of the T cell therapy if the subject has, prior to at or about 6 months, achieved a complete response (CR) following the treatment or the cancer has progressed or relapsed following remission after the treatment. In some instances, the period extends for at or about 6 months after initiation of administration of the T cell therapy if the subject has at 6 months achieved a complete response (CR).

In some embodiments, the further administration is continued for the duration of the period even if the subject has achieved a complete response (CR) at a time point prior to the end of the period. In some embodiments, the subject achieves a complete response (CR) at a time during the period and prior to the end of the period. In some embodiments, the method includes further including continuing the further administration after the end of the period, if, at the end of the period, the subject exhibits a partial response (PR) or stable disease (SD). In some embodiments, the further administration is continued for greater than six months if, at or about six months, the subject exhibits a partial response (PR) or stable disease (SD) after the treatment. In some cases, the further administration is continued until the subject has achieved a complete response (CR) following the treatment or until the cancer has progressed or relapsed following remission after the treatment.

In some embodiments, the kinase inhibitor inhibits Bruton's tyrosine kinase (BTK) and/or inhibits IL2 inducible T-cell kinase (ITK). In some embodiments, the kinase inhibitor inhibits ITK and the inhibitor inhibits ITK or inhibits ITK with a half-maximal inhibitory concentration (IC₅₀) of less than or less than about 1000 nM, 900 nM, 800 nM, 600 nM, 500 nM, 400 nM, 300 nM, 200 nM, 100 nM or less.

In some embodiments, the subject had previously been administered the kinase inhibitor prior to the administration of the kinase inhibitor in the provided methods. In some embodiments, the subject has not previously been administered the kinase inhibitor prior to the administration of the kinase inhibitor in the provided methods.

In some embodiments, the subject and/or the cancer (a) is resistant to inhibition of Bruton's tyrosine kinase (BTK) and/or (b) includes a population of cells that are resistant to inhibition by the kinase inhibitor, optionally wherein the population of cells is or includes a population of B cells and/or does not include T cells; the subject and/or the cancer contains a mutation in a nucleic acid encoding a BTK, optionally wherein the mutation is capable of reducing or preventing inhibition of the BTK by the kinase inhibitor, optionally wherein the mutation is C481S; the subject and/or the cancer contains a mutation in a nucleic acid encoding phospholipase C gamma 2 (PLCgamma2), optionally wherein the mutation results in constitutive signaling activity, optionally wherein the mutation is R665W or L845F; at the time of the initiation of administration of the kinase inhibitor, and optionally at the time of the initiation of administration of the T cell therapy, the subject has relapsed following remission after a previous treatment with, or been deemed refractory to a previous treatment with, the kinase inhibitor and/or with a BTK inhibitor therapy; at the time of the initiation of administration of the kinase inhibitor, and optionally at the time of the initiation of the T cell therapy, the subject has progressed following a previous treatment with the inhibitor and/or with a BTK inhibitor therapy, optionally wherein the subject exhibited progressive disease as the best response to the previous treatment or progression after previous response to the previous treatment; and/or at the time of the initiation of administration of the kinase inhibitor, and optionally at the time of the initiation of the T cell therapy, the subject exhibited a response less than a complete response (CR) following a previous treatment for at least 6 months with the inhibitor and/or with a BTK inhibitor therapy.

In some of any such embodiments, the cancer is a B cell malignancy. In some cases, the B cell malignancy is a lymphoma. In some cases, the lymphoma is a non-Hodgkin lymphoma (NHL). In some examples, the NHL includes aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma with MYC and BCL2 and/or BCL6 rearrangements with DLBCL histology (double/triple hit).

In some embodiments, the subject is or has been identified as having an Eastern Cooperative Oncology Group Performance Status (ECOG) status of less than or equal to 1.

In some if any such embodiments, the kinase inhibitor is administered orally.

In some embodiments, the CD19 is a human CD19. In some aspects, the chimeric antigen receptor (CAR) includes an extracellular antigen-recognition domain that specifically binds to the CD19 and an intracellular signaling domain including an ITAM. In some cases, the intracellular signaling domain includes a signaling domain of a CD3-zeta (CD3) chain, optionally a human CD3-zeta chain. In some embodiments, the chimeric antigen receptor (CAR) further contains a costimulatory signaling region. In some cases, the costimulatory signaling region includes a signaling domain of CD28 or 4-1BB, optionally human CD28 or human 4-1BB. In some embodiments, the costimulatory domain is or includes a signaling domain of human 4-1BB.

In some embodiments, the CAR contains an scFv specific for the CD19; a transmembrane domain; a cytoplasmic signaling domain derived from a costimulatory molecule, which optionally is or includes a 4-1BB, optionally human 4-1BB; and a cytoplasmic signaling domain derived from a primary signaling ITAM-containing molecule, which optionally is or includes a CD3zeta signaling domain, optionally a human CD3zeta signaling domain; and optionally wherein the CAR further includes a spacer between the transmembrane domain and the scFv; the CAR contains, in order, an scFv specific for the CD19; a transmembrane domain; a cytoplasmic signaling domain derived from a costimulatory molecule, which optionally is or includes a 4-1BB signaling domain, optionally a human 4-1BB signaling domain; and a cytoplasmic signaling domain derived from a primary signaling ITAM-containing molecule, which optionally is a CD3zeta signaling domain, optionally human CD3zeta signaling domain; or the CAR contains, in order, an scFv specific for the CD19; a spacer; a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or includes a CD3zeta signaling domain.

In some embodiments, the CAR contains a spacer and the spacer is a polypeptide spacer that (a) includes or consists of all or a portion of an immunoglobulin hinge or a modified version thereof or includes about 15 amino acids or less, and does not include a CD28 extracellular region or a CD8 extracellular region, (b) includes or consists of all or a portion of an immunoglobulin hinge, optionally an IgG4 hinge, or a modified version thereof and/or includes about 15 amino acids or less, and does not include a CD28 extracellular region or a CD8 extracellular region, or (c) is at or about 12 amino acids in length and/or includes or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) includes or consists of the formula X₁PPX₂P (SEQ ID NO:58), where X₁ is glycine, cysteine or arginine and X₂ is cysteine or threonine; and/or the costimulatory domain includes SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain includes SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or the scFv includes a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv includes a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv includes, in order, a V_{H}, a linker, optionally including SEQ ID NO: 41, and a V_{L}, and/or the scFv includes a flexible linker and/or includes the amino acid sequence set forth as SEQ ID NO: 42.

In some of any such embodiments, the dose of genetically engineered T cells contains from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive. In some embodiments, the dose of genetically engineered T cells contains at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells. In some cases, the dose of genetically engineered T cells contains at or about 5×10⁷ total CAR-expressing T cells. In some instances, the dose of genetically engineered T cells contains at or about 1×10⁸ CAR-expressing cells. In some embodiments, the dose of genetically engineered T cells includes CD4+ T cells expressing the CAR and CD8+ T cells expressing the CAR and the administration of the dose contains administering a plurality of separate compositions, said plurality of separate compositions including a first composition including one of the CD4+ T cells and the CD8+ T cells and the second composition including the other of the CD4+ T cells or the CD8+ T cells.

In some embodiments, the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or the initiation of administration of the first composition and the initiation of administration of the second composition are carried out between about 1 minute and about 1 hour apart or between about 5 minutes and about 30 minutes apart. In some aspects, the first composition and second composition are administered no more than 2 hours, no more than 1 hour, no more than 30 minutes, no more than 15 minutes, no more than 10 minutes or no more than 5 minutes apart.

In some embodiments, the first composition contains the CD4+ T cells. In some embodiments, the first composition contains the CD8+ T cells. In some embodiments, the first composition is administered prior to the second composition. In some embodiments, the dose of cells is administered parenterally, optionally intravenously. In some of any such embodiments, the T cells are primary T cells obtained from the sample from the subject. In some cases, the T cells are autologous to the subject.

In some of any such embodiments, the processing includes isolating T cells, optionally CD4+ and/or CD8+ T cells, from the sample obtained from the subject, thereby producing an input composition containing primary T cells; and introducing the nucleic acid molecule encoding the CAR into T cells of the input composition. In some cases, the isolating including carrying out immunoaffinity-based selection.

In some embodiments, the biological sample is or includes a whole blood sample, a buffy coat sample, a peripheral blood mononuclear cells (PBMC) sample, an unfractionated T cell sample, a lymphocyte sample, a white blood cell sample, an apheresis product, or a leukapheresis product.

In some embodiments, prior to the introducing, the process includes incubating the input composition under stimulating conditions, said stimulating conditions including the presence of a stimulatory reagent capable of activating one or more intracellular signaling domains of one or more components of a TCR complex and/or one or more intracellular signaling domains of one or more costimulatory molecules, thereby generating a stimulated composition, wherein the nucleic acid molecule encoding the CAR is introduced into the stimulated composition. In some examples, the stimulatory reagent includes a primary agent that specifically binds to a member of a TCR complex, optionally that specifically binds to CD3. In some cases, the stimulatory reagent further includes a secondary agent that specifically binds to a T cell costimulatory molecule, optionally wherein the costimulatory molecule is selected from CD28, CD137 (4-1-BB), OX40, or ICOS. In some examples, the primary and/or secondary agents include an antibody, optionally wherein the stimulatory reagent includes incubation with an anti-CD3 antibody and an anti-CD28 antibody, or an antigen-binding fragment thereof.

In some cases, the primary agent and/or secondary agent are present on the surface of a solid support. In some examples, the solid support is or includes a bead, optionally wherein the bead is magnetic or superparamagnetic. In some embodiments, the bead includes a diameter of greater than or greater than about 3.5 μm but no more than about 9 μm or no more than about 8 μm or no more than about 7 μm or no more than about 6 μm or no more than about 5 In some examples, the bead includes a diameter of or about 4.5 μm.

In some embodiments, the introducing includes transducing cells of the stimulated composition with a viral vector including a polynucleotide encoding the recombinant receptor. In some cases, the viral vector is a retroviral vector. In some examples, the viral vector is a lentiviral vector or gammaretroviral vector.

In some embodiments, the process further includes after the introducing cultivating the T cells, optionally wherein the cultivating is carried out under conditions to result in the proliferation or expansion of cells to produce an output composition containing the T cell therapy. In some instances, subsequent to the cultivating, the method further includes formulating cells of the output composition for cryopreservation and/or for administration of the T cell therapy to the subject, optionally wherein the formulating is in the presence of a pharmaceutically acceptable excipient.

In some of any such embodiments, the subject is a human.

In some embodiments, at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or at least 60, 70, 80, 90, or 95% of subjects achieving a CR by six months remain in response, remain in CR, and/or survive or survive without progression, for greater at or greater than 3 months and/or at or greater than 6 months and/or at greater than nine months; and/or at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or at least 60, 70, 80, 90, or 95% of subjects achieving an OR by six months remain in response or surviving for greater at or greater than 3 months and/or at or greater than 6 months.

In some embodiments, at or immediately prior to the time of the administration of the dose of cells the subject has relapsed following remission after treatment with, or become refractory to, one or more prior therapies for the lymphoma, optionally the NHL, optionally one, two or three prior therapies other than another dose of cells expressing the CAR. In some embodiments, at or prior to the administration of the T cell therapy containing the dose of cells, the subject is or has been identified as having a double/triple hit lymphoma; the subject is or has been identified as having a chemorefractory lymphoma, optionally a chemorefractory DLBCL; and/or the subject has not achieved a complete response (CR) in response to a prior therapy.

Provided herein are kits containing one or more unit doses of a kinase inhibitor that is or comprises the structure

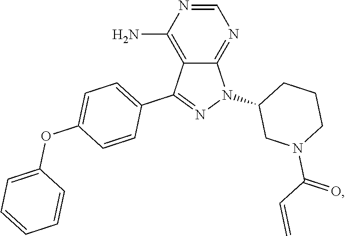 [SMILES: C=CC(=O)N1CCC[C@@H](n2nc(-c3ccc(Oc4ccccc4)cc3)c3c(N)ncnc32)C1]

or is a pharmaceutically acceptable salt thereof, and instructions for carrying out any of the methods provided herein.

Provided herein is a kit containing one or more unit doses of a kinase inhibitor that is or includes the structure

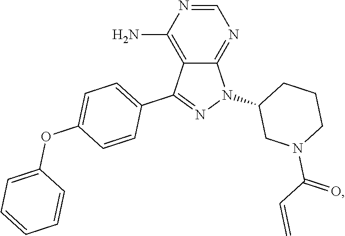 [SMILES: C=CC(=O)N1CCC[C@@H](n2nc(-c3ccc(Oc4ccccc4)cc3)c3c(N)ncnc32)C1]

or is a pharmaceutically acceptable salt thereof, and instructions for administering the one or more unit doses to a subject having a cancer that is a candidate for treatment with or who is to be treated with an autologous T cell therapy, said T cell therapy containing a dose of genetically engineered T cells expressing a chimeric antigen receptor (CAR) that specifically binds to a CD19, and in which, prior to administration of the T cell therapy, a biological sample is obtained from the subject and processed, the processing comprising genetically modifying T cells from the sample, optionally by introducing a nucleic acid encoding the CAR into the T cells, wherein the instructions specify initiating administration of a unit dose of the kinase inhibitor to the subject at or at least about 3 days prior to the obtaining of the sample and in a dosing regimen comprising repeat administrations of one or more unit doses at a dosing interval over a period of time that extends at least to include administration on or after the day the sample is obtained from the subject.

In some cases, the instructions further specify administering the T cell therapy to the subject. In some instances, the instructions further specify, subsequent to initiating the administration of the kinase inhibitor and prior to the administration of the T cell therapy, administering a lymphodepleting therapy to the subject. In some embodiments, the instructions specify administration of the kinase inhibitor is to be discontinued during administration of the lymphodepleting therapy. In some cases, the instructions specify the dosing regimen includes administration of the kinase inhibitor for a period of time that extends at least until the initiation of the lymphodepleting therapy.

In some embodiments, the instructions specify the dosing regimen includes administration of the kinase inhibitor over a period of time that includes administration up to the initiation of the lymphodepleting therapy, followed by discontinuing or halting administration of the kinase inhibitor during the lymphodepleting therapy and then further administration of the kinase inhibitor for a period that extends for at least 15 days after initiation of administration of the T cell therapy. In some embodiments, the instructions specify administration of the kinase inhibitor is initiated at least at or about 4 days, at least at or about 5 days, at least at or about 6 days, at least at or about 7 days, at least at or about 14 days or more prior to obtaining the sample from the subject. In some aspects, the instructions specify administration of the kinase inhibitor is initiated at least at or about 5 days to 7 days prior to obtaining the sample from the subject. In some cases, the instructions specify the administration of the lymphodepleting therapy is to be completed within 7 days prior to initiation of the administration of the T cell therapy. In some embodiments, the instructions specify the administration of the lymphodepleting therapy is to be completed 2 to 7 days prior to initiation of the administration of the T cell therapy.

In some embodiments, the instructions specify the further administration of the kinase inhibitor is for a period that extends at or about or greater than three months after initiation of administration of the T cell therapy. In some embodiments, the instructions specify the administration of each unit dose of the kinase inhibitor is carried out once per day on each day it is administered during the dosing regimen.

In some embodiments, the one or more unit doses each contains from or from about 140 mg to or to about 840 mg. In some embodiments, the one or more unit doses each contain from or from about 140 mg to or to about 560 mg per each day the kinase inhibitor is administered. In some cases, the one or more unit doses each contain from or from about 280 mg to or to about 560 mg.

In some embodiments, the instructions specify the administration of the kinase inhibitor is initiated a minimum of at or about 7 days prior to obtaining the sample from the subject. In some embodiments, the instructions specify the administration of the kinase inhibitor is initiated from or from about 30 to or to about 40 days prior to initiating the administration of the T cell therapy; the sample is obtained from the subject from or from about 23 to or to about 38 days prior to initiating the administration of the T cell therapy; and/or the lymphodepleting therapy is completed at or about 5 to 7 days prior to initiating administration of the T cell therapy. In some embodiments, the instructions specify the administration of the kinase inhibitor is initiated at or about 35 days prior to initiating the administration of the T cell therapy; the sample is obtained from the subject from or from about 28 to or to about 32 days prior to initiating the administration of the T cell therapy; and/or the lymphodepleting therapy is completed at or about 5 to 7 days prior to initiating administration of the T cell therapy.

In some embodiments, the lymphodepleting therapy includes the administration of fludarabine and/or cyclophosphamide. In some embodiments, the instructions specify administration of the lymphodepleting therapy includes administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days, or wherein the lymphodepleting therapy includes administration of cyclophosphamide at or about 500 mg/m². In some embodiments, the instructions specify the lymphodepleting therapy includes administration of cyclophosphamide at or about 300 mg/m² and fludarabine at or about 30 mg/m² daily for 3 days; and/or the lymphodepleting therapy includes administration of cyclophosphamide at or about 500 mg/m² and fludarabine at or about 30 mg/m² daily for 3 days.

In some embodiments, each unit dose of the kinase inhibitor is or is about 140 mg and/or the instructions specify administering the kinase inhibitor per day it is administered at an amount of at or about 140 mg. In some examples, each unit dose of the kinase inhibitor is or is about 280 mg and/or the instructions specify administering the kinase inhibitor per day it is administered is at an amount of at or about 280 mg. In some cases, each unit dose of the kinase inhibitor is or is about 420 mg and/or the instructions specify administering of the kinase inhibitor per day it is administered is at an amount of at or about 420 mg. In some embodiments, each unit dose of the kinase inhibitor is or is about 560 mg and/or the instructions specify administering the kinase inhibitor per day it is administered is at an amount of at or about 560 mg.

In some embodiments, the instructions specify the period extends for at or about or greater than four months after the initiation of the administration of the T cell therapy. In some cases, the instructions specify the period extends for at or about or greater than five months after the initiation of the administration of the T cell therapy. In some embodiments, the instructions specify the further administration is for a period that extends at or about or greater than six months.

In some embodiments, the instructions specify the further administration of the kinase inhibitor is stopped at the end of the period, if, at the end of the period, the subject exhibits a complete response (CR) following the treatment. In some embodiments, the instructions specify further administration of the kinase inhibitor is stopped at the end of the period if, at the end of the period, the cancer has progressed or relapsed following remission after the treatment. In some embodiments, the instructions specify the period extends for from or from at or about three months to at or six months. In some cases, the instructions specify the period extends for at or about three months after initiation of administration of the T cell therapy.

In some embodiments, the instructions specify the period extends for at or about 3 months after initiation of administration of the T cell therapy if the subject has, prior to at or about 3 months, achieved a complete response (CR) following the treatment or the cancer has progressed or relapsed following remission after the treatment. In some embodiments, the instructions specify the period extends for at or about 3 months after initiation of administration of the T cell therapy if the subject has at 3 months achieved a complete response (CR).

In some embodiments, the instructions specify the period extends for at or about six months after initiation of administration of the T cell therapy. In some embodiments, the instructions specify the period extends for at or about 6 months after initiation of administration of the T cell therapy if the subject has, prior to at or about 6 months, achieved a complete response (CR) following the treatment or the cancer has progressed or relapsed following remission after the treatment. In some cases, the instructions specify the period extends for at or about 6 months after initiation of administration of the T cell therapy if the subject has at 6 months achieved a complete response (CR).

In some embodiments, the instructions specify the further administration is continued for the duration of the period even if the subject has achieved a complete response (CR) at a time point prior to the end of the period. In some embodiments, the instructions specify further including continuing the further administration after the end of the period, if, at the end of the period, the subject exhibits a partial response (PR) or stable disease (SD). In some cases, the instructions specify the further administration is continued for greater than six months if, at or about six months, the subject exhibits a partial response (PR) or stable disease (SD) after the treatment. In some cases, the instructions specify the further administration is continued until the subject has achieved a complete response (CR) following the treatment or until the cancer has progressed or relapsed following remission after the treatment.

In some embodiments, the kinase inhibitor inhibits Bruton's tyrosine kinase (BTK) and/or inhibits IL2 inducible T-cell kinase (ITK). In some examples, the kinase inhibitor inhibits ITK and the inhibitor inhibits ITK or inhibits ITK with a half-maximal inhibitory concentration (IC₅₀) of less than or less than about 1000 nM, 900 nM, 800 nM, 600 nM, 500 nM, 400 nM, 300 nM, 200 nM, 100 nM or less.

In some embodiments, the instructions specify the subject has been or can have been previously administered the kinase inhibitor prior to the administration of the one or more unit doses of the kinase inhibitor. In some aspects, the instructions specify the subject has not been or is one who has not been previously administered the kinase inhibitor prior to the administration of the one or more unit doses of the kinase inhibitor.

In some embodiments, the subject and/or the cancer (a) is resistant to inhibition of Bruton's tyrosine kinase (BTK) and/or (b) contains a population of cells that are resistant to inhibition by the kinase inhibitor, optionally wherein the population of cells is or contains a population of B cells and/or does not contain T cells; the subject and/or the cancer includes a mutation in a nucleic acid encoding a BTK, optionally wherein the mutation is capable of reducing or preventing inhibition of the BTK by the kinase inhibitor, optionally wherein the mutation is C481S; the subject and/or the cancer includes a mutation in a nucleic acid encoding phospholipase C gamma 2 (PLCgamma2), optionally wherein the mutation results in constitutive signaling activity, optionally wherein the mutation is R665W or L845F; at the time of the initiation of administration of the kinase inhibitor and optionally at the time of the initiation of administration of the T cell therapy, the subject has relapsed following remission after a previous treatment with, or been deemed refractory to a previous treatment with, the kinase inhibitor and/or with a BTK inhibitor therapy; at the time of the initiation of administration of the kinase inhibitor, and optionally at the time of the initiation of the T cell therapy, the subject has progressed following a previous treatment with the inhibitor and/or with a BTK inhibitor therapy, optionally wherein the subject exhibited progressive disease as the best response to the previous treatment or progression after previous response to the previous treatment; and/or at the time of the initiation of administration of the kinase inhibitor in and optionally at the time of the initiation of the T cell therapy, the subject exhibited a response less than a complete response (CR) following a previous treatment for at least 6 months with the inhibitor and/or with a BTK inhibitor therapy.

In some embodiments, the cancer is a B cell malignancy. In some instances, the B cell malignancy is a lymphoma. In some cases, the lymphoma is a non-Hodgkin lymphoma (NHL). In some examples, the NHL includes aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma with MYC and BCL2 and/or BCL6 rearrangements with DLBCL histology (double/triple hit).

In some embodiments, the subject is or has been identified as having an Eastern Cooperative Oncology Group Performance Status (ECOG) status of less than or equal to 1. In some embodiments, the one or more unit doses of the kinase inhibitor is formulated for oral administration and/or the instructions further specify the one or more unit doses of the kinase inhibitor is administered orally.

In some embodiments, the CD19 is a human CD19. In some embodiments, the chimeric antigen receptor (CAR) includes an extracellular antigen-recognition domain that specifically binds to the CD19 and an intracellular signaling domain including an ITAM. In some examples, the intracellular signaling domain includes a signaling domain of a CD3-zeta (CD3) chain, optionally a human CD3-zeta chain. In some cases, the chimeric antigen receptor (CAR) further includes a costimulatory signaling region. In some examples, the costimulatory signaling region includes a signaling domain of CD28 or 4-1BB, optionally human CD28 or human 4-1BB. In some instances, the costimulatory domain is or includes a signaling domain of human 4-1BB.

In some embodiments, the CAR includes an scFv specific for the CD19; a transmembrane domain; a cytoplasmic signaling domain derived from a costimulatory molecule, which optionally is or includes a 4-1BB, optionally human 4-1BB; and a cytoplasmic signaling domain derived from a primary signaling ITAM-containing molecule, which optionally is or includes a CD3zeta signaling domain, optionally a human CD3zeta signaling domain; and optionally wherein the CAR further includes a spacer between the transmembrane domain and the scFv; the CAR includes, in order, an scFv specific for the CD19; a transmembrane domain; a cytoplasmic signaling domain derived from a costimulatory molecule, which optionally is or includes a 4-1BB signaling domain, optionally a human 4-1BB signaling domain; and a cytoplasmic signaling domain derived from a primary signaling ITAM-containing molecule, which optionally is a CD3zeta signaling domain, optionally human CD3zeta signaling domain; or the CAR includes, in order, an scFv specific for the CD19; a spacer; a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or includes a CD3zeta signaling domain.

In some embodiments, the CAR includes a spacer and the spacer is a polypeptide spacer that (a) includes or consists of all or a portion of an immunoglobulin hinge or a modified version thereof or includes about 15 amino acids or less, and does not include a CD28 extracellular region or a CD8 extracellular region, (b) includes or consists of all or a portion of an immunoglobulin hinge, optionally an IgG4 hinge, or a modified version thereof and/or includes about 15 amino acids or less, and does not include a CD28 extracellular region or a CD8 extracellular region, or (c) is at or about 12 amino acids in length and/or includes or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) includes or consists of the formula X₁PPX₂P (SEQ ID NO:58), where X₁ is glycine, cysteine or arginine and X₂ is cysteine or threonine; and/or the costimulatory domain includes SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain includes SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or the scFv includes a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv includes a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv includes, in order, a V_{H}, a linker, optionally including SEQ ID NO: 41, and a V_{L}, and/or the scFv includes a flexible linker and/or includes the amino acid sequence set forth as SEQ ID NO: 42.

In some embodiments, the dose of genetically engineered T cells contains from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive. In some embodiments, the dose of genetically engineered T cells contains at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells. In some cases, the dose of genetically engineered T cells contains at or about 5×10⁷ total CAR-expressing T cells. In some cases, the dose of genetically engineered T cells contains at or about 1×10⁸ CAR-expressing cells.

In some embodiments, the dose of genetically engineered T cells includes CD4+ T cells expressing the CAR and CD8+ T cells expressing the CAR and the instructions specify administration of the dose includes administering a plurality of separate compositions, said plurality of separate compositions including a first composition containing one of the CD4+ T cells and the CD8+ T cells and the second composition containing the other of the CD4+ T cells or the CD8+ T cells.

In some examples, the instructions specify the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or the initiation of administration of the first composition and the initiation of administration of the second composition are carried out between about 1 minute and about 1 hour apart or between about 5 minutes and about 30 minutes apart. In some cases, the instructions specify the first composition and second composition are administered no more than 2 hours, no more than 1 hour, no more than 30 minutes, no more than 15 minutes, no more than 10 minutes or no more than 5 minutes apart.

In some embodiments, the instructions specify the first composition contains the CD4+ T cells. In some embodiments, the instructions specify the first composition contains the CD8+ T cells. In some embodiments, the instructions specify the first composition is administered prior to the second composition. In some embodiments, the instructions specify the dose of cells is administered parenterally, optionally intravenously.

In some embodiments, the T cells are primary T cells obtained from the sample from the subject. In some cases, the T cells are autologous to the subject. In some embodiments, the instructions further specify the process for producing the T cell therapy. In some embodiments, the process for producing the T cell therapy includes isolating T cells, optionally CD4+ and/or CD8+ T cells, from the sample obtained from the subject, thereby producing an input composition containing primary T cells; and introducing the nucleic acid molecule encoding the CAR into the input composition.

In some cases, the isolating including carrying out immunoaffinity-based selection. In some examples, the biological sample is or contains a whole blood sample, a buffy coat sample, a peripheral blood mononuclear cells (PBMC) sample, an unfractionated T cell sample, a lymphocyte sample, a white blood cell sample, an apheresis product, or a leukapheresis product.

In some embodiments, prior to the introducing, the process includes incubating the input composition under stimulating conditions, said stimulating conditions including the presence of a stimulatory reagent capable of activating one or more intracellular signaling domains of one or more components of a TCR complex and/or one or more intracellular signaling domains of one or more costimulatory molecules, thereby generating a stimulated composition, wherein the nucleic acid molecule encoding the CAR is introduced into the stimulated composition. In some embodiments, the stimulatory reagent includes a primary agent that specifically binds to a member of a TCR complex, optionally that specifically binds to CD3. In some examples, the stimulatory reagent further includes a secondary agent that specifically binds to a T cell costimulatory molecule, optionally wherein the costimulatory molecule is selected from CD28, CD137 (4-1-BB), OX40, or ICOS. In some cases, the primary and/or secondary agents include an antibody, optionally wherein the stimulatory reagent includes incubation with an anti-CD3 antibody and an anti-CD28 antibody, or an antigen-binding fragment thereof.

In some embodiments, the primary agent and/or secondary agent are present on the surface of a solid support. In some examples, the solid support is or includes a bead, optionally wherein the bead is magnetic or superparamagnetic. In some cases, the bead includes a diameter of greater than or greater than about 3.5 μm but no more than about 9 μm or no more than about 8 μm or no more than about 7 μm or no more than about 6 μm or no more than about 5 μm. In some examples, the bead includes a diameter of or about 4.5 μm.

In some embodiments, the introducing includes transducing cells of the stimulated composition with a viral vector including a polynucleotide encoding the recombinant receptor. In some cases, the viral vector is a retroviral vector. In some instances, the viral vector is a lentiviral vector or gammaretroviral vector.

In some embodiments, the process further includes after the introducing cultivating the T cells, optionally wherein the cultivating is carried out under conditions to result in the proliferation or expansion of cells to produce an output composition containing the T cell therapy. In some examples, subsequent to the cultivating, the process further includes formulating cells of the output composition for cryopreservation and/or for administration of the T cell therapy to the subject, optionally wherein the formulating is in the presence of a pharmaceutically acceptable excipient.

In some embodiments, the instructions specify the subject is a human.

In some embodiments, the instructions specify, at or prior to the administration of the T cell therapy including the dose of cells, the subject is or has been identified as having a double/triple hit lymphoma; the subject is or has been identified as having a chemorefractory lymphoma, optionally a chemorefractory DLBCL; and/or the subject has not achieved a complete response (CR) in response to a prior therapy.

Provided herein is an article of manufacture containing any of the kits provided herein.

### BRIEF DESCRIPTION OF THE DRAWINGS

FIG. 1A shows graphs of normalized target cell numbers assessing target-specific cytolytic activity in triplicate wells co-cultured with CAR T cells with ibrutinib (mean±SEM). FIG. 1B shows a representative image of target cells (NucLight Red K562.CD19 cells) co-cultured with CAR T cells at an effector to target ratio (E:T) of 2.5:1 at the start and end of the cytotoxic assay. FIG. 1C shows dose effects of ibrutinib on the cytolytic activity of anti-CD19 CAR T cells. The graphs show data from three independent donors and are normalized to untreated control (100%). The mean±SEM are depicted and statistically significant differences are indicated as P<0.00001 (****).

FIG. 2A shows CAR T cell expression of CD25, CD28, CD39 and CD95 following culture of CD4+ and CD8+ cells in the presence or absence of indicated concentrations of ibrutinib. FIG. 2B shows representative results of CAR T cell from one donor-derived cells for the percentage of T_{CM} (CCR7⁺CD45RA⁻) and T_{EM} (CCR7⁻CD45RA⁻) over four days after initial stimulation in the presence of ibrutinib. FIG. 2C and FIG. 2D show CAR-T cell expression of CD69, CD107a and PD-1 following culture of CD4+ and CD8+ T cells, respectively, in the presence or absence of indicated concentrations of ibrutinib.

FIG. 3A depicts representative plots of kinetics of cytokine production over 4 days from CAR-T cells generated from one donor in the presence or absence of ibrutinib. FIG. 3B depicts percentage change in cytokine production after stimulation of CAR-T cells for 2 days in the presence of ibrutinib compared to its absence in 2 independent experiments.

FIG. 4A shows the fold change in CAR-T cell numbers after each round of restimulation in a serial stimulation assay in the absence of ibrutinib (control) or in the presence of 50 nM or 500 nM) ibrutinib. FIG. 4B shows the number of doublings of CAR-T cell numbers after each round of restimulation in the absence of ibrutinib (control) or in the presence of 50 nM or 500 nM ibrutinib in a serial stimulation assay. FIG. 4C shows the number of cells at day 4 and 18 after 1 and 5 rounds of restimulation, respectively, in the presence or absence of ibrutinib in a serial stimulation assay.

FIG. 5A shows a representative flow cytometry plot for expression of TH1 surface markers after stimulation of T cells in the presence of ibrutinib. FIG. 5B shows the percentage of TH1 cells observed over time, as measured by the flow cytometry assay, for T cells cultured in the presence or absence of ibrutinib. FIG. 5C shows the percentage of TH1 cells in T cell cultures stimulated in the presence of various concentrations of ibrutinib. FIG. 5D show expression of CD25, CD38, CD39 and CD45RO at days 0, 11, 18 and 21 of serial stimulation in the presence of ibrutinib. Representative results from CAR T cells from one donor-derived cells are shown. FIG. 5E shows expression of CD62L, CD69, CD107a and PD-1 at days 0, 11, 18 and 21 of serial stimulation in the presence of ibrutinib. Representative results from CAR T cells from one donor-derived cells are shown.

FIG. 6A shows the effect of ibrutinib treatment on tumor burden compared to vehicle treatment in a disseminated tumor xenograft mouse model identified to be resistant to BTK inhibition. FIG. 6B shows results of the same study at greater time points after post-tumor injection in mice that were treated with CAR+ T cells from two different donor-derived cells in the presence or absence of ibrutinib or vehicle control. The results in FIG. 6A and FIG. 6B depict tumor growth over time as indicated by measuring average radiance by bioluminescence. FIG. 6C shows a Kaplan meier curve depicting survival of tumor-bearing mice administered CAR-T cells in the presence or absence of iburtinib. FIG. 6D shows results of survival in the same study at greater time points after post-tumor injection in mice that were treated with CAR+ T cells from two different donor-derived cells in the presence or absence of ibrutinib or vehicle control.

FIG. 7A shows a Kaplan meier curve depicting observed survival of tumor-bearing mice administered CAR-T cells generated from two different donors, alone or in combination with administration of daily ibrutinib administered via drinking water. Statistically significant differences are shown as P<0.001 (***). FIG. 7B shows tumor growth over time as indicated by measuring average radiance by bioluminescence from mice administered CAR-T cells generated from two different donors and treated with ibrutinib administered via drinking water. Statistically significant differences are shown as two-way ANOVA P<0.05 (*), P<0.01 (**). FIG. 7C shows the level of CAR-T cells in the blood, bone marrow, and spleen of mice treated with or without ibrutinib. FIG. 7D shows the level of CAR-T cells in the blood at day 19 post CAR-T cell transfer after treatment with or without ibrutinb. Statistically significant differences are indicated as * p<0.05. FIG. 7E shows the tumor cell count in the blood, bone marrow, and spleen of mice treated with or without ibrutinib. Statistically significant differences are indicated as P<0.001 (***) and P<0.0001 (****).

FIG. 8A depicts T-distributed stochastic neighbor embedding (t-SNE) high dimensional analysis of surface markers on CAR-engineered T cells harvested from the bone marrow of animals at day 12 post-transfer with CAR-T cells and in combination with ibrutinib or control. FIG. 8B depicts four populations derived from T-distributed stochastic neighbor embedding (t-SNE) high dimensional analysis of CAR-engineered T cells harvested from the bone marrow of animals at day 12 post-transfer with CAR-T cells and ibrutinib or vehicle control. FIG. 8C depicts histograms showing the individual expression profiles of CD4, CD8, CD62L, CD45RA, CD44 and CXCR3 from the 4 gated t-SNE overlaid on the expression of the total population (shaded histogram). FIG. 8D depicts the percentage and fold change of each t-SNE population from control mice or mice treated with ibrutinib.

FIG. 9A shows the number of population doublings in a serial stimulation assay over a 21 day culture period of CAR-engineered cells, generated from cells obtained from subjects with diffuse large B-cell lymphoma (DLBCL), in the absence of ibrutinib (control) or in the presence of 50 nM or 500 nM ibrutinib. Arrows indicate the time point of each re-stimulation where CAR T cells were counted and new target cells along with ibrutinib was added. FIG. 9B shows the cytolytic activity of the genetically engineered CAR-T cells for CD19-expressing target cells after 16 days of serial restimulation in the presence or absence of ibrutinib. Percent killing was normalized to untreated control (100%). Data shown as mean±SEM from replicate wells. Statistically significant differences are indicated as P<0.001 (***), P<0.0001 (****).

FIG. 10A is a Volcano plot depicting differentially expressed genes from day 18 serially stimulated CAR T cells treated with 500 nM ibrutinib compared with control. Significantly differentially upregulated genes are on the right side of right dashed line and significantly differentially downregulated genes are on left side of left dashed line (FDR<0.05, abs log 2FC>0.5). FIG. 10B is a heat map depicting normalized expression (mean Transcripts per Million per donor+condition, z-score normalized per gene) of the 23 differentially expressed genes from FIG. 10A in the control and 500 nM ibrutinib groups. FIG. 10C depicts a Volcano plot of expressed genes from day 18 serially stimulated CAR T cells treated with 50 nM ibrutinib compared with control. FIG. 10D depicts a heat map of normalized gene expression changes (normalized as described in FIG. 10B) from day 18 serially stimulated CAR T cells in the control and 50 nM ibrutinib treated groups.

FIG. 11A-11E depict the expression (TPM, transcripts per million) box plot profiles of indicated genes summarized across donors and experiments per condition from serially stimulated CAR T cells treated with 50 nM (Ibr50) or 500 nM ibrutinib (Ibr500) compared with control (Ctrl).

FIG. 12A is a representative histogram of CD62L expression in CAR T cells from one donor-derived cells after 18 days of serial stimulation, as measured by flow cytometry. FIG. 12B depicts the fold change in the percentage of CD62L+ CAR T cells from one donor-derived cells after 18 days of serial stimulation normalized to control, as measured by flow cytometry. The data are from two independent experiments (mean±SEM).

### DETAILED DESCRIPTION

Provided are methods and uses of engineered cells, such as T cells (e.g., CAR-expressing T cells) and an inhibitor of a TEC family of kinases, such as a BTK or ITK inhibitor. In some aspects, the provided embodiments involve a combination therapy, e.g., a combination therapy involving administration of an inhibitor of a TEC family of kinases, such as a BTK inhibitor, e.g. ibrutinib, and administration of the adoptive cell therapy, such as a T cell therapy (e.g. CAR-expressing T cells), to a subject, e.g., for the treatment of subjects with a cancer or proliferative disease.

In some embodiments, provided are methods and uses of engineered cells, such as T cells (e.g., CAR-T cells) and a kinase inhibitor that is ibrutinib, which is a compound having the structure

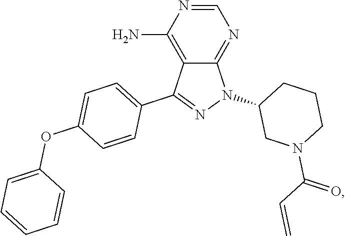 [SMILES: C=CC(=O)N1CCC[C@@H](n2nc(-c3ccc(Oc4ccccc4)cc3)c3c(N)ncnc32)C1]

or is a pharmaceutically acceptable salt, solvate, hydrate, stereoisomer, tautomer or racemic mixtures thereof, and compositions thereof, for the treatment of subjects with a cancer or proliferative disease. In some aspects, the T cell therapy is an adoptive T cell therapy comprising T cells that specifically recognize and/or target an antigen associated with the cancer or proliferative disease, such as an antigen associated with a B cell malignancy, e.g. Non Hodgkin Lymphoma (NHL) or a subtype thereof. In some aspects, the T cell therapy comprises T cells engineered with a chimeric antigen receptor (CAR) comprising an antigen binding domain that binds, such as specifically binds, to the antigen. In some cases, the antigen targeted by the T cell therapy is CD19. Also provided are combinations and articles of manufacture, such as kits, that contain a composition comprising the T cell therapy and/or a composition comprising a kinase inhibitor, such as a BTK/ITK inhibitor, e.g., ibrutinib, and uses of such compositions and combinations to treat or prevent diseases, conditions, and disorders, including cancers, such as a B cell malignancy.

Cell therapies, such as T cell-based therapies, for example, adoptive T cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of diseases and disorders such as a B cell malignancy. The engineered expression of recombinant receptors, such as chimeric antigen receptors (CARs), on the surface of T cells enables the redirection of T cell specificity. In clinical studies, CAR-T cells, for example anti-CD19 CAR-T cells, have produced durable, complete responses in both leukemia and lymphoma patients (Porter et al. (2015) Sci Transl Med., 7:303ra139; Kochenderfer (2015) J. Clin. Oncol., 33: 540-9; Lee et al. (2015) Lancet, 385:517-28; Maude et al. (2014) N Engl J Med, 371:1507-17).

In certain contexts, available approaches to adoptive cell therapy may not always be entirely satisfactory. For example, although CAR T cell persistence can be detected in many subjects with lymphoma, fewer complete responses (CRs) have been observed in subjects with NHL compared to subjects with ALL. More specifically, while higher overall response rates of up to 80% (CR rate 47% to 60%) have been reported after CAR T cell infusion, responses in some are transient and subjects have been shown to relapse in the presence of persistent CAR T cells (Neelapu, 58th Annual Meeting of the American Society of Hematology (ASH): 2016; San Diego, Calif., USA. Abstract No. LBA-6.2016; Abramson, Blood. 2016 Dec. 1; 128(22):4192). Another study reported a long term CR rate of 40% (Schuster, Ann Hematol. 2016 October; 95(11):1805-10).

In some aspects, an explanation for this is the immunological exhaustion of circulating CAR-expressing T cells and/or changes in T lymphocyte populations. This is because, in some contexts, optimal efficacy can depend on the ability of the administered cells to have the capability to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some cases, responses can be improved by administration or preconditioning with a lymphodepleting therapy, which in some aspects increases the persistence and/or efficacy of the cells following administration, as compared to methods in which the preconditioning is not carried out or is carried out using a different lymphodepleting therapy. The lymphodepleting therapy generally includes the administration of fludarabine, typically in combination with another chemotherapy or other agent, such as cyclophosphamide, which may be administered sequentially or simultaneously in either order. In a recent phase I/II clinical study, complete response (CR) in acute lymphoblastic leukemia (ALL), non-Hodgkin's lymphoma (NHL) and chronic lymphocytic leukemia (CLL) patients was 94%, 47% and 50% respectively, and disease free survival rates were greater in patients that received cyclophosphamide and fludarabine lymphodepletion compared to those who received cyclophosphamide but not fludarabine (Cameron et al. (2016) J Clin Oncol, 34 (suppl; abstr 102). In some aspects, however, even with lymphodepleting therapies, CAR-T cell therapies are not always consistently effective in all subjects.

In some embodiments, the exposure, persistence and functions of engineered cells is reduced or declines after administration to the subject. Yet, observations indicate that, in some cases, the administered cells expressing the recombinant receptors (e.g., increased number of cells or duration over time) can re-expand and/or be re-activated in vivo to improve efficacy and therapeutic outcomes in adoptive cell therapy.

The provided methods are based on observations that a kinase inhibitor, such as a BTK/ITK inhibitor, e.g., ibrutinib, improves T cell function of an engineered T cell therapy, including functions related to the expansion, proliferation and persistence of T cells. In some embodiments, the methods are advantageous by virtue of administering T cell therapy, such as a composition including cells for adoptive cell therapy, e.g., such as a T cell therapy (e.g. CAR-expressing T cells) in combination with a kinase inhibitor, e.g., ibrutinib. In some aspects, the provided methods and uses provide for or achieve improved or more durable responses or efficacy as compared to certain alternative methods. In some aspects, the provided methods enhance or modulate proliferation and/or activity of T cell activity associated with administration of the T cell therapy (e.g. CAR-expressing T cells).

In some aspects, the provided methods and uses provide for or achieve improved or more durable responses or efficacy as compared to certain alternative methods, such as in particular groups of subjects treated. In some embodiments, the methods are advantageous by virtue of administering an immunotherapy or immunotherapeutic agent, such as a composition including cells for adoptive cell therapy, e.g., such as a T cell therapy (e.g. CAR-expressing T cells), and an inhibitor of a TEC family kinase, e.g. BTK inhibitor or ITK inhibitor, e.g. ibrutinib.

The provided methods are based on observations that an inhibitor of a TEC family kinase, e.g. ibrutinib, improves T cell function, including functions related to the expansion, proliferation and persistence of T cells. Ibrutinib is an irreversible small molecule inhibitor (SMI) that block the activity of Bruton's tyrosine kinase (BTK) and also exhibits activity on ITK. Ibrutinib is approved for use in mantle cell lymphoma (MCL) and Waldenström's Macroglobulinemia in the relapsed refractory setting (Davids et al. (2014) Future Oncol., 10:957-67). In some cases, aberrant activation of the B-cell receptor (BCR) signaling pathway is the main mechanism underlying B cell malignancies such as MCL and CLL, whereby chronic BTK signaling can initiate a phosphorylation cascade through nuclear factor kappa-light-chain-enhancer of activated B cells (NFκB) and mitogen-activated protein kinases (MAP kinases) promoting B cell survival and aberrant activation. Thus, existing methods of employing TEC family kinase inhibitors, such as BTK/ITK inhibitors, e.g. ibrutinib, are used for treating B cell malignancies.

The provided findings indicate that combination therapy of the inhibitor in methods involving T cells, such as involving administration of adoptive T cell therapy, achieves improved function of the T cell therapy. In some embodiments, combination of the cell therapy (e.g., administration of engineered T cells) with the kinase inhibitor, e.g., BTK inhibitor and/or ITK inhibitor (such as a selective and/or irreversible inhibitor of such kinase), improves or enhances one or more functions and/or effects of the T cell therapy, such as persistence, expansion, cytotoxicity, and/or therapeutic outcomes, e.g., ability to kill or reduce the burden of tumor or other disease or target cell. In some embodiments, observations herein indicate that a TEC family kinase inhibitor, such as a BTK inhibitor and/or ITK inhibitor, e.g. ibrutinib, may dampen CAR T activation at higher concentrations while increasing activation at lower concentrations.

In some aspects, such effects are observed despite that the tumor or disease or target cell itself is insensitive, resistant and/or otherwise not sufficiently responsive to the inhibitor, to inhibitors targeting the kinase to which the inhibitor is selective, and/or is resistant to inhibition of a TEC family kinase, optionally is resistant to inhibition of the TEC family kinase by the inhibitor, and/or is resistant to inhibition of another TEC family kinase and/or is resistant to another inhibitor of a TEC family kinase, optionally a different TEC family kinase as compared to one or more targeted by (or that is the main target of) the inhibitor. For example, in some embodiments, the cancer is insensitive to or has become resistant to the inhibitor, or to inhibition of the TEC family kinase by the inhibitor and/or by another inhibitor.

In some embodiments, the provided methods, uses and combination therapies include administration of the kinase inhibitor, in combination with a T cell therapy, such as CAR+ T cells, in a subject that has already been administered the inhibitor or another kinase inhibitor, in a context in which such subject has been deemed refractory or resistant to the inhibitor, and/or not sufficiently responsive, to treatment with the previous administration of such inhibitor. In some embodiments, the combination therapy, methods and uses include continued administration of the kinase inhibitor, e.g., ibrutinib, in combination with a T cell therapy (e.g. CAR+ T cells) in a subject that has previously received administration of the kinase inhibitor, e.g., ibrutinib, but in the absence of (or not in combination with) a T cell therapy and/or in the absence of an engineered T cell therapy, and/or in the absence of an engineered T cell therapy directed to the same disease or target as that targeted by the provided therapy, method or use.

In some embodiments, the methods and combinations result in improvements in T cell function or phenotype, e.g., in intrinsic T cell functionality and/or intrinsic T cell phenotype, of T cells of the T cell therapy. Such improvements in some aspects result without compromising, or without substantially compromising, one or more other desired properties of functionality, e.g., of CAR-T cell functionality. In some embodiments, the combination with the inhibitor, while improving one or more outcomes or functional attributes of the T cells, does not reduce the ability of the cells to become activated, secrete one or more desired cytokines, expand and/or persist, e.g., as measured in an in vitro assay as compared to such cells cultured under conditions otherwise the same but in the absence of the inhibitor.

In some embodiments, the provided embodiments involve initiating the administration of a kinase inhibitor, such as a BTK/ITK inhibitor, e.g., ibrutinib, prior to administration of the T cell therapy and continue until the initiation of administration of the T cell therapy or after the initiation of administration of the T cell therapy. In some aspects, the provided embodiments involve extended treatment with a kinase inhibitor, such as a BTK/ITK inhibitor, e.g., ibrutinib, such as an extended pretreatment with the kinase inhibitor, e.g., ibrutinib. In some aspects, the provided embodiments, e.g., involving extended treatment with a kinase inhibitor, e.g., ibrutinib, can help restore T-cell function, reduce tumor burden, disrupt the tumor microenvironment, reduce the generation of myeloid-derived suppressor cells (MDSCs), thereby alleviating or overcoming the tumor microenvironment (TME)-specific immunosuppression. In some aspects, BTK or phospholipase C-γ2 (PLCγ2) mutations were not observed to detrimentally affect the efficacy of certain cell therapies.

In some aspects, the provided embodiments involve continued, resumed and/or further administration of a kinase inhibitor, such as a BTK/ITK inhibitor, e.g., ibrutinib, after the initiation of administration of the T cell therapy. In some aspects, the provided embodiments, e.g., involving continued, resumed and/or further administration after the initiation of administration of the cell therapy, can reduce the potential for exhaustion of the administered cells, reduce risk of toxicities such as cytokine release syndrome (CRS) or neurotoxicity (NT), reduce the risk of resistant mutations by orthogonal dual-targeting, and suppress the tumor microenvironment (e.g., counteracting immunosuppressive activities in the tumor microenvironment). In some aspects, advantages of the provided embodiments also include the ability to modulate the dosing or administration of the kinase inhibitor, e.g., ibrutinib, or removing or discontinuing the administration of the kinase inhibitor, e.g., ibrutinib, depending on the tolerability in the subject.

In some aspects, the kinase inhibitor, e.g., ibrutinib, can enhance intrinsic functions of the administered cells to result in improved performance of the cells. In some embodiments, the effects of the kinase inhibitors are modulated by off-target covalent and non-covalent inhibition. In some aspects, inhibition of ITK can result in Th1 biased polarization. In some aspects, administration of a kinase inhibitor, e.g., ibrutinib, can restore T cell functionality in subjects with CLL. In some embodiments, the lymphocytosis effects may disrupt the TME and can contribute to improved access to the tumor by the administered cells. In some aspects, toxicities such as cytokine release syndrome (CRS) can be reduced by limiting acute myeloid reactivity. In some aspects, administration of a kinase inhibitor, e.g., ibrutinib, in combination can result in enhanced proliferation, survival and/or expansion of administered engineered cells, and result in enhanced anti-tumor activity. In some embodiments, such improvements can be observed in cells from subjects that may not exhibit optimal activity. In some embodiments, treatment with a kinase inhibitor, e.g., ibrutinib, were observed to increase cells that express markers associated with memory-like subpopulations of the engineered cells after serial stimulation, and gene expression profiles were observed to be modified. Further, increased in vivo efficacy of administered CAR+ T cells were observed in combination therapy with a kinase inhibitor, e.g., ibrutinib. Due to ibrutinib's off-target activity to inhibit ITK, ibrutinib treatment in some cases had been thought to be consequential to T cell activity. In some aspects, the observation of improved activity and/or effector function of administered T cells in combination with ibrutinib treatment provides an unexpected advantage for improving T cell therapy. In some aspects, administration of a kinase inhibitor, such as a BTK/ITK inhibitor (e.g., ibrutinib) can restore T cell functionality, improve effector function of administered T cell therapy, limit tumor environment-mediated immune dysfunction, and result in reduced tumor burden, improved tumor clearance and prolonged survival of subjects treated with the combination.

In some embodiments, the provided methods can potentiate CAR-T cell therapy, which, in some aspects, can improve outcomes for treatment of subjects that have a cancer that is resistant or refractory to other therapies, is an aggressive or high-risk cancer, and/or that is or is likely to exhibit a relatively lower response rate to a CAR-T cell therapy administered without the inhibitor compared to another type of cancer.

In some embodiments of the provided methods, one or more properties of administered genetically engineered cells can be improved or increased or greater compared to administered cells of a reference composition, such as increased or longer expansion and/or persistence of such administered cells in the subject or an increased or greater recall response upon restimulation with antigen. In some embodiments, the increase can be at least a 1.2-fold, at least 1.5-fold, at least 2-fold, at last 3-fold, at least 4-fold, at least 5-fold, at least 6-fold, at least 7-fold, at least 8-fold, at least 9-fold, or at least 10-fold increase in such property or feature compared to the same property or feature upon administration of cell therapies using other methods, e.g., not having been incubated or administered in the presence of a kinase inhibitor, e.g., ibrutinib. In some embodiments, the increase in one or more of such properties or features can be observed or is present within one months, two months, three months, four months, five months, six months, or 12 months after administration of the genetically engineered cells.

The provided methods include administering a kinase inhibitor, such as a BTK/ITK inhibitor, e.g., ibrutinib, in an effective amount to exhibit a T cell modulatory effect. Particular dosages and/or dosing regimen of a kinase inhibitor, e.g., ibrutinib, can increase or enhance T cell function of a T cell therapy, e.g. CAR-T cell therapy. In some aspects, initiation of administration of the kinase inhibitor, e.g., ibrutinib, can be prior to the administration of the T cell therapy, e.g. CAR-T cell therapy. In some aspects, initiation of administration of the kinase inhibitor, e.g., ibrutinib, can be prior to obtaining cells from the subject for genetic engineering. In some aspects, administration of the kinase inhibitor, e.g., ibrutinib, is continued based on particular regimen, for a certain period of time. In some aspects, administration of the kinase inhibitor, e.g., ibrutinib, is continued until after the initiation of the T cell therapy, e.g. CAR-T cell therapy, such as for a certain period of time after the initiation of the T cell therapy. In some aspects, a kinase inhibitor, e.g., ibrutinib, is administered long-term. In some aspects, long-term administration of the kinase inhibitor, e.g., ibrutinib, including over several cycles of administration, can result in improved proliferation, survival, and/or activation of the administered T cells. In some aspects, administering a kinase inhibitor, e.g., ibrutinib, according to the provided methods could increase the activity of CAR-expressing cells for treating a cancer, e.g. B cell malignancy such as NHL, e.g. DLBCL, by restoring T cell function and activity of the engineered T cells, and, in some aspects, may also exhibit its cell autonomous antineoplastic effects. In some aspects, CAR+ T cells generated from DLBCL subjects in demonstrated increased cytolytic function in the presence of a kinase inhibitor, e.g., ibrutinib, after serial stimulation. In some aspects, anti-tumor activity of administered CAR+ T cells against mantle cell lymphoma (MCL) was observed to be improved and reduction of cytokine release syndrome (CRS) was observed, in certain contexts.

In some embodiments, the provided methods include administering an effective amount of a kinase inhibitor, e.g., ibrutinib, per day to a subject to modulate activity and/or function of the T cell therapy. In some embodiments, the effective amount is between at or approximately 140 mg/day and at or approximately 560 mg/day. In some embodiments, the amount of a kinase inhibitor, e.g., ibrutinib, is administered daily. The administration of a kinase inhibitor, e.g., ibrutinib, is carried out for a period of time, such as generally for more than one week, such as for at or greater than one month, at or greater than two months, at or greater than three months, at or greater than four months, at or greater than five months, at or greater than six months, at or greater than seven months or at or greater than eight months. Exemplary dosing regimens are described herein.

In some embodiments, the administration of a kinase inhibitor, e.g., ibrutinib, is initiated at a time before the initiation of administration of engineered T cells. In some embodiments, the administration of a kinase inhibitor, e.g., ibrutinib, is initiated before T cells to be engineered are obtained from the subject, e.g., before apheresis or leukapheresis of the subjects. In some aspects, initiation of administration of kinase inhibitor, e.g., ibrutinib, is at least 7, 6, 5, 4, 3, 2 or 1 day prior to apheresis or leukapheresis. In some aspects, the administration of the kinase inhibitor, e.g., ibrutinib, is continued until after administration of the engineered T cells. In some embodiments, administration of a kinase inhibitor, e.g., ibrutinib, is continued if the subject does not exhibit a severe toxicity following the administration of the cell therapy.

In some embodiments, the provided methods do not result in a high rate or likelihood of toxicity or toxic outcomes, or reduces the rate or likelihood of toxicity or toxic outcomes, such as neurotoxicity (NT), cytokine release syndrome (CRS), or hematological toxicities, such as neutropenia, such as compared to certain other cell therapies or immunomodulatory drug regimens.

In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxcity. In some embodiments, no more than 50% of subjects treated (e.g. at least 60%, at least 70%, at least 80%, at least 90% or more of the subjects treated) exhibit a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

In some cases, a kinase inhibitor, such as a BTK/ITK inhibitor, e.g., ibrutinib, administered at a time that it can efficiently/effectively boost or prime the cells. In some embodiments, the provided methods can potentiate T cell therapy, e.g. CAR-T cell therapy, which, in some aspects, can improve outcomes for treatment. In some embodiments, the methods are particularly advantageous in subjects in which the cells of the T cell therapy exhibit weak expansion, have become exhausted, exhibit a reduced or decreased persistence in the subject and/or in subjects that have a cancer that is resistant or refractory to other therapies, and/or is an aggressive or high-risk cancer.

In some embodiments, a subject having received administration of a T cell therapy, e.g. CAR-T cell, is monitored for the presence, absence or level of T cells of the therapy in the subject, such as in a biological sample of the subject, e.g. in the blood of the subject. In some embodiments, the provided methods result in genetically engineered cell with increased persistence and/or better potency in a subject to which it is administered. In some embodiments, the persistence of genetically engineered cells, such as CAR-expressing T cells, in the subject is greater as compared to that which would be achieved by alternative methods, such as those involving administration of a the T cell therapy but in the absence of administration of a kinase inhibitor, such as a BTK/ITK inhibitor, e.g., ibrutinib. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR-expressing cells) in the blood or serum or organ or tissue (e.g., disease site) of the subject. In some aspects, persistence is quantified as copies of DNA or plasmid encoding the receptor, e.g., CAR, per microgram of DNA, e.g., total DNA obtained from a sample, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

In some embodiments, a kinase inhibitor, such as a BTK/ITK inhibitor, e.g., ibrutinib, is administered for a period of time to enhance, increase or optimize durability of response. In some aspects, the provided methods are based on observations that subjects who achieve or are in complete remission or complete response (CR) at 3 months, such as generally at 6 months after initiation of administration of the T cell therapy, are more likely to sustain the response longer term, such as survive or survive without progression for greater than or greater than about three months, four months, five months, six months, seven months, eight months, nine months, ten months, eleven months or twelve months after ending the treatment or after first achieving a complete response (CR) following administration of the combination therapy. In some aspects, the methods are carried out to administer a kinase inhibitor, e.g., ibrutinib, such as in a particular cycling regimen as described, for a period of time that is at least 3 months, such as at least four months, at least five months or at least six months after initiation of administration of the T cell therapy. In some embodiments, a kinase inhibitor, e.g., ibrutinib, is administered, such as in a particular cycling regimen as described, for at least six months or at least 180 days after initiation of administration of the T cell therapy. In some embodiments, at the end of the period, administration of a kinase inhibitor, e.g., ibrutinib, is ended or stopped if the subject exhibits a CR or if the disease or condition has progressed or relapsed in the subject following remission after receiving the treatment (combination therapy). In some aspects, continued administration of a kinase inhibitor, e.g., ibrutinib, can be carried out in subjects who, at the end of the period of time (e.g. at or about 6 months) exhibit a partial response (PR) or stable disease (SD). In other aspects, the period of time is a fixed duration and no further administration of a kinase inhibitor, e.g., ibrutinib, is carried out.

In some aspects, the provided methods and uses provide for or achieve improved or more durable responses or efficacy as compared to certain alternative methods, e.g. methods that include administration of the T cell therapy or a kinase inhibitor, e.g., ibrutinib, as a monotherapy or without administration as a combination therapy together as described herein, such as in particular groups of subjects treated. In some embodiments, the methods are advantageous by virtue of administering T cell therapy, such as a composition including cells for adoptive cell therapy, e.g., such as a T cell therapy (e.g. CAR-expressing T cells), and a kinase inhibitor, e.g., ibrutinib. In some embodiments, such responses are observed in high risk patients with poor prognosis, such as those having high-risk disease, e.g., high-risk NHL. In some aspects, the methods treat subjects having a form of aggressive and/or poor prognosis B-cell non-Hodgkin lymphoma (NHL), such as NHL that has relapsed or is refractory (R/R) to standard therapy or has a poor prognosis. In some embodiments, subjects treated according to the provided methods have diffuse large B-cell lymphoma (DLBCL) or follicular lymphoma (FL).

In some embodiments, at least 35%, at least 40%, at least 50%, at least 55%, at least 60%, at least 65%, at least 70%, or at least 75% or more of the subjects treated according to the provided methods, and/or with the provided articles of manufacture or compositions, achieve a complete response (CR). In some embodiments, the subject is in CR and exhibits minimum residual disease (MRD). In some embodiments, the subject is in CR and is MRD−. In some embodiments, at least 50%, at least 60%, at least 70%, at least 80%, or at least 90% of the subjects treated according to the provided methods, and/or with the provided articles of manufacture or compositions, achieve an objective response of a partial response (PR). In some embodiments, at least 60%, at least 70%, at least 80%, at least 90%, at least 95% or more of the subjects treated according to the provided methods, and/or with the provided articles of manufacture or compositions, achieve a CR or PR at six months, at seven months, at eight months, at nine months, at ten months, at eleven months or a year after initiation of administration of the cell therapy.

In some embodiments, by three months, four months, five months, six months, seven months, eight months, nine months, ten months, eleven months or twelve months or more after initiation of administration of the cell therapy, at least 60%, at least 70%, at least 80%, at least 90%, at least 95% or more of the subjects treated according to the provided methods, and/or with the provided articles of manufacture or compositions, remain in response, such as remain in CR or an objective response (OR). In some embodiments, such response, such as CR or OR, is durable for at least three months, four months, five months, six months, seven months, eight months, nine months, ten months, eleven months, twelve months or more such as in at least or about at least 60%, at least 70%, at least 80%, at least 90%, at least 95% or more of the subjects treated according to the provided methods or in such subjects who achieve a CR by three months, four months, five months or six months. In some embodiments, at least 60%, at least 70%, at least 80%, at least 90%, at least 95% or more of the subjects treated according to the provided methods, and/or with the provided articles of manufacture or compositions, or such subjects who achieve a CR by three months, four months, five months or six months survive or survive without progression for greater than or greater than about six months, seven months, eight months, nine months, ten months, eleven months, twelve months or longer.

Also provided are methods for engineering, preparing, and producing the cells, compositions containing the cells and/or inhibitor, and kits and articles of manufacture for using, producing and administering the cells and/or inhibitor, such as in accord with the provided combination therapy methods.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Combination Therapy

Provided herein are methods for combination therapy for treating a disease or disorder, e.g. a cancer or proliferative disease, that includes administering to a subject a combination therapy of 1) a kinase inhibitor and 2) a cell therapy, e.g. T cell therapy (e.g. CAR-expressing T cells). Also provided are methods and uses of engineered cells, such as T cells (e.g., CAR-expressingT cells) and an inhibitor of a TEC family kinase, such as Bruton's tyrosine kinase (BTK). In some aspects, the inhibitor is an inhibitor of Bruton's tyrosine kinase (BTK) and/or IL-2 inducible T-cell kinase (ITK), such as ibrutinib. In some aspects, the inhibitor has the structure

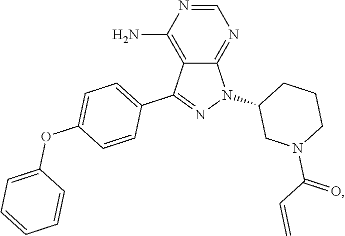 [SMILES: C=CC(=O)N1CCC[C@@H](n2nc(-c3ccc(Oc4ccccc4)cc3)c3c(N)ncnc32)C1]

or a pharmaceutically acceptable salt, solvate, hydrate, stereoisomer, tautomer or racemic mixtures thereof, including and compositions thereof, for the treatment of subjects with cancer.

The combination therapy, e.g., including engineered cells expressing a recombinant receptor, such as a chimeric antigen receptor (CAR) and a kinase inhibitor, e.g., ibrutinib, or compositions comprising the engineered cells and/or the kinase inhibitor, e.g., ibrutinib, described herein are useful in a variety of therapeutic, diagnostic and prophylactic indications. For example, the combinations are useful in treating a variety of diseases and disorders in a subject. Such methods and uses include therapeutic methods and uses, for example, involving administration of the engineered cells, kinase inhibitor, e.g., ibrutinib, and/or compositions containing one or both, to a subject having a disease, condition, or disorder, such as a tumor or cancer. In some embodiments, the engineered cells, kinase inhibitor, e.g., ibrutinib, and/or compositions containing one or both are administered in an effective amount to effect treatment of the disease or disorder. Uses include uses of the engineered cells, kinase inhibitor, e.g., ibrutinib, and/or compositions containing one or both in such methods and treatments, and in the preparation of a medicament in order to carry out such therapeutic methods. In some embodiments, the methods are carried out by administering the engineered cells, kinase inhibitor, e.g., ibrutinib, and/or compositions containing one or both, to the subject having or suspected of having the disease or condition. In some embodiments, the methods thereby treat the disease or condition or disorder in the subject.

In some embodiments, the methods are for treating a subject with a B cell malignancy. In some aspects, the methods are for treating a leukemia or a lymphoma, such as a non-Hodgkin lymphoma (NHL). In some aspects, the methods and uses provide for or achieve improved response and/or more durable responses or efficacy, e.g., in particular groups of subjects treated, as compared to certain alternative methods. In some embodiments, the cell therapy comprises administering T cells that specifically recognize and/or target an antigen associated with a disease or disorder, e.g. a cancer or proliferative disease. Also provided are combinations and articles of manufacture, such as kits, that contain a composition comprising the T cell therapy and/or a composition comprising the kinase inhibitor, such as a BTK/ITK inhibitor, e.g., ibrutinib, and uses of such compositions and combinations to treat or prevent diseases, conditions, and disorders, including cancers.

In some embodiments, the methods and uses include 1) administering to the subject a T cell therapy involving cells expressing genetically engineered cell surface receptors (e.g., recombinant antigen receptor), which generally are chimeric receptors such as chimeric antigen receptors (CARs), recognizing an antigen expressed by, associated with and/or specific to the B cell malignancy, such as a leukemia or lymphoma (e.g. NHL) and/or cell type from which it is derived, and 2) administering to the subject a kinase inhibitor, such as a BTK/ITK inhibitor, e.g., ibrutinib. The methods generally involve administering one or more doses of the cells and more than one dose of a kinase inhibitor, e.g., ibrutinib, to the subject.

In some embodiments, the provided combination therapy method involves administering to the subject a therapeutically effective amount of a kinase inhibitor, such as a BTK/ITK inhibitor, e.g., ibrutinib, and the cell therapy, such as a T cell therapy (e.g. CAR-expressing T cells). In some embodiments, the provided combination therapy methods involve initiating administration of a kinase inhibitor, such as a BTK/ITK inhibitor, e.g., ibrutinib, prior to, subsequently to, during, during the course of, simultaneously, near simultaneously, sequentially, concurrently and/or intermittently with the initiation of the cell therapy, such as a T cell therapy (e.g., CAR-expressing T cells).

In some embodiments, the provided embodiments involve initiating the administration of a kinase inhibitor, such as a BTK/ITK inhibitor, e.g., ibrutinib, prior to administration of the T cell therapy and continue until the initiation of administration of the T cell therapy or after the initiation of administration of the T cell therapy. In some aspects, the provided embodiments involve extended treatment with a kinase inhibitor, such as a BTK/ITK inhibitor, e.g., ibrutinib, such as an extended pretreatment with the kinase inhibitor, e.g., ibrutinib. In some embodiments, the method involves continuing administration of the kinase inhibitor, such as a BTK/ITK inhibitor, e.g., ibrutinib. In some embodiments, continuing administration of the kinase inhibitor, such as a BTK/ITK inhibitor, e.g., ibrutinib, involves administration of multiple doses of the kinase inhibitor, e.g., ibrutinib. In some embodiments, the kinase inhibitor, e.g., ibrutinib, is not continued or further administered after initiation of the T cell therapy. In some embodiments, the dosage schedule comprises administering the kinase inhibitor, e.g., ibrutinib, prior to and after initiation of the T cell therapy. In some embodiments, the dosage schedule comprises administering the kinase inhibitor, e.g., ibrutinib, simultaneously with the administration of the T cell therapy.

In some aspects, the methods involve administration of the kinase inhibitor, e.g., ibrutinib, that is initiated at or at least about 3 days or a minimum of at or about 3 days prior to obtaining a sample comprising T cells from the subject, e.g., for producing a T cell therapy for administration. In some aspects, the T cell therapy is produced by a process comprising obtaining a sample comprising T cells from the subject and introducing a nucleic acid molecule encoding the CAR into a composition comprising the T cells. In some embodiments, the kinase inhibitor, e.g., ibrutinib is administered in a dosing regimen comprising administration for a period of time that extends at least until the sample is obtained from the subject. In some aspects, the administration of the kinase inhibitor, e.g., ibrutinib, is initiated at or at least about 3 days prior to obtaining the sample from the subject and is carried out in a dosing regimen comprising administration for a period of time that extends at least until the sample is obtained from the subject.

In some embodiments, the methods and uses involve: (1) administering to a subject having a cancer an effective amount of a kinase inhibitor, e.g., a kinase inhibitor having the structure

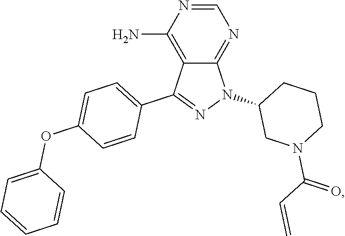 [SMILES: C=CC(=O)N1CCC[C@@H](n2nc(-c3ccc(Oc4ccccc4)cc3)c3c(N)ncnc32)C1]

or a pharmaceutically acceptable salt thereof; and (2) administering an autologous T cell therapy to the subject, said T cell therapy comprising a dose of genetically engineered T cells expressing a chimeric antigen receptor (CAR) that specifically binds to an antigen associated with a disease or disorder, e.g., a CD19. In some embodiments, prior to administering the T cell therapy, a biological sample has been obtained from the subject and processed, the processing comprising genetically modifying T cells from the sample, for example, by introducing a nucleic acid molecule encoding the CAR into said T cells. In some embodiments, the administration of the kinase inhibitor is initiated at least at or about 3 days prior to the obtaining of the sample and is carried out in a dosing regimen comprising repeat administrations of the inhibitor at a dosing interval, over a period of time that extends at least to include administration on or after the day that the sample is obtained from the subject.

In some embodiments, the methods and uses involve: (1) administering to a subject having a cancer an effective amount of a kinase inhibitor, e.g., a kinase inhibitor having the structure

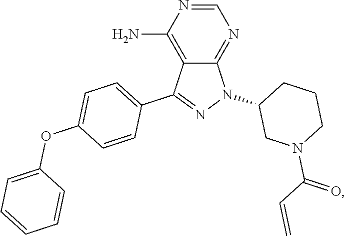 [SMILES: C=CC(=O)N1CCC[C@@H](n2nc(-c3ccc(Oc4ccccc4)cc3)c3c(N)ncnc32)C1]

or a pharmaceutically acceptable salt thereof; (2) obtaining from the subject a biological sample and processing T cells of said sample, thereby generating a composition comprising genetically engineered T cells that express a chimeric antigen receptor (CAR) that specifically binds to an antigen associated with a disease or a disorder, e.g. CD19, and (3) administering to the subject an autologous T cell therapy comprising a dose of the genetically engineered T cells. In some aspects, the administration of the kinase inhibitor is carried out in a dosing regimen that is initiated at least at or about 3 days prior to the obtaining of the sample and that comprises repeat administrations of the compound, at a dosing interval, over a period of time and extends at least to include administration of the compound on or after the day that the sample is obtained from the subject.

In some embodiments, the methods and uses involve: (1) administering to a subject having a cancer an effective amount of a kinase inhibitor, e.g., a kinase inhibitor having the structure

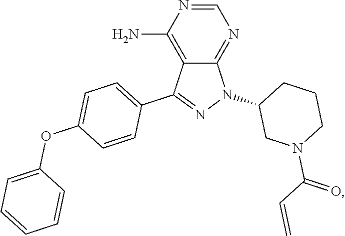 [SMILES: C=CC(=O)N1CCC[C@@H](n2nc(-c3ccc(Oc4ccccc4)cc3)c3c(N)ncnc32)C1]

or a pharmaceutically acceptable salt thereof; and (2) administering an autologous T cell therapy to the subject, said T cell therapy comprising a dose of genetically engineered T cells expressing a chimeric antigen receptor (CAR) that specifically binds to an antigen associated with a disease or disorder, e.g., a CD19. In some aspects, the T cell therapy is produced by a process comprising obtaining a sample comprising T cells from the subject and introducing a nucleic acid molecule encoding the CAR into a composition comprising the T cells, wherein the administration of the kinase inhibitor is initiated at or at least about 3 days or a minimum of at or about 3 days prior to obtaining the sample from the subject and is carried out in a dosing regimen comprising administration for a period of time that extends at least until the sample is obtained from the subject.

In some aspects, the provided methods and uses involve administering to a subject having a cancer an effective amount of a kinase inhibitor, e.g., a kinase inhibitor having the structure

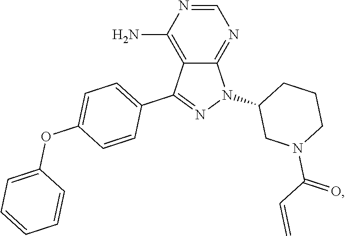 [SMILES: C=CC(=O)N1CCC[C@@H](n2nc(-c3ccc(Oc4ccccc4)cc3)c3c(N)ncnc32)C1]

or a pharmaceutically acceptable salt thereof, wherein the subject is a candidate for treatment or is to be treated with a T cell therapy to the subject. In some aspects, the T cell therapy comprising a dose of genetically engineered T cells expressing a chimeric antigen receptor (CAR) that specifically binds to an antigen associated with a disease or disorder, e.g., a CD19. In some aspects, the T cell therapy is produced by a process comprising obtaining a sample comprising T cells from the subject and introducing a nucleic acid molecule encoding the CAR into a composition comprising the T cells. In some aspects, the administration of the kinase inhibitor, e.g., ibrutinib, and is initiated at or at least about 3 days or a minimum of at or about 3 days prior to obtaining the sample from the subject and is carried out in a dosing regimen comprising administration for a period of time that extends at least until the sample is obtained from the subject. In some aspects, the methods and uses also involve administering to the subject the T cell therapy, e.g., a composition comprising T cells obtained from the subject that have been introduced with a nucleic acid molecule encoding a CAR. In some embodiments, the obtaining of a sample from the subject includes obtaining a sample that is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, the obtaining of a sample from the subject is also referred to as apheresis or leukapheresis.

In some aspects, subsequent to initiation the administration of the kinase inhibitor, e.g., ibrutinib, and prior to the administration of the T cell therapy, the subject has been preconditioned with a lymphodepleting therapy. In some aspects, the lymphodepleting therapy is or comprises any lymphodepleting therapy described herein, e.g., in Section I.C. In some aspects, the methods and uses involve administering a lymphodepleting therapy to the subject, subsequent to initiating the administration of the kinase inhibitor, e.g., ibrutinib, and prior to the administration of the T cell therapy. In some embodiments, the dosing regimen for administering the kinase inhibitor, e.g., ibrutinib, involves administration for a period of time that extends at least until the initiation of the lymphodepleting therapy.

In some embodiments of the methods and uses, the administration of the kinase inhibitor, e.g., ibrutinib, is discontinued or paused, during the lymphodepleting therapy. In some embodiments, the discontinuation can be a temporary discontinuation, e.g., a pause or temporary halting of administration. In some embodiments of the methods and uses, the administration of the kinase inhibitor, e.g., ibrutinib, can be optionally resumed after the lymphodepleting therapy. In some embodiments, the kinase inhibitor, e.g., ibrutinib, is further administered or the administration is resumed, after the lymphodepleting therapy. In some embodiments, the dosage amount, frequency, schedules or regimen of the initial administration of the kinase inhibitor, e.g., ibrutinib, prior to a lymphodepleting therapy and/or a discontinuation or a pause, is the same as the dosage amount, frequency, schedules or regimen of the further administration or resumed administration of the kinase inhibitor, e.g., ibrutinib, after lymphodepleting therapy and/or a discontinuation or a pause. In some embodiments, the dosage amount, frequency, schedules or regimen of the initial administration of the kinase inhibitor, e.g., ibrutinib, prior to a lymphodepleting therapy and/or a discontinuation or a pause, is different from or modified compared to the dosage amount, frequency, schedules or regimen of the further administration or resumed administration of the kinase inhibitor, e.g., ibrutinib, after lymphodepleting therapy and/or a discontinuation or a pause.

In some aspects, the dosing regimen for administering the kinase inhibitor, e.g., ibrutinib, involves administration of the kinase inhibitor up to the initiation of the lymphodepleting therapy, discontinuing administration of the kinase inhibitor during the lymphodepleting therapy and further administration of the kinase inhibitor for a period that extends for at least 15 days, such as at least 15, 30, 60, 90, 120, 150 or 180 days, after initiation of administration of the T cell therapy.

In some embodiments, the cell therapy is adoptive cell therapy. In some embodiments, the cell therapy is or comprises a tumor infiltrating lymphocytic (TIL) therapy, a transgenic TCR therapy or a recombinant-receptor expressing cell therapy (optionally T cell therapy), which optionally is a chimeric antigen receptor (CAR)-expressing cell therapy. In some embodiments, the therapy targets CD19 or is a B cell targeted therapy. In some embodiments, the cells and dosage regimens for administering the cells can include any as described herein.

In some embodiments, the kinase inhibitor, e.g., TEC family kinase inhibitor, inhibits one or more kinase of the TEC family, including Bruton's tyrosine kinase (BTK), IL-2 inducible T-cell kinase (ITK), tec protein tyrosine kinase (TEC), bone marrow tyrosine kinase gene in chromosome X protein (BMX) non-receptor tyrosine kinase (also known as Epithelial and endothelial tyrosine kinase; ETK), and TXK tyrosine kinase (TXK). In some embodiments, the inhibitor is a Bruton's tyrosine kinase (BTK) inhibitor. In some embodiments, the cells and dosage regimens for administering the inhibitor can include any as described herein.

In some embodiments, the immunotherapy, such as a T cell therapy (e.g. CAR-expressing T cells), and inhibitor are provided as pharmaceutical compositions for administration to the subject. In some embodiments, the pharmaceutical compositions contain therapeutically effective amounts of one or both of the agents for combination therapy, e.g., T cells for adoptive cell therapy and an inhibitor as described. In some embodiments, the agents are formulated for administration in separate pharmaceutical compositions. In some embodiments, any of the pharmaceutical compositions provided herein can be formulated in dosage forms appropriate for each route of administration.

In some embodiments, the combination therapy, which includes administering the immunotherapy (e.g. T cell therapy, including engineered cells, such as CAR-T cell therapy) and the inhibitor, is administered to a subject or patient having a disease or condition to be treated (e.g. cancer) or at risk for having the disease or condition (e.g. cancer). In some aspects, the methods treat, e.g., ameliorate one or more symptom of, the disease or condition, such as by lessening tumor burden in a cancer expressing an antigen recognized by the immunotherapy or immunotherapeutic agent, e.g. recognized by an engineered T cell.

In some embodiments, the disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by bacterial, viral or other pathogens. Exemplary antigens, which include antigens associated with various diseases and conditions that can be treated, include any of antigens described herein. In particular embodiments, the recombinant receptor expressed on engineered cells of a combination therapy, including a chimeric antigen receptor or transgenic TCR, specifically binds to an antigen associated with the disease or condition.

In some embodiments, the disease or condition is a tumor, such as a solid tumor, lymphoma, leukemia, blood tumor, metastatic tumor, or other cancer or tumor type.

In some embodiments, the combination therapy is administered to a subject having a particular B cell malignancy. The B cell malignancy that is treated can be any in which expression of an antigen is associated with and/or involved in the etiology of the B cell malignancy, e.g. causes, exacerbates or otherwise is involved in the B cell malignancy. Exemplary B cell malignancies can include diseases or conditions associated with malignancy or transformation of cells (e.g. a cancer). Exemplary antigens, which include antigens associated with various B cell malignancies that can be treated, are described herein. In particular embodiments, the chimeric antigen receptor specifically binds to an antigen associated with the disease or condition. In some embodiments, antigens targeted by the receptors include antigens associated with a B cell malignancy, such as any of a number of known B cell marker. In some embodiments, the antigen is expressed by or on B cells, including human B cells. In some embodiments, the antigen targeted by the receptor is CD20, CD19, CD22, ROR1, CD45, CD21, CD5, CD33, Igkappa, Iglambda, CD79a, CD79b or CD30. In some embodiments, the antigen is CD19 and the chimeric antigen receptor specifically binds CD19. In some embodiments, the CD19 antigen is a human CD19.

In some embodiments, the B cell malignancy to be treated include leukemia and lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma (FL), refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the methods involve treating a subject having a lymphoma or a leukemia, such as a non-Hodgkin lymphoma (NHL) by administering antigen receptor-expressing cells (e.g. CAR-expressing cells) and a kinase inhibitor, such as a BTK/ITK inhibitor, e.g., ibrutinib. In some embodiments, the initiation of administration of the kinase inhibitor, e.g., ibrutinib, is prior to administering the recombinant receptor-expressing cells (e.g. CAR-expressing cells), such as prior to initiating administration of the recombinant receptor-expressing cells (e.g. CAR-expressing cells).

In some embodiments, NHL can be staged based on the Lugano classification (see, e.g., Cheson et al., (2014) JCO 32(27):3059-3067; Cheson, B. D. (2015) Chin Clin Oncol 4(1):5). In some cases, the stages are described by Roman numerals I through IV (1-4), and limited stage (I or II) lymphomas that affect an organ outside the lymph system (an extranodal organ) are indicated by an E. Stage I represents involvement in one node or a group of adjacent nodes, or a single extranodal lesions without nodal involvement (IE). Stage 2 represents involvement in two or more nodal groups on the same side of the diaphragm or stage I or II by nodal extent with limited contiguous extranodal involvement (IIE). Stage III represents involvement in nodes on both sides of the diaphragm or nodes above the diaphragm with spleen involvement. Stage IV represents involvement in additional non-contiguous extralymphatic involvement. In addition, “bulky disease” can be used to describe large tumors in the chest, in particular for stage II. The extent of disease is determined by positron emission tomography (PET)-computed tomography (CT) for avid lymphomas, and CT for non-avid histologies.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). In some embodiments, the subject has an ECOG status of less than or equal to 1. The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sorensen et al., (1993) Br J Cancer 67(4) 773-775. The criteria reflective of the ECOG performance status are described in Table 1 below:

TABLE 1
ECOG Performance Status Criteria

| Grade | ECOG performance status |
|---|---|
| 0 | Fully active, able to carry on all pre-disease |
| | performance without restriction |
| 1 | Restricted in physically strenuous activity |
| | but ambulatory and able to carry out work |
| | of a light or sedentary nature, e.g., light |
| | house work, office work |
| 2 | Ambulatory and capable of all selfcare but |
| | unable to carry out any work activities; up |
| | and about more than 50% of waking hours |
| 3 | Capable of only limited selfcare; confined to |
| | bed or chair more than 50% of waking hours |
| 4 | Completely disabled; cannot carry on any |
| | selfcare; totally confined to bed or chair |
| 5 | Dead |

In some embodiments, the subject has or has been identified as having as having a double/triple hit lymphoma or a lymphoma of the double/triple hit molecular subtypes. In some embodiments, the lymphoma is a double hit lymphoma characterized by the presence of MYC (myelocytomatosis oncogene), BCL2 (B-cell lymphoma 2), and/or BCL6 (B-cell lymphoma 6) gene rearrangements (e.g., translocations). In some embodiments, the lymphoma is a triple hit lymphoma characterized by the presence of MYC, BCL2, and BCL6 gene rearrangements; see, e.g., Aukema et al., (2011) Blood 117:2319-2331. In some aspects of such embodiments the subject is ECOG 0-1. In aspects, the therapy is indicated for such subjects and/or the instructions indicate administration to a subject within such population. In some embodiments, based on the 2016 WHO criteria (Swerdlow et al., (2016) Blood 127(20):2375-2390), double/triple hit lymphoma can be considered high-grade B-cell lymphoma, with MYC and BCL2 and/or BCL6 rearrangements with DLBCL histology (double/triple hit).

In some embodiments, the combination therapy is administered to subjects who are or are likely to be or who are predicted to be poor responders and/or who do not, are likely not to and/or who are predicted not to respond or do not respond within a certain time and/or to a certain extent to treatment with a cell therapy (e.g. CAR+ T cells). In some embodiments, the combination therapy is administered to subjects who do not or are not likely to or are not predicted to exhibit a complete response or overall response, such as within 1 month, within two months or within three months after initiation of administration of a cell therapy. In some embodiments, the combination therapy is administered to subjects who exhibit or are likely to exhibit or who are predicted to exhibit progressive disease (PD), such as within 1 month, two months or three months, following administration of the cell therapy. In some embodiments, a subject is likely or predicted not to exhibit a response or a certain response based on a plurality of similarly situated subjects so treated or previously treated with the cell therapy.

In some embodiments, the provided methods involve treating a specific group or subset of subjects, e.g., subjects identified as having high-risk disease, e.g., high-risk NHL. In some aspects, the methods treat subjects having a form of aggressive and/or poor prognosis B-cell non-Hodgkin lymphoma (NHL), such as NHL that has relapsed or is refractory (R/R) to standard therapy has a poor prognosis. In some cases, the overall response rate (ORR) to available therapies, to a standard of care, or to a reference therapy for the disease and/or patient population for which the therapy is indicated, is less than 40% and/or the complete response (CR) is less than 20%. In some embodiments, in chemorefractory DLBCL, the ORR with a reference or available treatment or standard-of-care therapy is about 26% and the CR is about 8% (Crump et al. Outomes in refractory aggressive diffuse large B-cell lymphoma (DLBCL): Results from the international SCHOLAR study. ASCO 2016 [Abstract 7516]). In some aspects, the provided methods, compositions, uses and articles of manufacture achieve improved and superior responses to available therapies.

In some embodiments, the methods and uses for treatment of subjects described herein involves selecting or identifying a particular group or subset of subjects, e.g., based on specific types of disease, diagnostic criteria, prior treatments and/or response to prior treatments. In some embodiments, the methods involve treating a subject having relapsed following remission after treatment with, or become refractory to, one or more prior therapies; or a subject that has relapsed or is refractory (R/R) to one or more prior therapies, e.g., one or more lines of standard therapy including those as described herein.

In some embodiments, the subject has been subject to more than one, two three, four, five, or six prior therapies. In some embodiments, the subject has been subject to one prior therapy. In some embodiments, the subject has been subject to about two to four prior therapies. In some embodiments, the subject has been subject to about five to six prior therapies. In some embodiments, the subject has been subject to more than six prior therapies.

In some embodiments, the subject has been previously treated with a therapy or a therapeutic agent targeting the B cell malignancy, e.g., NHL, prior to administration of the cells expressing the recombinant receptor. In some embodiments, the subject has been previously treated with a cell therapy (e.g., CAR+ T cells). In some embodiments, the subject has been previously treated with a hematopoietic stem cell transplantation (HSCT), e.g., allogenic HSCT or autogenic HSCT. In some embodiments, the subject has had poor prognosis after treatment with standard therapy and/or has failed one or more lines of previous therapy. In some embodiments, the subject has been treated or has previously received at least or about at least or about 1, 2, 3, 4, 5, 6, or 7 other therapies for treating the NHL other than a lymphodepleting therapy. In some embodiments, the subject has been previously treated with chemotherapy or radiation therapy. In some aspects, the subject is refractory or non-responsive to the other therapy or therapeutic agent. In some embodiments, the subject has persistent or relapsed disease, e.g., following treatment with another therapy or therapeutic intervention, including chemotherapy or radiation.

In some embodiments, the combination therapy is administered to subjects that have progressed on a prior treatment. In some embodiments, the combination therapy is administered to subjects that have stopped responding to a prior therapy. In some embodiments, the combination therapy is administered to subjects that have relapsed following a remission after a prior treatment. In some embodiments, the combination therapy is administered to subjects that are refractory to a prior treatment. In some embodiments, the combination therapy is administered to subjects that have less than an optimal response (e.g., a complete response, a partial response or a stable disease) to a prior therapy.

In some embodiments, the subjects are refractory to last prior therapy. In some embodiments, the subjects have a relapse to last prior therapy. The status is refractory if a subject achieved less than a partial response to last prior therapy. In some embodiments, the subjects have a prior chemotherapy. In some embodiments, the subjects are chemorefractory to the prior chemotherapy. In some embodiments, the subjects are chemosensitive to the prior therapy. The status is chemorefractory is a subject achieved stable disease (SD) or progressive disease (PD) to last chemotherapy-containing regimen or relapsed less than 12 months after autologous stem cell transplant. Otherwise the status is chemosensitive.

In some embodiments, the methods can be used for treating B cell malignancies or hematological malignancies, and in particular such malignancies in which responses, e.g. complete response, to treatments with either a T cell therapy, such as CAR-T cells, or a kinase inhibitor, e.g., ibrutinib, alone or not as a combination therapy together as provided herein, have not been entirely satisfactory or have been relatively low compared to similar treatments of other B cell malignancies or in other subjects. In some embodiments, the B cell malignancy is one in which treatment with an immunotherapy or immunotherapeutic agent, such as a composition including cells for adoptive cell therapy (e.g. CAR-expressing T cells), when administered alone or in another combination that is distinct from a combination therapy as provided herein and/or is not a combination with a kinase inhibitor-based therapy, e.g., ibrutinib-based therapy, results in a CR in less than or less than about 60%, less than about 50% or less than about 45% of the subjects so treated. In some embodiments, the subject and/or the B cell malignancy is one that is not responsive to and/or has been deemed refractory to or resistant to treatment with the inhibitor and/or with a kinase inhibitor therapy, e.g., ibrutinib therapy, is an aggressive or high-risk cancer and/or more has one or more features (e.g. markers) indicative of poor prognosis and/or poor outcome following treatment with the inhibitor and/or with a kinase inhibitor therapy, e.g., ibrutinib therapy.

In some embodiments, the combination therapy provided herein is for use in a subject having a cancer in which at the time of the provided combination therapy, such as at the time of administration of the T cell therapy (e.g., CAR-expressing T cells) and at the time of administering the kinase inhibitor, such as a BTK/ITK inhibitor, e.g. ibrutinib, the subject is not responsive to and/or has been deemed refractory to or resistant to a previous treatment with the inhibitor and/or with a BTK inhibitor therapy. In some embodiments, the provided combination therapy with the inhibitor and immunotherapy is carried out in a subject having a disease or condition, e.g. B cell malignancy, in which, at the time of initiation of the combination therapy, the subject has a disease that is progressing following administration of such previous inhibitor but in the absence of a therapy involving T cells (e.g. CAR-T cells), such as has progressive disease (PD) as best response, or is progressing after a previous response.

In some embodiments, the provided combination therapy with a kinase inhibitor, e.g. ibrutinib, and a T cell therapy (e.g. CAR-T cells) is carried out in a subject having a disease or condition, e.g. B cell malignancy, in which, at the time of initiation of the provided combination therapy, the subject had a response less than a complete response (CR) after previously receiving the inhibitor and/or a kinase inhibitor, e.g. ibrutinib, for at least 6 months.

In some aspects, the subject for treatment with the provided combination therapy is or is identified as exhibiting one or more high-risk features of the disease or condition and/or exhibits an aggressive disease or a disease associated with poor prognosis or outcome. In some aspects, high-risk features of a B cell malignancy, such as a lymphoma or a leukemia, e.g. CLL or SLL, include the presence of one or more molecular markers, such as one or more genetic marker, indicative of the severity or prognosis of the disease (see e.g. Parker and Strout (2011) Discov. Med., 11:115-23). In some embodiments, the subject has a B cell malignancy that is or is identified as having one or more cytogenetic abnormalities, such as two or three or more chromosomal abnormalities, such as 17p deletion, 11q deletion, trisomy 12, and/or 13q deletion, for example as detected by fluorescence in situ hybridization (FISH). In some embodiments, the subject has a B cell malignancy that is or is identified as having one or more gene mutations, such as TP53 mutation, NOTCH1 mutation, SF3B1 mutation and BIRC3 mutation, such as assessed using single nucleotide array (SNP)-array based method, Denaturing High Performance Liquid Chromatography (DHPLC), functional analysis of separated alleles in yeast (FASAY), or by sequencing, including direct sequencing or next generating sequencing methods. In some embodiments, the subject has a B cell malignancy that is or is identified as having unmutated immunoglobulin heavy chain variable region (IGHV). Mutation status of the variable region of IGH has prognostic value where unmutated (<2% compared with germline) is associated with aggressive disease (Hamblin, Best Pract. Res. Clin. Haematol. 20:455-468 (2007)). CD38 and ZAP70 expression, as assessed by flow cytometry, are considered surrogates for IGH mutation status. In some embodiments, the subject has a B cell malignancy that exhibits high-risk features that include 3 or more chromosomal abnormalities, 17p deletion, TP53 mutation and/or or unmutated IGHV.

In some embodiments, the combination therapy provided herein is for use in a subject having a cancer in which the subject and/or the cancer is resistant to inhibition of BTK or comprises a population of cells that are resistant to inhibition by the inhibitor. In some embodiments, the subject exhibits a mutation in a target kinase, such as BTK, or in a downstream molecule of the pathway of the target kinase rendering the subject resistant to treatment with the inhibitor and/or a BTK inhibitor therapy. Mutations rendering a subject resistant to or refractory to treatment with a BTK inhibitor or another inhibitor of a TEC family kinase are known, see e.g. Woyach et al. (2014) N Engl J. Med. 370:2286-94 and Liu et al. (2015) Blood, 126:61-8. In some embodiments, the combination therapy provided herein is for use in a subject having a cancer in which the subject and/or the cancer comprises a mutation or disruption in a nucleic acid encoding BTK, such as a mutation that is capable of reducing or preventing inhibition of the BTK by the inhibitor, e.g. ibrutinib. In some embodiments, the subject contains the C481S mutation of BTK. In some embodiments, the combination therapy provided herein is for use in a subject having a cancer in which the subject and/or the cancer comprises a mutation or disruption in a nucleic acid encoding PLCγ2, such as a gain of function mutation that can lead to autonomous signaling. In some embodiments, the subject contains the R665W and/or L845F mutation in PLCγ2.

In some cases, following treatment with one or more prior therapies, such as at least two or three prior therapies, for treating the cancer, the subject has not achieved a complete response (CR), has stable or progressive disease and/or relapsed following a response to the one or more prior therapies. In some embodiments, at least one of the prior therapies was a previous treatment with the inhibitor or a BTK inhibitor therapy, such as ibrutinib. In some embodiments, the subject was receiving the inhibitor or a BTK inhibitor therapy for at least six months with a response less than a CR and/or exhibits high risk features such as complex cytogenetic abnormalities (3 or more chromosomal abnormalities), 17p deletion, TP53 mutation, or unmutated IGHV.

In some embodiments, certain cancers, such as NHL, e.g. high-risk or aggressive NHL, such as DLBCL, and/or chronic lymphocytic leukemia (CLL) can be associated with defects in or reduction in intrinsic T cell functionality, which, in some cases, is influenced by the disease itself. For example, the pathogenesis of many cancers, such as CLL and NHL, e.g. DLBCL, can be associated with immunodeficiency, leading to promotion of tumor growth and immune evasion, such as due to immunosuppression of T cells, e.g. driven by one or more factors in the tumor microenvironment. In some cases, alleviating intrinsic T cell defects obtained from cancers of such patients for use in connection with adoptive cell therapy could provide for more potent responses to adoptive T cell therapy, e.g. CAR-T cell therapy.

In some embodiments, the provided methods are for treating a cancer in a subject in which such subject's T cells display or have been observed to display a decreased level of a factor indicative of T cell function, health, or activity, as compared to a reference population of T cells or a reference or threshold level, e.g. T cells from a subject not having or suspected of having a cancer, such as from a healthy or normal subject. In some embodiments, the provided methods are for treating subjects identified as having high-risk NHL and/or aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL). For example, as shown herein, in the presence of the exemplary BTK inhibitor ibrutinib, T cells engineered from subjects having DLBCL exhibit a greater T cell functional activity, indicating that the function of the T cells is potentiated in the presence of the inhibitor. In some embodiments of the provided methods, the administered engineered T cells are autologous to the subject. In some embodiments, the subject has DLBCL. In some embodiments, the provided methods are for treating a subject having chronic lymphocytic leukemia (CLL). In some embodiments, the provided methods are for treating a subject having small lymphocytic lymphoma (SLL).

Among the provided methods herein are methods for treating CLL, which is a hematologic malignancy characterized by a progressive accumulation of clonally-derived B-lymphocytes, e.g. CD19+, in the blood, bone marrow and lymphatic tissue. Although considered the same disease as CLL, in some cases, small lymphocytic lymphoma (SLL) is used to refer to the disease when characterized by lymphadenopathy (cancer cells found in the lymph nodes) whereas in CLL cancer cells are found mostly in the blood and bone marrow. For purposes herein, reference to CLL can include SLL unless stated otherwise. In some embodiments, CLL includes subjects who have documented CLL according to IWCLL criteria (Hallek (2008) Blood, 111:5446-5456), measureable disease (e.g. lymphocytosis >5×10⁹/L, measurable lymph nodes, hepatic and/or splenomegaly). In some embodiments, SLL includes subjects with lymphadenopathy and/or splenomegaly and <5×10⁹ CD19+CD5+ clonal B lymphocytes/L (<5000/μL) in the peripheral blood at diagnosis with measurable disease as determined by at least one lesion >1.5 cm in the greatest transverse diameter that is biopsy-proven SLL. Patients with progressive CLL generally have a poor prognosis with an overall survival (OS) of less than 1 year as reported in some studies (Jain et al. (2016) Expt. Rev. Hematol., 9:793-801).

Treatment of CLL with BTK inhibitor therapy, and in particular ibrutinib, is a current first-line approved therapy for CLL patients. Although partial responses (PRs) can be sustained for a long duration, studies that found that around 25% of previously treated CLL patients discontinue ibrutinib (Jain et al. (2015) Blood, 125:2062-2067; Maddocks (2015) JAMA Oncol., 1:80-87; Jain et al. (2017) Cancer, 123:2268-2273). In some cases, discontinuation of ibrutinib is due to progression of CLL or Richter's transformation. The majority of patients who discontinue ibrutinib for progressive disease (PD) are those with high risk features such as del(17p) (17p deletion), complex karyotype or cytogenetic abnormalities and unmutated immunoglobulin heavy chain variable region (IGHV). Further, mutations in BTK or the downstream effector phospholipase Cγ2 (PLCγ2) can emerge during ibrutinib treatment and are associated with ibrutinib resistance and ultimately relapse (Woyach et al. (2014) N. Engl. J. Med., 370:2286-2294). Such mutations are observed in 87% of CLL patients relapsing on ibrutinib. There is a need for alternative therapies in such subjects.

In some embodiments, the kinase inhibitor, such as a BTK/ITK inhibitor, e.g. ibrutinib is initially administered prior to a T cell therapy, e.g. CAR-T cells. In some aspects, the administration of the kinase inhibitor, such as a BTK/ITK inhibitor, e.g. ibrutinib is continued and/or the kinase inhibitor, such as a BTK/ITK inhibitor, e.g. ibrutinib is further administered concurrently with and/or after initiation of administration of a T cell therapy, e.g. CAR-T cells. In some aspects, the inhibitor is administered daily. In some aspects, the administration, such as daily administration, of a kinase inhibitor, e.g. ibrutinib is initiated, prior to the initiation of administration of a T cell therapy, e.g. CAR-T cells and is continued for up to a predetermined number of days. In some aspects, the predetermined number of days is a predetermined number of days after initiation of administration of the T cell therapy. In some embodiments, the inhibitor is administered, such as is administered daily, until a time at which or until a time after a level of the T cell therapy, CAR-T cells, is at a peak or maximum, e.g. Cmax, level following the administration of the T cells, e.g., CAR-expressing T cells, in the blood or disease-site of the subject. In some aspects, the administration of the inhibitor, e.g. ibrutinib, is continued for at least or at least about 14 days, at least or at least about 30 days, at least or at least about 60 days, at least or at least about 90 days, at least or at least about 120 days or at least or at least about 180 days after initiation of administration of the T cell therapy. In some embodiments, administration of the kinase inhibitor, e.g. ibrutinib, is continued for at least or about at least or about or 90 days after initiation of administration of the T cell therapy, e.g. CAR-T cells. In some aspects, at the time of terminating the administration of the inhibitor, persistence of the T cell therapy in the subject is observed. In some embodiments, at the time of terminating the administration of the inhibitor, the subject can be evaluated to assess if the subject is receiving a benefit from administration of the kinase inhibitor, e.g. ibrutinib. In some embodiments, at the time of terminating the administration of the inhibitor, the subject is evaluated to assess whether the subject has achieved a response or a particular degree or outcome indicative of a response, such as in some embodiments a CR. In some such embodiments, if a subject has achieved a CR or other outcome indicative of response or indicative of a likelihood of CR or other outcome, the provided methods, compositions, articles of manufacture or uses, allow for, specify, or involve discontinuation of the inhibitor or administration thereof. In some such embodiments, if a subject has not achieved a CR, the provided methods allow for continuation of administration of the inhibitor. Thus, in some aspects, the provided methods and other embodiments avoid or reduce prolonged or excessively prolonged administration of the inhibitor. In some aspects, such prolonged administration otherwise may result in, or increase likelihood of, one or more undesirable outcomes such as side effects or disruption or reduction in quality of life for the subject to which the therapy is being administered, such as the patient. In some aspects, a set predetermined time period, such as minimal time period, of administration, may increase likelihood of patient compliance or likelihood that the inhibitor will be administered as instruction or according to the methods, particularly in the case of daily administration.

In some embodiments, the combination therapy is administered to a subject and/or a cancer that is resistant to inhibition of Bruton's tyrosine kinase (BTK) and/or comprises a population of cells that are resistant to inhibition by the inhibitor. In some embodiments, the combination therapy is administered to a subject and/or a cancer that comprises a mutation in a nucleic acid encoding a BTK, optionally wherein the mutation is capable of reducing or preventing inhibition of the BTK by the inhibitor and/or by ibrutinib, optionally wherein the mutation is C481S. In some embodiments, the combination therapy is administered to a subject and/or a cancer that comprises a mutation in a nucleic acid encoding phospholipase C gamma 2 (PLCgamma2), optionally wherein the mutation results in constitutive signaling activity, optionally wherein the mutation is R665W or L845. In some embodiments, the combination therapy is administered to a subject and/or a cancer where, at the time of the initiation of administration of the kinase inhibitor, such as a BTK/ITK inhibitor, e.g., ibrutinib and at the time of the initiation of administration of the T cell therapy, the subject has relapsed following remission after treatment with, or been deemed refractory to a previous treatment with the inhibitor and/or with a BTK inhibitor therapy. In some embodiments, the combination therapy is administered to a subject and/or a cancer where, at the time of the initiation of administration of the kinase inhibitor, such as a BTK/ITK inhibitor, e.g., ibrutinib and at the time of the initiation of administration of the T cell therapy, the subject has progressed following a previous treatment with the inhibitor and/or with a BTK inhibitor therapy, optionally wherein the subject exhibited progressive disease as the best response to the previous treatment or progression after previous response to the previous treatment. In some embodiments, the combination therapy is administered to a subject and/or a cancer where, at the time of the initiation of administration of the kinase inhibitor, such as a BTK/ITK inhibitor, e.g., ibrutinib and at the time of the initiation of administration of the T cell therapy, the subject exhibited a response less than a complete response (CR) following a previous treatment for at least 6 months with the inhibitor and/or with a BTK inhibitor therapy.

In some embodiments, the combination therapy is administered to (i) the subject and/or the cancer (a) is resistant to inhibition of Bruton's tyrosine kinase (BTK) and/or (b) comprises a population of cells that are resistant to inhibition by the inhibitor; (ii) the subject and/or the cancer comprises a mutation in a nucleic acid encoding a BTK, capable of reducing or preventing inhibition of the BTK by the inhibitor and/or by ibrutinib, optionally wherein the mutation is C481S; (iii) the subject and/or the cancer comprises a mutation in a nucleic acid encoding phospholipase C gamma 2 (PLCgamma2), optionally wherein the mutation results in constitutive signaling activity, optionally wherein the mutation is R665W or L845F; (iv) at the time of the initiation of administration of the kinase inhibitor, such as a BTK/ITK inhibitor, e.g., ibrutinib, and the initiation of administration of the composition comprising T cells, the subject has relapsed following remission after a previous treatment with, or been deemed refractory to a previous treatment with, the inhibitor and/or with a BTK inhibitor therapy; (v) at the time of the initiation of administration of the kinase inhibitor, such as a BTK/ITK inhibitor, e.g., ibrutinib, and the initiation of administration of the composition comprising T cells, the subject has progressed following a previous treatment with the inhibitor and/or with a BTK inhibitor therapy, optionally wherein the subject exhibited progressive disease as the best response to the previous treatment or progression after previous response to the previous treatment; and/or (vi) at the time of the initiation of administration of the kinase inhibitor, such as a BTK/ITK inhibitor, e.g., ibrutinib, and the initiation of administration of the composition comprising T cells, the subject exhibited a response less than a complete response (CR) following a previous treatment for at least 6 months with the inhibitor and/or with a BTK inhibitor therapy. In some embodiments, the subject is a subject who had previously received administration of a kinase inhibitor, such as a BTK/ITK inhibitor, e.g., ibrutinib, then discontinued the treatment with the kinase inhibitor.

In some embodiments, the methods, uses and articles of manufacture involve, or are used for treatment of subjects involving, selecting or identifying a particular group or subset of subjects, e.g., based on specific types of disease, diagnostic criteria, prior treatments and/or response to prior treatments, such as any group of subjects as described. In some embodiments, the methods involve treating a subject having relapsed following remission after treatment with, or become refractory to, one or more prior therapies; or a subject that has relapsed or is refractory (R/R) to one or more prior therapies, e.g., one or more lines of standard therapy, e.g., a cell therapy (e.g., CAR+ T cells). In some embodiments, the methods involve treating subjects having diffuse large B-cell lymphoma (DLBCL), not otherwise specified (NOS; de novo and transformed from indolent), primary mediastinal (thymic) large B-cell lymphoma (PMBCL) or follicular lymphoma (FL), optionally, follicular lymphoma Grade 3B (FL3B), EBV positive DLBCL, or EBV positive NOS. In some embodiments, the methods involve treating a subject that has an Eastern Cooperative Oncology Group Performance Status (ECOG) of less than 1, such as 0-1. In some embodiments, the methods treat a poor-prognosis population or of DLBCL patients or subject thereof that generally responds poorly to therapies or particular reference therapies, such as one having one or more, such as two or three, chromosomal translocations (such as so-called “double-hit” or “triple-hit” lymphoma, which is high grade B-cell lymphoma with MYC and BCL2 and/or BCL6 rearrangements with DLBCL histology; having translocations MYC/8q24 loci, usually in combination with the t (14; 18) (q32; q21) bc1-2 gene or/and BCL6/3q27 chromosomal translocation; see, e.g., Xu et al. (2013) Int J Clin Exp Pathol. 6(4): 788-794), and/or one having relapsed, optionally relapsed within 12 months, and/or one having been deemed chemorefractory.

In some embodiments, the subject has DLBCL that is a germinal center-like (GCB) DLBCL. In some embodiments, the subject has a non-germinal center-like (non-GCB) DLBCL. In some embodiments, the subject has double-hit lymphoma (DHL). In some embodiments, the subject has a triple-hit lymphoma (THL). In some embodiments, the subject is positive for the expression of a gene indicative of the responsiveness of the treatment with a kinase inhibitor, such as a BTK/ITK inhibitor, e.g., ibrutinib. In some embodiments, the subject is negative for the expression of the gene. See Blood 2017 130:4118.

In some embodiments, the antigen receptor (e.g. CAR) specifically binds to a target antigen associated with the disease or condition, such as associated with NHL. In some embodiments, the antigen associated with the disease or disorder is selected from CD20, CD19, CD22, ROR1, CD45, CD21, CD5, CD33, Igkappa, Iglambda, CD79a, CD79b or CD30. In some embodiments, the antigen is CD19. In some embodiments, the CD19 antigen is a human CD19.

In some embodiments, the methods include administration of the cell therapy and a kinase inhibitor, such as a BTK/ITK inhibitor, e.g., ibrutinib, to a subject, which is, at risk for, or suspected of having a B cell malignancy.

In some embodiments, the methods include administration of cells to a subject selected or identified as having a certain prognosis or risk of NHL. Non-Hodgkin lymphoma (NHL) can be a variable disease. Some subjects with NHL may survive without treatment while others may require immediate intervention. In some cases, subjects with NHL may be classified into groups that may inform disease prognosis and/or recommended treatment strategy. In some cases, these groups may be “low risk,” “intermediate risk,” “high risk,” and/or “very high risk” and patients may be classified as such depending on a number of factors including, but not limited to, genetic abnormalities and/or morphological or physical characteristics. In some embodiments, subjects treated in accord with the methods, and/or with the articles of manufacture or compositions, are classified or identified based on the risk of NHL. In some embodiments, the subject is one that has high risk NHL.

In some embodiments, the subject to be treated includes a group of subjects with aggressive NHL, in particular, with diffuse large B-cell lymphoma (DLBCL), not otherwise specified (NOS; de novo and transformed from indolent), T cell/histiocyte-rich large B-cell lymphoma, primary mediastinal (thymic) large B-cell lymphoma (PMBCL), follicular lymphoma (FL), optionally, follicular lymphoma Grade 3B (FL3B), EBV positive DLBCL, EBV positive NOS, or high grade B-cell lymphoma with MYC and BCL2 and/or BCL6 rearrangements with DLBCL histology (“double-hit” or “triple-hit” lymphoma). In some embodiments, the subject's disease has relapsed or been refractory to at least two prior lines of therapy. In some embodiments, the prior therapy comprises a CD20-targeted agent and/or an anthracycline. In some embodiments, the subject is or has been identified as having an Eastern Cooperative Oncology Group Performance Status (ECOG) status of less than or equal to 1. In some embodiments, the subjects have a ECOG score of 0-1 at screening. In some embodiments, the subjects have positron emission tomography (PET)-positive disease as per Lugano Classification (Cheson, 2014). In some embodiments, the subject may optionally have previously been treated with allogenic stem cell transplantation (SCT).

In some embodiments, the subject is an adult. In some embodiments, the subjects are male. In some embodiments, the subjects are female. In some embodiments, the subjects are at least 40 years old at the time they are administered the combination therapy (e.g., at the time they are administered the cell therapy). In some embodiments, the subjects are less than 40 years old at the time they are administered the combination therapy (e.g., at the time they are administered the cell therapy). In some embodiments, the subjects are about 40-65 years old at the time they are administered the combination therapy (e.g., at the time they are administered the cell therapy). In some embodiments, the subjects are at least 65 years old at the time they are administered the combination therapy (e.g., at the time they are administered the cell therapy).

In some embodiments of the provided methods, one or more properties of administered genetically engineered cells can be improved or increased or greater compared to administered cells of a reference composition, such as increased or longer expansion and/or persistence of such administered cells in the subject or an increased or greater recall response upon restimulation with antigen. In some embodiments, the increase can be at least a 1.2-fold, at least 1.5-fold, at least 2-fold, at last 3-fold, at least 4-fold, at least 5-fold, at least 6-fold, at least 7-fold, at least 8-fold, at least 9-fold, or at least 10-fold increase in such property or feature compared to the same property or feature upon administration of a reference cell composition. In some embodiments, the increase in one or more of such properties or features can be observed or is present within one months, two months, three months, four months, five months, six months, or 12 months after administration of the genetically engineered cells.

In some embodiments, a reference cell composition can be a composition of T cells from the blood of a subject not having or not suspected of having the cancer or is a population of T cells obtained, isolated, generated, produced, incubated and/or administered under the same or substantially the conditions, except not having been incubated or administered in the presence of a kinase inhibitor, e.g., ibrutinib. In some embodiments, the reference cell composition contains genetically engineered cells that are substantially the same, including expression of the same recombinant receptor, e.g. CAR. In some aspects, such T cells are treated identically or substantially identically, such as manufactured similarly, formulated similarly, administered in the same or about the same dosage amount and other similar factors.

A. Administration of a Kinase Inhibitor

The provided combination therapy methods, compositions, combinations, kits and uses involve administration of a kinase inhibitor, such as TEC family kinase inhibitor, e.g., ibrutinib, which can be administered prior to, subsequently to, during, simultaneously or near simultaneously, sequentially and/or intermittently with administration of the T cell therapy, e.g., administration of T cells expressing a chimeric antigen receptor (CAR), and/or whose administration can begin prior to administration of the T cell therapy and continue until the initiation of administration of the T cell therapy or after the initiation of administration of the T cell therapy.

In some embodiments, the kinase inhibitor in the combination therapy is an inhibitor of a tyrosine kinase, such as a member of the TEC family of kinases which, in some cases, are involved in the intracellular signaling mechanisms of cytokine receptors, lymphocyte surface antigens, heterotrimeric G-protein-coupled receptors, and integrin molecules. In some embodiments, the kinase inhibitor in the combination therapy is an inhibitor of one or more members of the TEC family of kinases, including Bruton's tyrosine kinase (BTK), IL-2 inducible T-cell kinase (ITK), tec protein tyrosine kinase (TEC), bone marrow tyrosine kinase gene in chromosome X protein (BMX) non-receptor tyrosine kinase (also known as Epithelial and endothelial tyrosine kinase; ETK), and TXK tyrosine kinase (TXK). In some embodiments, the kinase inhibitor is a Bruton's tyrosine kinase (BTK) inhibitor. In some embodiments, the kinase inhibitor is a IL-2 inducible T-cell kinase (ITK) inhibitor. In some embodiments, the kinase inhibitor is both a BTK and an ITK inhibitor, such as ibrutinib.

In some embodiments, the kinase inhibitor is an irreversible inhibitor of one or more TEC family kinases. In some embodiments, the kinase inhibitor is an irreversible inhibitor of BTK. In some embodiments, the kinase inhibitor is an irreversible inhibitor of ITK.

In some embodiments, the kinase inhibitor inhibits BTK with a half-maximal inhibitory concentration (IC₅₀) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the kinase inhibitor binds to BTK with an equilibrium dissociation constant (Kd) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the inhibition constant (Ki) of the kinase inhibitor for BTK is less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the kinase inhibitor inhibits ITK with a half-maximal inhibitory concentration (IC₅₀) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the kinase inhibitor binds to ITK with an equilibrium dissociation constant (Kd) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the inhibition constant (Ki) of the kinase inhibitor for ITK is less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the kinase inhibitor inhibits both BTK and ITK. In some embodiments, the kinase inhibitor inhibits both BTK and ITK with a half-maximal inhibitory concentration (IC₅₀) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the kinase inhibitor binds to both BTK and ITK with an equilibrium dissociation constant (Kd) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the inhibition constant (Ki) of the kinase inhibitor for both BTK and ITK is less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the IC₅₀, Kd and/or Ki is measured or determined using an in vitro assay. Assays to assess or quantitate or measure activity of protein tyrosine kinase inhibitors as described are known in the art. Such assays can be conducted in vitro and include assays to assess the ability of an agent to inhibit a specific biological or biochemical function. In some embodiments. In some embodiments, kinase activity studies can be performed. Protein tyrosine kinases catalyze the transfer of the terminal phosphate group from adenosine triphosphate (ATP) to the hydroxyl group of a tyrosine residue of the kinase itself or another protein substrate. In some embodiments, kinase activity can be measured by incubating the kinase with the substrate (e.g., inhibitor) in the presence of ATP. In some embodiments, measurement of the phosphorylated substrate by a specific kinase can be assessed by several reporter systems including colorimetric, radioactive, and fluorometric detection. (Johnson, S. A. & T. Hunter (2005) Nat. Methods 2:17.) In some embodiments, inhibitors can be assessed for their affinity for a particular kinase or kinases, such as by using competition ligand binding assays (Ma et al., Expert Opin Drug Discov. 2008 June; 3(6):607-621) From these assays, the half-maximal inhibitory concentration (IC₅₀) can be calculated. IC₅₀ is the concentration that reduces a biological or biochemical response or function by 50% of its maximum. In some cases, such as in kinase activity studies, IC₅₀ is the concentration of the compound that is required to inhibit the target kinase activity by 50%. In some cases, the equilibrium dissociation constant (Kd) and/or the inhibition constant (Ki values) can be determined additionally or alternatively. IC₅₀ and Kd can be calculated by any number of means known in the art. The inhibition constant (Ki values) can be calculated from the IC₅₀ and Kd values according to the Cheng-Prusoff equation: Ki=IC₅₀/(1+L/Kd), where L is the concentration of the kinase inhibitor (Biochem Pharmacol 22: 3099-3108, 1973). Ki is the concentration of unlabeled inhibitor that would cause occupancy of 50% of the binding sites present in the absence of ligand or other competitors.

In some embodiments, the kinase inhibitor is a small molecule.

In some embodiments, the kinase inhibitor is an inhibitor of a tyrosine protein kinase that has an accessible cysteine residue near the active site of the tyrosine kinase. In some embodiments, the kinase inhibitor of one or more TEC family kinases forms a covalent bond with a cysteine residue on the protein tyrosine kinase. In some embodiments, the cysteine residue is a Cys 481 residue. In some embodiments, the cysteine residue is a Cys 442 residue. In some embodiments, the kinase inhibitor is an irreversible BTK inhibitor that binds to Cys 481. In some embodiments, the kinase inhibitor is an ITK inhibitor that binds to Cys 442. In some embodiments, the kinase inhibitor comprises a Michael acceptor moiety that forms a covalent bond with the appropriate cysteine residue of the tyrosine kinase. In some embodiments, the Michael acceptor moiety preferentially binds with the appropriate cysteine side chain of the tyrosine kinase protein relative to other biological molecules that also contain an assessable —SH moiety.

In some embodiments, the kinase inhibitor is an ITK inhibitor compound described in PCT Application Numbers WO2002/0500071, WO2005/070420, WO2005/079791, WO2007/076228, WO2007/058832, WO2004/016610, WO2004/016611, WO2004/016600, WO2004/016615, WO2005/026175, WO2006/065946, WO2007/027594, WO2007/017455, WO2008/025820, WO2008/025821, WO2008/025822, WO2011/017219, WO2011/090760, WO2009/158571, WO2009/051822, WO2014/082085, WO2014/093383, WO2014/105958, and WO2014/145403, which are each incorporated by reference in their entireties. In some embodiments, the kinase inhibitor is an ITK inhibitor compound described in U.S. Application Numbers US20110281850, US2014/0256704, US20140315909, and US20140303161, which are each incorporated by reference in their entireties. In some embodiments, the kinase inhibitor is an ITK inhibitor compound described in U.S. Pat. No. 8,759,358, which is incorporated by reference in its entirety.

In some embodiments, the kinase inhibitor, such as a BTK/ITK inhibitor, has a structure selected from

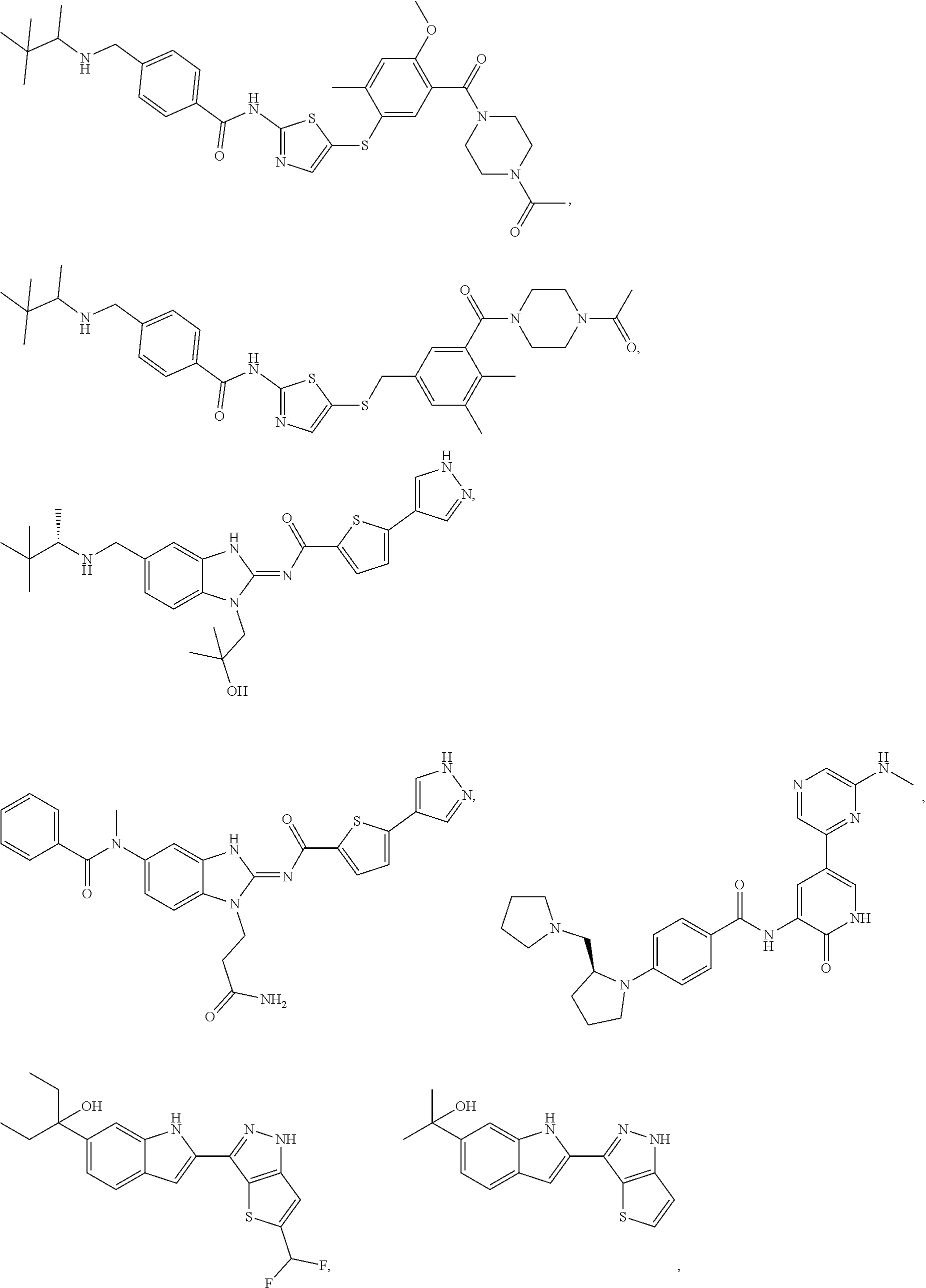

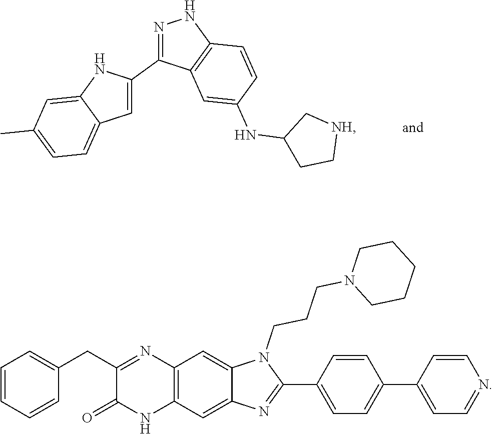 [SMILES: Cc1ccc2cc(-c3n[nH]c4ccc(NC5CCNC5)cc34)[nH]c2c1 ; O=c1[nH]c2cc3nc(-c4ccc(-c5ccncc5)cc4)n(CCCN4CCCCC4)c3cc2nc1Cc1ccccc1]

Exemplary inhibitors of BTK and/or ITK are known in the art. In some embodiments, the inhibitor is an inhibitor as described in Byrd et al., N Engl J Med. 2016; 374(4):323-32; Cho et al., J Immunol. 2015, doi:10.4049/jimmunol.1501828; Zhong et. al., J. Biol. Chem., 2015, 290(10): 5960-78; Hendriks et al., Nature, 2014, 14: 219-232; Akinleye et al., Journal of Hematology & Oncology 2013, 6:59; Wang et al., ACS Med Chem Lett. 2012 Jul. 26; 3(9): 705-9; Howard et al., J Med Chem. 2009 Jan. 22; 52(2):379-88; Anastassiasdis et al., Nat Biotechnol. 2011 Oct. 30; 29(11): 1039-45; Davis, et al., Nat Biotechnol, 2011; 29:1046-51; Bamborough et al., J Med Chem. 2008 Dec. 25; 51(24):7898-914; Roth et al., J Med Chem. 2015; 58:1053-63; Galkin et al., Proc Natl Acad Sci USA. 2007; 104:270-5; Singh et al., J Med Chem. 2012; 55:3614-43; Hall et al., J Med Chem. 2009 May 28; 52(10):3191-204; Zhou et al., Nature. 2009 Dec. 24; 462(7276):1070-4; Zapf et al., J Med Chem. 2012; 55:10047-63; Shi et al., Bioorg Med Chem Lett, 2014; 24:2206-11; Illig, et al., J Med Chem. 2011; 54:7860-83; and U.S. Patent Application Publication No: 20140371241.

Non-limiting examples of kinase inhibitor, such as a BTK/ITK inhibitor include Ibrutinib (PL-32765); PRN694; Spebrutinib (CC-292 or AVL-292); PCI-45292; RN-486; Compound 2c; AT9283; BML-275; Dovitinib (TKI258); Foretinib (GSK1363089); Gö6976; GSK-3 Inhibitor IX; GSK-3 Inhibitor XIII; Hesperadin; IDR E804; K-252a; Lestaurtinib (CEP701); Nintedanib (BIBF 1120); NVP-TAE684; R406; SB218078; Staurosporine (AM-2282); Sunitinib (SU11248); Syk Inhibitor; WZ3146; WZ4002; BDBM50399459 (CHEMBL2179805); BDBM50399460 (CHEMBL2179804); BDBM50399458 (CHEMBL2179806); BDBM50399461 (CHEMBL2179790); BDBM50012060 (CHEMBL3263640); BDBM50355504 (CHEMBL1908393); BDBM50355499 (CHEMBL1908395:CHEMBL1908842).

In some embodiments, the kinase inhibitor, such as a BTK/ITK inhibitor is or comprises ibrutinib. In some embodiments, the kinase inhibitor is has or comprises the following structure:

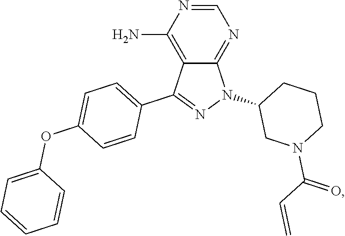 [SMILES: C=CC(=O)N1CCC[C@@H](n2nc(-c3ccc(Oc4ccccc4)cc3)c3c(N)ncnc32)C1]

or an enantiomer or mixture of enantiomers thereof, or a pharmaceutically acceptable salt, solvate, hydrate, co-crystal, clathrate, or polymorph thereof. In some embodiments, the kinase inhibitor is ibrutinib and has or comprises the following structure:

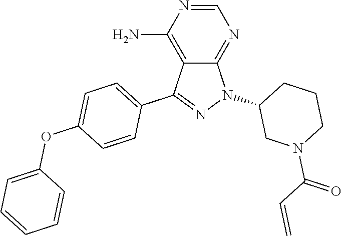 [SMILES: C=CC(=O)N1CCC[C@@H](n2nc(-c3ccc(Oc4ccccc4)cc3)c3c(N)ncnc32)C1]

or an enantiomer or mixture of enantiomers thereof, or a pharmaceutically acceptable salt, solvate, hydrate, co-crystal, clathrate, or polymorph thereof.

In some embodiments, the inhibitor is an inhibitor as described in U.S. Patent No. US 2014/0371241; US 2015/0140085; US 2015/0238490; US 2015/0352116; US 2015/0361504; US 2016/0022683; US 2016/0022684; US 2016/0038495; US 2016/0038496; US 2016/0287592; US 2017/0002009; US 2017/0079981; US 2017/0128448; US 2017/0209462; US 2017/0226108; US 2017/0226114; US 2017/0305914; US 2017/0360796; US 2017/0368173; US 2018/0009814; US 2018/0028537; US 2018/0051026; US 2018/0071293; US 2018/0071295; US 2018/0072737; U.S. Pat. Nos. 7,514,444; 8,008,309; 8,476,284; 8,497,277; 8,697,711; 8,703,780; 8,735,403; 8,754,090; 8,754,091; 8,957,079; 8,999,999; 9,125,889; 9,181,257; 9,296,753; 9,545,407; 9,655,857; 9,717,731; 9,725,455;

U.S. Pat. Nos. 9,730,938; 9,751,889; and 9,884,869. In some embodiments, the inhibitor is or comprises ibrutinib. In some aspects, the inhibitor is or comprises ibrutinib or 1-[(3R)-3-[4-amino-3-(4-phenoxyphenyl)pyrazolo[3,4-d]pyrimidin-1-yl]piperidin-1-yl]prop-2-en-1-one (also known as 1-[(3R)-3-[4-Amino-3-(4-phenoxyphenyl)-1H-pyrazolo[3,4-d]pyrimidin-1-yl]-1-piperidinyl]-2-propen-1-one; 1-[(3R)-3-[4-amino-3-(4-phenoxyphenyl)pyrazolo[3,4-d]pyrimidin-1-yl]piperidin-1-yl]prop-2-en-1-one; 1-((3R)-3-(4-amino-3-(4-phenoxyphenyl)-1H-pyrazolo(3,4-d)pyrimidin-1-yl)-1-piperidinyl)-2-Propen-1-one; 936563-96-1; PCI-32765; IMBRUVICA; UNIT-1X70OSD4VX; PCI32765; CRA-032765; 1X70OSD4VX; or CHEBI:76612). In some aspects, the inhibitor is or comprises 1-[(3R)-3-[4-amino-3-(4-phenoxyphenyl)pyrazolo[3,4-d]pyrimidin-1-yl]piperidin-1-yl]prop-2-en-1-one (also known as 1-[(3R)-3-[4-Amino-3-(4-phenoxyphenyl)-1H-pyrazolo[3,4-d]pyrimidin-1-yl]-1-piperidinyl]-2-propen-1-one; 1-[(3R)-3-[4-amino-3-(4-phenoxyphenyl)pyrazolo[3,4-d]pyrimidin-1-yl]piperidin-1-yl]prop-2-en-1-one; 1-((3R)-3-(4-amino-3-(4-phenoxyphenyl)-1H-pyrazolo(3,4-d)pyrimidin-1-yl)-1-piperidinyl)-2-Propen-1-one; 936563-96-1; PCI-32765; IMBRUVICA; UNIT-1X70OSD4VX; PCI32765; CRA-032765; 1X70OSD4VX; or CHEBI:76612).

In some embodiments, kinase inhibitor, such as a BTK/ITK inhibitor, the kinase inhibitor, such as a BTK/ITK inhibitor, is an enantiomer or a mixture of enantiomers of 1-[(3R)-3-[4-amino-3-(4-phenoxyphenyl)pyrazolo[3,4-d]pyrimidin-1-yl]piperidin-1-yl]prop-2-en-1-one, or a pharmaceutically acceptable salt, solvate, hydrate, co-crystal, clathrate, or polymorph of 1-[(3R)-3-[4-amino-3-(4-phenoxyphenyl)pyrazolo[3,4-d]pyrimidin-1-yl]piperidin-1-yl]prop-2-en-1-one. In some embodiments, kinase inhibitor, such as a BTK/ITK inhibitor, is a solvate of 1[(3R)-3-[4-amino-3-(4-phenoxyphenyl)pyrazolo[3,4-d]pyrimidin-1-yl]piperidin-1-yl]prop-2-en-1-one. In some embodiments, kinase inhibitor, such as a BTK/ITK inhibitor, is a hydrate of 11[(3R)-3-[4-amino-3-(4-phenoxyphenyl)pyrazolo[3,4-d]pyrimidin-1-yl]piperidin-1-yl]prop-2-en-1-one. In some embodiments, kinase inhibitor, such as a BTK/ITK inhibitor, is a pharmaceutically acceptable sale of 1-[(3R)-3-[4-amino-3-(4-phenoxyphenyl)pyrazolo[3,4-d]pyrimidin-1-yl]piperidin-1-yl]prop-2-en-1-one. In some embodiments, kinase inhibitor, such as a BTK/ITK inhibitor, is 1-[(3R)-3-[4-amino-3-(4-phenoxyphenyl)pyrazolo[3,4-d]pyrimidin-1-yl]piperidin-1-yl]prop-2-en-1-one. In some embodiments, Compound 1 has the structure of Formula I. In certain embodiments, a kinase inhibitor, e.g., ibrutinib, is a solid. In certain embodiments, a kinase inhibitor, e.g., ibrutinib, is hydrated. In certain embodiments, a kinase inhibitor, e.g., ibrutinib, is solvated. In certain embodiments, a kinase inhibitor, e.g., ibrutinib, is anhydrous. In certain embodiments, a kinase inhibitor, e.g., ibrutinib, is nonhygroscopic.

In certain embodiments, a kinase inhibitor, e.g., ibrutinib, is amorphous. In certain embodiments, a kinase inhibitor, e.g., ibrutinib, is crystalline. In certain embodiments, the solid a kinase inhibitor, e.g., ibrutinib, is in a crystalline form described in U.S. Pat. No. 9,751,889, which is incorporated herein by reference in its entirety.

The solid forms of a kinase inhibitor, e.g., ibrutinib, can be prepared according to the methods described in the disclosure of WO 2016/151438, U.S. Pat. No. 9,884,869, US 2017/0226108; WO 2016/151438; WO 2017/134684; WO 2015/145415; WO 2017/137446; WO 2016/088074; WO 2017/134684; WO 2015/145415; WO 2017/085628; and WO 2017/134588 or any one or combined available method(s).

In some embodiments, a kinase inhibitor, e.g., ibrutinib, provided herein contains one chiral center, and can exist as a mixture of enantiomers, e.g., a racemic mixture. This disclosure encompasses the use of stereomerically pure forms of such a compound, as well as the use of mixtures of those forms. For example, mixtures comprising equal or unequal amounts of the enantiomers of a kinase inhibitor, e.g., ibrutinib, provided herein may be used in methods and compositions disclosed herein. These isomers may be asymmetrically synthesized or resolved using standard techniques such as chiral columns or chiral resolving agents. See, e.g., Jacques, J., et al, Enantiomers, Racemates and Resolutions (Wiley-Interscience, New York, 1981); Wilen, S. H., et al, Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E L. Eliel, Ed., Univ. of Notre Dame Press, Notre Dame, Ind., 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

I. Compositions and Formulations

In some embodiments of the combination therapy methods, compositions, combinations, kits and uses provided herein, the combination therapy can be administered in one or more compositions, e.g., a pharmaceutical composition containing a kinase inhibitor, such as a BTK/ITK inhibitor, e.g., ibrutinib.

In some embodiments, the composition, e.g., a pharmaceutical composition containing a kinase inhibitor, such as a BTK/ITK inhibitor, e.g., ibrutinib, can include carriers such as a diluent, adjuvant, excipient, or vehicle with which a kinase inhibitor, such as a BTK/ITK inhibitor, e.g., ibrutinib, and/or the cells are administered. Examples of suitable pharmaceutical carriers are described in “Remington's Pharmaceutical Sciences” by E. W. Martin. Such compositions will contain a therapeutically effective amount of a kinase inhibitor, such as a BTK/ITK inhibitor, e.g., ibrutinib, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. The compositions containing a kinase inhibitor, such as a BTK/ITK inhibitor, e.g., ibrutinib, can also be lyophilized.

In some embodiments, the pharmaceutical compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration. In some embodiments, it is administered by multiple bolus administrations, for example, over a period of no more than 3 days, or by continuous infusion administration.

In some embodiments, the administration can be local, topical or systemic depending upon the locus of treatment. In some embodiments local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral.

In some embodiments, a kinase inhibitor, such as a BTK/ITK inhibitor, e.g., ibrutinib, are typically formulated and administered in unit dosage forms or multiple dosage forms. Each unit dose contains a predetermined quantity of therapeutically active a kinase inhibitor, e.g., ibrutinib, sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of a kinase inhibitor, e.g., ibrutinib. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

2. Dosing

In some embodiments, the provided combination therapy method involves administering to the subject a therapeutically effective amount of one or more doses of a kinase inhibitor, such as a BTK/ITK inhibitor, e.g., ibrutinib, and the cell therapy, such as a T cell therapy (e.g. CAR-expressing T cells). In some embodiments, the provided combination therapy methods involve initiating administration of a kinase inhibitor, such as a BTK/ITK inhibitor, e.g., ibrutinib, prior to, subsequently to, during, during the course of, simultaneously, near simultaneously, sequentially, concurrently and/or intermittently with the initiation of the cell therapy, such as a T cell therapy (e.g., CAR-expressing T cells). In some embodiments, the kinase inhibitor, e.g., ibrutinib is administered in multiple doses in regular intervals prior to, during, during the course of, and/or after the period of administration of the cell therapy (e.g. T cell therapy, such as CAR-T cell therapy). In some embodiments, the provided embodiments involve initiating the administration of a kinase inhibitor, such as a BTK/ITK inhibitor, e.g., ibrutinib, prior to administration of the T cell therapy and continue until the initiation of administration of the T cell therapy or after the initiation of administration of the T cell therapy.

In some embodiments, the method involves administering the kinase inhibitor, such as a BTK/ITK inhibitor, e.g., ibrutinib, prior to administration of the T cell therapy. In some embodiments, the method involves continuing to administer the kinase inhibitor, e.g., ibrutinib, after administration of the T cell therapy. In some embodiments, the method involves initiating the administration of the kinase inhibitor, such as a BTK/ITK inhibitor, e.g., ibrutinib, prior to initiation of administration of the T cell therapy. In some embodiments, the kinase inhibitor, e.g., ibrutinib, is further administered, after a discontinuation or pause during a lymphodepleting therapy, such as until after initiation of the T cell therapy. In some embodiments, continuing and/or further administration of the kinase inhibitor, e.g., ibrutinib, involves administration of multiple doses of the kinase inhibitor, e.g., ibrutinib. In some embodiments, the kinase inhibitor, e.g., ibrutinib, is not further administered and/or continued after initiation of the T cell therapy. In some embodiments, the dosage schedule comprises continuing to administer the kinase inhibitor, e.g., ibrutinib, prior to and after initiation of the T cell therapy. In some embodiments, the dosage schedule comprises administering the kinase inhibitor, e.g., ibrutinib, simultaneously with the administration of the T cell therapy. In some embodiments, the administration of the kinase inhibitor, e.g., ibrutinib, is continued and/or further administered over a period of time, e.g., until a determined time point or until a particular outcome is achieved. In some embodiments, the administration of the kinase inhibitor, e.g., ibrutinib, is discontinued or paused for a specific period of time or duration, e.g., during lymphodepleting therapy.

In some embodiments, the kinase inhibitor, e.g., ibrutinib, is administered multiple times in multiple doses. In some embodiments, kinase inhibitor, e.g., ibrutinib, is administered multiple times over a period of time, e.g., until a determined time point or until a particular outcome is achieved. In some embodiments, the kinase inhibitor, e.g., ibrutinib, is administered once. In some embodiments, the kinase inhibitor, e.g., ibrutinib, is administered six times daily, five times daily, four times daily, three times daily, twice daily, once daily, every other day, every three days, twice weekly, once weekly or once monthly prior to or subsequently to initiation of administration of the cell therapy (e.g. T cell therapy, such as CAR-T cell therapy). In some embodiments, the kinase inhibitor, e.g., ibrutinib is administered in multiple doses in regular intervals prior to, during, during the course of, and/or after the period of administration of the cell therapy (e.g. T cell therapy, such as CAR-T cell therapy). In some embodiments, the kinase inhibitor, e.g., ibrutinib, is administered in one or more doses in regular intervals prior to the administration of the cell therapy (e.g. T cell therapy, such as CAR-T cell therapy). In some embodiments, the kinase inhibitor, e.g., ibrutinib, is administered in one or more doses in regular intervals after the administration of the cell therapy (e.g. T cell therapy, such as CAR-T cell therapy). In some embodiments, one or more of the doses of the kinase inhibitor, e.g., ibrutinib, can occur simultaneously with the administration of a dose of the cell therapy (e.g. T cell therapy, such as CAR-T cell therapy). In some embodiments, such methods can include administration of the inhibitor prior to, simultaneously with, during, during the course of (including once and/or periodically during the course of), and/or subsequently to, the administration (e.g., initiation of administration) of the T cell therapy (e.g. CAR-expressing T cells). In some embodiments, the administrations can involve sequential or intermittent administrations of the inhibitor and/or the cell therapy, e.g. T cell therapy.

In some embodiments, the dose, frequency, duration, timing and/or order of administration of the kinase inhibitor, such as a BTK/ITK inhibitor, e.g., ibrutinib, is determined, based on particular thresholds or criteria of results of the screening step and/or assessment of treatment outcomes described herein, e.g., in Sections III and IV below.

In some embodiments, the methods involve administering the cell therapy to a subject that has been previously administered a therapeutically effective amount or one or more doses of the kinase inhibitor, e.g., ibrutinib. In some embodiments, the kinase inhibitor, e.g., ibrutinib, is administered to a subject before administering a dose of cells expressing a recombinant receptor to the subject. In some embodiments, one or more doses of the kinase inhibitor, e.g., ibrutinib, is administered at the same time as the initiation of the administration of the dose of cells. In some embodiments, the kinase inhibitor, e.g., ibrutinib, is administered after the initiation of the administration of the dose of cells. In some embodiments, the inhibitor is administered at a sufficient time prior to cell therapy so that the therapeutic effect of the combination therapy is increased. In some embodiments, the method involves administering the kinase inhibitor, such as a BTK/ITK inhibitor, e.g., ibrutinib, prior to administration of the T cell therapy. In some embodiments, the method involves administering the kinase inhibitor, e.g., ibrutinib, after administration of the T cell therapy. In some embodiments, the method involves initiating the administration of the kinase inhibitor, such as a BTK/ITK inhibitor, e.g., ibrutinib, prior to initiation of administration of the T cell therapy. In some embodiments, the kinase inhibitor, e.g., ibrutinib, is continued and/or further administered after a discontinuation or a pause during a lymphodepletion therapy, such as until after initiation of the T cell therapy. In some embodiments, the method involves continuing administration of the kinase inhibitor, such as a BTK/ITK inhibitor, e.g., ibrutinib. In some embodiments, continuing and/or further administration of the kinase inhibitor, e.g., ibrutinib, involves administration of multiple doses of the kinase inhibitor, e.g., ibrutinib. In some embodiments, the kinase inhibitor, e.g., ibrutinib, is not continued or further administered after initiation of the T cell therapy. In some embodiments, the dosage schedule comprises administering the kinase inhibitor, e.g., ibrutinib, prior to and after initiation of the T cell therapy. In some embodiments, the dosage schedule comprises administering the kinase inhibitor, e.g., ibrutinib, simultaneously with the administration of the T cell therapy.

In some aspects, the methods involve administration of the kinase inhibitor, e.g., ibrutinib, that is initiated at or at least about 3 days or a minimum of at or about 3 days prior to obtaining a sample comprising T cells from the subject, e.g., for producing a T cell therapy for administration. In some aspects, the T cell therapy is produced by a process that involves obtaining a sample comprising T cells from the subject and introducing a nucleic acid molecule encoding the CAR, such as any nucleic acid molecules described herein, e.g., in Section II.B, into a composition comprising the T cells. In some embodiments, the kinase inhibitor, e.g., ibrutinib is administered in a dosing regimen comprising administration for a period of time that extends at least until the sample is obtained from the subject. In some aspects, the administration of the kinase inhibitor, e.g., ibrutinib, is initiated at or at least about 3 days or a minimum of at or about 3 days prior to obtaining the sample from the subject, such as at least 3, 4, 5, 6 or 7 days prior to obtaining the sample from the subject, and is carried out in a dosing regimen comprising administration for a period of time that extends at least until the sample is obtained from the subject.

In some embodiments, the methods and uses involve: (1) administering to a subject having a cancer an effective amount of a kinase inhibitor, e.g., ibrutinib, or a pharmaceutically acceptable salt thereof; and (2) administering an autologous T cell therapy to the subject, said T cell therapy comprising a dose of genetically engineered T cells expressing a chimeric antigen receptor (CAR) that specifically binds to an antigen associated with a disease or disorder, e.g., any described herein. In some aspects, the T cell therapy is produced by a process comprising obtaining a sample comprising T cells from the subject and introducing a nucleic acid molecule encoding the CAR, such as any nucleic acid molecules described herein, e.g., in Section II.B, into a composition comprising the T cells. In some embodiments, the administration of the kinase inhibitor is initiated at or at least about 3 days or a minimum of at or about 3 days prior to obtaining the sample from the subject, such as at least 3, 4, 5, 6 or 7 days prior to obtaining the sample from the subject, and is carried out in a dosing regimen comprising administration for a period of time that extends at least until the sample is obtained from the subject.

In some aspects, the provided methods and uses involve administering to a subject having a cancer an effective amount of a kinase inhibitor, e.g., ibrutinib, or a pharmaceutically acceptable salt thereof, wherein the subject is a candidate for treatment or is to be treated with a T cell therapy to the subject. In some aspects, the T cell therapy comprising a dose of genetically engineered T cells expressing a chimeric antigen receptor (CAR) that specifically binds to an antigen associated with a disease or disorder, e.g., any described herein. In some aspects, the T cell therapy is produced by a process comprising obtaining a sample comprising T cells from the subject and introducing a nucleic acid molecule encoding the CAR, such as any nucleic acid molecules described herein, e.g., in Section II.B, into a composition comprising the T cells. In some aspects, the administration of the kinase inhibitor, e.g., ibrutinib, and is initiated at or at least about 3 days or a minimum of at or about 3 days prior to obtaining the sample from the subject, such as at least 3, 4, 5, 6 or 7 days prior to obtaining the sample from the subject, and is carried out in a dosing regimen comprising administration for a period of time that extends at least until the sample is obtained from the subject. In some aspects, the methods and uses also involve administering to the subject the T cell therapy, e.g., a composition comprising T cells obtained from the subject that have been introduced with a nucleic acid molecule encoding a CAR.

In some embodiments, the methods and uses involve (1) administering to a subject having a cancer an effective amount of kinase inhibitor, such as a BTK/ITK inhibitor, e.g., ibrutinib, or a pharmaceutically acceptable salt thereof; (2) administering a lymphodepleting therapy to the subject; and (3) administering an autologous T cell therapy to the subject. In some aspects, the T cell therapy includes a dose of genetically engineered T cells expressing a chimeric antigen receptor (CAR) that specifically binds to an antigen associated with a disease or disorder, e.g., any described herein. In some embodiments, the T cell therapy is produced by a process comprising obtaining a sample comprising T cells from the subject and introducing a nucleic acid molecule encoding the CAR, such as any nucleic acid molecules described herein, e.g., in Section II.B into a composition comprising the T cells. In some embodiments, the administration of the kinase inhibitor, e.g., ibrutinib, is initiated at least 3 days, such as at least 3, 4, 5, 6 or 7 days, prior to obtaining the sample and is carried out in a dosing regimen comprising administration of the kinase inhibitor up to the initiation of the lymphodepleting therapy, discontinuing or pausing administration of the kinase inhibitor during the lymphodepleting therapy and resuming or further administering the kinase inhibitor for a period that extends for at least 15 days after initiation of administration of the T cell therapy, such as at least at or about 15, 30, 60, 90, 120, 150 or 180 days or more after initiation of administration of the T cell therapy.

In some aspects, the administration of the kinase inhibitor, e.g., ibrutinib, is initiated at or at least about 3 days, at or at least about 4 days, at or at least about 5 days, at or at least 6 days, a minimum of at or about 7 days, at or least 14 days or more prior to obtaining the sample from the subject. In some embodiments, administration of the kinase inhibitor, e.g., ibrutinib, is initiated at or at least about 5 days to 7 days prior to obtaining the sample from the subject. In some aspects, the administration of the kinase inhibitor, e.g., ibrutinib, is initiated a minimum of at or about 3 days, a minimum of at or about 4 days, a minimum of at or about 5 days, a minimum of at or about 6 days, a minimum of at or about 7 days, a minimum of at or about 14 days or more prior to obtaining the sample from the subject. In some embodiments, the administration of the kinase inhibitor, e.g., ibrutinib, is initiated at or at least about 4 days, at or at least about 5 days, at or at least 6 days, a minimum of at or about 7 days, at or at least 14 days or more prior to obtaining the sample from the subject. In some embodiments, administration of the kinase inhibitor, e.g., ibrutinib, is initiated at or at least about or a minimum of at or about of 5 days to 7 days prior to obtaining the sample from the subject.

In some aspects, subsequent to initiation the administration of the kinase inhibitor, e.g., ibrutinib, and prior to the administration of the T cell therapy, the subject has been preconditioned with a lymphodepleting therapy. In some embodiments, the lymphodepleting therapy includes any described herein, e.g., in Section I.C. In some aspects, the methods and uses involve administering a lymphodepleting therapy to the subject, subsequent to initiating the administration of the kinase inhibitor, e.g., ibrutinib, and prior to the administration of the T cell therapy. In some embodiments of the methods and uses, the administration of the kinase inhibitor, e.g., ibrutinib, is discontinued during the lymphodepleting therapy. In some aspects, the dosing regimen for administering the kinase inhibitor, e.g., ibrutinib, involves administration for a period of time that extends at least until the initiation of the lymphodepleting therapy. In some aspects, the dosing regimen for administering the kinase inhibitor, e.g., ibrutinib, involves administration of the kinase inhibitor up to the initiation of the lymphodepleting therapy, discontinuing administration of the kinase inhibitor during the lymphodepleting therapy and resumed and/or further administration of the kinase inhibitor for a period that extends for at least 15 days after initiation of administration of the T cell therapy, such as at least at or about 15, 30, 60, 90, 120, 150 or 180 days or more after initiation of administration of the T cell therapy.

In some embodiments, administration of the lymphodepleting therapy is completed 2 to 7 days, such as within about 2, 3, 4, 5, 6, or 7 days, prior to initiation of the administration of the T cell therapy. In some embodiments, administration of the lymphodepleting therapy is completed within 7 days prior to initiation of the administration of the T cell therapy.

In some embodiments, the kinase inhibitor, e.g., ibrutinib, is administered prior to and/or concurrently with the administration of the cell therapy (e.g. T cell therapy, such as CAR-T cell therapy), and/or subsequently to the initiation of administration of the cell therapy. In some embodiments, the administration of the kinase inhibitor, e.g., ibrutinib is initiated from or from about 21 to at or about 49 days prior to initiation of the administration of the cell therapy, such as from or from about 25 to at or about 35 days, from at or about 28 to about 35 days, from at or about 28 to at or about about 31 days, or at or about 21, 21, 22, 23, 24, 25, 26, 27, 28, 29, 30, 31, 32, 33, 34, 35, 36, 37, 38, 39, 40, 41, 42, 43, 44, 45, 46, 47, 48 or 49 days prior to initiating the administration of the T cell therapy. In some embodiments, the administration of a kinase inhibitor, e.g., ibrutinib, is initiated at or about 14 to at or about 35 days before initiation of administration of the T cell therapy. In some embodiments, the administration of a kinase inhibitor, e.g., ibrutinib, is initiated at or about 21 to at or about 35 days before initiation of administration of the T cell therapy. In some embodiments, the administration of a kinase inhibitor, e.g., ibrutinib, is initiated at or about 21 to at or about 28 days before initiation of administration of the T cell therapy. In some embodiments, the administration of a kinase inhibitor, e.g., ibrutinib, is initiated at or about 14 days, at or about 15 days, at or about 16 days, at or about 17 days, at or about 18 days, at or about 19 days, at or about 20 days, at or about 21 days, at or about 22 days, at or about 23 days, at or about 24 days, at or about 25 days, at or about 26 days, at or about 27 days, at or about 28 days, at or about 29 days, at or about 30 days, at or about 31 days, at or about 32 days, at or about 33 days, at or about 34 days, or at or about 35 days before initiation of administration of the T cell therapy.

In some embodiments, the kinase inhibitor, such as a BTK/ITK inhibitor, e.g., ibrutinib, is administered for a duration or a period of at least or at least about 12 days, at least or about at least 14 days, at least or at least about 15 days, at least or about at least 21 days, at least or at least about 24 days, at least or about at least 28 days, at least or about at least 30 days, at least or about at least 35 days, at least or about at least 42 days, or at least or at least about 49 days prior to initiation of the administration of the cell therapy (e.g. T cell therapy, such as a CAR-T cell therapy).

In some embodiments, initiation of administration of a kinase inhibitor, e.g., ibrutinib, in the provided combination therapy methods is prior to initiation of administration of the T cell therapy, such as prior to obtaining the sample for cell engineering from the subject, e.g., at least at or about 3, 4, 5, 6, 7 days or more prior to obtaining the sample. In some aspects, the sample for cell engineering is obtained from the subject for generation or production of a composition for T cell therapy. In some aspects, the T cell therapy comprising a dose of genetically engineered T cells expressing a chimeric antigen receptor (CAR), such as a CAR that specifically binds to a CD19, wherein the T cell therapy is produced by a process comprising obtaining a sample comprising T cells from the subject and introducing a nucleic acid molecule encoding the CAR into a composition comprising the T cells. In some embodiments, initiation of administration of a kinase inhibitor, e.g., ibrutinib, in the provided combination therapy methods is carried out prior to, such as immediately prior to, or at least at or about 3, 4, 5, 6, 7 days or more prior to obtaining the sample from the subject. In some embodiments, administration of a kinase inhibitor, e.g., ibrutinib, in the provided combination therapy methods is continued after or subsequent to initiation of administration of the T cell therapy.

In some embodiments, the obtaining of a sample from the subject includes obtaining a sample that is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some aspects, T cells, such as CD4+ and/or CD8+ T cells, can be obtained from the sample from the subject. In some embodiments, the obtaining of a sample from the subject is also referred to as apheresis or leukapheresis. In some aspects, the obtaining of a sample from the subject and/or subsequent engineering of the cells, e.g., by introducing a nucleic acid molecule encoding the CAR, such as any nucleic acid molecules described herein, e.g., in Section II.B into a composition comprising the T cells in the sample obtained from the subject, are carried out according to the processes described herein, e.g., in Section II.C. In some embodiments, the sample is obtained from the subject from or from about 23 days to at or about 38 days, such as at or about 28 days to at or about 32 days, or at or about 23, 24, 25, 26, 27, 28, 29, 30, 31, 32, 33, 34, 35, 36, 37 or 38 days, prior to initiating the administration of the T cell therapy. In some embodiments, apheresis or leukapheresis from or from about 23 days to at or about 38 days, such as at or about 28 days to at or about 32 days, or at or about 23, 24, 25, 26, 27, 28, 29, 30, 31, 32, 33, 34, 35, 36, 37 or 38 days, prior to initiating the administration of the T cell therapy.

In some embodiments, the administration of the kinase inhibitor, e.g., ibrutinib, is initiated at or at least about 3 days and/or a minimum of at or about 3 days prior to obtaining the sample from the subject, such as at least at or about 3, 4, 5, 6 or 7 days or more prior to obtaining the sample from the subject (e.g., apheresis or leukapheresis). In some embodiments, administration of the kinase inhibitor, e.g., ibrutinib, is carried out in a dosing regimen comprising administration for a period of time that extends at least until the sample is obtained from the subject. In some embodiments, the administration of the kinase inhibitor, e.g., ibrutinib, is initiated from or from about 26 days to about 45 days, such as about 28 days to about 35 days, or at or about 26, 27, 28, 29, 30, 31, 32, 33, 34, 35, 36, 37, 38, 39, 40, 41, 42, 43, 44 or 45 days, prior to initiating the administration of the T cell therapy.

In some embodiments, the kinase inhibitor, such as a BTK/ITK inhibitor, e.g., ibrutinib is administered several times a day, twice a day, daily, every other day, three times a week, twice a week, or once a week during the dosing regimen. In some embodiments, the kinase inhibitor, e.g., ibrutinib is administered daily. In some embodiments the kinase inhibitor, e.g., ibrutinib is administered twice a day. In some embodiments, the kinase inhibitor, e.g., ibrutinib is administered three times a day. In other embodiments, the kinase inhibitor, e.g., ibrutinib is administered every other day. In some embodiments, the administration of the kinase inhibitor is carried out once per day on each day it is administered during the dosing regimen.

In some embodiments, an effective amount of the kinase inhibitor, such as a BTK/ITK inhibitor, e.g., ibrutinib, is administered. In some embodiments, an effective amount of the kinase inhibitor, such as a BTK/ITK inhibitor, e.g., ibrutinib is administered at each dose, when multiple doses of the kinase inhibitor is administered. In some embodiments, the effective amount of the kinase inhibitor, e.g., ibrutinib, includes any of the dosage amounts described herein, administered as a single dose, or divided over 2, 3, 4, 5 or 6 doses. In some embodiments, each dose is administered several times a day, twice a day, daily, every other day, three times a week, twice a week, or once a week. In some embodiments, the dosage amount is administered daily.

In some embodiments, the kinase inhibitor, e.g., ibrutinib, is administered in a dosage amount of from or from about 25 mg to at or about 2000 mg, from at or about 25 mg to at or about 1000 mg, from at or about 25 mg to at or about 500 mg, from at or about 25 mg to at or about 200 mg, from at or about 25 mg to at or about 100 mg, from at or about 25 mg to at or about 50 mg, from at or about 50 mg to at or about 2000 mg, from at or about 50 mg to at or about 1000 mg, from at or about 50 mg to at or about 500 mg, from at or about 50 mg to at or about 200 mg, from at or about 50 mg to at or about 100 mg, from at or about 100 mg to at or about 2000 mg, from at or about 100 mg to at or about 1000 mg, from at or about 100 mg to at or about 500 mg, from at or about 100 mg to at or about 200 mg, from at or about 200 mg to at or about 2000 mg, from at or about 200 mg to at or about 1000 mg, from at or about 200 mg to at or about 500 mg, from at or about 500 mg to at or about 2000 mg, from at or about 500 mg to at or about 1000 mg or from at or about 1000 mg to at or about 2000 mg, each inclusive. In some embodiments, the dosage amount can include any of the foregoing dosage amount administered daily.

In some embodiments, the kinase inhibitor, e.g., ibrutinib, is administered, in a dosage amount of from or from about 50 mg to at or about 560 mg, from at or about 50 mg to at or about 420 mg, from at or about 50 mg to at or about 400 mg, from at or about 50 mg to at or about 380 mg, from at or about 50 mg to at or about 360 mg, from at or about 50 mg to at or about 340 mg, from at or about 50 mg to at or about 320 mg, from at or about 50 mg to at or about 300 mg, from at or about 50 mg to at or about 280 mg, from at or about 100 mg to at or about 560 mg, from at or about 100 mg to at or about 420 mg, from at or about 100 mg to at or about 400 mg, from at or about 100 mg to at or about 380 mg, from at or about 100 mg to at or about 360 mg, from at or about 100 mg to at or about 340 mg, from at or about 100 mg to at or about 320 mg, from at or about 100 mg to at or about 300 mg, from at or about 100 mg to at or about 280 mg, from at or about 100 mg to at or about 200 mg, from at or about 140 mg to at or about 560 mg, from at or about 140 mg to at or about 420 mg, from at or about 140 mg to at or about 400 mg, from at or about 140 mg to at or about 380 mg, from at or about 140 mg to at or about 360 mg, from at or about 140 mg to at or about 340 mg, from at or about 140 mg to at or about 320 mg, from at or about 140 mg to at or about 300 mg, from at or about 140 mg to at or about 280 mg, from at or about 140 mg to at or about 200 mg, from at or about 180 mg to at or about 560 mg, from at or about 180 mg to at or about 420 mg, from at or about 180 mg to at or about 400 mg, from at or about 180 mg to at or about 380 mg, from at or about 180 mg to at or about 360 mg, from at or about 180 mg to at or about 340 mg, from at or about 180 mg to at or about 320 mg, from at or about 180 mg to at or about 300 mg, from at or about 180 mg to at or about 280 mg, from at or about 200 mg to at or about 560 mg, from at or about 200 mg to at or about 420 mg, from at or about 200 mg to at or about 400 mg, from at or about 200 mg to at or about 380 mg, from at or about 200 mg to at or about 360 mg, from at or about 200 mg to at or about 340 mg, from at or about 200 mg to at or about 320 mg, from at or about 200 mg to at or about 300 mg, from at or about 200 mg to at or about 280 mg, from at or about 220 mg to at or about 560 mg, from at or about 220 mg to at or about 420 mg, from at or about 220 mg to at or about 400 mg, from at or about 220 mg to at or about 380 mg, from at or about 220 mg to at or about 360 mg, from at or about 220 mg to at or about 340 mg, from at or about 220 mg to at or about 320 mg, from at or about 220 mg to at or about 300 mg, from at or about 220 mg to at or about 280 mg, from at or about 240 mg to at or about 560 mg, from at or about 240 mg to at or about 420 mg, from at or about 240 mg to at or about 400 mg, from at or about 240 mg to at or about 380 mg, from at or about 240 mg to at or about 360 mg, from at or about 240 mg to at or about 340 mg, from at or about 240 mg to at or about 320 mg, from at or about 240 mg to at or about 300 mg, from at or about 240 mg to at or about 280 mg, from at or about 280 mg to at or about 560 mg, from at or about 280 mg to at or about 420 mg, from at or about 300 mg to at or about 560 mg, from at or about 300 mg to at or about 420 mg, from at or about or from at or about 300 mg to at or about 400 mg, each inclusive. In some embodiments, the dosage amount can include any of the foregoing dosage amount administered daily.

In some embodiments, the kinase inhibitor, such as a BTK/ITK inhibitor, e.g., ibrutinib, is administered at a total daily dosage amount of at least or at least about 50 mg/day, 100 mg/day, 140 mg/day, 150 mg/day, 175 mg/day, 200 mg/day, 250 mg/day, 280 mg/day, 300 mg/day, 350 mg/day, 400 mg/day, 420 mg/day, 440 mg/day, 460 mg/day, 480 mg/day, 500 mg/day, 520 mg/day, 540 mg/day, 560 mg/day, 580 mg/day, 600 mg/day, 700 mg/day, 750 mg/day, 800 mg/day, 850 mg/day or 960 mg/day. In some embodiments, the inhibitor is administered in an amount of or about 420 mg/day. In some embodiments, the inhibitor is administered in an amount that is less than or less than about 560 mg/day and at least about or at least 140 mg/day. In some embodiments, the inhibitor is administered in an amount that is less than or less than about 420 mg/day and at least about or at least 280 mg/day. In some embodiments, the inhibitor is administered in an amount of at or about, or at least at or about, 140 mg/day, 280 mg/day, 420 mg/day or 560 mg/day. In some embodiments, the inhibitor is administered in an amount of at or about, or at least at or about, 420 mg/day or 560 mg/day. In some embodiments, the inhibitor is administered in an amount of no more than 140 mg/day, 280 mg/day, 420 mg/day or 560 mg/day. In some embodiments, the inhibitor is administered in an amount of no more than 420 mg/day or 560 mg/day. In some embodiments, the effective amount comprises from or from about 140 mg to at or about 840 mg per each day the kinase inhibitor, e.g., ibrutinib, is administered. In some embodiments, the effective amount comprises from or from about 140 mg to or to about 560 mg per each day the kinase inhibitor, e.g., ibrutinib is administered.

In some embodiments, the methods or uses involve: (1) administering to a subject having a cancer a kinase inhibitor, such as a BTK/ITK inhibitor, e.g., ibrutinib, or a pharmaceutically acceptable salt thereof; and (2) administering an autologous T cell therapy to the subject. In some aspects, the T cell therapy includes a dose of genetically engineered T cells expressing a chimeric antigen receptor (CAR) that specifically binds to an antigen associated with a disease or disorder, e.g., any described herein. In some embodiments, the T cell therapy is produced by a process comprising obtaining a sample comprising T cells from the subject and introducing a nucleic acid molecule encoding the CAR, such as any nucleic acid molecules described herein, e.g., in Section II.B into a composition comprising the T cells.

In some aspects, the administration of the kinase inhibitor is initiated at or at least about 5 to at or about 7 days, such as at or about 5, 6 or 7 days, prior to obtaining the sample from the subject and is carried out in a dosing regimen comprising administration of the kinase inhibitor at least until the sample is obtained from the subject and continued and/or further administration of the kinase inhibitor that extends for at or about or greater than three months after initiation of administration of the T cell therapy. In some embodiments, the kinase inhibitor, e.g., ibrutinib, is administered in an amount from or from about 140 mg to or to about 560 mg once per day each day it is administered during the dosing regimen. In some embodiments, subsequent to initiating administration of the kinase inhibitor and prior to the administration of the T cell therapy, the subject has been preconditioned with a lymphodepleting therapy. In some embodiments, the methods further include, administering a lymphodepleting therapy to the subject subsequent to the administration of the kinase inhibitor and prior to the administration of the T cell therapy. In some embodiments, the administration of the lymphodepleting therapy is completed within 7 days prior to initiation of the administration of the T cell therapy. In some embodiments, the administration of the lymphodepleting therapy is completed in at or about 2 to at or about 7 days, such as at or about 7 days, prior to initiation of the administration of the T cell therapy. In some embodiments, the dosing regimen comprises discontinuing or pausing administration of the kinase inhibitor during the lymphodepleting therapy. In some embodiments, the dosing regimen comprises resuming or further administering the kinase inhibitor after completion of the lymphodepleting therapy.

In some embodiments, the methods or uses involve: (1) administering to a subject having a cancer a kinase inhibitor, such as a BTK/ITK inhibitor, e.g., ibrutinib, or a pharmaceutically acceptable salt thereof; and (2) administering a lymphodepleting therapy to the subject; and (3) administering an autologous T cell therapy to the subject within 2 to 7 days, after completing the lymphodepleting therapy. In some aspects, the T cell therapy includes a dose of genetically engineered T cells expressing a chimeric antigen receptor (CAR) that specifically binds to an antigen associated with a disease or disorder, e.g., any described herein. In some embodiments, the T cell therapy is produced by a process comprising obtaining a sample comprising T cells from the subject and introducing a nucleic acid molecule encoding the CAR, such as any nucleic acid molecules described herein, e.g., in Section II.B into a composition comprising the T cells. In some aspects, the administration of the kinase inhibitor is initiated at or at least about 5 to 7 days, such as 7 days, prior to obtaining the sample from the subject and is carried out in a dosing regimen comprising administration of the kinase inhibitor up to the initiation of the lymphodepleting therapy, discontinuing or pausing administration of the kinase inhibitor during the lymphodepleting therapy and resuming or further administering the kinase inhibitor for a period that extends for at or greater than three months after initiation of administration of the T cell therapy, wherein the kinase inhibitor is administered in an amount from or from about 140 mg to or to about 560 mg once per day each day it is administered during the dosing regimen. In some embodiments, the administration of the kinase inhibitor per day it is administered is from or from about 280 mg to or to about 560 mg. In some embodiments, administration of the kinase inhibitor is initiated at or at least about 7 days prior to obtaining the sample from the subject.

In some embodiments, the administration of the kinase inhibitor is initiated from or from about 30 to about 40 days prior to initiating the administration of the T cell therapy; the sample is obtained from the subject from or from about 23 days to about 38 days prior to initiating the administration of the T cell therapy; and/or the lymphodepleting therapy is completed at or about 5 to 7 days, such as 7 days, prior to initiating administration of the T cell therapy.

In some embodiments, the administration of the kinase inhibitor is initiated at or about 35 days prior to initiating the administration of the T cell therapy; the sample is obtained from the subject from or from about 28 days to about 32 days prior to initiating the administration of the T cell therapy; and/or the lymphodepleting therapy is completed about 5 to about 7 days, such as 7 days, prior to initiating administration of the T cell therapy.

In some of any such embodiments in which the inhibitor of a TEC family kinase is given prior to the cell therapy (e.g. T cell therapy, such as CAR-T cell therapy), the administration of the kinase inhibitor, e.g., ibrutinib, continues at regular intervals until the initiation of the cell therapy and/or for a time after the initiation of the cell therapy.

In some of any such above embodiments, the kinase inhibitor, e.g., ibrutinib, is administered prior to and after initiation of administration of the cell therapy (e.g. T cell therapy, such as CAR-T cell therapy). In some embodiments, the kinase inhibitor, e.g., ibrutinib is administered, or is continued and/or further administered, after administration of the cell therapy. In some embodiments, the kinase inhibitor, e.g., ibrutinib is administered simultaneously, or within or within about 1 hours, 2 hours, 6 hours, 12 hours, 24 hours, 48 hours, 96 hours, 4 days, 5 days, 6 days or 7 days, 14 days, 15 days, 21 days, 24 days, 28 days, 30 days, 36 days, 42 days, 60 days, 72 days, 90 days, 120 days, 180 days, 210 days, 240 days, 270 days, 300 days, 330 days, 360 days or 720 days after initiation of administration of the cell therapy (e.g. T cell therapy). In some embodiments, the provided methods involve continued and/or further administration, such as at regular intervals, of the kinase inhibitor, e.g., ibrutinib, after initiation of administration of the cell therapy, e.g., for a duration of any of the foregoing periods after initiation of the T cell therapy.

In some embodiments, the kinase inhibitor, such as a BTK/ITK inhibitor, e.g., ibrutinib, is continued and/or further administered, such as is administered daily, for up to or up to about 1 day, up to or up to about 2 days, up to or up to about 3 days, up to or up to about 4 days, up to or up to about 5 days, up to or up to about 6 days, up to or up to about 7 days, up to or up to about 12 days, up to or up to about 14 days, up to or up to about 21 days, up to or up to about 24 days, up to or up to about 28 days, up to or up to about 30 days, up to or up to about 35 days, up to or up to about 42 days, up to or up to about 60 days or up to or up to about 90 days, up to or up to about 120 days, up to or up to about 180 days, up to or up to about 240 days, up to or up about 360 days, or up to or up to about 720 days or more after the administration of the cell therapy (e.g. T cell therapy, such as CAR-T cell therapy). In some embodiments, the kinase inhibitor, such as a BTK/ITK inhibitor, e.g., ibrutinib, is continued and/or further administered, such as is administered daily, for up to or up to about 1 week, 2 weeks, 3 weeks, 4 weeks, 1 month, 2 months, 3 months, 4 months, 5 months, 6 months, 7 months, 8 months, 9 months, 10 months, 11 months, 12 months, 1 year or 2 years or more after the administration of the cell therapy (e.g. T cell therapy, such as CAR-T cell therapy). In some embodiments, the period, e.g., for continued and/or further administration of the kinase inhibitor, e.g., ibrutinib, extends for at or about or greater than four months after the initiation of the administration of the T cell therapy. In some embodiments, the period, e.g., for continued administration of the kinase inhibitor, e.g., ibrutinib, extends for at or about or greater than five months after the initiation of the administration of the T cell therapy. In some embodiments, the period, e.g., for continued administration of the kinase inhibitor, e.g., ibrutinib, extends for at or about or greater than six months after the initiation of the administration of the T cell therapy.

In some embodiments, the dosing regimen for administering a kinase inhibitor, such as a BTK/ITK inhibitor, e.g., ibrutinib, is carried out for a period of time subsequent to initiation of administration of the T cell therapy. In some embodiments, administration of a kinase inhibitor, e.g., ibrutinib, extends for a period of more than one week after initiation of administration of the T cell therapy. In some embodiments, administration of a kinase inhibitor, e.g., ibrutinib, extends for a period of about or at least about one month after initiation of administration of the T cell therapy. In some embodiments, administration of a kinase inhibitor, e.g., ibrutinib, extends for a period of about or at least about two months after initiation of administration of the T cell therapy. In some embodiments, administration of a kinase inhibitor, e.g., ibrutinib, extends for a period of about or at least about three months after initiation of administration of the T cell therapy. In some embodiments, administration of a kinase inhibitor, e.g., ibrutinib, extends for a period of about or at least about four months after initiation of administration of the T cell therapy. In some embodiments, administration of a kinase inhibitor, e.g., ibrutinib, extends for a period of about or at least about five months after initiation of administration of the T cell therapy. In some embodiments, administration of a kinase inhibitor, e.g., ibrutinib, extends for a period of about or at least about six months after initiation of administration of the T cell therapy.

In some embodiments, administration of a kinase inhibitor, e.g., ibrutinib, extends for a period of at least three months. In some embodiments, administration of a kinase inhibitor, e.g., ibrutinib, extends for a period of at or about 90 days, at or about 100 days, at or about 105 days, at or about 110 days, at or about 115 days, at or about 120 days, at or about 125 days, at or about 130 days, at or about 135 days, at or about 140 days, at or about 145 days, at or about 150 days, at or about 155 days, at or about 160 days, at or about 165 days, at or about 170 days, at or about 175 days, at or about 180 days, at or about 185 days, at or about 190 days, at or about 195 days, at or about 200 days or more after initiation of administration of the T cell therapy.

In some embodiments, administration of a kinase inhibitor, e.g., ibrutinib, extends for a period of at or about 90 days or at or about three months after initiation of administration of the T cell therapy (e.g., CAR T cell therapy). In some embodiments, administration of a kinase inhibitor, e.g., ibrutinib, extends for a period of at or about 120 days or four months after initiation of administration of the T cell therapy (e.g., CAR T cell therapy). In some embodiments, administration of a kinase inhibitor, e.g., ibrutinib, extends for a period of at or about 150 days or five months after initiation of administration of the T cell therapy (e.g., CAR T cell therapy). In some embodiments, administration of a kinase inhibitor, e.g., ibrutinib, extends for a period of at or about 180 days or six months after initiation of administration of the T cell therapy (e.g., CAR T cell therapy).

In some aspects, the kinase inhibitor, such as a BTK/ITK inhibitor, e.g., ibrutinib, is administered, such as is administered daily, for up to or up to about 180 days after the administration of the cell therapy. In some embodiments, the continued and/or further administration of the kinase inhibitor, e.g., ibrutinib, is for a period that extends 15 days to 29 days after initiation of administration of the T cell therapy. In some embodiments, the continued and/or further administration the kinase inhibitor, e.g., ibrutinib, is for a period that extends at or about or greater than three months after initiation of administration of the T cell therapy.

In some embodiments, administration of a kinase inhibitor, e.g., ibrutinib, is ended or stopped at the end of the period (e.g. at or about 3, 4, 5, or 6 months) after initiation of administration of the T cell therapy (e.g., CAR T cell therapy) if the subject has, prior to or at or about 6 months, achieved a complete response (CR) following the treatment or the cancer (e.g. B cell malignancy) has progressed or relapsed following remission after the treatment. In some embodiments, the period is of a fixed duration such that the administration of a kinase inhibitor, e.g., ibrutinib, is continued for the period even if the subject has achieved a complete response (CR) at a time point prior to the end of the period. In some embodiments the subject is has a CR with minimal residual disease (MRD). In some embodiments, the subject has a CR that is MRD−.

In some embodiments, administration of a kinase inhibitor, e.g., ibrutinib, is continued after the end of the period, e.g. continued for longer than at or about 3, 4, 5 or 6 months after initiation of administration of the T cell therapy (e.g. CAR T cells), if the subject exhibits a partial response (PR) or stable disease (SD) after the treatment. In some embodiments, administration of a kinase inhibitor, e.g., ibrutinib, is continued for greater than 6 months after initiation of administration of the T cell therapy (e.g., CAR T cell therapy). In some embodiments, for subjects that exhibited a PR or SD at the end of the initial period, administration of a kinase inhibitor, e.g., ibrutinib, is continued until the subject has achieved a complete response (CR) following the treatment or until the cancer (e.g. B cell malignancy, such as an NHL, e.g. DLBCL) has progressed or relapsed following remission after the treatment.

In some embodiments, at the time of administering a kinase inhibitor, e.g., ibrutinib, the subject does not exhibit a severe toxicity following administration of the T cell therapy (e.g. CAR T cells). In some embodiments, the B cell malignancy is NHL, such as relapsing/refractory aggressive NHL or DLBCL. In some embodiments, the cell therapy, such as CAR-expressing T cells, comprise a chimeric antigen receptor specifically binding to a B cell antigen. In some embodiments, the B cell antigen is CD19.

In some embodiments, at the time of administering a kinase inhibitor, e.g., ibrutinib, the subject does not exhibit a severe toxicity following administration of the cell therapy. In some embodiments, the administration of a kinase inhibitor, e.g., ibrutinib, is ended or stopped, if the subject has, prior to at or about the end of the period, achieved a complete response (CR) following the treatment or the cancer, e.g. B cell malignancy, has progressed or relapsed following remission after the treatment. In some embodiments, administration of a kinase inhibitor, e.g., ibrutinib, is continued for the period even if the subject has achieved a complete response (CR) at a time point prior to the end of the period. In some embodiments, the administration of a kinase inhibitor, e.g., ibrutinib, is continued after the end of the initial period if, after initiation of administration of the T cell therapy, the subject exhibits a partial response (PR) or stable disease (SD) after the treatment. In some embodiments, the administration of a kinase inhibitor, e.g., ibrutinib, is repeated until the subject has achieved a complete response (CR) following the treatment or until the cancer, e.g. B cell malignancy, has progressed or relapsed following remission after the treatment. In some embodiments, the B cell malignancy is NHL, such as relapsing/refractory aggressive NHL or DLBCL. In some embodiments, the T cell therapy, such as CAR-expressing T cells, comprise a chimeric antigen receptor specifically binding to a B cell antigen. In some embodiments, the B cell antigen is CD19.

In some embodiments, administration of the kinase inhibitor, e.g., ibrutinib, is continued and/or resumed or further administered after (subsequent to) the initiation of the cell therapy, such as a T cell therapy (e.g., CAR-expressing T cells), for a period or a duration of time until a specific time point for termination. The time point for termination can be any defined time points described above, or at a time point where specific criteria, results or outcome is observed or achieved.

In some aspects, continued and/or further administration of the kinase inhibitor, e.g., ibrutinib, is stopped at the end of the period, if, at the end of the period, the subject exhibits a complete response (CR) following the treatment. In some embodiments, continued and/or further administration of the kinase inhibitor, e.g., ibrutinib, is stopped at the end of the period if, at the end of the period, the cancer has progressed or relapsed following remission after the treatment. In some embodiments, the period extends for from or from at or about three months to at or six months. In some embodiments, the period extends for at or about three months after initiation of administration of the T cell therapy. In some embodiments, the period extends for at or about 3 months after initiation of administration of the T cell therapy if the subject has, prior to at or about 3 months, achieved a complete response (CR) following the treatment or the cancer has progressed or relapsed following remission after the treatment. In some embodiments, the period extends for at or about 3 months after initiation of administration of the T cell therapy if the subject has at 3 months achieved a complete response (CR). In some embodiments, the period extends for at or about six months after initiation of administration of the T cell therapy. In some embodiments, the period extends for at or about 6 months after initiation of administration of the T cell therapy if the subject has, prior to at or about 6 months, achieved a complete response (CR) following the treatment or the cancer has progressed or relapsed following remission after the treatment. In some embodiments, the period extends for at or about 6 months after initiation of administration of the T cell therapy if the subject has at 6 months achieved a complete response (CR). In some embodiments, the continued and/or further administration is continued for the duration of the period even if the subject has achieved a complete response (CR) at a time point prior to the end of the period. In some embodiments, the subject achieves a complete response (CR) at a time during the period and prior to the end of the period.

In some embodiments, the methods and uses also involve continued and/or further administration after the end of the period, if, at the end of the period, the subject exhibits a partial response (PR) or stable disease (SD). In some embodiments, the continued and/or further administration is continued for greater than six months if, at or about six months, the subject exhibits a partial response (PR) or stable disease (SD) after the treatment. In some embodiments, the continued and/or further administration is continued until the subject has achieved a complete response (CR) following the treatment or until the cancer has progressed or relapsed following remission after the treatment.

In some embodiments, administration of a kinase inhibitor, e.g., ibrutinib, is continued, until or after peak or maximum level of the cells of the T cell therapy is detectable in the blood of the subject. In some cases, initiation of administration a kinase inhibitor, e.g., ibrutinib, is carried out at or within a week, such as within 1, 2 or 3 days after (i) a time in which peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (iv) the subject exhibits increased tumor burden as compared to tumor burden at a time prior to or after administration of the cells and prior to initiation of administration of a kinase inhibitor, e.g., ibrutinib. In certain aspects, the provided methods are carried out to enhance, increase or potentiate T cell therapy in subjects in which a peak response to the T cell therapy has been observed but in which the response, e.g. presence of T cells and/or reduction in tumor burden, has become reduced or is no longer detectable.

In some embodiments, at the time at which the subject is first administered a kinase inhibitor, e.g., ibrutinib, and/or at any subsequent time after initiation of the administration, the subject does not exhibit a sign or symptom of a severe toxicity, such as severe cytokine release syndrome (CRS) or severe toxicity. In some embodiments, the administration of a kinase inhibitor, e.g., ibrutinib, is at a time at which the subject does not exhibit a sign or symptom of severe CRS and/or does not exhibit grade 3 or higher CRS, such as prolonged grade 3 CRS or grade 4 or 5 CRS. In some embodiments, the administration of a kinase inhibitor, e.g., ibrutinib, is at a time at which the subject does not exhibit a sign or symptom of severe neurotoxicity and/or does not exhibit grade 3 or higher neurotoxicity, such as prolonged grade 3 neurotoxicity or grade 4 or grade 5 neurotoxicity. In some aspects, between the time of the initiation of the administration of the T cell therapy and the time of the administration of a kinase inhibitor, e.g., ibrutinib, the subject has not exhibited severe CRS and/or has not exhibited grade 3 or higher CRS, such as prolonged grade 3 CRS or grade 4 or 5 CRS. In some instances, between the time of the initiation of the administration of the T cell therapy and the time of the administration of a kinase inhibitor, e.g., ibrutinib, the subject has not exhibited severe neurotoxicity and/or does not exhibit grade 3 or higher neurotoxicity, such as prolonged grade 3 neurotoxicity or grade 4 or 5 neurotoxicity.

In some embodiments, a kinase inhibitor, e.g., ibrutinib, is administered in an amount that achieves a maximum concentration (Cₘₐₓ) of a kinase inhibitor, e.g., ibrutinib, in the blood, after oral administration, such as within at or about 1, 2, 3, 4, 5, 6, 7, 8, 9, 10, 11 or 12 hours after oral administration, in a range of at or about 10 ng/mL to at or about 100 ng/mL, at or about 20 ng/mL to at or about 100 ng/mL, at or about 30 ng/mL to at or about 100 ng/mL, at or about 40 ng/mL to at or about 100 ng/mL, at or about 50 ng/mL to at or about 100 ng/mL, at or about 60 ng/mL to at or about 100 ng/mL, at or about 70 ng/mL to at or about 100 ng/mL, at or about 80 ng/mL to at or about 100 ng/mL, at or about 90 ng/mL to at or about 100 ng/mL, at or about 10 ng/mL to at or about 80 ng/mL, at or about 20 ng/mL to at or about 80 ng/mL, at or about 30 ng/mL to at or about 80 ng/mL, at or about 40 ng/mL to at or about 80 ng/mL, at or about 50 ng/mL to at or about 80 ng/mL, at or about 60 ng/mL to at or about 80 ng/mL, at or about 70 ng/mL to at or about 80 ng/mL, at or about 10 ng/mL to at or about 60 ng/mL, at or about 20 ng/mL to at or about 60 ng/mL, at or about 30 ng/mL to at or about 60 ng/mL, at or about 40 ng/mL to at or about 60 ng/mL, at or about 50 ng/mL to at or about 60 ng/mL, at or about 10 ng/mL to at or about 40 ng/mL, at or about 20 ng/mL to at or about 40 ng/mL, at or about 30 ng/mL to at or about 40 ng/mL, such as at or about 10 ng/mL, 20 ng/mL, 30 ng/mL, 40 ng/mL, 50 ng/mL, 60 ng/mL, 70 ng/mL, 80 ng/mL, 90 ng/mL, or 100 ng/mL. In some embodiments, a kinase inhibitor, such as a BTK/ITK inhibitor, e.g., ibrutinib, is administered at an amount that achieves a Cₘₐₓ of a kinase inhibitor, e.g., ibrutinib, in the blood at about or at least about 10 ng/mL, 20 ng/mL, 30 ng/mL, 40 ng/mL, 50 ng/mL, 60 ng/mL, 70 ng/mL, 80 ng/mL, 90 ng/mL, or 100 ng/mL. In some embodiments, a kinase inhibitor, such as a BTK/ITK inhibitor, e.g., ibrutinib, is administered at an amount that maintains the Cₘₐₓ in the range for at least about 30 minutes or 1, 2, 3, 4, 5, 6, 7, 8, 9, 10, 11 or 12 hours

In some embodiments, administration of a kinase inhibitor, such as a BTK/ITK inhibitor, e.g., ibrutinib, is carried out in a dosing regimen comprising administering the kinase inhibitor, e.g., ibrutinib, in an amount from or from about 140 mg to about 560 mg per day for a period of about or greater than three months (e.g., for a period of at or about three months, four months, five months, or six months) after initiation of administration of the T cell therapy (e.g., CAR T cell therapy). In some embodiments, the administration of a kinase inhibitor, e.g., ibrutinib, is initiated greater than about 14 to about 35 days (e.g., about 28 to about 35 days, e.g., at or about 31, 32, 33, 34 or 35 days) after initiation of the administration of the cell therapy. In some embodiments, at the time of administering a kinase inhibitor, e.g., ibrutinib, the subject does not exhibit a severe toxicity following administration of the cell therapy. In some embodiments, the B cell malignancy is NHL, such as relapsing/refractory aggressive NHL or DLBCL. In some embodiments, administration of a kinase inhibitor, e.g., ibrutinib, is ended or stopped at or about 6 months after initiation of administration of the T cell therapy if the subject has, prior to at or about 6 months, achieved a complete response (CR) following the treatment or the cancer, e.g. B cell malignancy, has progressed or relapsed following remission after the treatment. In some embodiments, the cycling regimen is continued for the entire period even if the subject has achieved a complete response (CR) at a time point prior to the end of the period. In some embodiments, the administration of a kinase inhibitor, e.g., ibrutinib, is further continued after the end of the period, such as is continued for greater than 6 months after initiation of administration of the cell therapy, if, at or about six months, the subject exhibits a partial response (PR) or stable disease (SD) after the treatment. In some embodiments, the administration of a kinase inhibitor, e.g., ibrutinib, is continued until the subject has achieved a complete response (CR) following the treatment or until the cancer, e.g. B cell malignancy, has progressed or relapsed following remission after the treatment. In some embodiments, the cell therapy, such as CAR-expressing T cells, comprise a chimeric antigen receptor specifically binding to a B cell antigen. In some embodiments, the B cell antigen is CD19.

In some cases, the dosing regimen can be interrupted at any time, and/or for one or more times. In some cases, the dosing regimen is interrupted or modified if the subject develops one or more adverse event, dose-limiting toxicity (DLT), neutropenia or febrile neutropenia, thrombocytopenia, cytokine release syndrome (CRS) and/or neurotoxicity (NT), such as those as described in Section IV. In some embodiments, the amount of a kinase inhibitor, e.g., ibrutinib, for each administration or per day in certain days of a week is altered after the subject develops one or more adverse event, dose-limiting toxicity (DLT), neutropenia or febrile neutropenia, thrombocytopenia, cytokine release syndrome (CRS) and/or neurotoxicity (NT), such as those as described in Section IV.

In some embodiments, the kinase inhibitor, e.g., ibrutinib is administered daily for a cycle of 7, 14, 21, 28, 35, or 42 days. In some embodiments, the kinase inhibitor, e.g., ibrutinib is administered twice a day for a cycle of 7, 14, 21, 28, 35, or 42 days. In some embodiments, the kinase inhibitor, e.g., ibrutinib is administered three times a day for a cycle of 7, 14, 21, 28, 35, or 42 days. In some embodiments, the kinase inhibitor, e.g., ibrutinib is administered every other day for a cycle of 7, 14, 21, 28, 35, or 42 days. In some embodiments, the kinase inhibitor, e.g., ibrutinib is administered in multiple cycles, e.g., more than one cycle. In some aspects, each cycle can be of approximately 7, 14, 21, 28, 35, or 42 days. In some embodiments, the kinase inhibitor, e.g., ibrutinib is administered for 1, 2, 3, 4, 5, 6, 7, 8, 9, 10, 11, 12, 13, 14, 15, 16, 17, 18, 19, 20, 21, 22, 23, or 24 or more cycles.

In some embodiments of the methods provided herein, the kinase inhibitor, e.g., ibrutinib, and the cell therapy (e.g. T cell therapy, such as CAR-T cell therapy) are administered simultaneously or near simultaneously.

In some embodiments, the kinase inhibitor, e.g., ibrutinib, is administered in a dosage amount of from or from about 0.2 mg per kg body weight of the subject (mg/kg) to 200 mg/kg, 0.2 mg/kg to 100 mg/kg, 0.2 mg/kg to 50 mg/kg, 0.2 mg/kg to 10 mg/kg, 0.2 mg/kg to 1.0 mg/kg, 1.0 mg/kg to 200 mg/kg, 1.0 mg/kg to 100 mg/kg, 1.0 mg/kg to 50 mg/kg, 1.0 mg/kg to 10 mg/kg, 10 mg/kg to 200 mg/kg, 10 mg/kg to 100 mg/kg, 10 mg/kg to 50 mg/kg, 50 mg/kg to 200 mg/kg, 50 mg/kg to 100 mg/kg or 100 mg/kg to 200 mg/kg. In some embodiments, the inhibitor is administered at a dose of about 0.2 mg per kg body weight of the subject (mg/kg) to 50 mg/kg, 0.2 mg/kg to 25 mg/kg, 0.2 mg/kg to 10 mg/kg, 0.2 mg/kg to 5 mg/kg, 0.2 mg/kg to 1.0 mg/kg, 1.0 mg/kg to 50 mg/kg, 1.0 mg/kg to 25 mg/kg, 1.0 mg/kg to 10 mg/kg, 1.0 mg/kg to 5 mg/kg, 5 mg/kg to 50 mg/kg, 5 mg/kg to 25 mg/kg, 5 mg/kg to 10 mg/kg, or 10 mg/kg to 25 mg/kg.

In any of the aforementioned embodiments, the ibrutinib may be administered orally.

In some embodiments, dosages, such as daily dosages, are administered in one or more divided doses, such as 2, 3, or 4 doses, or in a single formulation. The inhibitor can be administered alone, in the presence of a pharmaceutically acceptable carrier, or in the presence of other therapeutic agents.

One skilled in the art will recognize that higher or lower dosages of the inhibitor could be used, for example depending on the particular agent and the route of administration. In some embodiments, the inhibitor may be administered alone or in the form of a pharmaceutical composition wherein the compound is in admixture or mixture with one or more pharmaceutically acceptable carriers, excipients, or diluents. In some embodiments, the inhibitor may be administered either systemically or locally to the organ or tissue to be treated. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes. In some embodiments, the route of administration is oral, parenteral, rectal, nasal, topical, or ocular routes, or by inhalation. In some embodiments, the inhibitor is administered orally. In some embodiments, the inhibitor is administered orally in solid dosage forms, such as capsules, tablets and powders, or in liquid dosage forms, such as elixirs, syrups and suspensions.

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. If symptoms have been alleviated to an appropriate level, treatment may cease. Patients may, however, require intermittent treatment on a long-term basis upon any recurrence of symptoms. Patients may also require chronic treatment on a long-term basis.

B. Administration of Cell Therapy

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors. Exemplary engineered cells for administering as a cell therapy in the provided methods are described in Section II.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4 vs. CD8 T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

I. Compositions and Formulations

In some embodiments, the dose of cells of the cell therapy, such as a T cell therapy comprising cells engineered with a recombinant antigen receptor, e.g. CAR or TCR, is provided as a composition or formulation, such as a pharmaceutical composition or formulation. Such compositions can be used in accord with the provided methods and/or with the provided articles of manufacture or compositions, such as in the treatment of a B cell malignancy.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the cell therapy, such as engineered T cells (e.g. CAR T cells), are formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat the disease or condition, such as a therapeutically effective or prophylactically effective amount. Therapeutic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

2. Dosing

In some embodiments, a dose of cells is administered to subjects in accord with the provided methods, and/or with the provided articles of manufacture or compositions. In some embodiments, the size or timing of the doses is determined as a function of the particular disease or condition (e.g., cancer, e.g., B cell malignancy) in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about one million to at or about 100 billion cells and/or that amount of cells per kilogram of body weight, such as, e.g., 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges and/or per kilogram of body weight. Dosages may vary depending on attributes particular to the disease or disorder and/or patient and/or other treatments.

In some embodiments, the dose of cells comprises from at or about 1×10⁵ to at or about 5×10⁸ total CAR-expressing T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells.

In some embodiments, the dose of cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the dose of cells is a flat dose of cells or fixed dose of cells such that the dose of cells is not tied to or based on the body surface area or weight of a subject.

In some embodiments, for example, where the subject is a human, the dose includes fewer than at or about 5×10⁸ total recombinant receptor (e.g., CAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸ 2×10⁸, 3×10⁸, 4×10⁸ or 5×10⁸ total such cells, or the range between any two of the foregoing values. In some embodiments, where the subject is a human, the dose includes between at or about 1×10⁶ and at or 3×10⁸ total recombinant receptor (e.g., CAR)-expressing cells, e.g., in the range of at or about 1×10⁷ to at or about 2×10⁸ such cells, such as at or about 1×10⁷, 5×10⁷, 1×10⁸ or 1.5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁵ to at or about 5×10⁸ total recombinant receptor (e.g. CAR)-expressing T cells or total T cells, from at or about 1×10⁵ to at or about 1×10⁸ total recombinant receptor (e.g. CAR)-expressing T cells or total T cells, from at or about 5×10⁵ to at or about 1×10⁷ total recombinant receptor (e.g. CAR)-expressing T cells or total T cells, or from at or about 1×10⁶ to at or about 1×10⁷ total recombinant receptor (e.g. CAR)-expressing T cells or total T cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between at or about 1×10⁶ and at or about 1×10⁸ total recombinant receptor (e.g., CAR)-expressing CD8+ cells, e.g., in the range of at or about 5×10⁶ to at or about 1×10⁸ such cells, such cells at or about 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁷ to at or about 0.75×10⁸ total recombinant receptor-expressing CD8+ T cells, from at or about 1×10⁷ to at or about 2.5×10⁷ total recombinant receptor-expressing CD8+ T cells, from at or about 1×10⁷ to at or about 0.75×10⁸ total recombinant receptor-expressing CD8+ T cells, each inclusive. In some embodiments, the dose of cells comprises the administration of at or about 1×10⁷, 2.5×10⁷, 5×10⁷, 7.5×10⁷, 1×10⁸, 1.5×10⁸, or 5×10⁸ total recombinant receptor-expressing CD8+ T cells.

In some embodiments, for example, where the subject is human, the CD4+ T cells of the dose, including in a dose including CD4+ and CD8+ T cells, includes between at or about 1×10⁶ and at or about 1×10⁸ total recombinant receptor (e.g., CAR)-expressing CD4+ cells, e.g., in the range of at or about 5×10⁶ to 1×10⁸ such cells, such at or about 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁷ to at or about 0.75×10⁸ total recombinant receptor-expressing CD4+ T cells, from at or about 1×10⁷ to at or about 2.5×10⁷ total recombinant receptor-expressing CD4+ T cells, from at or about 1×10⁷ to at or about 0.75×10⁸ total recombinant receptor-expressing CD4+ T cells, each inclusive. In some embodiments, the dose of cells comprises the administration of at or about 1×10⁷, 2.5×10⁷, 5×10⁷ 7.5×10⁷, 1×10⁸, 1.5×10⁸, or 5×10⁸ total recombinant receptor-expressing CD4+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered from at or about 0 to at or about 12 hours apart, from at or about 0 to at or about 6 hours apart or from at or about 0 to at or about 2 hours apart. In some embodiments, the initiation of administration of the first composition and the initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart. In some embodiments, the initiation and/or completion of administration of the first composition and the completion and/or initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart.

In some embodiments, the first composition and the second composition is mixed prior to the administration into the subject. In some embodiments, the first composition and the second composition is mixed shortly (e.g., within at or about 6 hours, 5 hours, 4 hours, 3 hours, 2 hours, 1.5 hours, 1 hour, or 0.5 hour) before the administration, In some embodiments, the first composition and the second composition is mixed immediately before the administration.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose.] In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

C. Lymphodepleting Treatment

In some aspects, the provided methods can further include administering one or more lymphodepleting therapies, such as prior to or simultaneous with initiation of administration of the immunotherapy, such as a T cell therapy (e.g. CAR-expressing T cells). In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54 (2002); Rosenberg et al., Clin Cancer Res, 17(13):4550-4557 (2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119: 2709-2720 (2012); Kalos et al., Sci Transl Med, 3(95):95ra73 (2011); Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the initiation of the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the initiation of the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the initiation of administration of the dose of cells. In some embodiments, the subject is administered a preconditioning agent between 2 and 7, inclusive, such as at 2, 3, 4, 5, 6, or 7 days prior to the initiation of the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and at or about 500 mg/m², such as between or between about 200 mg/m² and at or about 400 mg/m², or between at or about 250 mg/m² and at or about 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and at or about 100 mg/m², such as between or between about 10 mg/m² and 75 mg/m², between at or about 15 mg/m² and at or about 50 mg/m², between at or about 20 mg/m² and at or about 40 mg/m², between at or about 24 mg/m² and at or about 35 mg/m², 20 mg/m² and at or about 30 mg/m², or between at or about 24 mg/m² and at or about 26 mg/m². In some instances, the subject is administered 25 mg/m² of fludarabine. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells. In some embodiments, the subject is administered about 300 mg/m² cyclophosphamide and about 30 mg/m² fludarabine each daily for 3 days. In some embodiments, the preconditioning administration schedule ends between 2 and 7, inclusive, such as at 2, 3, 4, 5, 6, or 7, days prior to the initiation of the administration of the dose of cells.

In one exemplary dosage regime, prior to receiving the first dose, subjects receive a kinase inhibitor 1 day before the administration of cells and an lymphodepleting preconditioning chemotherapy of cyclophosphamide and fludarabine (CY/FLU), which is administered at least two days before the first dose of CAR-expressing cells and generally no more than 7 days before administration of cells. In some cases, for example, cyclophosphadmide is given from 24 to 27 days after the administration of the BTK inhibitor. After preconditioning treatment, subjects are administered the dose of CAR-expressing T cells as described above.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., CAR-expressing cells, such as CAR-expressing T cells) in the subject. Therefore, in some embodiments, the dose of preconditioning agent given in the method which is a combination therapy with the BTK inhibitor and cell therapy is higher than the dose given in the method without the BTK inhibitor.

### II. Cell Therapy and Engineering Cells

In some embodiments, the cell therapy (e.g., T cell therapy) for use in accord with the provided combination therapy methods includes administering engineered cells expressing recombinant receptors designed to recognize and/or specifically bind to antigens associated with the disease or condition, such as a cancer, e.g., B cell malignancy. In some embodiments, binding to the antigen results in a response, such as an immune response against such antigens. In some embodiments, the cells contain or are engineered to contain an engineered receptor or recombinant receptor, e.g., an engineered antigen receptor, such as a chimeric antigen receptor (CAR). The recombinant receptor, such as a CAR, generally includes an extracellular antigen (or ligand) binding domain linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Chimeric Antigen Receptors

In some embodiments of the provided methods and uses, the engineered cells, such as T cells, express a chimeric receptors, such as a chimeric antigen receptors (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the engineered cells, such as T cells, express a recombinant receptor such as a chimeric antigen receptor (CAR) with specificity for a particular antigen (or marker or ligand), such as an antigen expressed on the surface of a particular cell type. In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In particular aspects, the antigen is CD19. In some embodiments, any of such antigens are antigens expressed on human B cells.

The chimeric receptors, such as CARs, generally include an extracellular antigen binding domain that is an antigen-binding portion or portions of an antibody molecule. In some embodiments, the antigen-binding domain is a portion of an antibody molecule, generally a variable heavy (V_{H}) chain region and/or variable light (V_{L}) chain region of the antibody, e.g., an scFv antibody fragment. In some embodiments, the antigen-binding domain is a single domain antibody (sdAb), such as sdFv, nanobody, V_{H}H and V_{NAR}. In some embodiments, an antigen-binding fragment comprises antibody variable regions joined by a flexible linker.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NO: 38 and 39, respectively, and CDR-H3 set forth in SEQ ID NO: 40 or 54 and CDR-L1 set forth in SEQ ID NO: 35 and CDR-L2 set forth in SEQ ID NO: 36 or 55 and CDR-L3 sequences set forth in SEQ ID NO: 37 or 56. In some embodiments, the FMC63 antibody comprises the heavy chain variable region (V_{H}) comprising the amino acid sequence of SEQ ID NO: 41 and the light chain variable region (V_{L}) comprising the amino acid sequence of SEQ ID NO: 42.

In some embodiments, the scFv comprises a variable light chain containing the CDR-L1 sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of FMC63 set forth in SEQ ID NO:41 and a variable light chain region of FMC63 set forth in SEQ ID NO:42, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:59. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:57 or a sequence that exhibits at least 85%, 86%, 87%, 88%, 89%, 90%, 91%, 92%, 93%, 94%, 95%, 96%, 97%, 98%, or 99% sequence identity to SEQ ID NO:57. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the antigen-binding domain includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 47-49, respectively, and CDR-L1, CDR-L2 and CDR-L3 sequences set forth in SEQ ID NOS: 44-46, respectively. In some embodiments, the SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51. In some embodiments, the svFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:50 and a variable light chain region of SJ25C1 set forth in SEQ ID NO:51, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody, V_{H}H or V_{NAR}) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. In some aspects, the CAR is a bispecific CAR, e.g., containing two antigen-binding domains with different specificities.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

The terms “complementarity determining region,” and “CDR,” synonymous with “hypervariable region” or “HVR,” are known, in some cases, to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known, in some cases, to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 2, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 2
Boundaries of CDRs according to various numbering schemes.

| CDR | Kabat | Chothia | AbM | Contact |
|---|---|---|---|---|
| CDR-L1 | L24 . . . L34 | L24 . . . L34 | L24 . . . L34 | L30 . . . L36 |
| CDR-L2 | L50 . . . L56 | L50 . . . L56 | L50 . . . L56 | L46 . . . L55 |
| CDR-L3 | L89 . . . L97 | L89 . . . L97 | L89 . . . L97 | L89 . . . L96 |
| CDR-H1 | H31 . . . H35B | H26 . . . H32 . . . 34 | H26 . . . H35B | H30 . . . H35B |
| (Kabat Numbering¹) | | | | |
| CDR-H1 | H31 . . . H35 | H26 . . . H32 | H26 . . . H35 | H30 . . . H35 |
| (Chothia Numbering²) | | | | |
| CDR-H2 | H50 . . . H65 | H52 . . . H56 | H50 . . . H58 | H47 . . . H58 |
| CDR-H3 | H95 . . . H102 | H95 . . . H102 | H95 . . . H102 | H93 . . . H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273,927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some aspects, the recombinant receptor, e.g., a chimeric antigen receptor, includes an extracellular portion containing one or more ligand- (e.g., antigen-) binding domains, such as an antibody or fragment thereof, and one or more intracellular signaling region or domain (also interchangeably called a cytoplasmic signaling domain or region). In some aspects, the recombinant receptor, e.g., CAR, further includes a spacer and/or a transmembrane domain or portion. In some aspects, the spacer and/or transmembrane domain can link the extracellular portion containing the ligand- (e.g., antigen-) binding domain and the intracellular signaling region(s) or domain(s)

In some embodiments, the recombinant receptor such as the CAR, further includes a spacer, which may be or include at least a portion of an immunoglobulin constant region or variant or modified version thereof, such as a hinge region, e.g., an IgG4 hinge region, and/or a C_{H}1/CL and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687.

In some embodiments, the spacer contains only a hinge region of an IgG, such as only a hinge of IgG4 or IgG1, such as the hinge only spacer set forth in SEQ ID NO: 1, and encoded by the sequence set forth in SEQ ID NO: 2. In some embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 3. In some embodiments, the spacer the spacer is an Ig hinge, e.g., an IgG4 hinge, linked to a C_{H}3 domain only, such as set forth in SEQ ID NO: 4. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5, 27-34 or 58, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof. The extracellular domain and transmembrane can be linked directly or indirectly. In some embodiments, the extracellular domain and transmembrane are linked by a spacer, such as any described herein.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8. In some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the recombinant receptor, e.g., CAR, includes at least one intracellular signaling component or components, such as an intracellular signaling region or domain. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the intracellular signaling regions, e.g., comprising intracellular domain or domains, include the cytoplasmic sequences of a region or domain that is involved in providing costimulatory signal.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8alpha, CD8beta, CD4, CD25, or CD16. For example, in some aspects, the CAR or other chimeric receptor includes a chimeric molecule between CD3-zeta (CD3-ζ) or Fc receptor γ and CD8alpha, CD8beta, CD4, CD25 or CD16.

In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40 (CD134), CD27, DAP10, DAP12, ICOS and/or other costimulatory receptors. In some embodiments, the CAR includes a costimulatory region or domain of CD28 or 4-1BB, such as of human CD28 or human 4-1BB.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some aspects, the same CAR includes both the primary (or activating) cytoplasmic signaling regions and costimulatory signaling components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. An exemplary T2A linker sequence comprises the sequence of amino acids set forth in SEQ ID NO: 6 or 17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 17.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the chimeric antigen receptor includes an extracellular portion containing the antibody or fragment described herein. In some aspects, the chimeric antigen receptor includes an extracellular portion containing the antibody or fragment described herein and an intracellular signaling domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3ζ) chain. In some embodiments, the CD3-zeta chain is a human CD3-zeta chain. In some embodiments, the intracellular signaling region further comprises a CD28 and CD137 (4-1BB, TNFRSF9) co-stimulatory domains, linked to a CD3 zeta intracellular domain. In some embodiments, the CD28 is a human CD28. In some embodiments, the 4-1BB is a human 4-1BB. In some embodiments, the chimeric antigen receptor includes a transmembrane domain disposed between the extracellular domain and the intracellular signaling region. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. For example, in some embodiments, the CAR includes an antibody such as an antibody fragment, including scFvs, e.g. specific for CD19 such as any described above, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain.

In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer. In some embodiments, the CAR includes an antibody or fragment, such as scFv, e.g. specific for CD19 such as any described above, a spacer such as any of the Ig-hinge containing spacers, a CD28-derived transmembrane domain, a 4-1BB-derived intracellular signaling domain, and a CD3 zeta-derived signaling domain.

B. Nucleic Acids, Vectors and Methods for Genetic Engineering

In some embodiments, the cells, e.g., T cells, are genetically engineered to express a recombinant receptor. In some embodiments, the engineering is carried out by introducing polynucleotides that encode the recombinant receptor. Also provided are polynucleotides encoding a recombinant receptor, and vectors or constructs containing such nucleic acids and/or polynucleotides.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO:25 and encoded by the nucleotide sequence set forth in SEQ ID NO:24. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 25 and encoded by the nucleotide sequence set forth in SEQ ID NO:24, or the CD8 alpha signal peptide set forth in SEQ ID NO:26.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, e.g., a recombinant receptor and a marker, each of the polypeptide chains can be encoded by a separate nucleic acid molecule. For example, two separate nucleic acids are provided, and each can be individually transferred or introduced into the cell for expression in the cell. In some embodiments, the nucleic acid encoding the recombinant receptor and the nucleic acid encoding the marker are operably linked to the same promoter and are optionally separated by an internal ribosome entry site (IRES), or a nucleic acid encoding a self-cleaving peptide or a peptide that causes ribosome skipping, which optionally is a T2A, a P2A, an E2A or an F2A. In some embodiments, the nucleic acids encoding the marker and the nucleic acid encoding the recombinant receptor are operably linked to two different promoters. In some embodiments, the nucleic acid encoding the marker and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products ((e.g. encoding the marker and encoding the recombinant receptor) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

Any of the recombinant receptors described herein can be encoded by polynucleotides containing one or more nucleic acid sequences encoding recombinant receptors, in any combinations or arrangements. For example, one, two, three or more polynucleotides can encode one, two, three or more different polypeptides, e.g., recombinant receptors. In some embodiments, one vector or construct contains a nucleic acid sequence encoding marker, and a separate vector or construct contains a nucleic acid sequence encoding a recombinant receptor, e.g., CAR. In some embodiments, the nucleic acid encoding the marker and the nucleic acid encoding the recombinant receptor are operably linked to two different promoters. In some embodiments, the nucleic acid encoding the recombinant receptor is present downstream of the nucleic acid encoding the marker.

In some embodiments, the vector backbone contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16.

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy, 2014 Apr. 3. doi: 10.1038/gt.2014.25; Carlens et al. (2000) Exp. Hematol., 28(10): 1137-46; Alonso-Camino et al. (2013) Mol. Ther. Nucl. Acids., 2, e93; Park et al., Trends Biotechnol., 2011 November 29(11): 550-557.

In some embodiments, the viral vector is an adeno-associated virus (AAV).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV) or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

C. Cells and Preparation of Cells for Genetic Engineering

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood, 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (fluorescence activated cell sorting, FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° C. per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating or stimulating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/mL). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### III. Exemplary Treatment Outcomes and Methods for Assessing Same

In some embodiments of the methods, compositions, combinations, uses, kits and articles of manufacture provided herein, the provided combination therapy results in one or more treatment outcomes, such as a feature associated with any one or more of the parameters associated with the therapy or treatment, as described below. In some embodiments, the method includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells, e.g. T cell therapy, before, during, or after administering the combination therapy provided herein.

In some embodiments, the combination therapy can further include one or more screening steps to identify subjects for treatment with the combination therapy and/or continuing the combination therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the combination therapy provided herein.

In some embodiments, any of the screening steps and/or assessment of treatment of outcomes described herein can be used prior to, during, during the course of, or subsequent to administration of one or more steps of the provided combination therapy, e.g., administration of the T cell therapy (e.g. CAR-expressing T cells), and/or a kinase inhibitor, such as a BTK/ITK inhibitor, e.g., ibrutinib. In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of administration of one or more steps of the provided combination therapy, for example, to screen and identify patients suitable and/or susceptible to receive the combination therapy. In some embodiments, the assessment is performed during, during the course of, or subsequent to administration of one or more steps of the provided combination therapy, for example, to assess the intermediate or final treatment outcome, e.g., to determine the efficacy of the treatment and/or to determine whether to continue or repeat the treatments and/or to determine whether to administer the remaining steps of the combination therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g. CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known in the art, and can be performed one or more times, e.g., prior to, during, during the course of, or subsequently to administration of one or more steps of the combination therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the persistence, expansion, and/or presence of recombinant receptor-expressing, e.g., CAR-expressing, cells in the subject following administration of the dose of cells in the method with a kinase inhibitor, such as a BTK/ITK inhibitor, e.g., ibrutinib, is greater as compared to that achieved via a method without the administration of a kinase inhibitor, e.g., ibrutinib. In some embodiments, expansion and/or persistence in the subject of the administered T cell therapy, e.g., CAR-expressing T cells is assessed as compared to a method in which the T cell therapy is administered to the subject in the absence of a kinase inhibitor, e.g., ibrutinib. In some embodiments, the methods result in the administered T cells exhibiting increased or prolonged expansion and/or persistence in the subject as compared to a method in which the T cell therapy is administered to the subject in the absence of a kinase inhibitor, e.g., ibrutinib.

In some embodiments, the administration of a kinase inhibitor, e.g., ibrutinib, decreases disease burden, e.g., tumor burden, in the subject as compared to a method in which the dose of cells expressing the recombinant receptor is administered to the subject in the absence of a kinase inhibitor, e.g., ibrutinib. In some embodiments, the administration of a kinase inhibitor, e.g., ibrutinib, decreases blast marrow in the subject as compared to a method in which the dose of cells expressing the recombinant receptor is administered to the subject in the absence of a kinase inhibitor, e.g., ibrutinib. In some embodiments, the administration of a kinase inhibitor, e.g., ibrutinib, results in improved clinical outcomes, e.g., objective response rate (ORR), progression-free survival (PFS) and overall survival (OS), compared to a method in which the dose of cells expressing the recombinant receptor is administered to the subject in the absence of a kinase inhibitor, e.g., ibrutinib.

In some embodiments, the subject can be screened prior to the administration of one or more steps of the combination therapy. For example, the subject can be screened for characteristics of the disease and/or disease burden, e.g., tumor burden, prior to administration of the combination therapy, to determine suitability, responsiveness and/or susceptibility to administering the combination therapy. In some embodiments, the screening step and/or assessment of treatment outcomes can be used to determine the dose, frequency, duration, timing and/or order of the combination therapy provided herein.

In some embodiments, the subject can be screened after administration of one of the steps of the combination therapy, to determine and identify subjects to receive the remaining steps of the combination therapy and/or to monitor efficacy of the therapy. In some embodiments, the number, level or amount of administered T cells and/or proliferation and/or activity of the administered T cells is assessed prior to administration and/or after administration of a kinase inhibitor, such as a BTK/ITK inhibitor, e.g., ibrutinib.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. For example, in some embodiments, a fold change, e.g., an increase or decrease, in particular parameters, e.g., number of engineered T cells in a sample, compared to the same parameter in a different condition, e.g., before administration of a kinase inhibitor, such as a BTK/ITK inhibitor, e.g., ibrutinib, can be determined. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

Assessment and determination of parameters associated with T cell health, function, activity, and/or outcomes, such as response, efficacy and/or toxicity outcomes, can be assessed at various time points. In some aspects, the assessment can be performed multiple times, e.g., prior to administration of the cell therapy, prior to, during or after manufacturing of the cells, and/or at the initiation of administration of the kinase inhibitor, e.g., ibrutinib, during the continued, resumed and/or further administration of the kinase inhibitor, e.g., ibrutinib, at the initiation of administration of the cell therapy and/or prior to, during or after the initiation of administration of the cell therapy.

In some embodiments, functional attributes of the administered cells and/or cell compositions include monitoring pharmacokinetic (PK) parameters, expansion and persistence of the cells, cell functional assays (e.g., any described herein, such as cytotoxicity assay, cytokine secretion assay and in vivo assays), high-dimensional T cell signaling assessment, and assessment of exhaustion phenotypes and/or signatures of the T cells. In some aspects, other attributes that can be assessed or monitored include monitoring and assessment of minimal residual disease (MRD). In some aspects, other attributes that can be assessed or monitored include pharmacodynamics parameters of the kinase inhibitor, e.g., ibrutinib. In some aspects, such parameters can be assessed using active site occupancy assays, e.g., BTK occupancy assays or ITK occupancy assays.

A. T Cell Exposure, Persistence and Proliferation

In some embodiments, the parameter associated with therapy or a treatment outcome, which include parameters that can be assessed for the screening steps and/or assessment of treatment of outcomes and/or monitoring treatment outcomes, is or includes assessment of the exposure, persistence and proliferation of the T cells, e.g., T cells administered for the T cell based therapy. In some embodiments, the increased exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells used for the immunotherapy, e.g. T cell therapy, before or after administering one or more steps of the combination therapy provided herein.

In some embodiments, the administration of a kinase inhibitor, such as a BTK/ITK inhibitor, e.g., ibrutinib, is designed to promote exposure of the subject to the cells, e.g., T cells administered for T cell based therapy, such as by promoting their expansion and/or persistence over time. In some embodiments, the T cell therapy exhibits increased or prolonged expansion and/or persistence in the subject as compared to a method in which the T cell therapy is administered to the subject in the absence of a kinase inhibitor, e.g., ibrutinib.

In some embodiments, the provided methods increase exposure of the subject to the administered cells (e.g., increased number of cells or duration over time) and/or improve efficacy and therapeutic outcomes of the immunotherapy, e.g. T cell therapy. In some aspects, the methods are advantageous in that a greater and/or longer degree of exposure to the cells expressing the recombinant receptors, e.g., CAR-expressing cells, improves treatment outcomes as compared with other methods. Such outcomes may include patient survival and remission, even in individuals with severe tumor burden.

In some embodiments, the administration of a kinase inhibitor, e.g., ibrutinib, can increase the maximum, total, and/or duration of exposure to the cells, e.g. T cells administered for the T cell based therapy, in the subject as compared to administration of the T cells alone in the absence of a kinase inhibitor, e.g., ibrutinib. In some aspects, administration of a kinase inhibitor, e.g., ibrutinib, in the context of high disease burden (and thus higher amounts of antigen) and/or a more aggressive or resistant B cell malignancy enhances efficacy as compared with administration of the T cells alone in the absence of a kinase inhibitor, e.g., ibrutinib, in the same context, which may result in immunosuppression, anergy and/or exhaustion which may prevent expansion and/or persistence of the cells.

In some embodiments, the presence and/or amount of cells expressing the recombinant receptor (e.g., CAR-expressing cells administered for T cell based therapy) in the subject following the administration of the T cells and before, during and/or after the administration of a kinase inhibitor, e.g., ibrutinib, is detected. In some aspects, quantitative PCR (qPCR) is used to assess the quantity of cells expressing the recombinant receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample.

In some embodiments, the cells are detected in the subject at or at least at 4, 7, 10, 14, 18, 21, 24, 27, or 28 days following the administration of the T cells, e.g., CAR-expressing T cells. In some aspects, the cells are detected at or at least at 2, 4, or 6 weeks following, or 3, 6, or 12, 18, or 24, or 30 or 36 months, or 1, 2, 3, 4, 5, or more years, following the administration of the T cells.

In some embodiments, the persistence of receptor-expressing cells (e.g. CAR-expressing cells) in the subject by the methods, following the administration of the T cells, e.g., CAR-expressing T cells and/or a kinase inhibitor, such as a BTK/ITK inhibitor, e.g., ibrutinib, is greater as compared to that which would be achieved by alternative methods such as those involving the administration of the immunotherapy alone, e.g., administration the T cells, e.g., CAR-expressing T cells, in the absence of a kinase inhibitor, e.g., ibrutinib.

The exposure, e.g., number of cells, e.g. T cells administered for T cell therapy, indicative of expansion and/or persistence, may be stated in terms of maximum numbers of the cells to which the subject is exposed, duration of detectable cells or cells above a certain number or percentage, area under the curve for number of cells over time, and/or combinations thereof and indicators thereof. Such outcomes may be assessed using known methods, such as qPCR to detect copy number of nucleic acid encoding the recombinant receptor compared to total amount of nucleic acid or DNA in the particular sample, e.g., blood, serum, plasma or tissue, such as a tumor sample, and/or flow cytometric assays detecting cells expressing the receptor generally using antibodies specific for the receptors. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor.

In some aspects, increased exposure of the subject to the cells includes increased expansion of the cells. In some embodiments, the receptor expressing cells, e.g. CAR-expressing cells, expand in the subject following administration of the T cells, e.g., CAR-expressing T cells, and/or following administration of a kinase inhibitor, such as a BTK/ITK inhibitor, e.g., ibrutinib. In some aspects, the methods result in greater expansion of the cells compared with other methods, such as those involving the administration of the T cells, e.g., CAR-expressing T cells, in the absence of administering a kinase inhibitor, e.g., ibrutinib.

In some aspects, the method results in high in vivo proliferation of the administered cells, for example, as measured by flow cytometry. In some aspects, high peak proportions of the cells are detected. For example, in some embodiments, at a peak or maximum level following the administration of the T cells, e.g., CAR-expressing T cells and/or a kinase inhibitor, e.g., ibrutinib, in the blood or disease-site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some embodiments, the method results in a maximum concentration, in the blood or serum or other bodily fluid or organ or tissue of the subject, of at least 100, 500, 1000, 1500, 2000, 5000, 10,000 or 15,000 copies of or nucleic acid encoding the receptor, e.g., the CAR, per microgram of DNA, or at least 0.1, 0.2, 0.3, 0.4, 0.5, 0.6, 0.7, 0.8, or 0.9 receptor-expressing, e.g., CAR-expressing cells per total number of peripheral blood mononuclear cells (PBMCs), total number of mononuclear cells, total number of T cells, or total number of microliters. In some embodiments, the cells expressing the receptor are detected as at least 10, 20, 30, 40, 50, or 60% of total PBMCs in the blood of the subject, and/or at such a level for at least 1, 2, 3, 4, 5, 6, 7, 8, 9, 10, 11, 12, 24, 36, 48, or 52 weeks following the T cells, e.g., CAR-expressing T cells and/or the a kinase inhibitor, e.g., ibrutinib, or for 1, 2, 3, 4, or 5, or more years following such administration.

In some aspects, the method results in at least a 2-fold, at least a 4-fold, at least a 10-fold, or at least a 20-fold increase in copies of nucleic acid encoding the recombinant receptor, e.g., CAR, per microgram of DNA, e.g., in the serum, plasma, blood or tissue, e.g., tumor sample, of the subject.

In some embodiments, cells expressing the receptor are detectable in the serum, plasma, blood or tissue, e.g., tumor sample, of the subject, e.g., by a specified method, such as qPCR or flow cytometry-based detection method, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days following administration of the T cells, e.g., CAR-expressing T cells, or after administration of a kinase inhibitor, such as a BTK/ITK inhibitor, e.g., ibrutinib, for at least at or about 2, 3, 4, 5, 6, 7, 8, 9, 10, 11, 12, 13, 14, 15, 16, 17, 18, 19, 20, 21, 22, 23, or 24 or more weeks following the administration of the T cells, e.g., CAR-expressing T cells, and/or a kinase inhibitor, e.g., ibrutinib.

In some aspects, at least about 1×10², at least about 1×10³, at least about 1×10⁴, at least about 1×10⁵, or at least about 1×10⁶ or at least about 5×10⁶ or at least about 1×10⁷ or at least about 5×10⁷ or at least about 1×10⁸ recombinant receptor-expressing, e.g., CAR-expressing cells, and/or at least 10, 25, 50, 100, 200, 300, 400, or 500, or 1000 receptor-expressing cells per microliter, e.g., at least 10 per microliter, are detectable or are present in the subject or fluid, plasma, serum, tissue, or compartment thereof, such as in the blood, e.g., peripheral blood, or disease site, e.g., tumor, thereof. In some embodiments, such a number or concentration of cells is detectable in the subject for at least about 20 days, at least about 40 days, or at least about 60 days, or at least about 3, 4, 5, 6, 7, 8, 9, 10, 11, or 12 months, or at least 2 or 3 years, following administration of the T cells, e.g., CAR-expressing T cells, and/or following the administration of a kinase inhibitor, e.g., ibrutinib. Such cell numbers may be as detected by flow cytometry-based or quantitative PCR-based methods and extrapolation to total cell numbers using known methods. See, e.g., Brentjens et al., Sci Transl Med. 2013 5(177), Park et al, Molecular Therapy 15(4):825-833 (2007), Savoldo et al., JCI 121(5):1822-1826 (2011), Davila et al., (2013) PLoS ONE 8(4):e61338, Davila et al., Oncoimmunology 1(9):1577-1583 (2012), Lamers, Blood 2011 117:72-82, Jensen et al., Biol Blood Marrow Transplant 2010 September; 16(9): 1245-1256, Brentjens et al., Blood 2011 118(18):4817-4828.

In some aspects, the copy number of nucleic acid encoding the recombinant receptor, e.g., vector copy number, per 100 cells, for example in the peripheral blood or bone marrow or other compartment, as measured by immunohistochemistry, PCR, and/or flow cytometry, is at least 0.01, at least 0.1, at least 1, or at least 10, at about 1 week, about 2 weeks, about 3 weeks, about 4 weeks, about 5 weeks, or at least about 6 weeks, or at least about 2, 3, 4, 5, 6, 7, 8, 9, 10, 11, or 12 months or at least 2 or 3 years following administration of the cells, e.g., CAR-expressing T cells, and/or a kinase inhibitor, such as a BTK/ITK inhibitor, e.g., ibrutinib. In some embodiments, the copy number of the vector expressing the receptor, e.g. CAR, per microgram of genomic DNA is at least 100, at least 1000, at least 5000, or at least 10,000, or at least 15,000 or at least 20,000 at a time about 1 week, about 2 weeks, about 3 weeks, or at least about 4 weeks following administration of the T cells, e.g., CAR-expressing T cells, or a kinase inhibitor, e.g., ibrutinib, or at least 2, 3, 4, 5, 6, 7, 8, 9, 10, 11, or 12 months or at least 2 or 3 years following such administration.

In some aspects, the receptor, e.g. CAR, expressed by the cells, is detectable by quantitative PCR (qPCR) or by flow cytometry in the subject, plasma, serum, blood, tissue and/or disease site thereof, e.g., tumor site, at a time that is at least about 3 months, at least about 6 months, at least about 12 months, at least about 1 year, at least about 2 years, at least about 3 years, or more than 3 years, following the administration of the cells, e.g., following the initiation of the administration of the T cells, e.g., CAR-expressing T cells, and/or a kinase inhibitor, e.g., ibrutinib.

In some embodiments, the area under the curve (AUC) for concentration of receptor- (e.g., CAR-) expressing cells in a fluid, plasma, serum, blood, tissue, organ and/or disease site, e.g. tumor site, of the subject over time following the administration of the T cells, e.g., CAR-expressing T cells and/or a kinase inhibitor, e.g., ibrutinib, is greater as compared to that achieved via an alternative dosing regimen where the subject is administered the T cells, e.g., CAR-expressing T cells, in the absence of administering a kinase inhibitor, e.g., ibrutinib.

In some aspects, the method results in high in vivo proliferation of the administered cells, for example, as measured by flow cytometry. In some aspects, high peak proportions of the cells are detected. For example, in some embodiments, at a peak or maximum level following the T cells, e.g., CAR-expressing T cells and/or a kinase inhibitor, e.g., ibrutinib, in the blood, plasma, serum, tissue or disease site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some aspects, the increased or prolonged expansion and/or persistence of the dose of cells in the subject administered with a kinase inhibitor, e.g., ibrutinib, is associated with a benefit in tumor related outcomes in the subject. In some embodiments, the tumor related outcome includes a decrease in tumor burden or a decrease in blast marrow in the subject. In some embodiments, the tumor burden is decreased by or by at least at or about 10, 20, 30, 40, 50, 60, 70, 80, 90, or 100 percent after administration of the method. In some embodiments, disease burden, tumor size, tumor volume, tumor mass, and/or tumor load or bulk is reduced following the dose of cells by at least at or about 50%, 60%, 70%, 80%, 90% or more compared a subject that has been treated with a method that does not involve the administration of a kinase inhibitor, e.g., ibrutinib.

B. T Cell Functional Activity

In some embodiments, parameters associated with therapy or a treatment outcome, which include parameters that can be assessed for the screening steps and/or assessment of treatment of outcomes and/or monitoring treatment outcomes, includes one or more of activity, phenotype, proliferation or function of T cells. In some embodiments, any of the known assays in the art for assessing the activity, phenotypes, proliferation and/or function of the T cells, e.g., T cells administered for T cell therapy, can be used. Prior to and/or subsequent to administration of the cells and/or a kinase inhibitor, such as a BTK/ITK inhibitor, e.g., ibrutinib, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al., J. Immunological Methods, 285(1): 25-40 (2004). In certain embodiments, the biological activity of the cells is measured by assaying expression and/or secretion of one or more cytokines, such as CD107a, IFNγ, IL-2, GM-CSF and TNFα, and/or by assessing cytolytic activity.

In some embodiments, assays for monitoring the cell health, activity phenotype and/or functions, e.g., signaling functions, can include T cell signaling assessment based on mass cytometry (CyTOF), using inductively coupled plasma mass spectrometry and time of flight mass spectrometry, T cell phenotyping, immunophenotyping, e.g., using a panel of antibodies, and other functional assays, such as any described herein.

In some embodiments, assays for the activity, phenotypes, proliferation and/or function of the T cells, e.g., T cells administered for T cell therapy include, but are not limited to, ELISPOT, ELISA, cellular proliferation, cytotoxic lymphocyte (CTL) assay, binding to the T cell epitope, antigen or ligand, or intracellular cytokine staining, proliferation assays, lymphokine secretion assays, direct cytotoxicity assays, and limiting dilution assays. In some embodiments, proliferative responses of the T cells can be measured, e.g. by incorporation of ³H-thymidine, BrdU (5-Bromo-2′-Deoxyuridine) or 2′-deoxy-5-ethynyluridine (EdU) into their DNA or dye dilution assays, using dyes such as carboxyfluorescein diacetate succinimidyl ester (CFSE), CellTrace Violet, or membrane dye PKH26.

In some embodiments, assessing the activity, phenotypes, proliferation and/or function of the T cells, e.g., T cells administered for T cell therapy, include measuring cytokine production from T cells, and/or measuring cytokine production in a biological sample from the subject, e.g., plasma, serum, blood, and/or tissue samples, e.g., tumor samples. In some cases, such measured cytokines can include, without limitation, interlekukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known in the art, and include but are not limited to, ELISA, multiplexed cytokine assay, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample.

In some embodiments, assessing the activity, phenotypes, proliferation and/or function of the T cells, e.g., T cells administered for T cell therapy, include assessing cell phenotypes, e.g., expression of particular cell surface markers. In some embodiments, the T cells, e.g., T cells administered for T cell therapy, are assessed for expression of T cell activation markers, T cell exhaustion markers, and/or T cell differentiation markers. In some embodiments, the cell phenotype is assessed before administration. In some embodiments, the cell phenotype is assessed during, or after administration of cell therapy and/or a kinase inhibitor, such as a BTK/ITK inhibitor, e.g., ibrutinib. T cell activation markers, T cell exhaustion markers, and/or T cell differentiation markers for assessment include any markers known in the art for particular subsets of T cells, e.g., CD25, CD38, human leukocyte antigen-DR (HLA-DR), CD69, CD44, CD137, KLRG1, CD62L^{low}, CCR7^{low}, CD71, CD2, CD54, CD58, CD244, CD160, programmed cell death protein 1 (PD-1), lymphocyte activation gene 3 protein (LAG-3), T-cell immunoglobulin domain and mucin domain protein 3 (TIM-3), cytotoxic T lymphocyte antigen-4 (CTLA-4), band T lymphocyte attenuator (BTLA) and/or T-cell immunoglobulin and immunoreceptor tyrosine-based inhibitory motif domain (TIGIT) (see, e.g., Liu et al., Cell Death and Disease (2015) 6, e1792). In some embodiments, the assessed cell surface marker is CD25, PD-1 and/or TIM-3. In some embodiments, the assessed cell surface marker is CD25.

In some aspects, detecting the expression levels includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more factors, effectors, enzymes and/or surface markers are detected by an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay. In some embodiments, detection of cytokines and/or surface markers is determined using a binding reagent that specifically binds to at least one biomarker. In some cases, the binding reagent is an antibody or antigen-binding fragment thereof, an aptamer or a nucleic acid probe.

In some embodiments, the administration of a kinase inhibitor, such as a BTK/ITK inhibitor, e.g., ibrutinib, increases the level of circulating CAR T cells.

C. Response, Efficacy and Survival

In some embodiments, parameters associated with therapy or a treatment outcome, which include parameters that can be assessed for the screening steps and/or assessment of treatment of outcomes and/or monitoring treatment outcomes, includes tumor or disease burden. The administration of the immunotherapy, such as a T cell therapy (e.g. CAR-expressing T cells) and/or a kinase inhibitor, such as a BTK/ITK inhibitor, e.g., ibrutinib, can reduce or prevent the expansion or burden of the disease or condition in the subject. For example, where the disease or condition is a tumor, the methods generally reduce tumor size, bulk, metastasis, percentage of blasts in the bone marrow or molecularly detectable B cell malignancy and/or improve prognosis or survival or other symptom associated with tumor burden.

In some aspects, the administration in accord with the provided methods, and/or with the provided articles of manufacture or compositions, generally reduces or prevents the expansion or burden of the disease or condition in the subject. For example, where the disease or condition is a tumor, the methods generally reduce tumor size, bulk, metastasis, percentage of blasts in the bone marrow or molecularly detectable B cell malignancy and/or improve prognosis or survival or other symptom associated with tumor burden.

In some embodiments, the provided methods result in a decreased tumor burden in treated subjects compared to alternative methods in which the immunotherapy, such as a T cell therapy (e.g. CAR-expressing T cells) is given without administration of a kinase inhibitor, such as a BTK/ITK inhibitor, e.g., ibrutinib. It is not necessary that the tumor burden actually be reduced in all subjects receiving the combination therapy, but that tumor burden is reduced on average in subjects treated, such as based on clinical data, in which a majority of subjects treated with such a combination therapy exhibit a reduced tumor burden, such as at least 50%, 60%, 70%, 80%, 90%, 95% or more of subjects treated with the combination therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In some embodiments, the subject has a myeloma, a lymphoma or a leukemia. The extent of disease burden can be determined by assessment of residual leukemia in blood or bone marrow. In some embodiments, the subject has a non-Hodgkin lymphoma (NHL), an acute lymphoblastic leukemia (ALL), a chronic lymphocytic leukemia (CLL), a small lymphocytic lymphoma (SLL) a diffuse large B-cell lymphoma (DLBCL) or a myeloma, e.g., a multiple myeloma (MM). In some embodiments, the subject has a MM or a DBCBL. In some embodiments, the subject has a leukemia. In some embodiments, the leukemia is a CLL or a SLL.

In some aspects, response rates in subjects, such as subjects with NHL, are based on the Lugano criteria. (Cheson et al., (2014) JCO., 32(27):3059-3067; Johnson et al., (2015) Radiology 2:323-338; Cheson, B. D. (2015) Chin. Clin. Oncol. 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake≤mediastinum; 3, uptake>mediastinum but ≤liver; 4, uptake moderately>liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measureable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, nonmeasured lesions and new lesions are assessed, which in the case of CR should be absent (Cheson et al., (2014) JCO., 32(27):3059-3067; Johnson et al., (2015) Radiology 2:323-338; Cheson, B. D. (2015) Chin. Clin. Oncol. 4(1):5).

In some aspects, a partial response (PR) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease. In some aspects, response is assessed in the lymph nodes using CT, wherein a PR is described as ≥50% decrease in SPD of up to 6 target measureable nodes and extranodal sites. If a lesion is too small to measure on CT, 5 mm×5 mm is assigned as the default value; if the lesion is no longer visible, the value is 0 mm×0 mm; for a node >5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, nonmeasured lesions and new lesions are assessed, which in the case of PR should be absent/normal, regressed, but no increase. No response/stable disease (SD) or progressive disease (PD) can also be measured using PET-CT and/or CT based assessments. (Cheson et al., (2014) JCO., 32(27):3059-3067; Johnson et al., (2015) Radiology 2:323-338; Cheson, B. D. (2015) Chin. Clin. Oncol., 4(1):5).

In some respects, progression-free survival (PFS) is described as the length of time during and after the treatment of a disease, such as a B cell malignancy, that a subject lives with the disease but it does not get worse. In some aspects, objective response (OR) is described as a measurable response. In some aspects, objective response rate (ORR) is described as the proportion of patients who achieved CR or PR. In some aspects, overall survival (OS) is described as the length of time from either the date of diagnosis or the start of treatment for a disease, such as a B cell malignancy, that subjects diagnosed with the disease are still alive. In some aspects, event-free survival (EFS) is described as the length of time after treatment for a B cell malignancy ends that the subject remains free of certain complications or events that the treatment was intended to prevent or delay. These events may include the return of the B cell malignancy or the onset of certain symptoms, such as bone pain from B cell malignancy that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some aspects, the RECIST criteria is used to determine objective tumor response. (Eisenhauer et al., European Journal of Cancer 45 (2009) 228-247.) In some aspects, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/1); renal insufficiency attributable to myeloma; anemia (hemoglobin <10 g/dl); and/or bone lesions (lytic lesions or osteoporosis with compression fractures)).

In the case of DLBCL, exemplary parameters to assess the extent of disease burden include such parameters as cellular morphology (e.g., centroblastic, immunoblastic, and anaplastic cells), gene expression, miRNA expression and protein expression (e.g., expression of BCL2, BCL6, MUM1, LMO2, MYC, and p21).

In some aspects, response rates in subjects, such as subjects with CLL, are based on the International Workshop on Chronic Lymphocytic Leukemia (IWCLL) response criteria (Hallek, et al., Blood 2008, Jun. 15; 111(12): 5446-5456). In some aspects, these criteria are described as follows: complete remission (CR), which in some aspects requires the absence of peripheral blood clonal lymphocytes by immunophenotyping, absence of lymphadenopathy, absence of hepatomegaly or splenomegaly, absence of constitutional symptoms and satisfactory blood counts; complete remission with incomplete marrow recovery (CRi), which in some aspects is described as CR above, but without normal blood counts; partial remission (PR), which in some aspects is described as ≥50% fall in lymphocyte count, >50% reduction in lymphadenopathy or ≥50% reduction in liver or spleen, together with improvement in peripheral blood counts; progressive disease (PD), which in some aspects is described as ≥50% rise in lymphocyte count to >5×10⁹/L, ≥50% increase in lymphadenopathy, ≥50% increase in liver or spleen size, Richter's transformation, or new cytopenias due to CLL; and stable disease, which in some aspects is described as not meeting criteria for CR, CRi, PR or PD.

In some embodiments, the subjects exhibits a CR or OR if, within 1 month of the administration of the dose of cells, lymph nodes in the subject are less than at or about 20 mm in size, less than at or about 10 mm in size or less than at or about 10 mm in size.

In some embodiments, an index clone of the CLL is not detected in the bone marrow of the subject (or in the bone marrow of greater than 50%, 60%, 70%, 80%, 90% or more of the subjects treated according to the methods. In some embodiments, an index clone of the CLL is assessed by IgH deep sequencing. In some embodiments, the index clone is not detected at a time that is at or about or at least at or about 1, 2, 3, 4, 5, 6, 12, 18 or 24 months following the administration of the cells.

In some embodiments, a subject exhibits morphologic disease if there are greater than or equal to 5% blasts in the bone marrow, for example, as detected by light microscopy, such as greater than or equal to 10% blasts in the bone marrow, greater than or equal to 20% blasts in the bone marrow, greater than or equal to 30% blasts in the bone marrow, greater than or equal to 40% blasts in the bone marrow or greater than or equal to 50% blasts in the bone marrow. In some embodiments, a subject exhibits complete or clinical remission if there are less than 5% blasts in the bone marrow.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable B cell malignancy. In some embodiments, molecularly detectable B cell malignancy can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify B cell malignancy cell based on leukemia-specific immunophenotypes. In some embodiments, molecular detection of B cell malignancy can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In the case of leukemia, the extent of disease burden can be determined by assessment of residual leukemia in blood or bone marrow. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow.

In some embodiments, for leukemia, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable B cell malignancy. In some embodiments, molecularly detectable B cell malignancy can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify B cell malignancy cell based on leukemia-specific immunophenotypes. In some embodiments, molecular detection of B cell malignancy can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In some embodiments, the methods and/or administration of a cell therapy, such as a T cell therapy (e.g. CAR-expressing T cells) and/or a kinase inhibitor, such as a BTK/ITK inhibitor, e.g., ibrutinib, decrease(s) disease burden as compared with disease burden at a time immediately prior to the administration of the immunotherapy, e.g., T cell therapy and/or a kinase inhibitor, e.g., ibrutinib.

In some aspects, administration of the immunotherapy, e.g. T cell therapy and/or a kinase inhibitor, e.g., ibrutinib, may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives immunotherapy, e.g. T cell therapy alone, in the absence of administration of a kinase inhibitor, e.g., ibrutinib. In some embodiments, disease burden is reduced to a greater extent or for a greater duration following the combination therapy of administration of the immunotherapy, e.g., T cell therapy, and a kinase inhibitor, e.g., ibrutinib, compared to the reduction that would be effected by administering each of the agent alone, e.g., administering a kinase inhibitor, e.g., ibrutinib, to a subject having not received the immunotherapy, e.g. T cell therapy; or administering the immunotherapy, e.g. T cell therapy, to a subject having not received a kinase inhibitor, e.g., ibrutinib.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

In some aspects, disease burden is measured or detected prior to administration of the immunotherapy, e.g. T cell therapy, following the administration of the immunotherapy, e.g. T cell therapy but prior to administration of a kinase inhibitor, e.g., ibrutinib, and/or following the administration of both the immunotherapy, e.g. T cell therapy and a kinase inhibitor, e.g., ibrutinib. In the context of multiple administration of one or more steps of the combination therapy, disease burden in some embodiments may be measured prior to, or following administration of any of the steps, doses and/or cycles of administration, or at a time between administration of any of the steps, doses and/or cycles of administration. In some embodiments, the administration of a kinase inhibitor, e.g., ibrutinib, is carried out at least two cycles (e.g., 28-day cycle), and disease burden is measured or detected prior to, during, and/or after each cycle.

In some embodiments, the burden is decreased by or by at least at or about 10, 20, 30, 40, 50, 60, 70, 80, 90, or 100 percent by the provided methods compared to immediately prior to the administration of a kinase inhibitor, e.g., ibrutinib, and the immunotherapy, e.g. T cell therapy. In some embodiments, disease burden, tumor size, tumor volume, tumor mass, and/or tumor load or bulk is reduced following administration of the immunotherapy, e.g. T cell therapy and a kinase inhibitor, e.g., ibrutinib, by at least at or about 10, 20, 30, 40, 50, 60, 70, 80, 90% or more compared to that immediately prior to the administration of the immunotherapy, e.g. T cell therapy and/or a kinase inhibitor, e.g., ibrutinib.

In some embodiments, reduction of disease burden by the method comprises an induction in morphologic complete remission, for example, as assessed at 1 month, 2 months, 3 months, 4 months, 5 months, 6 months, or more than 6 months, after administration of, e.g., initiation of, the combination therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of combination therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the method of combination therapy, is less than about 80%, less than about 70%, less than about 60%, less than about 50%, less than about 40%, less than about 30%, less than about 20%, or less than about 10%.

In some cases, the pharmacokinetics of administered cells, e.g., adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (e.g., a CAR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR+ T cells in a biological sample obtained from the patient, e.g., blood, can be determined at a period of time after administration of the cell therapy, e.g., to determine the pharmacokinetics of the cells. In some embodiments, number of CAR+ T cells, optionally CAR+ CD8+ T cells and/or CAR+ CD4+ T cells, detectable in the blood of the subject, or in a majority of subjects so treated by the method, is greater than 1 cells per μL, greater than 5 cells per μL or greater than per 10 cells per μL.

### IV. Toxicity and Adverse Outcomes

In embodiments of the provided methods, the subject is monitored for toxicity or other adverse outcome, including treatment related outcomes, e.g., development of neutropenia, cytokine release syndrome (CRS) or neurotoxicity (NT), in subjects administered the provided combination therapy comprising a cell therapy (e.g., a T cell therapy) and a kinase inhibitor, such as a BTK/ITK inhibitor, e.g., ibrutinib. In some embodiments, the provided methods are carried out to reduce the risk of a toxic outcome or symptom, toxicity-promoting profile, factor, or property, such as a symptom or outcome associated with or indicative of severe neutropenia, severe cytokine release syndrome (CRS) or severe neurotoxicity.

In some embodiments, the provided methods do not result in a high rate or likelihood of toxicity or toxic outcomes, or reduces the rate or likelihood of toxicity or toxic outcomes, such as severe neurotoxicity (NT) or severe cytokine release syndrome (CRS), such as compared to certain other cell therapies. In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxcity. In some embodiments, no more than 50% of subjects treated (e.g. at least 60%, at least 70%, at least 80%, at least 90% or more of the subjects treated) a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

In some embodiments, the provided methods do not result in a high rate or likelihood of toxicity or toxic outcomes, or reduces the rate or likelihood of toxicity or toxic outcomes, such as severe neurotoxicity (NT) or severe cytokine release syndrome (CRS), such as compared to certain other cell therapies. In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any any grade of CRS or any grade of neurotoxcity. In some embodiments, no more than 50% of subjects treated (e.g. at least 60%, at least 70%, at least 80%, at least 90% or more of the subjects treated) a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

A. Cytokine Release Syndrome (CRS) and Neurotoxicity

In some aspects, the toxic outcome is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. CRS may be treated using anti-inflammatory therapy such as an anti-IL-6 therapy, e.g., anti-IL-6 antibody, e.g., tocilizumab, or antibiotics or other agents as described.

Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, IL-6, IL-8, and IL-10.

Exemplary outcomes associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly.

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (P02) levels of less than at or about 90%); and/or one or more neurologic disorders (including mental status changes, obtundation, and seizures).

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

In some embodiments, the CRS-associated serum factors or CRS-related outcomes include inflammatory cytokines and/or chemokines, including interferon gamma (IFN-γ), TNF-α, IL-1β, IL-2, IL-6, IL-7, IL-8, IL-10, IL-12, sIL-2Ra, granulocyte macrophage colony stimulating factor (GM-CSF), macrophage inflammatory protein (MIP)-1, tumor necrosis factor alpha (TNFα), IL-6, and IL-10, IL-1β, IL-8, IL-2, MIP-1, Flt-3L, fracktalkine, and/or IL-5. In some embodiments, the factor or outcome includes C reactive protein (CRP). In addition to being an early and easily measurable risk factor for CRS, CRP also is a marker for cell expansion. In some embodiments, subjects that are measured to have high levels of CRP, such as ≥15 mg/dL, have CRS. In some embodiments, subjects that are measured to have high levels of CRP do not have CRS. In some embodiments, a measure of CRS includes a measure of CRP and another factor indicative of CRS.

In some embodiments, one or more inflammatory cytokines or chemokines are monitored before, during, or after CAR treatment and/or a kinase inhibitor, such as a BTK/ITK inhibitor, e.g., ibrutinib, treatment. In some aspects, the one or more cytokines or chemokines include IFN-γ, TNF-α, IL-2, IL-1β, IL-6, IL-7, IL-8, IL-10, IL-12, sIL-2Rα, granulocyte macrophage colony stimulating factor (GM-CSF), or macrophage inflammatory protein (MIP). In some embodiments, IFN-γ, TNF-α, and IL-6 are monitored.

CRS criteria that appear to correlate with the onset of CRS to predict which patients are more likely to be at risk for developing sCRS have been developed (see Davilla et al. Science translational medicine. 2014; 6(224):224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014; 124(2):188-95). In some embodiments, the criteria reflective of CRS grade are those detailed in Table 3 below.

TABLE 3
Exemplary Grading Criteria for CRS

| Grade | Description of Symptoms |
|---|---|
| 1 | Not life-threatening, require only symptomatic |
| Mild | treatment such as antipyretics and anti-emetics |
| | (e.g., fever, nausea, fatigue, headache, myalgias, |
| | malaise) |
| 2 | Require and respond to moderate intervention: |
| Moderate | Oxygen requirement <40%, or |
| | Hypotension responsive to fluids or low dose |
| | of a single vasopressor, or |
| | Grade 2 organ toxicity (by CTCAE v4.0) |
| 3 | Require and respond to aggressive intervention: |
| Severe | Oxygen requirement ≥40%, or |
| | Hypotension requiring high dose of a single |
| | vasopressor (e.g., norepinephrine ≥20 μg/kg/min, |
| | dopamine ≥10 μg/kg/min, phenylephrine |
| | ≥200 μg/kg/min, or epinephrine ≥10 μg/kg/min), or |
| | Hypotension requiring multiple vasopressors |
| | (e.g., vasopressin + one of the above agents, |
| | or combination vasopressors equivalent to |
| | ≥20 μg/kg/min norepinephrine), or |
| | Grade 3 organ toxicity or Grade 4 transaminitis |
| | (by CTCAE v4.0) |
| 4 | Life-threatening: |
| Life- | Requirement for ventilator support, or |
| threatening | Grade 4 organ toxicity (excluding transaminitis) |
| 5 | Death |
| Fatal | |

In some embodiments, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater, such as set forth in Table 3.

In some embodiments, outcomes associated with severe CRS or grade 3 CRS or greater, such as grade 4 or greater, include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, severe CRS includes CRS that requires management or care in the intensive care unit (ICU).

In some embodiments, the CRS, such as severe CRS, encompasses a combination of (1) persistent fever (fever of at least 38 degrees Celsius for at least three days) and (2) a serum level of CRP of at least at or about 20 mg/dL. In some embodiments, the CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor.

The method of measuring or detecting the various outcomes may be specified.

In some aspects, the toxic outcome of a therapy, such as a cell therapy, is or is associated with or indicative of neurotoxicity or severe neurotoxicity. In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03).

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 4. In some embodiments, a severe neurotoxicity is deemed to be a prolonged grade 3 if symptoms or grade 3 neurotoxicity last for 10 days or longer.

TABLE 4
Exemplary Grading Criteria for neurotoxicity

| Grade | Description of Symptoms |
|---|---|
| 1 | Mild or asymptomatic symptoms |
| Asymptomatic | |
| or Mild | |
| 2 | Presence of symptoms that limit instrumental |
| Moderate | activities of daily living (ADL), such as |
| | preparing meals, shopping for groceries or |
| | clothes, using the telephone, managing |
| | money |
| 3 | Presence of symptoms that limit self-care |
| Severe | ADL, such as bathing, dressing and |
| | undressing, feeding self, using the toilet, |
| | taking medications |
| 4 | Symptoms that are life-threatening, requiring |
| Life-threatening | urgent intervention |
| 5 | Death |
| Fatal | |

In some embodiments, the methods reduce symptoms associated with CRS or neurotoxicity compared to other methods. In some aspects, the provided methods reduce symptoms, outcomes or factors associated with CRS, including symptoms, outcomes or factors associated with severe CRS or grade 3 or higher CRS, compared to other methods. For example, subjects treated according to the present methods may lack detectable and/or have reduced symptoms, outcomes or factors of CRS, e.g. severe CRS or grade 3 or higher CRS, such as any described, e.g. set forth in Table 3. In some embodiments, subjects treated according to the present methods may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods. In some embodiments, subjects treated according to the present methods may have reduced symptoms associated with peripheral motor neuropathy, peripheral sensory neuropathy, dysethesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals.

In some embodiments, the toxicity outcome is a dose-limiting toxicity (DLT). In some embodiments, the toxic outcome is the absence of a dose-limiting toxicity. In some embodiments, a dose-limiting toxicity (DLT) is defined as any grade 3 or higher toxicity as described or assessed by any known or published guidelines for assessing the particular toxicity, such as any described above and including the National Cancer Institute (NCI) Common Terminology Criteria for Adverse Events (CTCAE) version 4.0. In some embodiments, a dose-limiting toxicity (DLT) is defined when any of the events discussed below occurs following administration of the cell therapy (e.g., T cell therapy) and/or a kinase inhibitor, such as a BTK/ITK inhibitor, e.g., ibrutinib, the events including a) febrile neutropenia; b) Grade 4 neutropenia lasting about or more than about 7 days; c) Grade 3 or 4 thrombocytopenia with clinically significant bleeding; and d) Grade 4 thrombocytopenia lasting more than 24 hours.

In some embodiments, the provided embodiments result in a low rate or risk of developing a toxicity, e.g. CRS or neurotoxicity or severe CRS or neurotoxicity, e.g. grade 3 or higher CRS or neurotoxicity, such as observed with administering a dose of T cells in accord with the provided combination therapy, and/or with the provided articles of manufacture or compositions. In some cases, this permits administration of the cell therapy on an outpatient basis. In some embodiments, the administration of the cell therapy, e.g. dose of T cells (e.g. CAR+ T cells) in accord with the provided methods, and/or with the provided articles of manufacture or compositions, is performed on an outpatient basis or does not require admission to the subject to the hospital, such as admission to the hospital requiring an overnight stay.

In some aspects, subjects administered the cell therapy, e.g. dose of T cells (e.g. CAR+ T cells) in accord with the provided methods, and/or with the provided articles of manufacture or compositions, including subjects treated on an outpatient basis, are not administered an intervention for treating any toxicity prior to or with administration of the cell dose, unless or until the subject exhibits a sign or symptom of a toxicity, such as of a neurotoxicity or CRS.

In some embodiments, if a subject administered the cell therapy, e.g. dose of T cells (e.g. CAR+ T cells), including subjects treated on an outpatient basis, exhibits a fever the subject is given or is instructed to receive or administer a treatment to reduce the fever. In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about or at least at or about 38, 39, 40, 41, or 42 degrees Celsius, and/or may be a range of at or about 38 degrees Celsius to at or about 39 degrees Celsius, a range of at or about 39 degrees Celsius to at or about 40 degrees Celsius, a range of at or about 40 degrees Celsius to at or about 41 degrees, or a range of at or about 41 degrees Celsius to at or about 42 degrees Celsius.

In some embodiments, the treatment designed to reduce fever includes treatment with an antipyretic. An antipyretic may include any agent, composition, or ingredient, that reduces fever, such as one of any number of agents known to have antipyretic effects, such as NSAIDs (such as ibuprofen, naproxen, ketoprofen, and nimesulide), salicylates, such as aspirin, choline salicylate, magnesium salicylate, and sodium salicylate, paracetamol, acetaminophen, Metamizole, Nabumetone, Phenaxone, antipyrine, febrifuges. In some embodiments, the antipyretic is acetaminophen. In some embodiments, acetaminophen can be administered at a dose of 12.5 mg/kg orally or intravenously up to every four hours. In some embodiments, it is or comprises ibuprofen or aspirin.

In some embodiments, if the fever is a sustained fever, the subject is administered an alternative treatment for treating the toxicity. For subjects treated on an outpatient basis, the subject is instructed to return to the hospital if the subject has and/or is determined to or to have a sustained fever. In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1° C., and generally does not fluctuate by about, or by more than about, 0.5° C., 0.4° C., 0.3° C., or 0.2° C.), following a specified treatment, such as a treatment designed to reduce fever such as treatment with an antipyreticm, e.g. NSAID or salicylates, e.g. ibuprofen, acetaminophen or aspirin. For example, a subject is considered to have a sustained fever if he or she exhibits or is determined to exhibit a fever of at least at or about 38 or 39 degrees Celsius, which is not reduced by or is not reduced by more than at or about 0.5° C., 0.4° C., 0.3° C., or 0.2° C., or by at or about 1%, 2%, 3%, 4%, or 5%, over a period of 6 hours, over a period of 8 hours, or over a period of 12 hours, or over a period of 24 hours, even following treatment with the antipyretic such as acetaminophen. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, the subject has, and/or is determined to or considered to have, a sustained fever if he or she exhibits a fever at or above the relevant threshold temperature, and where the fever or body temperature of the subject does not fluctuate by about, or by more than about, 1° C., and generally does not fluctuate by about, or by more than about, 0.5° C., 0.4° C., 0.3° C., or 0.2° C. Such absence of fluctuation above or at a certain amount generally is measured over a given period of time (such as over a 24-hour, 12-hour, 8-hour, 6-hour, 3-hour, or 1-hour period of time, which may be measured from the first sign of fever or the first temperature above the indicated threshold). For example, in some embodiments, a subject is considered to or is determined to exhibit sustained fever if he or she exhibits a fever of at least at or about or at least at or about 38 or 39 degrees Celsius, which does not fluctuate in temperature by more than at or about 0.5° C., 0.4° C., 0.3° C., or 0.2° C., over a period of 6 hours, over a period of 8 hours, or over a period of 12 hours, or over a period of 24 hours.

In some embodiments, the fever is a sustained fever; in some aspects, the subject is treated at a time at which a subject has been determined to have a sustained fever, such as within one, two, three, four, five six, or fewer hours of such determination or of the first such determination following the initial therapy having the potential to induce the toxicity, such as the cell therapy, such as dose of T cells, e.g. CAR+ T cells.

In some embodiments, one or more interventions or agents for treating the toxicity, such as a toxicity-targeting therapies, is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

B. Other Toxicities

In some aspects, the toxic outcome is or is associated with or indicative of one or more non-hematologic toxicity following administration of a kinase inhibitor, such as a BTK/ITK inhibitor, e.g., ibrutinib. Examples of non-hematologic toxicities include, but are not limited to, tumor flare reaction, infections, tumor lysis syndrome, cardiac laboratory abnormalities, thromboembolic event(s) (such as deep vein thrombosis and pulmonary embolism), and/or pneumonitis.

In some aspects, the non-hematologic toxicity is tumor flare reaction (TFR) (sometimes also referred to pseudoprogression). TFR is a sudden increase in the size of the disease-bearing sites, including the lymph nodes, spleen and/or the liver often accompanied by a low-grade fever, tenderness and swelling, diffuse rash and in some cases, an increase in the peripheral blood lymphocyte counts. In some embodiments, TFR is graded according to Common Terminology Criteria for Adverse Events (Version 3.0; US National Cancer Institute, Bethesda, Md., USA). In some embodiments, TFR is graded as follows: grade 1, mild pain not interfering with function; grade 2, moderate pain, pain or analgesics interfering with function but not interfering with activities of daily living (ADL); grade 3, severe pain, pain or analgestics interfering with function and interfering with ADL; grade 4, disabling; grade 5, death. In some embodiments, one or more agents can be administered to the subject to treat, ameliorate or lessen one or more symptoms associated with TFR, such as corticosteroids, NSAIDs and/or narcotic analgesic.

In some aspects, the non-hematologic toxicity is tumor lysis syndrome (TLS). In some embodiments, TLS can be graded according to criteria specified by the Cairo-Bishop grading system (Cairo and Bishop (2004) Br J Haematol, 127:3-11). In some embodiments, subjects can be given intravenous hydration to reduce hyperuricemia.

In some embodiments, subjects can be monitored for cardiac toxicity, such as by monitoring ECGS, LVEF and monitoring levels of troponin-T and BNP. In some embodiments, a cardiac toxicity that potentially may necessitate holding or suspending a kinase inhibitor, e.g., ibrutinib, may occur if elevated levels of troponin-T and/or BNP with one or more cardiac symptoms is observed.

In some embodiments of the provided methods, if a subject is determined to exhibit a non-hematological toxicity, such as TFR or other non-hematological toxicity or a particular grade thereof, the cycling therapy with a kinase inhibitor, e.g., ibrutinib, can be altered. In some aspects, the cycling therapy is altered if, after administration of a kinase inhibitor, e.g., ibrutinib, the subject has a grade 3 or higher non-hematological toxicity, such as grade 3 or higher TFR. In some embodiments, administration of a kinase inhibitor, e.g., ibrutinib, is halted permanently or suspended until signs or symptoms of the toxicity is resolved, lessened or reduced. Continued monitoring of the subject can be carried out to assess one or more signs or symptoms of the toxicity. In some cases, if the toxicity resolves or is reduced, administration of a kinase inhibitor, e.g., ibrutinib, can be restarted at the same dose or dosing regimen prior to suspending the cycling therapy, at a lower or reduced dose, and/or in a dosing regimen involving less frequent dosing. In some embodiments, in instances of restarting the cycling therapy, the dose is lowered or reduced at least or at least about or about 10%, 15%, 20%, 25%, 30%, 40%, 50%, or 60%. In some embodiments, if the dose prior to suspending the cell therapy is 2 mg (e.g. given 5/7 days), the dose is reduced to 1 mg (given 5/7 days). In some embodiments, if a grade 3 toxicity recurs even after a dose reduction, the dose can be further reduced. In some embodiments, if a grade 4 toxicity recurs even after a dose reduction, the cycling therapy can be permanently discontinued. In some aspects, if a hematological toxicity is of such severity that suspension of the cycling therapy is for greater than 4 weeks, the cycling therapy can be permanently discontinued.

### V. Articles of Manufacture and Kits

Also provided are articles of manufacture containing a kinase inhibitor, such as a BTK/ITK inhibitor, e.g., ibrutinib, and components for the immunotherapy, e.g., antibody or antigen binding fragment thereof or T cell therapy, e.g. engineered cells, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the engineered cells used for the immunotherapy, e.g. T cell therapy; and (b) a second container with a composition contained therein, wherein the composition includes a kinase inhibitor, such as a BTK/ITK inhibitor, e.g., ibrutinib.

In some embodiments, the first container comprises a first composition and a second composition, wherein the first composition comprises a first population of the engineered cells used for the immunotherapy, e.g., CD4+ T cell therapy, and the second composition comprises a second population of the engineered cells, wherein the second population may be engineered separately from the first population, e.g., CD8+ T cell therapy. In some embodiments, the first and second cell compositions contain a defined ratio of the engineered cells, e.g., CD4+ and CD8+ cells (e.g., 1:1 ratio of CD4+:CD8+ CAR+ T cells).

The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, a “subject” is a mammal, such as a human or other animal, and typically is human. In some embodiments, the subject, e.g., patient, to whom a kinase inhibitor, such as a BTK/ITK inhibitor, e.g., ibrutinib, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as B cell malignancy). This delay can be of varying lengths of time, depending on the history of the disease and/or individual being treated. As is evident, a sufficient or significant delay can, in effect, encompass prevention, in that the individual does not develop the disease. For example, a late stage B cell lymphoma, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., engineered cells or anti-PD-L1 or antigen-binding fragment, or a pharmaceutical formulation or composition thereof, in the context of administration, refers to an amount effective, at dosages/amounts and for periods of time necessary, to achieve a desired result, such as a therapeutic or prophylactic result.

A “therapeutically effective amount” of an agent, e.g., engineered cells or anti-PD-L1 or antigen-binding fragment, or a pharmaceutical formulation or composition thereof, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering a kinase inhibitor, such as a BTK/ITK inhibitor, e.g., ibrutinib, engineered cells (e.g. cell therapy), or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or a kinase inhibitor, such as a BTK/ITK inhibitor, e.g., ibrutinib, including cells. It may be a solution, a suspension, liquid, powder, a paste, aqueous, non-aqueous or any combination thereof.

As used herein, a “subject” is a mammal, such as a human or other animal, and typically is human.

### VII. Exemplary Embodiments

Among the provided embodiments are:

1. A method of treatment, the method comprising:

(1) administering to a subject having a cancer an effective amount of a kinase inhibitor that is or comprises the structure

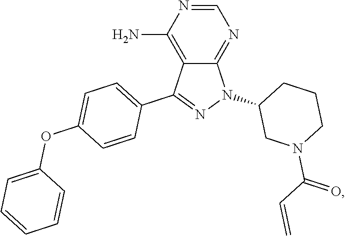 [SMILES: C=CC(=O)N1CCC[C@@H](n2nc(-c3ccc(Oc4ccccc4)cc3)c3c(N)ncnc32)C1]

or a pharmaceutically acceptable salt thereof; and

(2) administering an autologous T cell therapy to the subject, said T cell therapy comprising a dose of genetically engineered T cells expressing a chimeric antigen receptor (CAR) that specifically binds to a CD19, wherein, prior to administering the T cell therapy, a biological sample has been obtained from the subject and processed, the processing comprising genetically modifying T cells from the sample, optionally by introducing a nucleic acid molecule encoding the CAR into said T cells,

wherein the administration of the kinase inhibitor is initiated at least at or about 3 days prior to the obtaining of the sample and is carried out in a dosing regimen comprising repeat administrations of the kinase inhibitor at a dosing interval, over a period of time that extends at least to include administration on or after the day that the sample is obtained from the subject.

2. A method of treatment, the method comprising:

(1) administering to a subject having a cancer an effective amount of a kinase inhibitor that is or comprises the structure

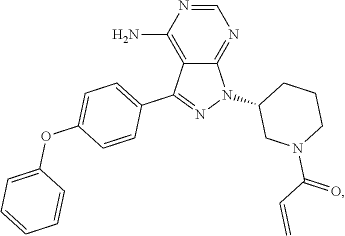 [SMILES: C=CC(=O)N1CCC[C@@H](n2nc(-c3ccc(Oc4ccccc4)cc3)c3c(N)ncnc32)C1]

or a pharmaceutically acceptable salt thereof;

(2) obtaining from the subject a biological sample and processing T cells of said sample, thereby generating a composition comprising genetically engineered T cells that express a chimeric antigen receptor (CAR) that specifically binds to a CD19; and

(3) administering to the subject an autologous T cell therapy comprising a dose of the genetically engineered T cells,

wherein the administration of the kinase inhibitor is carried out in a dosing regimen that is initiated at least at or about 3 days prior to the obtaining of the sample and that comprises repeat administrations of the inhibitor, at a dosing interval, over a period of time and extends at least to include administration of the compound on or after the day that the sample is obtained from the subject.

3. A method of treatment, the method comprising administering to a subject having a cancer an effective amount of a kinase inhibitor having the structure

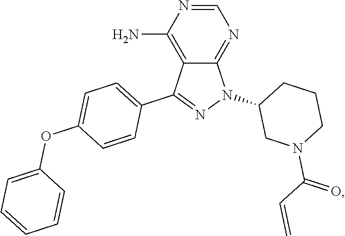 [SMILES: C=CC(=O)N1CCC[C@@H](n2nc(-c3ccc(Oc4ccccc4)cc3)c3c(N)ncnc32)C1]

or a pharmaceutically acceptable salt thereof, wherein the subject is a candidate for treatment or is to be treated with an autologous T cell therapy, said T cell therapy comprising a dose of genetically engineered T cells expressing a chimeric antigen receptor (CAR) that specifically binds to a CD19, wherein:

prior to administering the T cell therapy a biological sample has been obtained from the subject and processed, the processing comprising genetically modifying T cells from the sample, optionally by introducing a nucleic acid molecule encoding the CAR into said T cells; and

the administration of the kinase inhibitor is initiated at least at or about 3 days prior to the obtaining of the sample and is carried out in a dosing regimen comprising repeat administrations of the inhibitor at a dosing interval for a period of time that extends at least to include administration on or after the day that the sample is obtained from the subject.

4. The method of embodiment 3, further comprising administering to the subject the T cell therapy.

5. The method of any of embodiments 1, 2 and embodiment 4, wherein, subsequent to initiation the administration of the kinase inhibitor and prior to the administration of the T cell therapy, the subject has been preconditioned with a lymphodepleting therapy.

6. The method of any of embodiments 1, 2 and embodiment 4, further comprising, subsequent to initiating the administration of the kinase inhibitor and prior to the administration of the T cell therapy, administering a lymphodepleting therapy to the subject.

7. The method of embodiment 5 or embodiment 6, wherein the administration of the kinase inhibitor is discontinued or halted during the lymphodepleting therapy.

8. The method of any of embodiments 5-7, wherein the dosing regimen comprises administration of the kinase inhibitor over a period of time that extends at least to include administration up to the initiation of the lymphodepleting therapy.

9. The method of any of embodiments 5-7, wherein the dosing regimen comprises administration of the kinase inhibitor over a period of time that includes administration up to the initiation of the lymphodepleting therapy, followed by discontinuing or halting administration of the kinase inhibitor during the lymphodepleting therapy and then further administration of the kinase inhibitor for a period that extends for at least 15 days after initiation of administration of the T cell therapy.

10. A method of treatment, the method comprising:

(1) administering to a subject having a cancer an effective amount of a kinase inhibitor having the structure

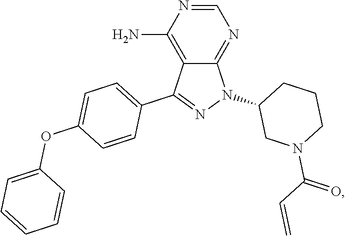 [SMILES: C=CC(=O)N1CCC[C@@H](n2nc(-c3ccc(Oc4ccccc4)cc3)c3c(N)ncnc32)C1]

or a pharmaceutically acceptable salt thereof;

(2) administering a lymphodepleting therapy to the subject; and

(3) administering an autologous T cell therapy to the subject, said T cell therapy comprising a dose of genetically engineered T cells expressing a chimeric antigen receptor (CAR) that specifically binds to a CD19, wherein, prior to administering the T cell therapy comprising biological sample has been obtained from the subject and processed, the processing comprising genetically modifying T cells from the sample, optionally by introducing a nucleic acid molecule encoding the CAR into said T cells,

wherein the administration of the kinase inhibitor is initiated at least at or about 3 days prior to obtaining the obtaining of the sample and is carried out in a dosing regimen comprising repeat administration of the kinase inhibitor at a dosing interval over a period of time that includes administration up to the initiation of the lymphodepleting therapy, followed by discontinuing or halting administration of the kinase inhibitor during the lymphodepleting therapy, and then further administration of the kinase inhibitor for a period that extends for at least 15 days after initiation of administration of the T cell therapy.

11. The method of embodiment 10, wherein the method further comprises obtaining from the subject the biological sample and processing T cells of said sample, thereby generating a composition comprising the genetically engineered T cells that express the chimeric antigen receptor (CAR) that specifically binds to a CD19

12. The method of any of embodiments 1-11, wherein the administration of the kinase inhibitor is initiated at least at or about 4 days, at least at or about 5 days, at least at or 6 days, at least at about 7 days, at least at or about 14 days or more prior to the obtaining the sample from the subject.

13. The method of any of embodiments 1-12, wherein the administration of the kinase inhibitor is initiated at least or at or about 5 days to 7 days prior to the obtaining the sample from the subject.

14. The method of any of embodiments 5-13, wherein administration of the lymphodepleting therapy is completed within 7 days prior to initiation of the administration of the T cell therapy.

15. The method of any of embodiments 5-14, wherein administration of the lymphodepleting therapy is completed 2 to 7 days prior to initiation of the administration of the T cell therapy.

16. The method of any of embodiments 9-15, wherein the further administration is for a period that extends for 15 days to 29 days after initiation of administration of the T cell therapy.

17. The method of any of embodiments 9-16, wherein the further administration of the kinase inhibitor is for a period that extends at or about or greater than three months after initiation of administration of the T cell therapy.

18. The method of any of embodiments 1-17, wherein the administration of the kinase inhibitor is carried out once per day on each day it is administered during the dosing regimen.

19. The method of any of embodiments 1-18, wherein the effective amount comprises from or from about 140 mg to about 840 mg or 140 mg to about 560 mg per each day the kinase inhibitor is administered.

20. A method of treatment, the method comprising:

(1) administering to a subject having a cancer a kinase inhibitor, wherein the kinase inhibitor is or comprises the structure

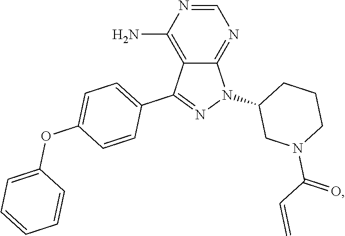 [SMILES: C=CC(=O)N1CCC[C@@H](n2nc(-c3ccc(Oc4ccccc4)cc3)c3c(N)ncnc32)C1]

or is a pharmaceutically acceptable salt thereof; and

(2) administering an autologous T cell therapy to the subject, said T cell therapy comprising a dose of genetically engineered T cells expressing a chimeric antigen receptor (CAR) that specifically binds to a CD19, wherein, prior to administering the T cell therapy a biological sample has been obtained from the subject and processed, the processing comprising genetically modifying T cells from the sample, optionally by introducing a nucleic acid molecule encoding the CAR into said T cells,

wherein the administration of the kinase inhibitor is initiated at least at or about 5 to 7 days prior to the obtaining of the sample and is carried out in a dosing regimen comprising repeat administration of the kinase inhibitor at a dosing interval over a period of time that extends at least to include administration on or after the day that the sample is obtained from the subject and further administration that extends for at or about or greater than three months after initiation of administration of the T cell therapy, wherein the kinase inhibitor is administered in an amount from or from about 140 mg to about 560 mg once per day each day it is administered during the dosing regimen.

21. The method of embodiment 20, wherein, subsequent to initiating administration of the kinase inhibitor and prior to the administration of the T cell therapy, the subject has been preconditioned with a lymphodepleting therapy.

22. The method of embodiment 20, further comprising, subsequent to the administration of the kinase inhibitor and prior to the administration of the T cell therapy, administering a lymphodepleting therapy to the subject.

23. The method of any of embodiments 20-22, wherein the administration of the lymphodepleting therapy is completed within 7 days prior to initiation of the administration of the T cell therapy.

24. The method of any of embodiments 20-23, wherein the administration of the lymphodepleting therapy is completed 2 to 7 days prior to initiation of the administration of the T cell therapy.

25. The method of any of embodiments 22-24, wherein the dosing regimen comprises discontinuing or halting administration of the kinase inhibitor during the lymphodepleting therapy.

26. A method of treatment, the method comprising:

(1) administering to a subject having a cancer a kinase inhibitor, wherein the kinase inhibitor has the structure

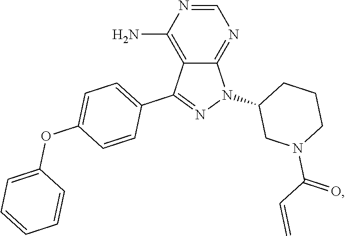 [SMILES: C=CC(=O)N1CCC[C@@H](n2nc(-c3ccc(Oc4ccccc4)cc3)c3c(N)ncnc32)C1]

or is a pharmaceutically acceptable salt thereof; and

(2) administering a lymphodepleting therapy to the subject; and

(3) administering an autologous T cell therapy to the subject within 2 to 7 days after completing the lymphodepleting therapy, said T cell therapy comprising a dose of genetically engineered T cells expressing a chimeric antigen receptor (CAR) that specifically binds to a CD19, wherein, prior to administering the T cell therapy a biological sample has been obtained from the subject and processed, the processing comprising genetically modifying T cells from the sample, optionally by introducing a nucleic acid molecule encoding the CAR into said T cells,

wherein the administration of the kinase inhibitor is initiated at least at or about 5 to 7 days prior to the obtaining of the sample and is carried out in a dosing regimen comprising repeat administration of the kinase inhibitor at a dosing interval that includes administration up to the initiation of the lymphodepleting therapy, followed by discontinuing or halting administration of the kinase inhibitor during the lymphodepleting therapy, and then further administration for a period that extends for at or greater than three months after initiation of administration of the T cell therapy, wherein the kinase inhibitor is administered in an amount from or from about 140 mg to about 560 mg once per day each day it is administered during the dosing regimen.

27. The method of any of embodiments 20-26, wherein the method further comprises obtaining from the subject the biological sample and processing T cells of said sample, thereby generating a composition comprising the genetically engineered T cells that express the chimeric antigen receptor (CAR) that specifically binds to a CD19

28. The method of any of embodiments 1-27, wherein the administration of the kinase inhibitor per day it is administered is from or from about 280 mg to 560 mg.

29. The method of any of embodiments 1-28, wherein administration of the kinase inhibitor is initiated at least at or about 7 days prior to obtaining the sample from the subject.

30. The method of any of embodiments 1-29, wherein:

the administration of the kinase inhibitor is initiated from or from about 30 to 40 days prior to initiating the administration of the T cell therapy;

the sample is obtained from the subject from or from about 23 days to 38 days prior to initiating the administration of the T cell therapy; and/or the lymphodepleting therapy is completed 5 to 7 days prior to initiating administration of the T cell therapy.

31. The method of any of embodiments 1-30, wherein:

the administration of the kinase inhibitor is initiated at or about 35 days prior to initiating the administration of the T cell therapy;

the sample is obtained from the subject from or from about 28 days to 32 days prior to initiating the administration of the T cell therapy; and/or

the lymphodepleting therapy is completed 5 to 7 days prior to initiating administration of the T cell therapy.

32. The method of any of embodiments 5-31, wherein the lymphodepleting therapy comprises the administration of fludarabine and/or cyclophosphamide.

33. The method of any of embodiments 5-32, wherein the lymphodepleting therapy comprises administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days, or wherein the lymphodepleting therapy comprises administration of cyclophosphamide at about 500 mg/m².

34. The method of any one of embodiments 5-33, wherein:

the lymphodepleting therapy comprises administration of cyclophosphamide at or about 300 mg/m² and fludarabine at about 30 mg/m² daily for 3 days; and/or

the lymphodepleting therapy comprises administration of cyclophosphamide at or about 500 mg/m² and fludarabine at about 30 mg/m² daily for 3 days.

35. The method of any of embodiments 1-34, wherein the administration of the kinase inhibitor per day it is administered is at an amount of at or about 140 mg.

36. The method of any of embodiments 1-34, wherein the administration of the kinase inhibitor per day it is administered is at an amount of at or about 280 mg.

37. The method of any of embodiments 1-34, wherein the administration of the kinase inhibitor per day it is administered is at an amount of at or about 420 mg.

38. The method of any of embodiments 1-34, wherein the administration of the kinase inhibitor per day it is administered is at an amount of at or about 560 mg.

39. The method of any of embodiments 9-38, wherein the period extends for at or about or greater than four months after the initiation of the administration of the T cell therapy or at or about or greater than five months after the initiation of the administration of the T cell therapy.

40. The method of any of embodiments 9-39, wherein the further administration is for a period that extends at or about or greater than six months.

41. The method of any of embodiments 9-40, wherein:

the further administration of the kinase inhibitor is stopped at the end of the period, if, at the end of the period, the subject exhibits a complete response (CR) following the treatment; or

the further administration of the kinase inhibitor is stopped at the end of the period if, at the end of the period, the cancer has progressed or relapsed following remission after the treatment.

42. The method of any of embodiments 9-41, wherein the period extends for from or from at or about three months to at or six months.

43. The method of any of embodiments 9-42, wherein the period extends for at or about three months after initiation of administration of the T cell therapy.

44. The method of any of embodiments 9-42, wherein the period extends for at or about 3 months after initiation of administration of the T cell therapy if the subject has, prior to at or about 3 months, achieved a complete response (CR) following the treatment or the cancer has progressed or relapsed following remission after the treatment.

45. The method of embodiment 44, wherein the period extends for at or about 3 months after initiation of administration of the T cell therapy if the subject has at 3 months achieved a complete response (CR).

46. The method of any of embodiments 9-42, wherein the period extends for at or about six months after initiation of administration of the T cell therapy.

47. The method of any of embodiments 9-42, wherein the period extends for at or about 6 months after initiation of administration of the T cell therapy if the subject has, prior to at or about 6 months, achieved a complete response (CR) following the treatment or the cancer has progressed or relapsed following remission after the treatment.

48. The method of embodiment 47, wherein the period extends for at or about 6 months after initiation of administration of the T cell therapy if the subject has at 6 months achieved a complete response (CR).

49. The method of any of embodiments 9-48, wherein the further administration is continued for the duration of the period even if the subject has achieved a complete response (CR) at a time point prior to the end of the period.

50. The method of any of embodiments 9-49, wherein the subject achieves a complete response (CR) at a time during the period and prior to the end of the period.

51. The method of any of embodiments 9-40, 42, 43, 44, 46 and 47, further comprising continuing the further administration after the end of the period, if, at the end of the period, the subject exhibits a partial response (PR) or stable disease (SD).

52. The method of any of embodiments 9-40, 42, 43, 44, 46, 47 and 51, wherein the further administration is continued for greater than six months if, at or about six months, the subject exhibits a partial response (PR) or stable disease (SD) after the treatment.

53. The method of embodiment 51 or embodiment 52, wherein the further administration is continued until the subject has achieved a complete response (CR) following the treatment or until the cancer has progressed or relapsed following remission after the treatment.

54. The method of any of embodiments 1-53, wherein the kinase inhibitor inhibits Bruton's tyrosine kinase (BTK) and/or inhibits IL2 inducible T-cell kinase (ITK).

55. The method of any of embodiments 1-54, wherein the kinase inhibitor inhibits ITK and the inhibitor inhibits ITK or inhibits ITK with a half-maximal inhibitory concentration (IC₅₀) of less than or less than about 1000 nM, 900 nM, 800 nM, 600 nM, 500 nM, 400 nM, 300 nM, 200 nM, 100 nM or less.

56. The method of any of embodiments 1-55, wherein the subject had previously been administered the kinase inhibitor prior to the administration of the kinase inhibitor in (1).

57. The method of any of embodiments 1-55, wherein the subject has not previously been administered the kinase inhibitor prior to the administration of the kinase inhibitor in (1).

58. The method of any of embodiments 1-57, wherein:

(i) the subject and/or the cancer (a) is resistant to inhibition of Bruton's tyrosine kinase (BTK) and/or (b) comprises a population of cells that are resistant to inhibition by the kinase inhibitor, optionally wherein the population of cells is or comprises a population of B cells and/or does not comprise T cells;

(ii) the subject and/or the cancer comprises a mutation in a nucleic acid encoding a BTK, optionally wherein the mutation is capable of reducing or preventing inhibition of the BTK by the kinase inhibitor, optionally wherein the mutation is C481S;

(iii) the subject and/or the cancer comprises a mutation in a nucleic acid encoding phospholipase C gamma 2 (PLCgamma2), optionally wherein the mutation results in constitutive signaling activity, optionally wherein the mutation is R665W or L845F;

(iv) at the time of the initiation of administration of the kinase inhibitor in (1), and optionally at the time of the initiation of administration of the T cell therapy, the subject has relapsed following remission after a previous treatment with, or been deemed refractory to a previous treatment with, the kinase inhibitor and/or with a BTK inhibitor therapy;

(v) at the time of the initiation of administration of the kinase inhibitor in (1), and optionally at the time of the initiation of the T cell therapy, the subject has progressed following a previous treatment with the inhibitor and/or with a BTK inhibitor therapy, optionally wherein the subject exhibited progressive disease as the best response to the previous treatment or progression after previous response to the previous treatment; and/or

(vi) at the time of the initiation of administration of the kinase inhibitor in (1), and optionally at the time of the initiation of the T cell therapy, the subject exhibited a response less than a complete response (CR) following a previous treatment for at least 6 months with the inhibitor and/or with a BTK inhibitor therapy.

59. The method of any one of embodiments 1-58, wherein the cancer is a B cell malignancy.

60. The method of embodiment 59, wherein the B cell malignancy is a lymphoma.

61. The method of embodiment 60, wherein the lymphoma is a non-Hodgkin lymphoma (NHL).

62. The method of embodiment 61, wherein the NHL comprises aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma with MYC and BCL2 and/or BCL6 rearrangements with DLBCL histology (double/triple hit).

63. The method of any one of embodiments 1-62, wherein the subject is or has been identified as having an Eastern Cooperative Oncology Group Performance Status (ECOG) status of less than or equal to 1.

64. The method of any of embodiments 1-63, wherein the kinase inhibitor is administered orally.

65. The method of any of embodiments 1-64, wherein the CD19 is a human CD19.

66. The method of any of embodiments 1-65, wherein the chimeric antigen receptor (CAR) comprises an extracellular antigen-recognition domain that specifically binds to the CD19 and an intracellular signaling domain comprising an ITAM.

67. The method of embodiment 66, wherein the intracellular signaling domain comprises a signaling domain of a CD3-zeta (CD3) chain, optionally a human CD3-zeta chain.

68. The method of embodiment 66 or embodiment 67, wherein the chimeric antigen receptor (CAR) further comprises a costimulatory signaling region.

69. The method of embodiment 68, wherein the costimulatory signaling region comprises a signaling domain of CD28 or 4-1BB, optionally human CD28 or human 4-1BB.

70. The method of embodiment 68 or embodiment 69, wherein the costimulatory domain is or comprises a signaling domain of human 4-1BB.

71. The method of any of embodiments 1-70, wherein:

the CAR comprises an scFv specific for the CD19; a transmembrane domain; a cytoplasmic signaling domain derived from a costimulatory molecule, which optionally is or comprises a 4-1BB, optionally human 4-1BB; and a cytoplasmic signaling domain derived from a primary signaling ITAM-containing molecule, which optionally is or comprises a CD3zeta signaling domain, optionally a human CD3zeta signaling domain; and optionally wherein the CAR further comprises a spacer between the transmembrane domain and the scFv;

the CAR comprises, in order, an scFv specific for the CD19; a transmembrane domain; a cytoplasmic signaling domain derived from a costimulatory molecule, which optionally is or comprises a 4-1BB signaling domain, optionally a human 4-1BB signaling domain; and a cytoplasmic signaling domain derived from a primary signaling ITAM-containing molecule, which optionally is a CD3zeta signaling domain, optionally human CD3zeta signaling domain; or

the CAR comprises, in order, an scFv specific for the CD19; a spacer; a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

72. The method of embodiment 71, wherein

the CAR comprises a spacer and the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X1PPX2P (SEQ ID NO:58), where X1 is glycine, cysteine or arginine and X2 is cysteine or threonine; and/or

the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or

the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or

the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a V_{H}, a linker, optionally comprising SEQ ID NO: 41, and a V_{L}, and/or the scFv comprises a flexible linker and/or comprises the amino acid sequence set forth as SEQ ID NO: 42.

73. The method of any of embodiments 1-72, wherein the dose of genetically engineered T cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

74. The method of any of embodiments 1-73, wherein the dose of genetically engineered T cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

75. The method of any of embodiments 1-74, wherein the dose of genetically engineered T cells comprises at or about 5×10⁷ total CAR-expressing T cells.

76. The method of any of embodiments 1-75, wherein the dose of genetically engineered T cells comprises at or about 1×10⁸ CAR-expressing cells.

77. The method of any of embodiments 1-76, wherein the dose of genetically engineered T cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and the second composition comprising the other of the CD4+ T cells or the CD8+ T cells.

78. The method of embodiment 77, wherein:

the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or

the initiation of administration of the first composition and the initiation of administration of the second composition are carried out between about 1 minute and about 1 hour apart or between about 5 minutes and about 30 minutes apart.

79. The method of embodiment 77 or embodiment 78, wherein the first composition and second composition are administered no more than 2 hours, no more than 1 hour, no more than 30 minutes, no more than 15 minutes, no more than 10 minutes or no more than 5 minutes apart.

80. The method of any of embodiments 77-79, wherein the first composition comprises the CD4+ T cells.

81. The method of any of embodiments 77-79, wherein the first composition comprises the CD8+ T cells.

82. The method of any of embodiments 77-81, wherein the first composition is administered prior to the second composition.

83. The method of any of embodiments 1-82, wherein the dose of cells is administered parenterally, optionally intravenously.

84. The method of any of embodiments 1-83, wherein the T cells are primary T cells obtained from the sample from the subject.

85. The method of any of embodiments 1-82, wherein the T cells are autologous to the subject.

86. The method of any of embodiments 1-85, wherein the processing comprises:

isolating T cells, optionally CD4+ and/or CD8+ T cells, from the sample obtained from the subject, thereby producing an input composition comprising primary T cells; and

introducing the nucleic acid molecule encoding the CAR into T cells of the input composition.

87. The method of embodiment 86, wherein the isolating comprising carrying out immunoaffinity-based selection.

88. The method of any of embodiments 1-87, wherein the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.

89. The method of any of embodiments 86-88, wherein prior to the introducing, the processing comprises incubating the input composition under stimulating conditions, said stimulating conditions comprising the presence of a stimulatory reagent capable of activating one or more intracellular signaling domains of one or more components of a TCR complex and/or one or more intracellular signaling domains of one or more costimulatory molecules, thereby generating a stimulated composition, wherein the nucleic acid molecule encoding the CAR is introduced into the stimulated composition.

90. The method of embodiment 89, wherein the stimulatory reagent comprises a primary agent that specifically binds to a member of a TCR complex, optionally that specifically binds to CD3.

91. The method of embodiment 90, wherein the stimulatory reagent further comprises a secondary agent that specifically binds to a T cell costimulatory molecule, optionally wherein the costimulatory molecule is selected from CD28, CD137 (4-1-BB), OX40, or ICOS.

92. The method of embodiment 90 or embodiment 91, wherein the primary and/or secondary agents comprise an antibody, optionally wherein the stimulatory reagent comprises incubation with an anti-CD3 antibody and an anti-CD28 antibody, or an antigen-binding fragment thereof.

93. The method of any of embodiments 90-92, wherein the primary agent and/or secondary agent are present on the surface of a solid support.

94. The method of embodiment 93, wherein the solid support is or comprises a bead, optionally wherein the bead is magnetic or superparamagnetic.

95. The method of embodiment 94, wherein the bead comprises a diameter of greater than or greater than about 3.5 μm but no more than about 9 μm or no more than about 8 μm or no more than about 7 μm or no more than about 6 μm or no more than about 5 μm.

96. The method of embodiment 94 or embodiment 95, wherein the bead comprises a diameter of or about 4.5 μm.

97. The method of any of embodiments 1-96, wherein the introducing comprises transducing cells of the stimulated composition with a viral vector comprising a polynucleotide encoding the recombinant receptor.

98. The method of embodiment 97, wherein the viral vector is a retroviral vector.

99. The method of embodiment 97 or embodiment 98, wherein the viral vector is a lentiviral vector or gammaretroviral vector.

100. The method of any of embodiments 86-99, wherein the processing further comprises after the introducing cultivating the T cells, optionally wherein the cultivating is carried out under conditions to result in the proliferation or expansion of cells to produce an output composition comprising the T cell therapy.

101. The method of embodiment 100, wherein subsequent to the cultivating, the method further comprises formulating cells of the output composition for cryopreservation and/or for administration of the T cell therapy to the subject, optionally wherein the formulating is in the presence of a pharmaceutically acceptable excipient.

102. The method of any of embodiments 1-101, wherein the subject is a human.

103. The method of any of embodiments 1-102, wherein:

at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or

wherein at least 60, 70, 80, 90, or 95% of subjects achieving a CR by six months remain in response, remain in CR, and/or survive or survive without progression, for greater at or greater than 3 months and/or at or greater than 6 months and/or at greater than nine months; and/or

at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or

wherein at least 60, 70, 80, 90, or 95% of subjects achieving an OR by six months remain in response or surviving for greater at or greater than 3 months and/or at or greater than 6 months.

104. The method of any of embodiments 60-103, wherein, at or immediately prior to the time of the administration of the dose of cells the subject has relapsed following remission after treatment with, or become refractory to, one or more prior therapies for the lymphoma, optionally the NHL, optionally one, two or three prior therapies other than another dose of cells expressing the CAR.

105. The method of any of embodiment 60-104, wherein, at or prior to the administration of the T cell therapy comprising the dose of cells:

the subject is or has been identified as having a double/triple hit lymphoma;

the subject is or has been identified as having a chemorefractory lymphoma, optionally a chemorefractory DLBCL; and/or

the subject has not achieved a complete response (CR) in response to a prior therapy.

106. A kit comprising one or more unit doses of a kinase inhibitor that is or comprises the structure

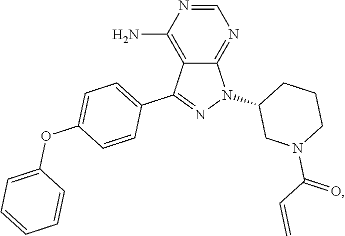 [SMILES: C=CC(=O)N1CCC[C@@H](n2nc(-c3ccc(Oc4ccccc4)cc3)c3c(N)ncnc32)C1]

or is a pharmaceutically acceptable salt thereof, and instructions for administering the one or more unit doses to a subject having a cancer that is a candidate for treatment with or who is to be treated with an autologous T cell therapy, said T cell therapy comprising a dose of genetically engineered T cells expressing a chimeric antigen receptor (CAR) that specifically binds to a CD19, and in which, prior to administration of the T cell therapy, a biological sample is obtained from the subject and processed, the processing comprising genetically modifying T cells from the sample, optionally by introducing a nucleic acid encoding the CAR into the T cells,

wherein the instructions specify initiating administration of a unit dose of the kinase inhibitor to the subject at or at least about 3 days prior to the obtaining of the sample and in a dosing regimen comprising repeat administrations of one or more unit doses at a dosing interval over a period of time that extends at least to include administration on or after the day the sample is obtained from the subject.

107. The kit of embodiment 106, wherein the instructions further specify administering the T cell therapy to the subject.

108. The kit of embodiment 106 or embodiment 107, wherein the instructions further specify, subsequent to initiating the administration of the kinase inhibitor and prior to the administration of the T cell therapy, administering a lymphodepleting therapy to the subject.

109. The kit of embodiment 108, wherein the instructions specify administration of the kinase inhibitor is to be discontinued during administration of the lymphodepleting therapy.

110. The kit of embodiment 108 or embodiment 109, wherein the instructions specify the dosing regimen comprises administration of the kinase inhibitor for a period of time that extends at least until the initiation of the lymphodepleting therapy.

111. The kit of any of embodiments 108-110, wherein the instructions specify the dosing regimen comprises administration of the kinase inhibitor over a period of time that includes administration up to the initiation of the lymphodepleting therapy, followed by discontinuing or halting administration of the kinase inhibitor during the lymphodepleting therapy, and then further administration of the kinase inhibitor for a period that extends for at least 15 days after initiation of administration of the T cell therapy.

112. The kit of any of embodiments 106-111, wherein the instructions specify administration of the kinase inhibitor is initiated at least at or about 4 days, at least at or about 5 days, at least at or about 6 days, at least at or about 7 days, at least at or about 14 days or more prior to obtaining the sample from the subject.

113. The kit of any of embodiments 106-112, wherein the instructions specify administration of the kinase inhibitor is initiated at least at or about 5 days to 7 days prior to obtaining the sample from the subject.

114. The kit of any of embodiments 108-113, wherein the instructions specify the administration of the lymphodepleting therapy is to be completed within 7 days prior to initiation of the administration of the T cell therapy.

115. The kit of any of embodiments 108-1114, wherein the instructions specify the administration of the lymphodepleting therapy is to be completed 2 to 7 days prior to initiation of the administration of the T cell therapy.

116. The kit of any of embodiments 111-115, wherein the instructions specify the further administration of the kinase inhibitor is for a period that extends at or about or greater than three months after initiation of administration of the T cell therapy.

117. The kit of any of embodiments 106-116, wherein the instructions specify the administration of each unit dose of the kinase inhibitor is carried out once per day on each day it is administered during the dosing regimen.

118. The kit of any of embodiments 106-117, wherein the one or more unit doses each comprises from or from about 140 mg to about 840 mg.

119. The kit of any of embodiments 106-118, wherein the one or more unit doses each comprise from or from about 140 mg to about 560 mg per each day the kinase inhibitor is administered.

120. The kit of any of embodiments 106-119, wherein the one or more unit doses each comprise from or from about 280 mg to 560 mg.

121. The kit of any of embodiments 106-120, wherein the instructions specify the administration of the kinase inhibitor is initiated a minimum of at or about 7 days prior to obtaining the sample from the subject.

122. The kit of any of embodiments 106-121, wherein the instructions specify:

the administration of the kinase inhibitor is initiated from or from about 30 to 40 days prior to initiating the administration of the T cell therapy;

the sample is obtained from the subject from or from about 23 days to 38 days prior to initiating the administration of the T cell therapy; and/or

the lymphodepleting therapy is completed 5 to 7 days prior to initiating administration of the T cell therapy.

123. The kit of any of embodiments 106-122, wherein the instructions specify:

the administration of the kinase inhibitor is initiated at or about 35 days prior to initiating the administration of the T cell therapy;

the sample is obtained from the subject from or from about 28 days to 32 days prior to initiating the administration of the T cell therapy; and/or

the lymphodepleting therapy is completed 5 to 7 days prior to initiating administration of the T cell therapy.

124. The kit of any of embodiments 108-123, wherein the lymphodepleting therapy comprises the administration of fludarabine and/or cyclophosphamide.

125. The kit of any of embodiments 108-124, wherein the instructions specify administration of the lymphodepleting therapy comprises administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days, or wherein the lymphodepleting therapy comprises administration of cyclophosphamide at about 500 mg/m².

126. The kit of any of embodiments 108-125, wherein the instruction specify:

the lymphodepleting therapy comprises administration of cyclophosphamide at or about 300 mg/m² and fludarabine at about 30 mg/m² daily for 3 days; and/or

the lymphodepleting therapy comprises administration of cyclophosphamide at or about 500 mg/m² and fludarabine at about 30 mg/m² daily for 3 days.

127. The kit of any of embodiments 106-126, wherein each unit dose of the kinase inhibitor is or is about 140 mg and/or the instructions specify administering the kinase inhibitor per day it is administered at an amount of at or about 140 mg.

128. The kit of any of embodiments 106-127, wherein each unit dose of the kinase inhibitor is or is about 280 mg and/or the instructions specify administering the kinase inhibitor per day it is administered is at an amount of at or about 280 mg.

129. The kit of any of embodiments 106-128, wherein each unit dose of the kinase inhibitor is or is about 420 mg and/or the instructions specify administering of the kinase inhibitor per day it is administered is at an amount of at or about 420 mg.

130. The method of any of embodiments 106-129, wherein each unit dose of the kinase inhibitor is or is about 560 mg and/or the instructions specify administering the kinase inhibitor per day it is administered is at an amount of at or about 560 mg.

131. The kit of any of embodiments 111-130, wherein the instructions specify the period extends for at or about or greater than four months after the initiation of the administration of the T cell therapy.

132. The kit of any of embodiments 111-131, wherein the instructions specify the period extends for at or about or greater than five months after the initiation of the administration of the T cell therapy.

133. The kit of any of embodiments 111-132, wherein the instructions specify the further administration is for a period that extends at or about or greater than six months.

134. The kit of any of embodiments 111-133, wherein the instructions specify the further administration of the kinase inhibitor is stopped at the end of the period, if, at the end of the period, the subject exhibits a complete response (CR) following the treatment.

135. The kit of any of embodiments 111-134, wherein the instructions specify further administration of the kinase inhibitor is stopped at the end of the period if, at the end of the period, the cancer has progressed or relapsed following remission after the treatment.

136. The kit of any of embodiments 111-135, wherein the instructions specify the period extends for from or from at or about three months to at or six months.

137. The kit of any of embodiments 111-136, wherein the instructions specify the period extends for at or about three months after initiation of administration of the T cell therapy.

138. The kit of any of embodiments 111-136, wherein the instructions specify the period extends for at or about 3 months after initiation of administration of the T cell therapy if the subject has, prior to at or about 3 months, achieved a complete response (CR) following the treatment or the cancer has progressed or relapsed following remission after the treatment.

139. The kit of embodiment 138, wherein the instructions specify the period extends for at or about 3 months after initiation of administration of the T cell therapy if the subject has at 3 months achieved a complete response (CR).

140. The kit of any of embodiments 111-136, wherein the instructions specify the period extends for at or about six months after initiation of administration of the T cell therapy.

141. The kit of any of embodiments 111-136, wherein the instructions specify the period extends for at or about 6 months after initiation of administration of the T cell therapy if the subject has, prior to at or about 6 months, achieved a complete response (CR) following the treatment or the cancer has progressed or relapsed following remission after the treatment.

142. The kit of embodiment 141, wherein the instructions specify the period extends for at or about 6 months after initiation of administration of the T cell therapy if the subject has at 6 months achieved a complete response (CR).

143. The kit of any of embodiments 111-142, wherein the instructions specify the further administration is continued for the duration of the period even if the subject has achieved a complete response (CR) at a time point prior to the end of the period.

144. The kit of any of embodiments 111-133, 136, 137, 138, 140 and 141, wherein the instructions specify further comprising continuing the further administration after the end of the period, if, at the end of the period, the subject exhibits a partial response (PR) or stable disease (SD).

145. The kit of any of embodiments 111-133, 136, 137, 138, 140, 141 and 144, wherein the instructions specify the further administration is continued for greater than six months if, at or about six months, the subject exhibits a partial response (PR) or stable disease (SD) after the treatment.

146. The kit of embodiment 144 or embodiment 144, wherein the instructions specify the further administration is continued until the subject has achieved a complete response (CR) following the treatment or until the cancer has progressed or relapsed following remission after the treatment.

147. The kit of any of embodiments 106-146, wherein the kinase inhibitor inhibits Bruton's tyrosine kinase (BTK) and/or inhibits IL2 inducible T-cell kinase (ITK).

148. The kit of any of embodiments 106-147, wherein the kinase inhibitor inhibits ITK and the inhibitor inhibits ITK or inhibits ITK with a half-maximal inhibitory concentration (IC₅₀) of less than or less than about 1000 nM, 900 nM, 800 nM, 600 nM, 500 nM, 400 nM, 300 nM, 200 nM, 100 nM or less.

149. The kit of any of embodiments 106-148, wherein the instructions specify the subject has been or can have been previously administered the kinase inhibitor prior to the administration of the one or more unit doses of the kinase inhibitor.

150. The kit of any of embodiments 106-148, wherein the instructions specify the subject has not been or is one who has not been previously administered the kinase inhibitor prior to the administration of the one or more unit doses of the kinase inhibitor.

151. The kit of any of embodiments 106-150, wherein the instructions specify:

(i) the subject and/or the cancer (a) is resistant to inhibition of Bruton's tyrosine kinase (BTK) and/or (b) comprises a population of cells that are resistant to inhibition by the kinase inhibitor, optionally wherein the population of cells is or comprises a population of B cells and/or does not comprise T cells;

(ii) the subject and/or the cancer comprises a mutation in a nucleic acid encoding a BTK, optionally wherein the mutation is capable of reducing or preventing inhibition of the BTK by the kinase inhibitor, optionally wherein the mutation is C481S;

(iii) the subject and/or the cancer comprises a mutation in a nucleic acid encoding phospholipase C gamma 2 (PLCgamma2), optionally wherein the mutation results in constitutive signaling activity, optionally wherein the mutation is R665W or L845F;

(iv) at the time of the initiation of administration of the kinase inhibitor in (1), and optionally at the time of the initiation of administration of the T cell therapy, the subject has relapsed following remission after a previous treatment with, or been deemed refractory to a previous treatment with, the kinase inhibitor and/or with a BTK inhibitor therapy;

(v) at the time of the initiation of administration of the kinase inhibitor in (1), and optionally at the time of the initiation of the T cell therapy, the subject has progressed following a previous treatment with the inhibitor and/or with a BTK inhibitor therapy, optionally wherein the subject exhibited progressive disease as the best response to the previous treatment or progression after previous response to the previous treatment; and/or

(vi) at the time of the initiation of administration of the kinase inhibitor in (1), and optionally at the time of the initiation of the T cell therapy, the subject exhibited a response less than a complete response (CR) following a previous treatment for at least 6 months with the inhibitor and/or with a BTK inhibitor therapy.

152. The kit of any of embodiments 106-151, wherein the cancer is a B cell malignancy.

153. The kit of embodiment 152, wherein the B cell malignancy is a lymphoma.

154. The kit of embodiment 153, wherein the lymphoma is a non-Hodgkin lymphoma (NHL).

155. The kit of embodiment 154, wherein the NHL comprises aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma with MYC and BCL2 and/or BCL6 rearrangements with DLBCL histology (double/triple hit).

156. The kit of any of embodiments 106-155, wherein the subject is or has been identified as having an Eastern Cooperative Oncology Group Performance Status (ECOG) status of less than or equal to 1.

157. The kit of any of embodiments 106-156, wherein the one or more unit doses of the kinase inhibitor is formulated for oral administration and/or the instructions further specify the one or more unit doses of the kinase inhibitor is administered orally.

158. The kit of any of embodiments 106-157, wherein the CD19 is a human CD19.

159. The kit of any of embodiments 106-158, wherein the chimeric antigen receptor (CAR) comprises an extracellular antigen-recognition domain that specifically binds to the CD19 and an intracellular signaling domain comprising an ITAM.

160. The kit of embodiment 159, wherein the intracellular signaling domain comprises a signaling domain of a CD3-zeta (CD3) chain, optionally a human CD3-zeta chain.

161. The kit of embodiment 159 or embodiment 160, wherein the chimeric antigen receptor (CAR) further comprises a costimulatory signaling region.

162. The kit of embodiment 161, wherein the costimulatory signaling region comprises a signaling domain of CD28 or 4-1BB, optionally human CD28 or human 4-1BB.

163. The kit of embodiment 161 or embodiment 162, wherein the costimulatory domain is or comprises a signaling domain of human 4-1BB.

164. The kit of any of embodiments 106-163, wherein:

the CAR comprises an scFv specific for the CD19; a transmembrane domain; a cytoplasmic signaling domain derived from a costimulatory molecule, which optionally is or comprises a 4-1BB, optionally human 4-1BB; and a cytoplasmic signaling domain derived from a primary signaling ITAM-containing molecule, which optionally is or comprises a CD3zeta signaling domain, optionally a human CD3zeta signaling domain; and optionally wherein the CAR further comprises a spacer between the transmembrane domain and the scFv;

the CAR comprises, in order, an scFv specific for the CD19; a transmembrane domain; a cytoplasmic signaling domain derived from a costimulatory molecule, which optionally is or comprises a 4-1BB signaling domain, optionally a human 4-1BB signaling domain; and a cytoplasmic signaling domain derived from a primary signaling ITAM-containing molecule, which optionally is a CD3zeta signaling domain, optionally human CD3zeta signaling domain; or

the CAR comprises, in order, an scFv specific for the CD19; a spacer; a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

165. The kit of embodiment 164, wherein

the CAR comprises a spacer and the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X1PPX2P (SEQ ID NO:58), where X1 is glycine, cysteine or arginine and X2 is cysteine or threonine; and/or

the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or

the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or

the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a V_{H}, a linker, optionally comprising SEQ ID NO: 41, and a V_{L}, and/or the scFv comprises a flexible linker and/or comprises the amino acid sequence set forth as SEQ ID NO: 42.

166. The kit of any of embodiments 106-165, wherein the dose of genetically engineered T cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

167. The kit of any of embodiments 106-166, wherein the dose of genetically engineered T cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

168. The kit of any of embodiments 106-167, wherein the dose of genetically engineered T cells comprises at or about 5×10⁷ total CAR-expressing T cells.

169. The kit of any of embodiments 106-168, wherein the dose of genetically engineered T cells comprises at or about 1×10⁸ CAR-expressing cells.

170. The kit of any of embodiments 106-169, wherein the dose of genetically engineered T cells comprises CD4+ T cells expressing the CAR and CD8+ T cells expressing the CAR and the instructions specify administration of the dose comprises administering a plurality of separate compositions, said plurality of separate compositions comprising a first composition comprising one of the CD4+ T cells and the CD8+ T cells and the second composition comprising the other of the CD4+ T cells or the CD8+ T cells.

171. The kit of embodiment 170, wherein the instructions specify:

the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or

the initiation of administration of the first composition and the initiation of administration of the second composition are carried out between about 1 minute and about 1 hour apart or between about 5 minutes and about 30 minutes apart.

172. The kit of embodiment 170 or embodiment 171, wherein the instructions specify the first composition and second composition are administered no more than 2 hours, no more than 1 hour, no more than 30 minutes, no more than 15 minutes, no more than 10 minutes or no more than 5 minutes apart.

173. The kit of any of embodiments 170-172, wherein the instructions specify the first composition comprises the CD4+ T cells.

174. The kit of any of embodiments 170-172, wherein the instructions specify the first composition comprises the CD8+ T cells.

175. The kit of any of embodiments 170-174, wherein the instructions specify the first composition is administered prior to the second composition.

176. The kit of any of embodiments 106-175, wherein the instructions specify the dose of cells is administered parenterally, optionally intravenously.

177. The kit of any of embodiments 106-176, wherein the T cells are primary T cells obtained from the sample from the subject.

178. The kit of any of embodiments 106-177, wherein the T cells are autologous to the subject.

179. The kit of any of embodiments 106-178, wherein the instructions further specify the process for producing the T cell therapy.

180. The kit of any of embodiments 106-179, wherein the process for producing the T cell therapy comprises:

isolating T cells, optionally CD4+ and/or CD8+ T cells, from the sample obtained from the subject, thereby producing an input composition comprising primary T cells; and

introducing the nucleic acid molecule encoding the CAR into the input composition.

181. The kit of embodiment 180, wherein the isolating comprising carrying out immunoaffinity-based selection.

182. The kit of any of embodiments 106-181, wherein the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.

183. The kit of any of embodiments 180-182, wherein prior to the introducing, the process comprises incubating the input composition under stimulating conditions, said stimulating conditions comprising the presence of a stimulatory reagent capable of activating one or more intracellular signaling domains of one or more components of a TCR complex and/or one or more intracellular signaling domains of one or more costimulatory molecules, thereby generating a stimulated composition, wherein the nucleic acid molecule encoding the CAR is introduced into the stimulated composition.

184. The kit of embodiment 183, wherein the stimulatory reagent comprises a primary agent that specifically binds to a member of a TCR complex, optionally that specifically binds to CD3.

185. The kit of embodiment 184, wherein the stimulatory reagent further comprises a secondary agent that specifically binds to a T cell costimulatory molecule, optionally wherein the costimulatory molecule is selected from CD28, CD137 (4-1-BB), OX40, or ICOS.

186. The kit of embodiment 184 or embodiment 185, wherein the primary and/or secondary agents comprise an antibody, optionally wherein the stimulatory reagent comprises incubation with an anti-CD3 antibody and an anti-CD28 antibody, or an antigen-binding fragment thereof.

187. The kit of any of embodiments 184-186, wherein the primary agent and/or secondary agent are present on the surface of a solid support.

188. The kit of embodiment 187, wherein the solid support is or comprises a bead, optionally wherein the bead is magnetic or superparamagnetic.

189. The kit of embodiment 188, wherein the bead comprises a diameter of greater than or greater than about 3.5 μm but no more than about 9 μm or no more than about 8 μm or no more than about 7 μm or no more than about 6 μm or no more than about 5 μm.

190. The kit of embodiment 188 or embodiment 189, wherein the bead comprises a diameter of or about 4.5 μm.

191. The kit of any of embodiments 106-190, wherein the introducing comprises transducing cells of the stimulated composition with a viral vector comprising a polynucleotide encoding the recombinant receptor.

192. The kit of embodiment 191, wherein the viral vector is a retroviral vector.

193. The kit of embodiment 191 or embodiment 192, wherein the viral vector is a lentiviral vector or gammaretroviral vector.

194. The kit of any of embodiments 180-193, wherein the process further comprises after the introducing cultivating the T cells, optionally wherein the cultivating is carried out under conditions to result in the proliferation or expansion of cells to produce an output composition comprising the T cell therapy.

195. The kit of embodiment 194, wherein subsequent to the cultivating, the process further comprises formulating cells of the output composition for cryopreservation and/or for administration of the T cell therapy to the subject, optionally wherein the formulating is in the presence of a pharmaceutically acceptable excipient.

196. The kit of any of embodiments 106-195, wherein the instructions specify the subject is a human.

197. The kit of any of embodiment 106-196, wherein the instructions specify, at or prior to the administration of the T cell therapy comprising the dose of cells:

the subject is or has been identified as having a double/triple hit lymphoma;

the subject is or has been identified as having a chemorefractory lymphoma, optionally a chemorefractory DLBCL; and/or

the subject has not achieved a complete response (CR) in response to a prior therapy.

198. A kit comprising one or more unit doses of a kinase inhibitor that is or comprises the structure

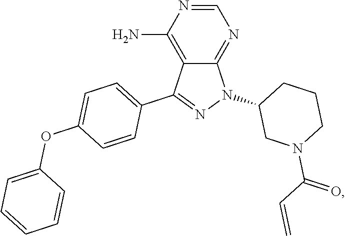 [SMILES: C=CC(=O)N1CCC[C@@H](n2nc(-c3ccc(Oc4ccccc4)cc3)c3c(N)ncnc32)C1]

or is a pharmaceutically acceptable salt thereof, and instructions for carrying out the methods of any of claims 1-105.

199. An article of manufacture comprising the kit of any of embodiments 106-198.

### VIII. Examples

The following examples are included for illustrative purposes only and are not intended to limit the scope of the invention.

### Example 1: Assessment of CAR-Expressing T Cell Phenotype and Function in the Presence of Ibrutinib

Properties of CAR-expressing T cells in the presence of a Btk inhibitor, ibrutinib (having the structure:

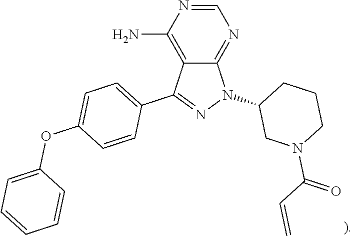 [SMILES: C=CC(=O)N1CCC[C@@H](n2nc(-c3ccc(Oc4ccccc4)cc3)c3c(N)ncnc32)C1]

were assessed in in vitro studies.

To generate CAR-expressing T cells, T cells were isolated by immunoaffinity-based enrichment from three healthy human donor subjects, and cells from each donor were activated and transduced with a viral vector encoding an anti-CD19 CAR. The CAR contained an anti-CD19 scFv, an Ig-derived spacer, a human CD28-derived transmembrane domain, a human 4-1BB-derived intracellular signaling domain and a human CD3 zeta-derived signaling domain. The nucleic acid construct encoding the CAR also included a truncated EGFR (tEGFR) sequence for use as a transduction marker, separated from the CAR sequence by a self-cleaving T2A sequence.

CAR-expressing CD4+ and CD8+ cells were mixed 1:1 for each donor, individually, and the pooled cells for each donor assessed in vitro under various conditions.

A. Cytolytic Activity

CAR T cells generated as described above were plated in triplicate on Poly-D-Lysine plates and then co-cultured with ibrutinib-resistant CD19-expressing target cells (K562 cells transduced to express CD19, K562-CD19) at an effector to target (E:T) ratio of 2.5:1. The target cells were labeled with NucLight Red (NLR), to permit tracking of target cells by microscopy. Ibrutinib was added to the cultures at concentrations of 5000, 500, 50, 5 and 0.5 nM (reflecting a dosage range covering doses observed to be supraphysiologic (500 nM) and Cmax (227 nM). CAR-T cells incubated in the presence of target cells in the absence of ibrutinib were used as an “untreated” control. Cytolytic activity was assessed by measuring the loss of viable target cells over a period of four days, as determined by red fluorescent signal (using the IncuCyte® Live Cell Analysis System, Essen Bioscience). Percent (%) of target killing was assessed by measuring area under the curve (AUC) for normalized target cell count over time and normalizing the inverse AUC (1/AUC) values by defining a 0% value (target cells alone) and a 100% value (CAR+ T cells co-cultured with target cells in vehicle control).

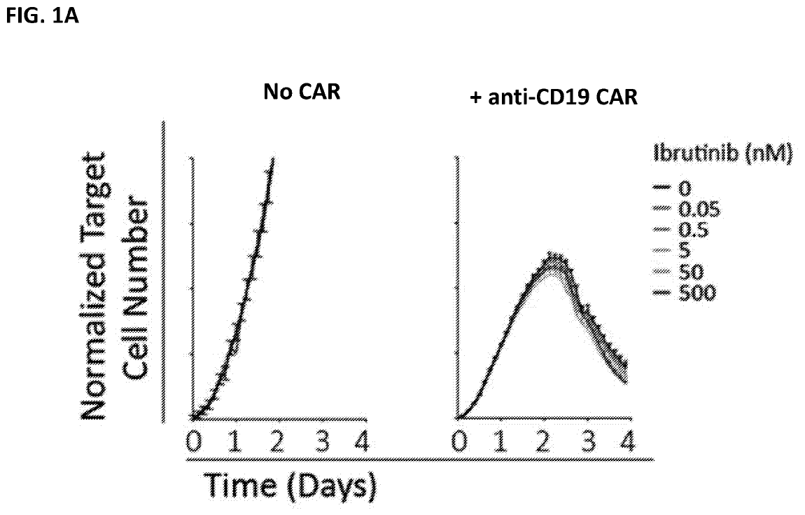

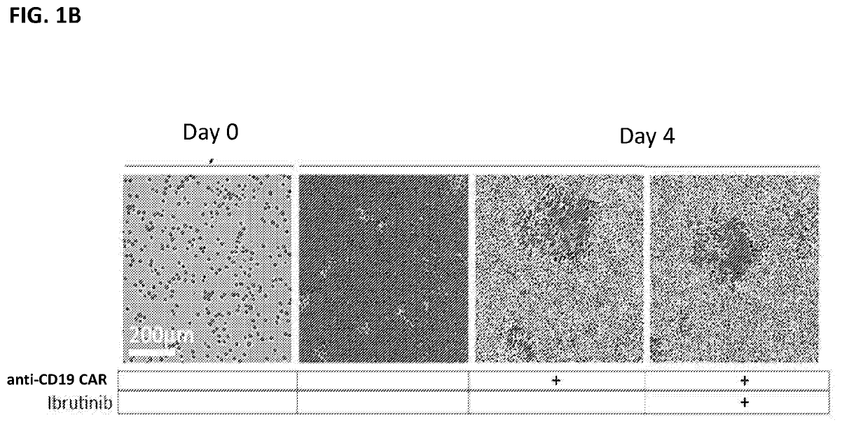

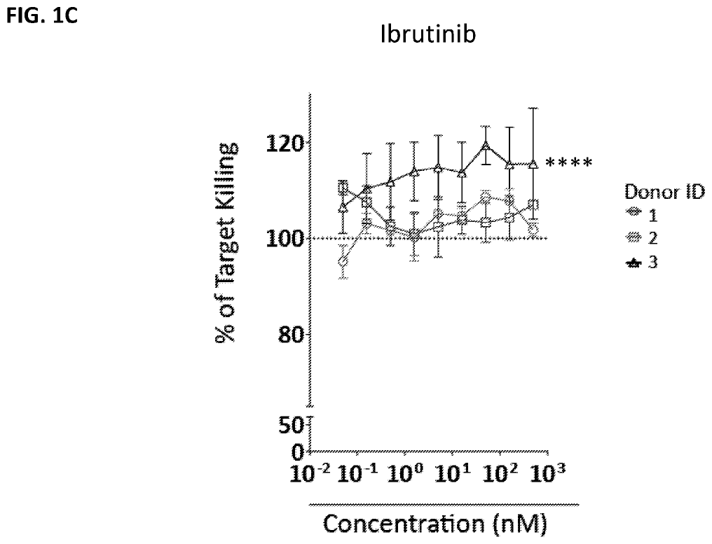

As shown by microscopy, after an initial period of target cell growth, anti-CD19 CAR T cells from all donors were observed to reduce the target cell number over a period of four days, thus demonstrating effective killing in the assay (FIG. 1A). A representative image of target cells co-cultured with CAR T cells at the start and end of the cytotoxic assay is shown in FIG. 1B. As shown in FIG. 1C, normalization of target cell killing by CAR-T cells treated with ibrutinib to untreated controls using area under the curve (AUC) calculations showed that ibrutinib, even when the concentration was increased to supra-physiological levels (500 nM), did not significantly impact cytolytic activity of the anti-CD19 CAR-expressing T cells in this assay for two donors. The addition of ibrutinib, at all concentrations tested during the co-culture, did not inhibit the cytolytic function of the anti-CD19 CAR T cells. However, a modestly increased target cell killing was observed for one donor treated with ibrutinib (P<0.0001) (FIG. 1C).

B. Expression of CAR-T Cell Surface Markers.

To assess various phenotypic markers of anti-CD19 CAR T cells cultured in the presence of ibrutinib, a panel of activation markers on CAR+, CD4+ and CD8+ cells (from three donors) were tracked over 4 days following stimulation with irradiated K562 target cells expressing CD19. CAR-T cells generated as described above were plated at 100,000 cells/well on 96 well Poly-D-Lysine coated plates. Irradiated K562-CD19 target cells were added at an effector to target ratio of 2.5:1. Cells were cultured for up to 4 days in the absence of ibrutinib or in the presence ibrutinib at concentrations of 5000, 500, 50, 5 and 0.5 nM for the duration of the culture. Cells were harvested at 1, 2, 3, and 4 days, and were analyzed by flow cytometry for T cell activation and differentiation surface markers CD69, CD107a, PD-1, CD25, CD38, CD39, CD95, CD62L, CCR7, CD45RO and for truncated EGFR (a surrogate marker for CAR-transduced cells).

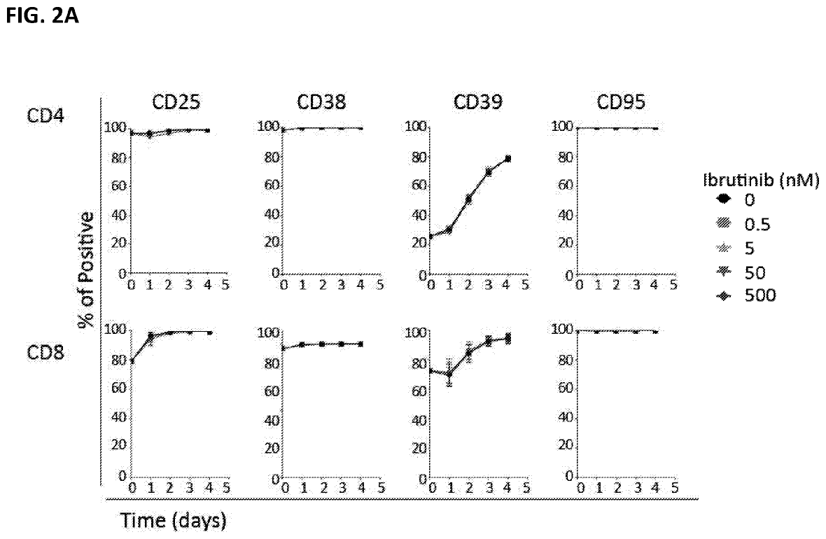

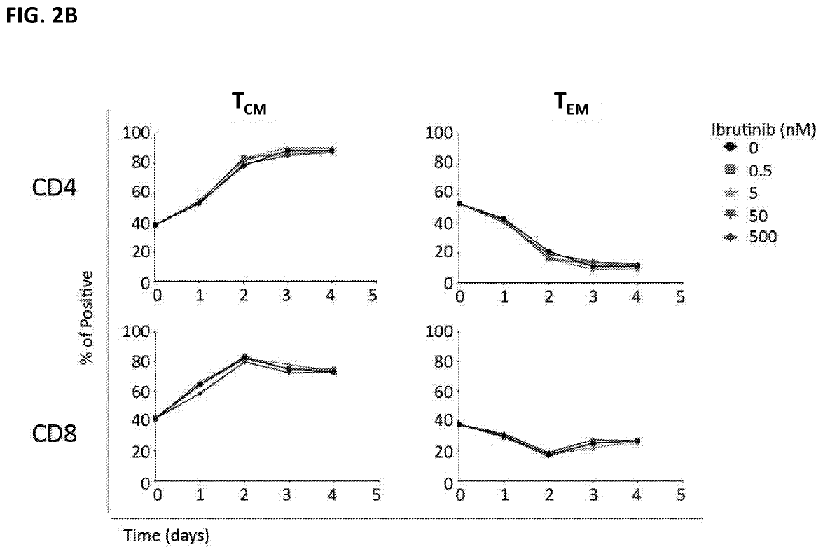

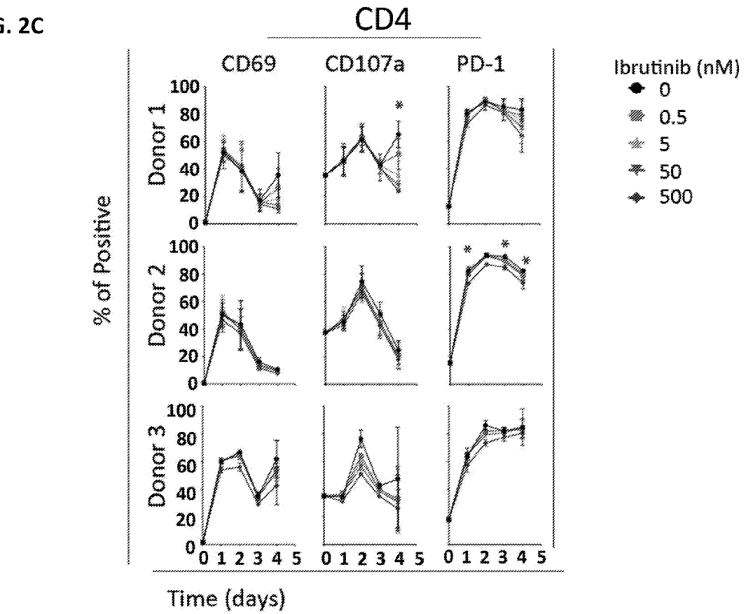

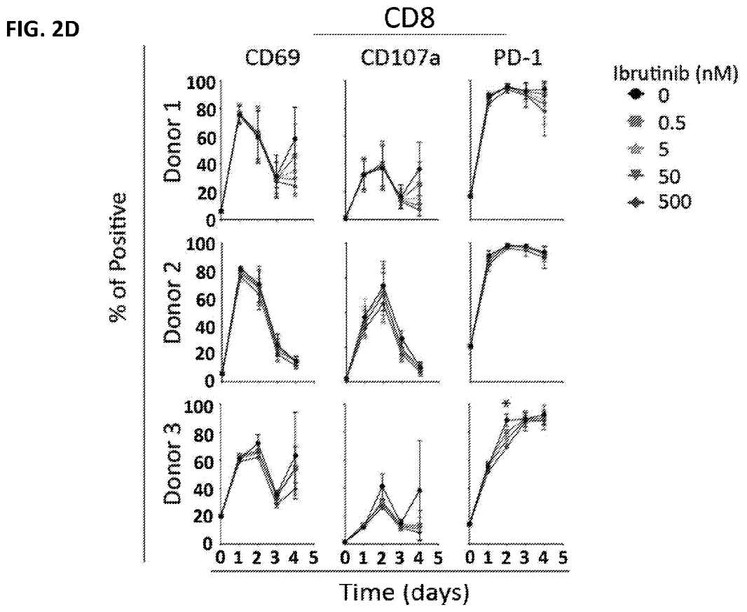

Across the 3 different anti-CD19 CAR T cell donors, ibrutinib at concentrations of 5000, 500, 50, 5 and 0.5 nM had no significant effect on expression of the truncated EGFR surrogate marker, on any of the activation markers CD25, CD38, CD39, CD95 and CD62L, or on any of the T cell phenotypic markers assessed in this study (CCR7, CD62L and CD45RO), consistent with a conclusion that the ibrutinib did not significantly impact the activation state and/or differentiation/subtype of the T cells in this assay. FIG. 2A depicts results for exemplary markers. The results in FIG. 2B show that treatment with ibrutinib did not affect the phenotype of cells as central memory (T_{CM}) or effector memory (T_{EM}) subsets as assessed by the expression of CCR7 and CD45RA. As shown in FIG. 2C and FIG. 2D, there was a subtle decrease in expression levels of CD69, CD107a or PD-1 when CD4+ or CD8+ cells, respectively, were cultured in the presence of ibrutinib. A subtle decrease in the percentage of anti-CD19 CAR T cells expressing such markers were observed at the highest (supraphysiological) concentration of the inhibitor tested.

C. Cytokine Production

The production of cytokines by anti-CD19 CAR T cells cultured in the presence or absence of ibrutinib were assessed by assessing cytokine levels in the supernatants of co-cultures of CAR-T cells and irradiated K562-CD19 target cells. CAR-T cells generated as described above were plated at 100,000 cells/well on 96 well Poly-D-Lysine coated plates to which irradiated target cells (K562-CD19) were added at an effector to target ratio of 2.5:1. Cells were cultured for up to 4 days in the absence of ibrutinib or in the presence of 0.5, 5, 50 or 500 nM ibrutinib for the duration of the culture up to 4 days. Culture supernatants were harvested every 24 hrs at days 1, 2, 3 and 4, and IFNγ, IL-2, TNFα, IL-4 and IL-10 were measured from the culture supernatants using cytokine kits from Meso Scale Discovery (MSD).

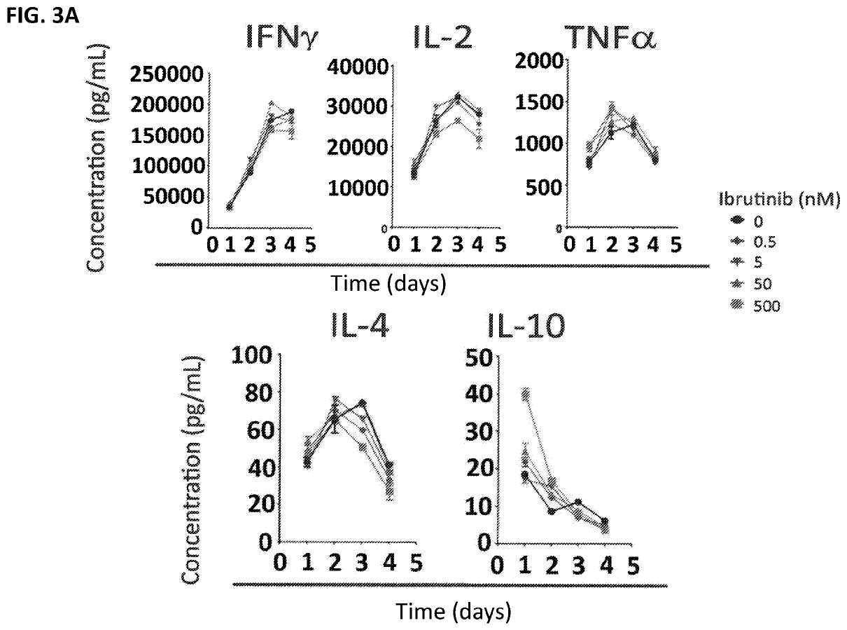

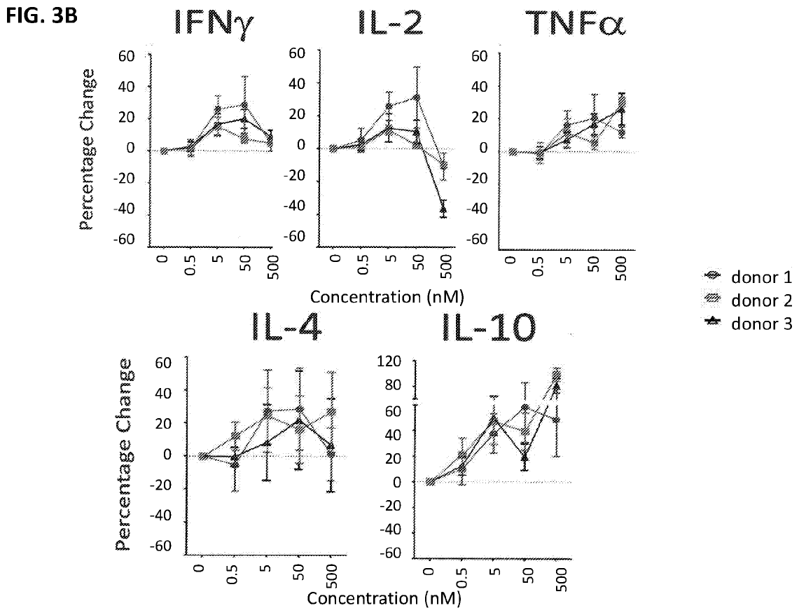

FIG. 3A depicts representative plots of kinetics of cytokine production over 4 days from CAR-T cells generated from donor 2. FIG. 3B depicts absolute change in cytokine production after stimulation for 2 days in 2 independent experiments. As shown in FIG. 3A and FIG. 3B, physiological concentrations of ibrutinib did not significantly decrease cytokine concentrations. In response to 50 nM ibrutinib, some increase in IFN-γ and IL-2 was observed. Ibrutinib at 50 nM modestly increased cytokine production in some donors, and a mean decrease in IL-2 of 19.6% or 1200 pg/mL was observed with 500 nM ibrutinib (P<0.05) (FIG. 3B).

D. Serial Restimulation

The ability of cells to expand ex vivo following repeated stimulations in some aspects can indicates capacity of CAR-T cells to persist (e.g., following initial activation) and/or is indicative of function and/or fitness in vivo (Zhao et al. (2015) Cancer Cell, 28:415-28). Anti-CD19 CAR+ T cells generated as described above were plated in triplicate at 100,000 cells/well on 96 well Poly-D-Lysine coated plates, and irradiated target cells (K562-CD19) were added at an effector to target ratio of 2.5:1. Cells were stimulated in the presence of 500 and 50 nM ibrutinib, harvested every 3-4 days, counted, and cultured for restimulation with new target cells using the same culture conditions and added concentration of ibrutinib after resetting cell number to initial seeding density for each round. A total of 7 rounds of stimulation during a 25 day culture period were carried out.

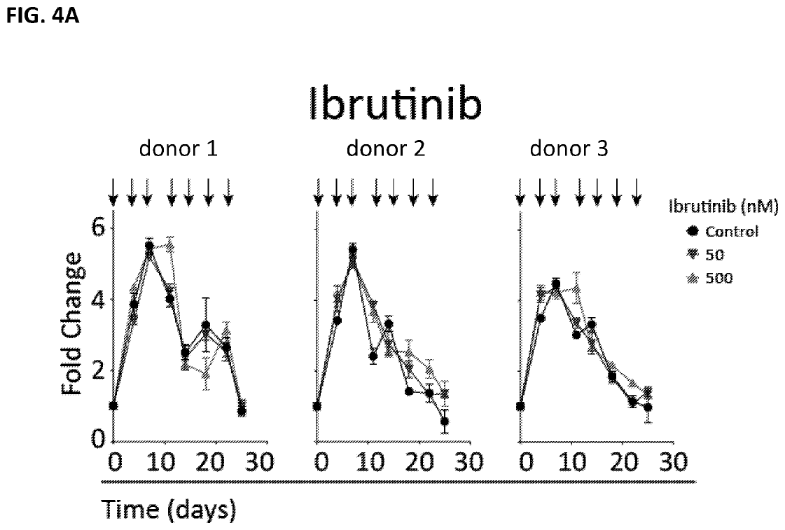

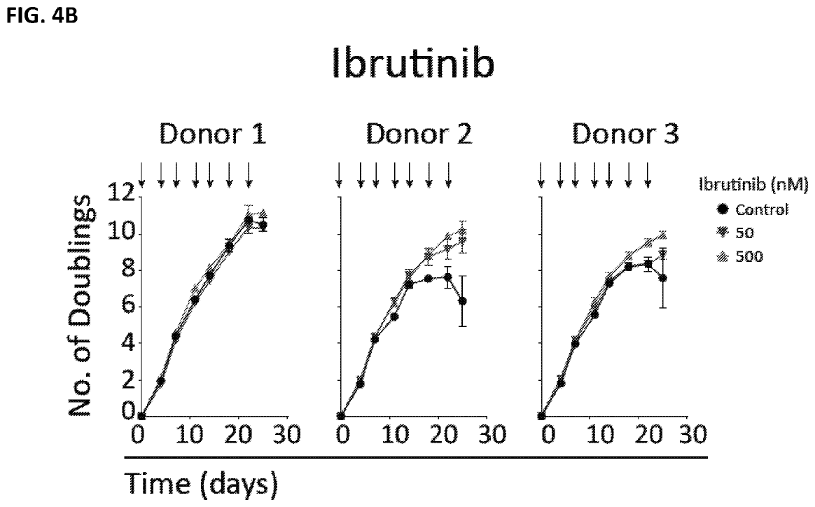

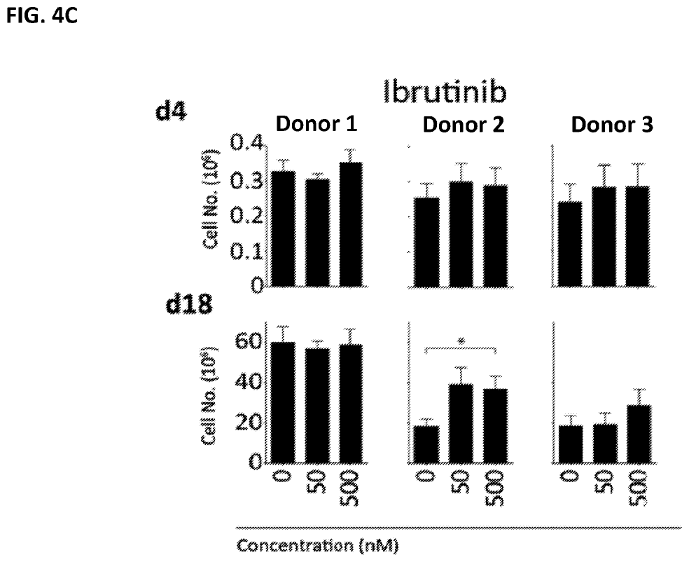

For each round of stimulation, the fold change in cell number (FIG. 4A) and the number of doublings (FIG. 4B) was determined. As shown in FIG. 4A and FIG. 4B, the presence of ibrutinib did not impact (e.g., did not inhibit) the initial growth of anti-CD19 CAR T cells as observed in fold change in cell number or number of population doublings. As shown in FIG. 4B, by day 18 of stimulation, following multiple rounds of restimulation, however, ibrutinib at both concentrations assessed was observed to lead to enhanced cell numbers and population doublings of anti-CD19 CAR T cells generated by engineering T cells derived from two of the three donors assessed. The cells derived from these two donors, generally, as compared to those derived from the other donor, performed less well in the serial restimulation assay in the absence of ibrutinib. FIG. 4C summarizes the results of the number of cells in culture at day 4 (1 round or restimulation) and day 18 (5 rounds of restimulation) after stimulation for the three donors in the presence of ibrutinib. As shown, a statistically significant increase in cell number after 18 days of the serial stimulation assay was observed. In particular, after five rounds of stimulation (day 18), CAR T cells from donor 2 treated with ibrutinib at the highest concentrations had significantly (P<0.05) increased cell counts relative to control cells. Non-significant, increased cell counts were also observed for donor 3 with ibrutinib treatment at the highest concentration tested. In this context increased cell counts could indicate superior proliferative capacity or survival and were not distinguished. When assessing cell counts across control conditions, cells derived from donors 2 and 3 exhibited inferior performance to donor 1 cells in this assay. Also, the cells derived from the two donors in which these differences were observed, generally, as compared to those derived from the other donor, performed less well in the serial restimulation assay in the absence of ibrutinib. Notably, these donors with inferior performance benefited from treatment with ibrutinib in this assay. The results indicate that for T cells that are impaired in one or more factors indicative of or important for survival and/or proliferative capacity may benefit from combination with a kinase inhibitor, e.g., TEC family kinase inhibitor or BTK/ITK inhibitor, such as ibrutinib. For example, combinations of such T cells with a kinase inhibitor such as ibrutinib may improve T cell function and/or persistence following antigen encounter.

E. TH1 Phenotype

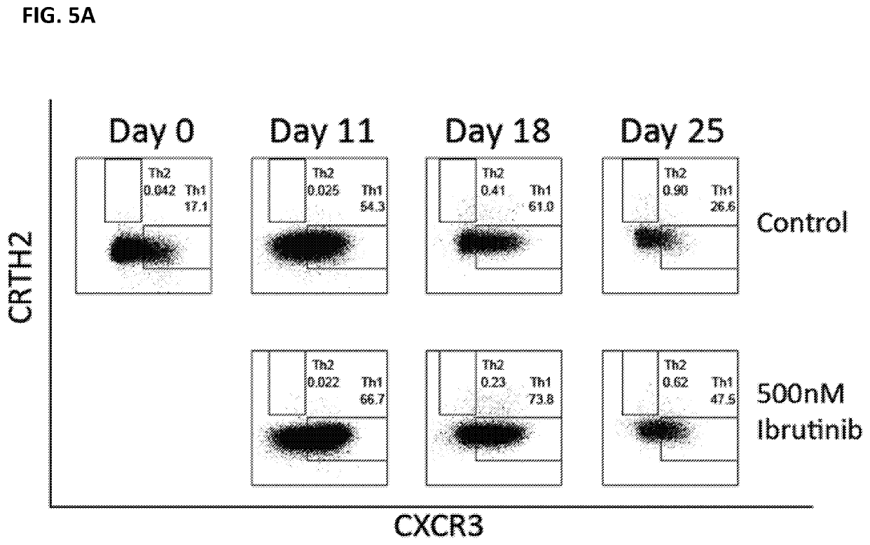

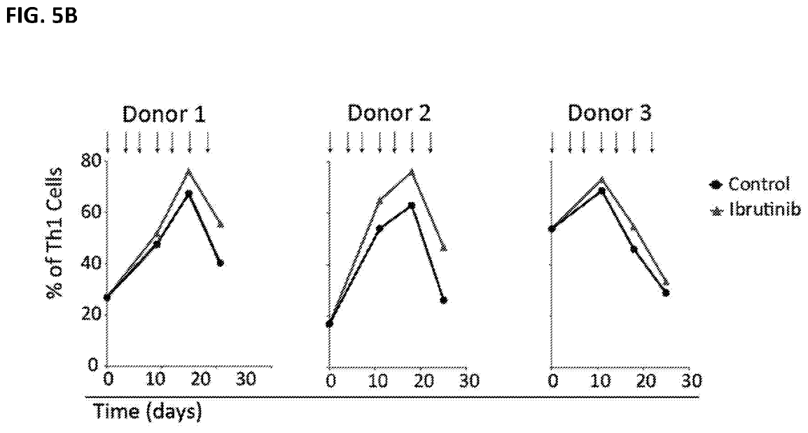

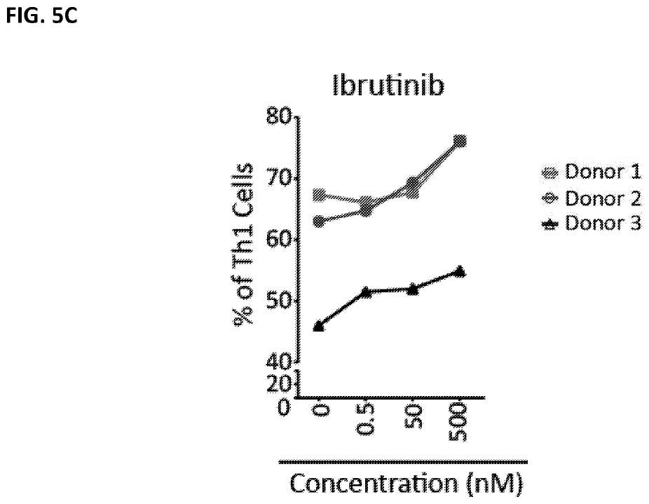

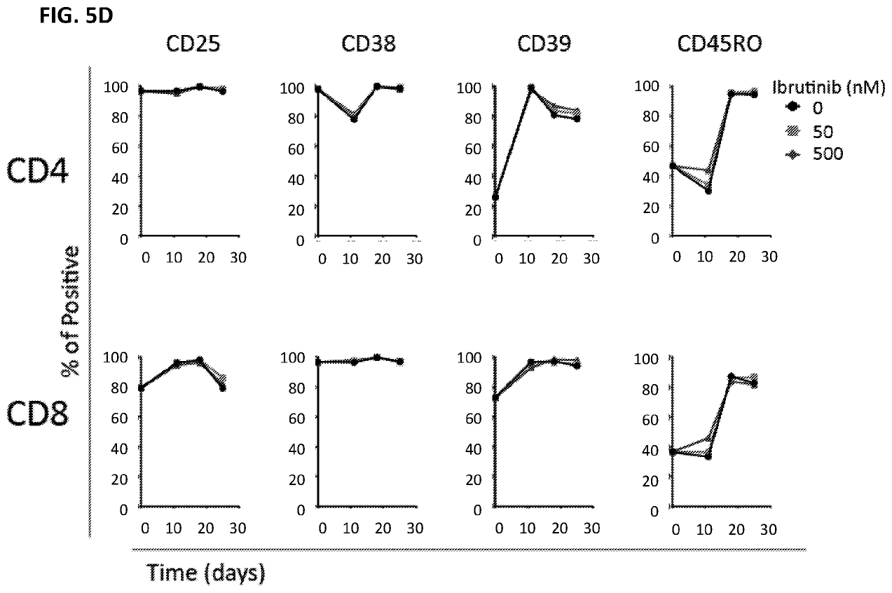

An assay was carried out demonstrating the skewing of anti-CD19 CAR T cells towards a TH1 phenotype when cultured in the presence of ibrutinib. Ibrutinib has been observed to limit TH2 CD4 T cell activation and proliferation through the inhibition of ITK (Honda, F., et al. (2012) Nat Immunol, 13(4): 369-78). A serial restimulation assay was performed as described above and cells were harvested at various times and analyzed by flow cytometry to assess percentage of TH1-phenotype (assessed as CD4+CXCR3+CRTH2−) T cells or TH2-phenotype (assessed as CD4+CXCR3−CRTH2+). Representative plots for cells cultured with and without the indicated concentration of ibrutinib, respectively, are shown in FIG. 5A, and percentage of TH1 cells following culture over the course of the serial restimulation, and under various concentrations of ibrutinib is shown in FIG. 5B and FIG. 5C, respectively.

The presence of ibrutinib in this assay was observed to increase the percentage of CAR+ T cells observed to exhibit a TH1 phenotype, after serial stimulation, and the effect was observed to be greater with increasing concentrations of ibrutinib. During the 18-day serial stimulation period, the percentage of CAR T TH1 cells increased from cells derived from each of three different donors (FIG. 5B). 500 nM ibrutinib further enhanced the percentage of TH1 cells (P<0.01) (FIG. 5C).

No significant effects of ibrutinib on additional CAR T activation or memory markers were observed in CAR T cells isolated from the serial stimulation assay (FIGS. 5D and 5E).

F. Gene Expression Analysis

Expression of various genes was assessed in anti-CD19 CART cells cultured in the presence or absence of ibrutinib (50 nM or 500 nM), during serial stimulation for 18 days as described above. At day 18 after serial stimulation, RNA was isolated from anti-CD19 CAR T cells and Nanostring Immune V2 panel tests were run across 594 genes. The log 2 (fold change) of each gene was plotted against the −log 10(Raw p-value) derived from ANOVA tests of unscaled housekeeping gene normalized to count data for treatment versus control. The results indicated that treatment with ibrutinib during serial restimulation did not significantly alter gene expression.

### Example 2: Enhancement of Anti-Tumor Activity of CAR-Expressing T Cells in the Presence of a Bruton's Tyrosine Kinase Inhibitor

A disseminated tumor xenograft mouse model was generated by injecting NOD/Scid/gc−/− (NSG) mice with cells of a CD19+ Nalm-6 disseminated tumor line, identified to be resistant to BTK inhibition.

On day zero (0), NSG mice were intravenously injected with 5×10⁵ Nalm-6 cells expressing firefly luciferase. Beginning at day 4 and daily for the duration of the study, mice were treated with vehicle control or were treated with ibrutinib, in each case by daily oral gavage (P.O.) at 25 mg/kg qd. To permit assessment of the effect of a combination therapy with the inhibitor, a suboptimal dose of anti-CD19 CAR T cells from two different donors (generated by transducing cells derived from samples of human donor subjects essentially as described above) were i.v. injected into each mouse at a concentration of 5×10⁵ CAR+ T cells per mouse at day 5. Mice in control groups were administered the vehicle control or ibrutinib but not administered the CAR-T cells. Eight (N=8) mice per group were monitored.

Following treatment as described above, tumor growth over time was measured by bioluminescence imaging and the average radiance (p/s/cm²/sr) was measured. Survival of treated mice also was assessed over time.

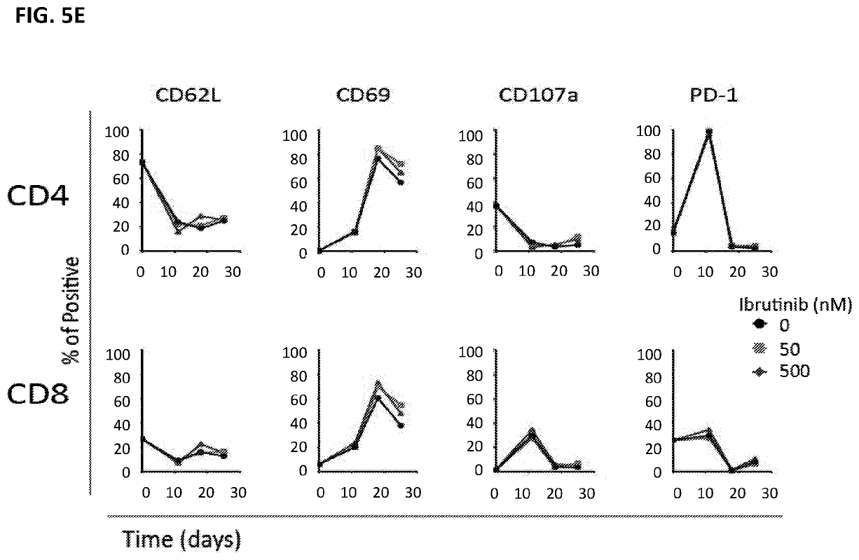

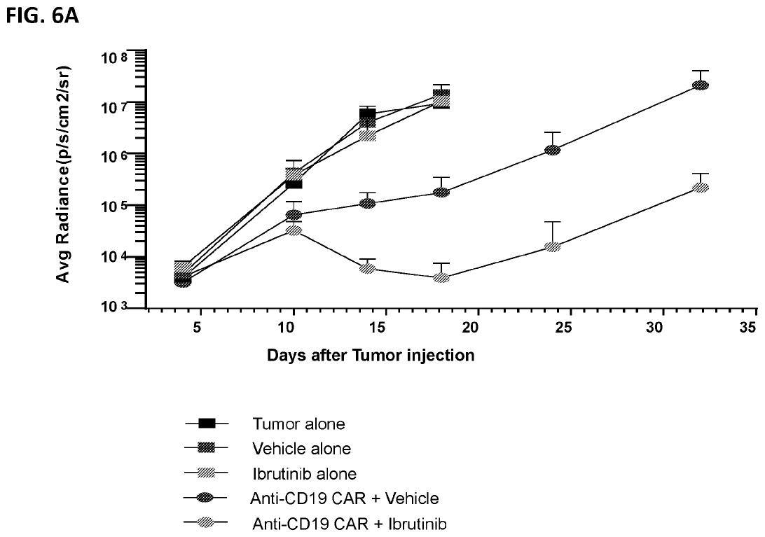

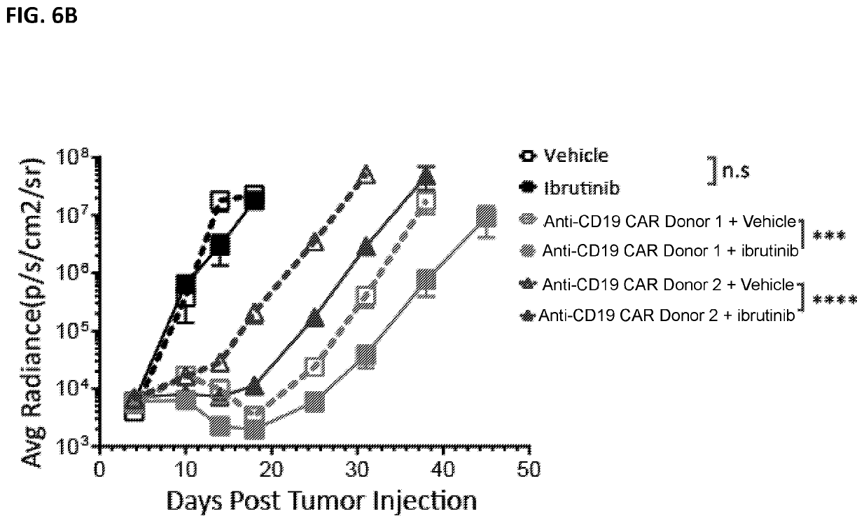

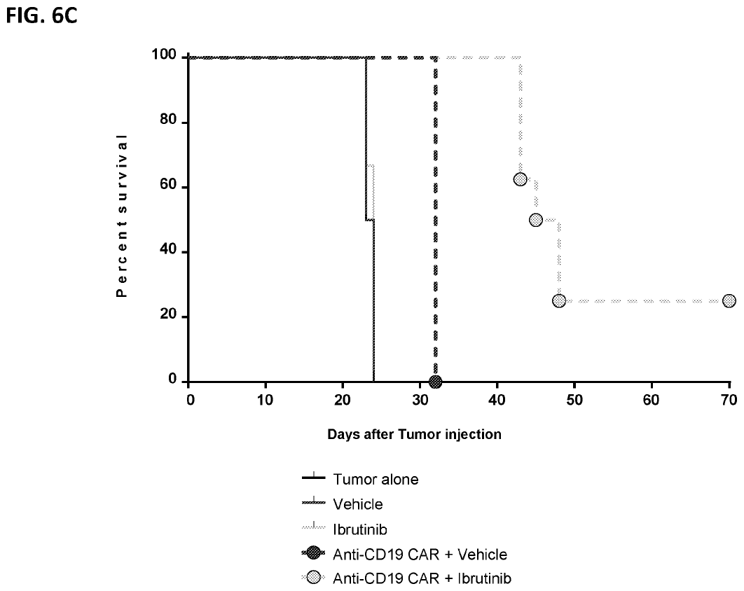

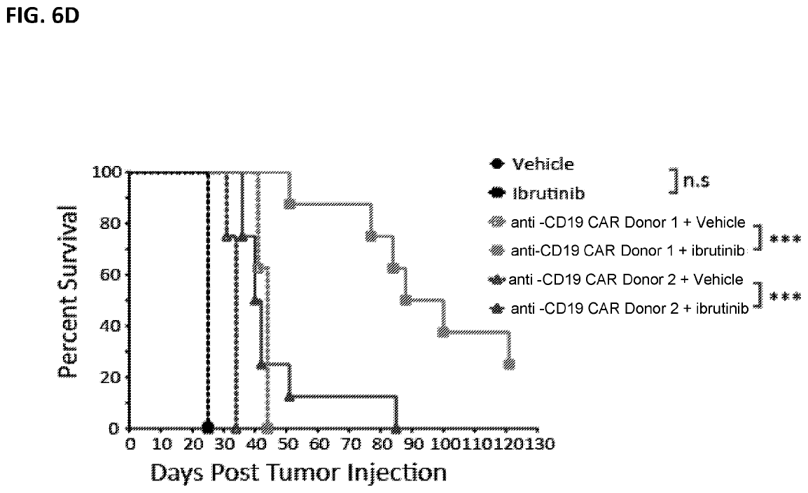

Results are shown in FIG. 6A for tumor growth over time from mice treated with ibrutinib and CAR T cells. Analysis of the results from the same study monitoring tumor growth at greater time points post-tumor injection from two different donors is shown in FIG. 6B. As shown, ibrutinib treatment alone had no effect on tumor burden in this ibrutinib-resistant model compared to vehicle treatment. In contrast, mice administered CAR-T cells and ibrutinib exhibited a significantly decreased tumor growth compared to mice treated with CAR-T cells and vehicle control (p<0.001, ***; p<0.0001. ***).

The combination of CAR T and ibrutinib increased survival of tumor-bearing mice as shown by Kaplan Meier curves showing survival of tumor bearing mice treated with ibrutinb and CAR T cells. As shown in FIG. 6C, representative results showed that mice treated with CAR-T cells and ibrutinib exhibited an increased median survival compared to the group receiving the suboptimal anti-CD19 CAR T cell dose+vehicle. Similar effects were seen in replicate studies using anti-CD19 CAR T cells produced by transducing T cells isolated from blood of other donor subjects. Analysis of the results from the same study monitoring survival at greater time points post-tumor injection from two different donors is shown in FIG. 6D, which showed that the combined administration of CAR T and ibrutinib also was observed to result in significantly increased survival compared with the CAR T and vehicle condition, (p<0.001, ***).

### Example 3: Assessment of CAR-Expressing T Cell Phenotype, Function and Anti-Tumor Activity In Vivo in the Presence of an Inhibitor of a TEC Family Kinase

NSG mice described in Example 2 were intravenously injected on day 0 with 5×10⁵ Nalm-6 cells expressing firefly luciferase. Beginning at day 4 and daily for the duration of the study, mice were treated with a vehicle control or were treated daily with ibrutinib in drinking water (D.W.) at 25 mg/kg/day. A bridging experiment confirmed that administration of ibrutinib by drinking water was equivalent to oral gavage administration (data not shown). To permit assessment of the effect of a combination therapy with the inhibitor, a suboptimal dose of anti-CD19 CAR T cells was i.v. injected into the mice at 5×10⁵/mouse at day 5. As a control, mice were administered the vehicle control without administration of the CAR-T cells or inhibitor.

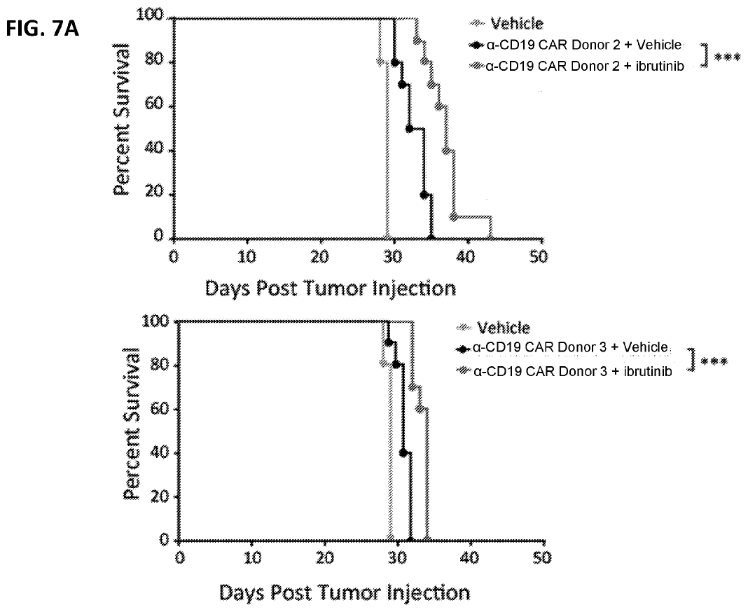

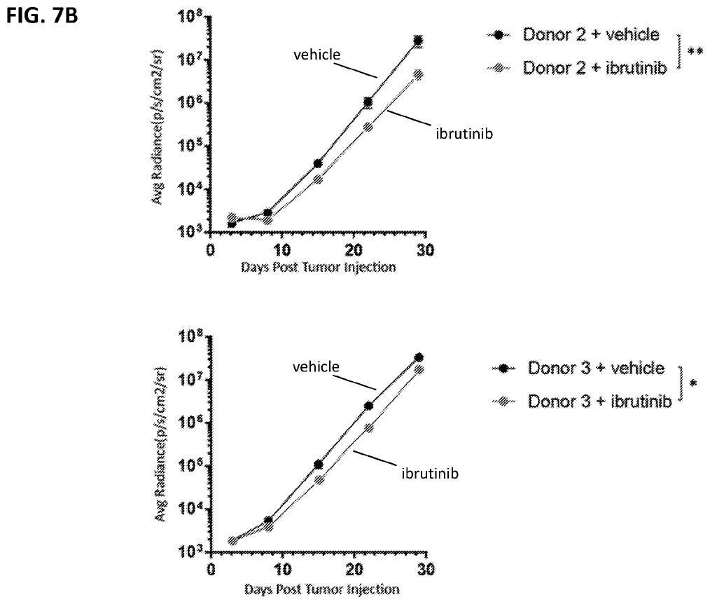

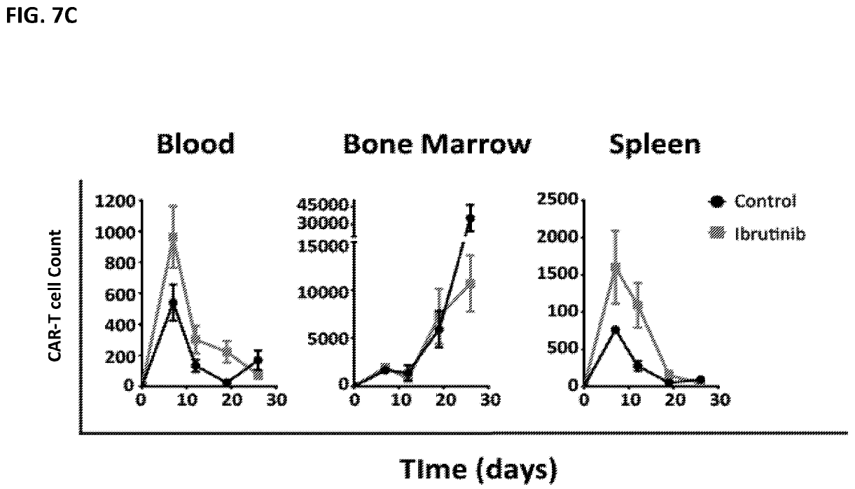

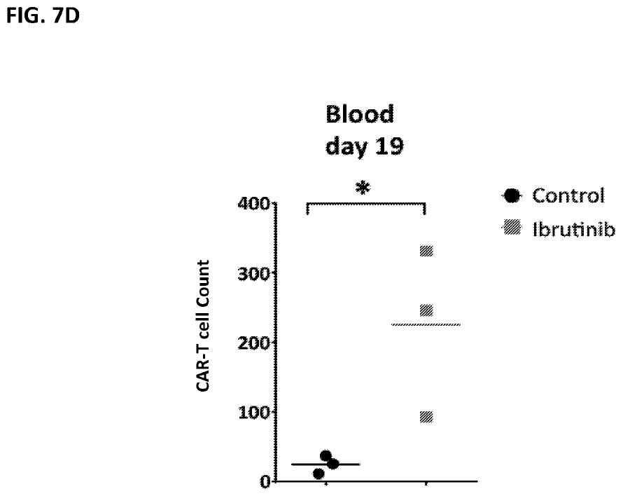

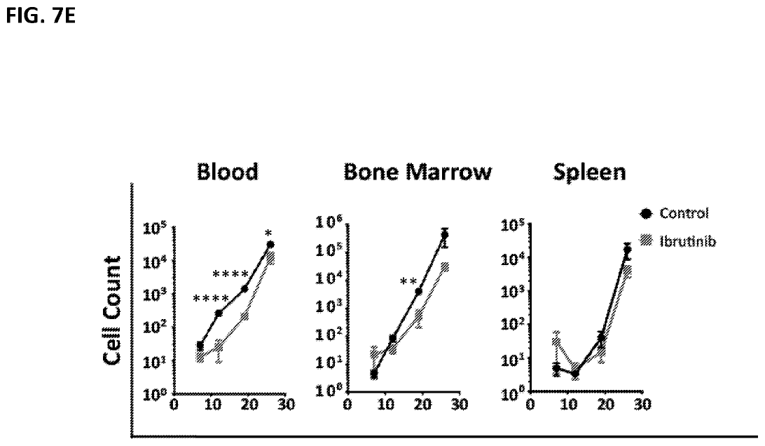

Following treatment as described above, the tumor growth and percent survival of treated mice was determined. As shown in FIG. 7A, mice treated with anti-CD19 CAR-T cells and ibrutinib exhibited an increased median survival compared to the group receiving the suboptimal anti-CD19 CAR T cell dose+vehicle (p<0.001). Ibrutinib administered in combination with CAR T, also significantly (P<0.001) decreased tumor growth (FIG. 7B) compared with the CAR T administered with vehicle alone. The results were similar using anti-CD19 CAR-T cells generated by engineering T cells derived from two different donors.

Pharmacokinetic analysis of CAR+ T cells was analyzed in blood, bone marrow and spleen from mice having received anti-CD19 CAR+ T cells from one donor-derived cells, and that had been treated with vehicle or ibrutinib (3 mice per group). Samples were analyzed to assess presence and levels of CAR T cells (based on expression of the surrogate marker using an anti-EGFR antibody) and/or tumor cells at days 7, 12, 19 and 26 post CAR+ T cell transfer. As shown in FIG. 7C, a significant increase in circulating CAR+ T cells was observed in mice treated with ibrutinib as compared to those treated with CAR+ T cells and vehicle, consistent with a greater expansion of CAR-T cells in the blood in the presence of ibrutinib. At day 19 post CAR-T cell transfer, a significant increase in the number of cells in the blood was observed after treatment with ibrutinb (FIG. 7D: * p<0.05). As shown in FIG. 7E, significantly fewer tumor cells were detected in the blood, bone marrow or spleen in mice in which the CAR+ cell treatment was combined with treatment with ibrutinib, as compared to with vehicle alone.

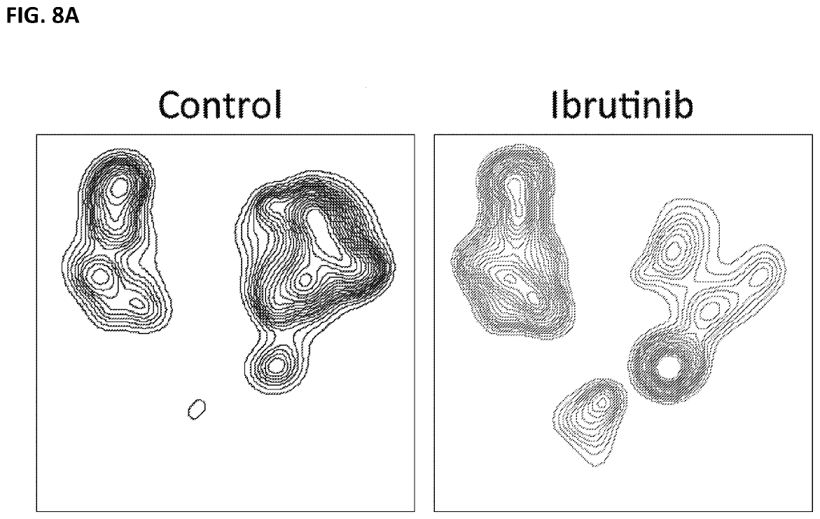

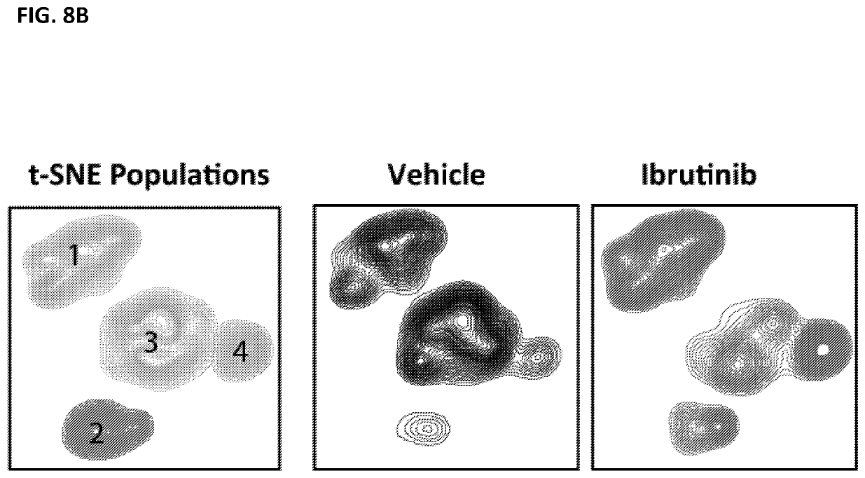

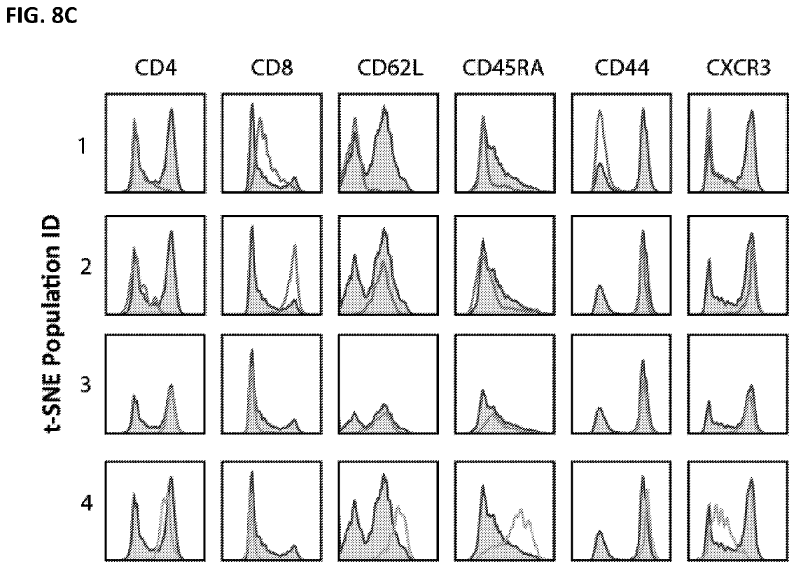

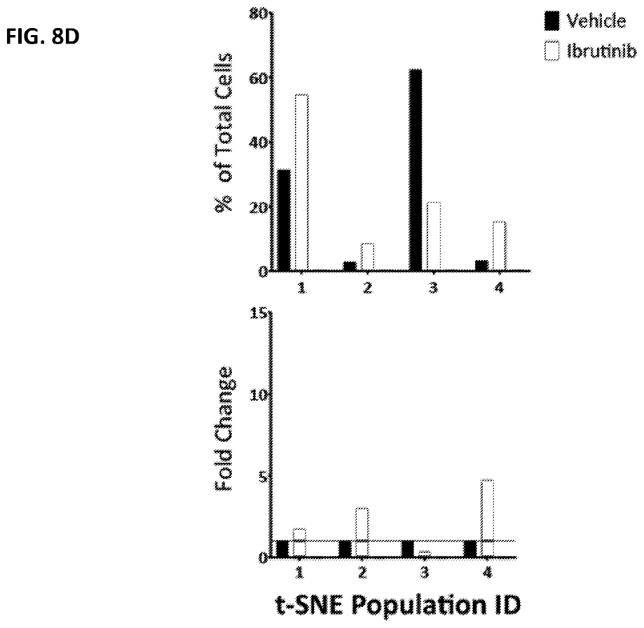

Ex vivo immunophenotyping also was performed on blood, bone marrow and spleen cells harvested at day 12 post-CAR T administration from mice that had received CAR+ T cells and that had been treated with vehicle or ibrutinib (n=3 mice per group). Cells were assessed for surface markers CD44, CD45RA, CD62L, CD154, CXCR3, CXCR4, and PD-1 by flow cytometry and T-distributed stochastic neighbor embedding (t-SNE) high dimensional analysis was performed using FlowJo software. As shown in FIG. 8A, phenotypic changes were observed in CAR+ T cells isolated from the bone marrow of animals having received CAR-T cells in combination with ibrutinib, as compared to with vector alone (control). Using multivariate t-SNE flow cytometry analysis based on pooled analysis from three mice per group, 4 distinct population clusters were identified (FIG. 8B). Flow cytometry histograms showing the individual expression profiles of CD4, CD8, CD62L, CD45RA, CD44 and CXCR3 from the 4 gated t-SNE in FIG. 8B overlaid on the expression of the total population (shaded) is shown in FIG. 8C.

The percentage and fold change of each t-SNE population in control mice or mice treated with ibrutinib is shown in FIG. 8D. Statistically significant differences are indicated as P<0.95 (*), P<0.01 (**), P<0.001 (***), P<0.0001 (****).

An increase in CD8+CD44^{hi} CXCR3^{hi} CD45RA^{lo} CD62L^{hi} (population 2) and CD4+CD44^{hi} CXCR3^{int} CD45RA^{hi} CD62L^{hi} (population 4) was observed in the bone marrow of CAR T-treated mice also administered ibrutinib as compared to control mice, at day 12 post CAR T transfer (FIGS. 8A-8C). A greater enhancement of population 4 was observed in ibrutinib-treated animals (15.2% compared to 4.4% of CAR-T cells) (FIG. 8C).

### Example 4: Bruton's Tyrosine Kinase (BTK) Inhibitor Enhances Cytolytic Function of CAR-Expressing T Cells Manufactured from Diffuse Large B-Cell Lymphoma (DLBCL) Patients

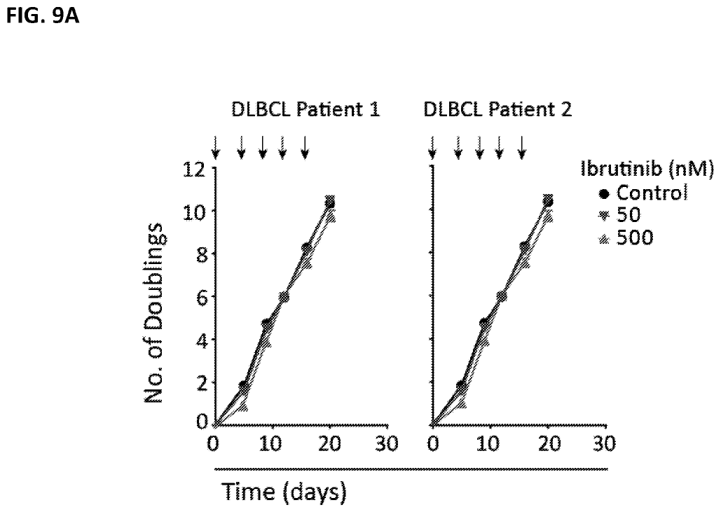

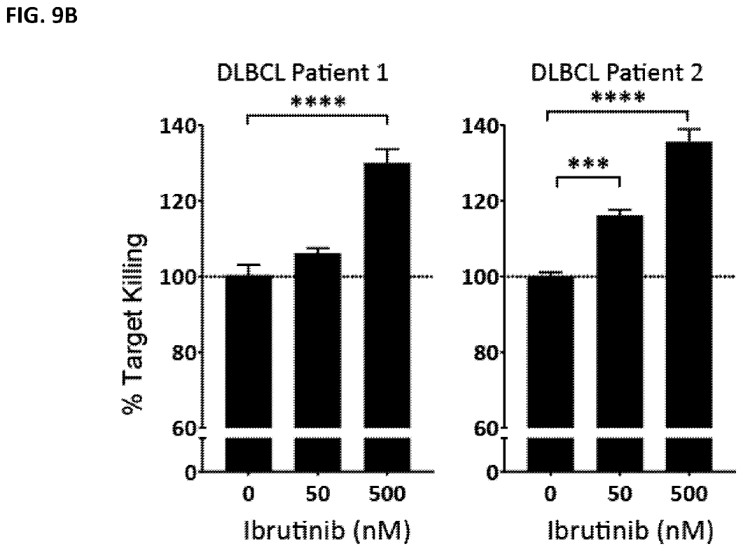

Anti-CD19 CAR-T cells were generated substantially as described in Example 1, except that T cells were isolated from two different human subjects having diffuse large B-cell lymphoma (DLBCL). Cells were subjected to serial restimulation as described in Example 1.D, by co-culturing CAR-T cells with K562-CD19 targets cells at an effector to target ratio of 2.5:1 in the presence of 500 and 50 nM ibrutinib, harvesting cells every 3-4 days and restimulating under the same conditions after resetting cell number. Cells were subjected to serial restimulation over a 21 day culture period and monitored for cell expansion and cytotoxic activity. As shown in FIG. 9A, cell expansion, as determined by the number of cell doublings, was observed during the 21 day culture period for cells derived from each individual subject. Ibrutinib did not inhibit the proliferation of CAR T cells derived from either patient (FIG. 9A), an observation consistent with previous data from healthy donor-derived CAR T cells. As shown in FIG. 9B, CAR-T cells manufactured from cells derived from each individual subject demonstrated an increase in cytolytic function in the presence of 500 nM ibrutinib after 16 days of serial stimulation (FIG. 9B). In cells derived from one patient, an increase in cytolytic activity after 16 days of serial stimulation was observed with 50 nM ibrutinib (P<0.01) (FIG. 9B). This increase in cytolytic activity is consistent with results from healthy donor cells (FIGS. 1C-1D).

### Example 5: Assessment of Molecular Signature by RNA-Sea of CAR-Expressing T Cells Treated with Ibrutinib

RNA was isolated from individual CAR-expressing cells, derived from three different donors, that had been treated for 18 days in a serial stimulation assay in the presence of ibrutinib (50 nM, 500 nM) or control (0 nM). RNA isolation was performed using the RNEasy Micro Kit (Qiagen). Samples were sequenced and RNASeq reads were mapped to the human genome (GRCh38) and aligned to the GENCODE release 24 gene model. RNAseq quality metrics were generated and evaluated to confirm consistency across samples. Differentially expressed genes were identified by imposing a log₂ fold change cutoff of 0.5 and a Benjamini-Hochberg adjusted false discovery rate (FDR) cutoff of 0.05.

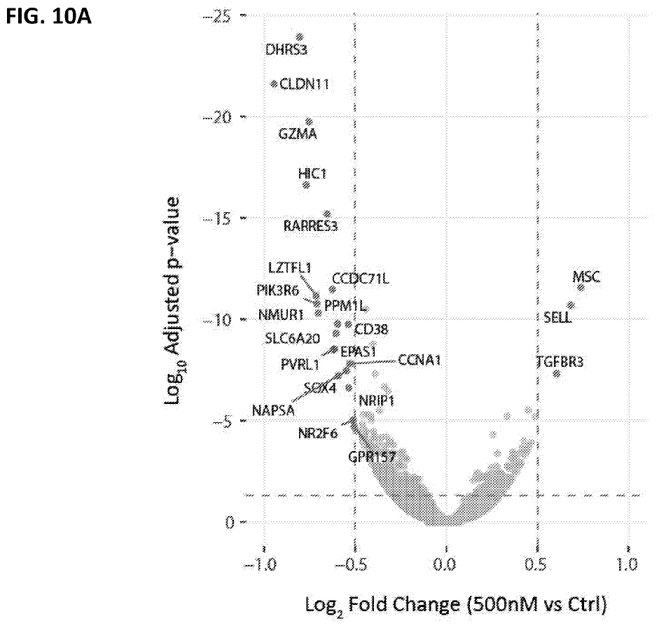

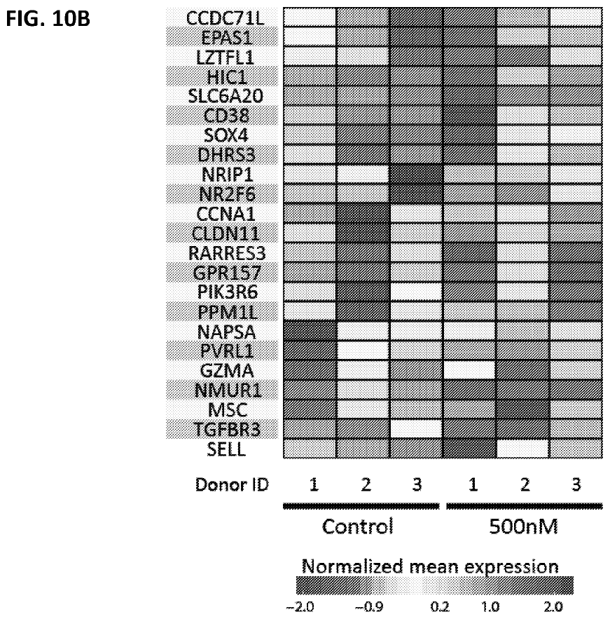

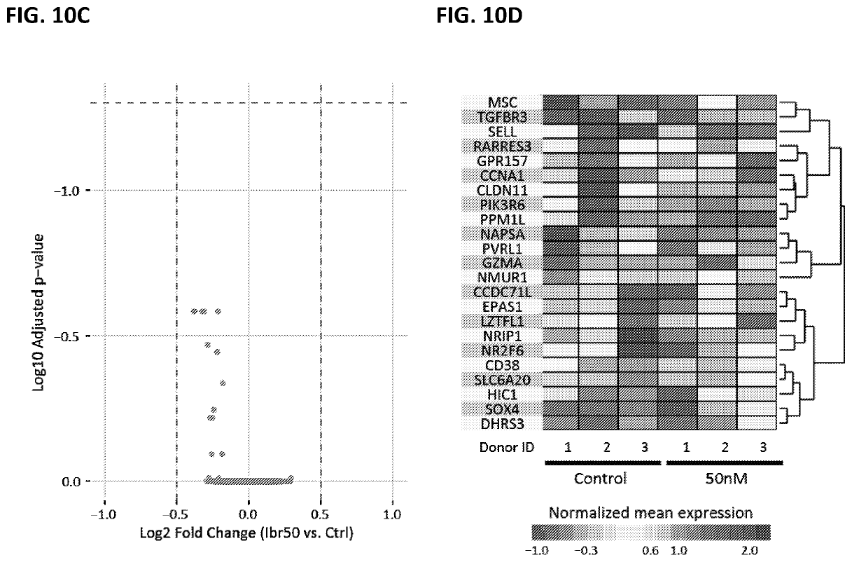

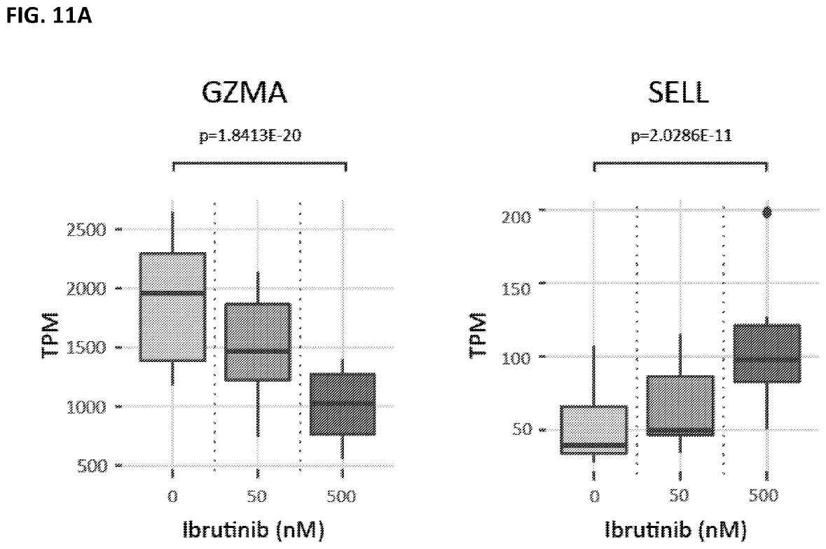

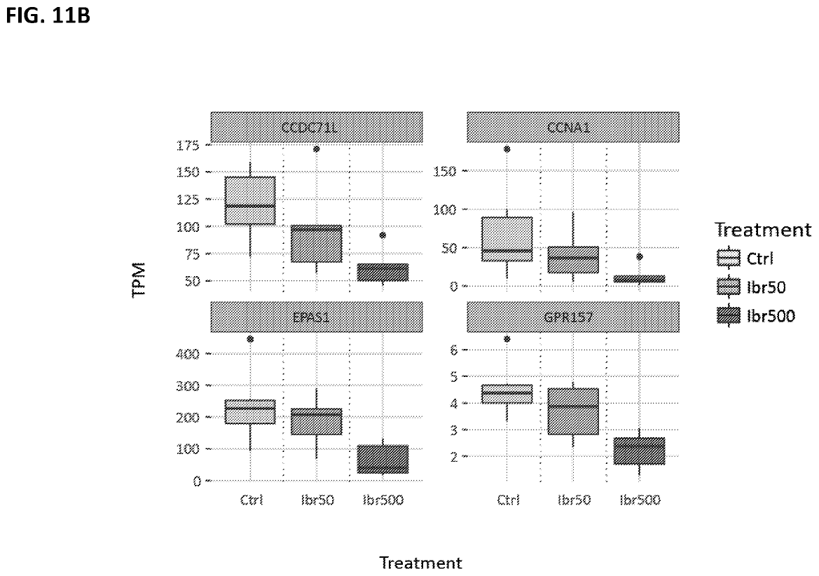

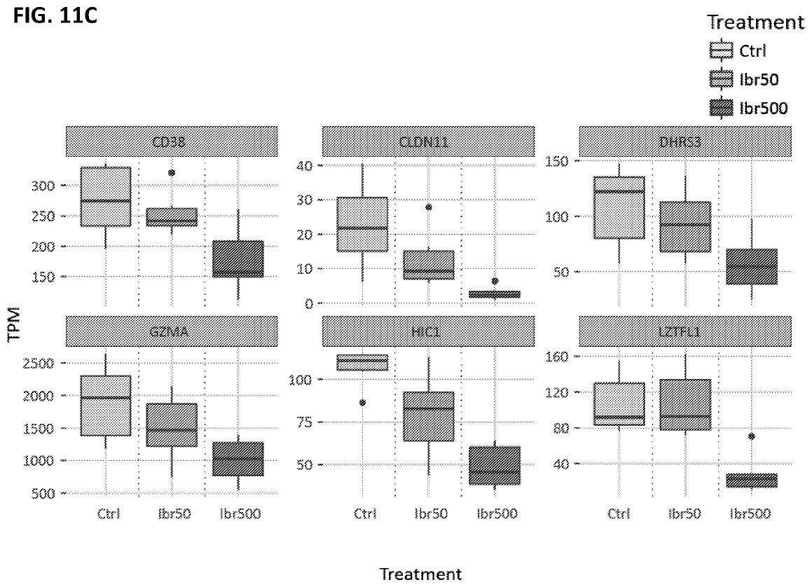

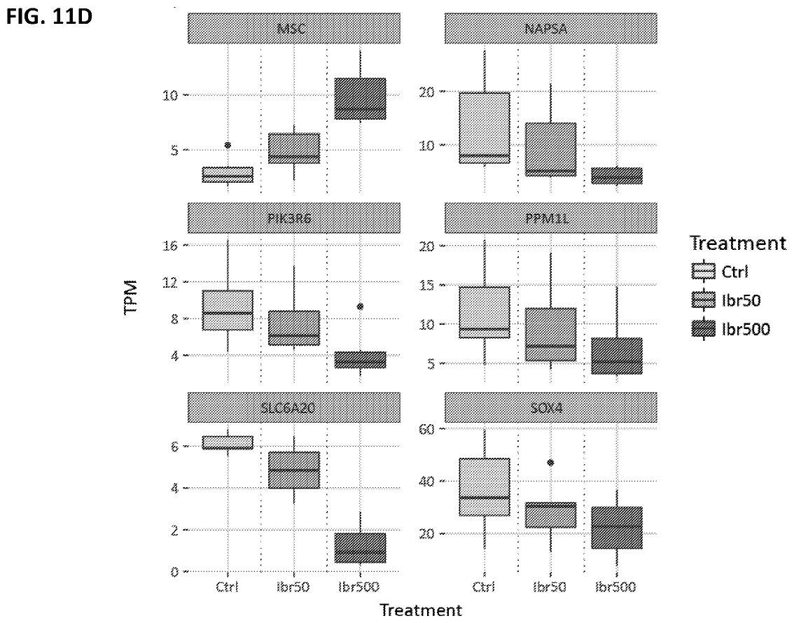

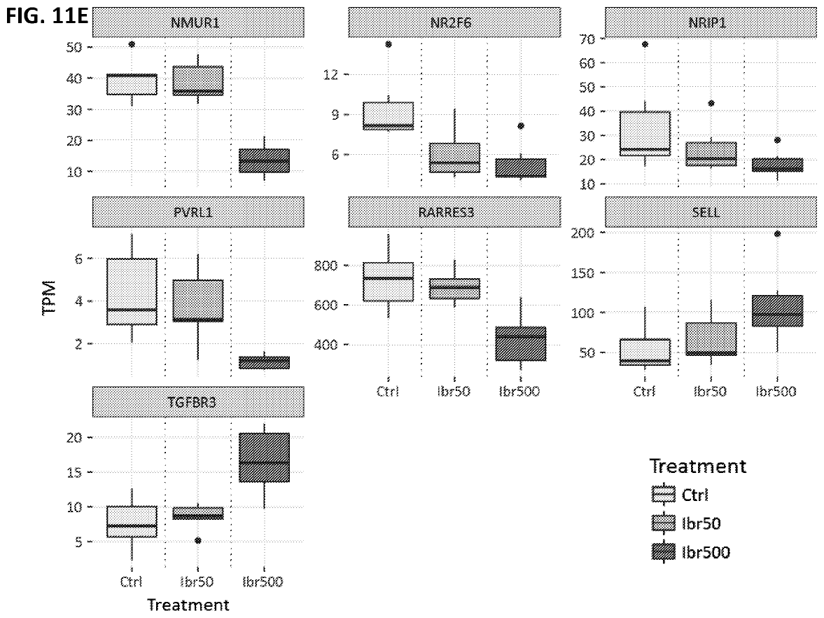

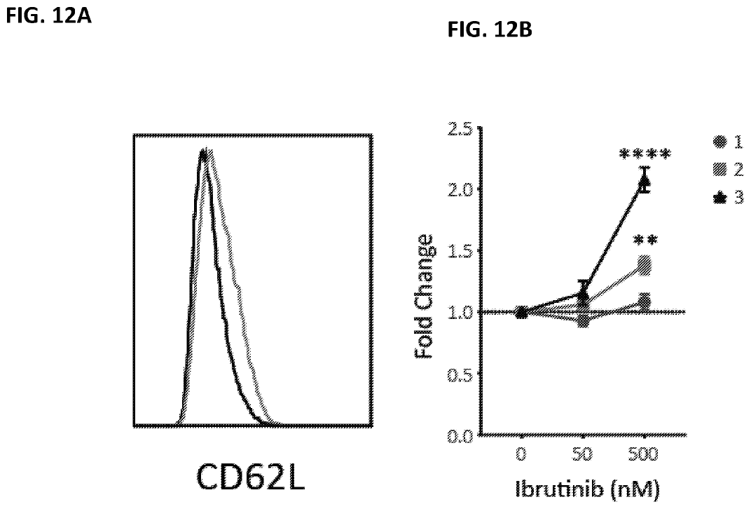

As shown in the volcano plot in FIG. 10A, 500 nM ibrutinib significantly (FDR<0.05, abs Log 2FC>0.5) altered the expression of 23 protein-coding genes. FIG. 10B shows a heat map of gene expression changes for the 23 genes identified in FIG. 10A. Although not significant, similar trends were seen with 50 nM (FIGS. 10C and 10D). Box plots of gene expression for exemplary genes following treatment the different concentrations of inhibitor (50 nM or 500 nM) or control are shown in FIGS. 11A-11E, Among the differentially expressed genes, decreases in genes such as granzyme A (FIG. 11A) and CD38 (FIG. 11C), and increases in SELL/CD62L (FIG. 11A) are consistent with an effect of ibrutinib to dampen terminal-effector-like genes while enhancing genes associated with memory development. Furthermore, RNA-Seq revealed that genes associated with promoting TH1 differentiation were altered by ibrutinib, including upregulation of MSC, known to suppress TH2 programing (Wu, C., et al. (2017) Nat Immunol, 18(3): 344-353), and downregulation of HES6, HIC1, LZTFL1, NRIP1, CD38 and RARRES3, associated with the ATRA/Retinoic acid signaling pathway identified to inhibit TH1 development (Britschgi, C., et al. (2008) Br J Haematol, 141(2): 179-87; Jiang, H., et al. (2016) J Immunol, 196(3): 1081-90; Heim, K. C., et al. (2007) Mol Cancer, 6: 57; Nijhof, I. S., et al. (2015) Leukemia, 29(10): 2039-49; Zirn, B., et al. (2005) Oncogene, 24(33): 5246-51) (FIGS. 11E, 11B and 11D). In support of the RNA-Seq results, a significant increase in CD62L expression was observed by flow cytometry after 18 days of serial stimulation in donors 2 and 3 (FIGS. 12A and 12B). Taken together, these results support that long term ibrutinib treatment may result in an increased TH1 and memory-like phenotype in CAR T.

### Example 6: Administration of Anti-CD19 CAR-Expressing Cells in Combination with Compound 1 to Subjects with Relapsed and Refractory Non-Hodgkin's Lymphoma (NHL)

Anti-CD19 CAR-expressing T cell compositions are produced substantially as described in Example 1, and generated CD4+ CAR-expressing T cell compositions and CD8+ CAR-expressing T cell compositions are separately administered to subjects with relapsed/refractory (R/R) B cell non-Hodgkin lymphoma (NHL) in combination with administration with ibrutinib (having the structure:

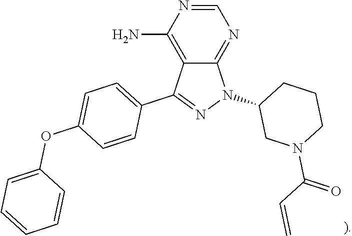 [SMILES: C=CC(=O)N1CCC[C@@H](n2nc(-c3ccc(Oc4ccccc4)cc3)c3c(N)ncnc32)C1]

Groups of subjects selected for treatment include subjects with diffuse large B-cell lymphoma (DLBCL); de novo or transformed from indolent lymphoma (NOS); high-grade B-cell lymphoma, with MYC and BCL2 and/or BCL6 rearrangements with DLBCL histology (double/triple hit lymphoma); follicular lymphoma grade 3b (FLG3B); T cell/histiocyte-rich large B-cell lymphoma; EBV positive DLBCL, NOS; and primary mediastinal (thymic) large B-cell lymphoma (PMBCL). Subjects treated also include those that have relapsed following or are refractory to at least two prior lines of therapy, including a CD20-targeted agent and an anthracycline, and have an Eastern Cooperative Oncology Group (ECOG) score of less than or equal to 1 at screening.

To generate CAR-expressing T cells, samples comprising cells from the circulating blood of a human subject are obtained by apheresis or leukapheresis, and T cells are isolated by immunoaffinity-based enrichment. Cells are obtained at 28 days (±7 days) prior to the CAR+ T cell infusion. Isolated cells are activated and transduced with a viral vector encoding an anti-CD19 CAR.

Prior to CAR+ T cell infusion, subjects receive a lymphodepleting chemotherapy with fludarabine (flu, 30 mg/m²/day) and cyclophosphamide (Cy, 300 mg/m²/day) for three (3) days.

Ibrutinib is administered orally to subjects beginning at 7 days prior to apheresis or leukapheresis (35 days (±7 days) prior to the CAR+ T cell infusion), daily, at 140 mg, 280 mg, 420 mg or 560 mg dose/day, until the initiation of lymphodepleting chemotherapy. During the time that the subject receives the lymphodepleting chemotherapy, ibrutinib is not administered. Administration of ibrutinib is resumed after the completion of lymphodepleting chemotherapy.

The subjects receive CAR-expressing T cells 2-7 days after lymphodepletion. Subjects are administered a single dose of 1×10⁸ CAR-expressing T cells (each single dose via separate infusions at a 1:1 ratio of CD4+ CAR-expressing T cells and CD8+ CAR-expressing T cells, respectively). Administration of ibrutinib is continued, after the completion of the lymphodepleting chemotherapy and the infusion of engineered CAR-expressing cells.

Response to treatment is assessed based on radiographic tumor assessment by positron emission tomography (PET) and/or computed tomography (CT) or magnetic resonance imaging (MRI) scans at baseline prior to treatment and at various times following treatment (e.g. based on Lugano classification, see, e.g., Cheson et al., (2014) JCO 32(27):3059-3067). The presence or absence of treatment-emergent adverse events (TEAE) following treatment also is assessed. Subjects also are assessed and monitored for neurotoxicity (neurological complications including symptoms of confusion, aphasia, encephalopathy, myoclonus seizures, convulsions, lethargy, and/or altered mental status), graded on a 1-5 scale, according to the National Cancer Institute—Common Toxicity Criteria (CTCAE) scale, version 4.03 (NCI-CTCAE v4.03). Common Toxicity Criteria (CTCAE) scale, version 4.03 (NCI-CTCAE v4.03). See Common Terminology for Adverse Events (CTCAE) Version 4, U.S. Department of Health and Human Services, Published: May 28, 2009 (v4.03: Jun. 14, 2010); and Guido Cavaletti & Paola Marmiroli Nature Reviews Neurology 6, 657-666 (December 2010). Cytokine release syndrome (CRS) also is determined and monitored, graded based on severity. See Lee et al, Blood. 2014; 124(2):188-95. Subjects also are assessed for pharmacokinetics (PK) of anti-CD19 CAR+ T cells pre- and post-treatment with ibrutinib and for PK and pharmacodynamics (PD) parameters of ibrutinib.

The dosing of ibrutinib is stopped after Day 180 (6 months post CAR+ T-cell infusion), unless the subject achieves a partial response (PR) in which case further administration of ibrutinib may continue until disease progression.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | ESKYGPPCPPCP | spacer (IgG4hinge) |
| | | (aa) |
| 2 | GAATCTAAGTACGGACCGCCCTGCCCCCCTTGCCCT | spacer (IgG4hinge) |
| | | (nt) |
| 3 | ESKYGPPCPPCPGQPREPQVYTLPPSQEEMTKNQVSLTCLVKGFYPSDIA | Hinge-CH3 spacer |
| | VEWESNGQPENNYKTTPPVLDSDGSFFLYSRLTVDKSRWQEGNVFSCSVM | |
| | HEALHNHYTQKSLSLSLGK | |
| 4 | ESKYGPPCPPCPAPEFLGGPSVFLFPPKPKDTLMISRTPEVTCVVVDVSQ | Hinge-CH2-CH3 |
| | EDPEVQFNWYVDGVEVHNAKTKPREEQFNSTYRVVSVLTVLHQDWLNGKE | spacer |
| | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGK | |
| 5 | RWPESPKAQASSVPTAQPQAEGSLAKATTAPATTRNTGRGGEEKKKEKEK | IgD-hinge-Fc |
| | EEQEERETKTPECPSHTQPLGVYLLTPAVQDLWLRDKATFTCFVVGSDLK | |
| | DAHLTWEVAGKVPTGGVEEGLLERHSNGSQSQHSRLTLPRSLWNAGTSVT | |
| | CTLNHPSLPPQRLMALREPAAQAPVKLSLNLLASSDPPEAASWLLCEVSG | |
| | FSPPNILLMWLEDQREVNTSGFAPARPPPQPGSTTFWAWSVLRVPAPPSP | |
| | QPATYTCVVSHEDSRTLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIKHFKN | tEGFR |
| | CTSISGDLHILPVAFRGDSFTHTPPLDPQELDILKTVKEITGFLLIQAWP | |
| | ENRTDLHAFENLEIIRGRTKQHGQFSLAVVSLNITSLGLRSLKEISDGDV | |
| | IISGNKNLCYANTINWKKLFGTSGQKTKIISNRGENSCKATGQVCHALCS | |
| | PEGCWGPEPRDCVSCRNVSRGRECVDKCNLLEGEPREFVENSECIQCHPE | |
| | CLPQAMNITCTGRGPDNCIQCAHYIDGPHCVKTCPAGVMGENNTLVWKYA | |
| | DAGHVCHLCHPNCTYGCTGPGLEGCPTNGPKIPSIATGMVGALLLLLVVA | |
| | LGIGLFM | |
| 8 | FWVLVVVGGVLACYSLLVTVAFIIFWV | CD28 (amino acids |
| | | 153-179 of |
| | | Accession No. |
| | | P10747) |
| 9 | IEVMYPPPYLDNEKSNGTIIHVKGKHLCPSPLFPGPSKP | CD28 (amino acids |
| | FWVLVVVGGVLACYSLLVTVAFIIFWV | 114-179 of |
| | | Accession No. |
| | | P10747) |
| 10 | RSKRSRLLHSDYMNMTPRRPGPTRKHYQPYAPPRDFAAYRS | CD28 (amino acids |
| | | 180-220 of |
| | | P10747) |
| 11 | RSKRSRGGHSDYMNMTPRRPGPTRKHYQPYAPPRDFAAYRS | CD28 (LL to GG) |
| 12 | KRGRKKLLYIFKQPFMRPVQTTQEEDGCSCRFPEEEEGGCEL | 4-1BB (amino |
| | | acids 214-255 of |
| | | Q07011.1) |
| 13 | RVKFSRSADAPAYQQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | |
| | YDALHMQALPPR | |
| 14 | RVKFSRSAEPPAYQQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | |
| | YDALHMQALPPR | |
| 15 | RVKFSRSADAPAYKQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | |
| | YDALHMQALPPR | |
| 16 | RKVCNGIGIGEFKDSLSINATNIKHFKNCTSISGDLHILPVAFRGDSFTH | tEGFR |
| | TPPLDPQELDILKTVKEITGFLLIQAWPENRTDLHAFENLEIIRGRTKQH | |
| | GQFSLAVVSLNITSLGLRSLKEISDGDVIISGNKNLCYANTINWKKLFGT | |
| | SGQKTKIISNRGENSCKATGQVCHALCSPEGCWGPEPRDCVSCRNVSRGR | |
| | ECVDKCNLLEGEPREFVENSECIQCHPECLPQAMNITCTGRGPDNCIQCA | |
| | HYIDGPHCVKTCPAGVMGENNTLVWKYADAGHVCHLCHPNCTYGCTGPGL | |
| | EGCPTNGPKIPSIATGMVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | -PGGG-(SGGGG)₅-P- wherein P is proline, G is | Linker |
| | glycine and S is serine | |
| 23 | GSADDAKKDAAKKDGKS | Linker |
| 24 | atgcttctcctggtgacaagccttctgctctgtgagttaccacacccagc | GMCSFR alpha |
| | attcctcctgatccca | chain signal |
| | | sequence |
| 25 | MLLLVTSLLLCELPHPAFLLIP | GMCSFR alpha |
| | | chain signal |
| | | sequence |
| 26 | MALPVTALLLPLALLLHA | CD8 alpha signal |
| | | peptide |
| 27 | Glu Pro Lys Ser Cys Asp Lys Thr His Thr Cys Pro | Hinge |
| | Pro Cys Pro | |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPCPRCPEPKS | Hinge |
| | CDTPPPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro | Hinge |
| | Cys Pro | |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPPRKGLEWLGV | VH |
| | IWGSETTYYNSALKSRLTIIKDNSKSQVFLKMNSLQTDDTAIYYCAKHYY | |
| | YGGSYAMDYWGQGTSVTVSS | |
| 42 | DIQMTQTTSSLSASLGDRVTISCRASQDISKYLNWYQQKPDGTVKLLIYH | VL |
| | TSRLHSGVPSRFSGSGSGTDYSLTISNLEQEDIATYFCQQGNTLPYTFGG | |
| | GTKLEIT | |
| 43 | DIQMTQTTSSLSASLGDRVTISCRASQDISKYLNWYQQKPDGTVKLLIYH | scFv |
| | TSRLHSGVPSRFSGSGSGTDYSLTISNLEQEDIATYFCQQGNTLPYTFGG | |
| | GTKLEITGSTSGSGKPGSGEGSTKGEVKLQESGPGLVAPSQSLSVTCTVS | |
| | GVSLPDYGVSWIRQPPRKGLEWLGVIWGSETTYYNSALKSRLTIIKDNSK | |
| | SQVFLKMNSLQTDDTAIYYCAKHYYYGGSYAMDYWGQGTSVTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSVVDFYFDY | CDR H3 |
| 50 | EVKLQQSGAELVRPGSSVKISCKASGYAFSSYWMNWVKQRPGQGLEWIGQ | VH |
| | IYPGDGDTNYNGKFKGQATLTADKSSSTAYMQLSGLTSEDSAVYFCARKT | |
| | ISSVVDFYFDYWGQGTTVTVSS | |
| 51 | DIELTQSPKFMSTSVGDRVSVTCKASQNVGTNVAWYQQKPGQSPKPLIYS | VL |
| | ATYRNSGVPDRFTGSGSGTDFTLTITNVQSKDLADYFCQQYNRYPYTSGG | |
| | GTKLEIKR | |
| 52 | GGGGSGGGGSGGGGS | Linker |
| 53 | EVKLQQSGAELVRPGSSVKISCKASGYAFSSYWMNWVKQRPGQGLEWIGQ | scFv |
| | IYPGDGDTNYNGKFKGQATLTADKSSSTAYMQLSGLTSEDSAVYFCARKT | |
| | ISSVVDFYFDYWGQGTTVTVSSGGGGSGGGGSGGGGSDIELTQSPKFMST | |
| | SVGDRVSVTCKASQNVGTNVAWYQQKPGQSPKPLIYSATYRNSGVPDRFT | |
| | GSGSGTDFTLTITNVQSKDLADYFCQQYNRYPYTSGGGTKLEIKR | |
| 54 | HYYYGGSYAMDY | HC-CDR3 |
| 55 | HTSRLHS | LC-CDR2 |
| 56 | QQGNTLPYT | LC-CDR3 |
| 57 | gacatccagatgacccagaccacctccagcctgagcgccagcctgggcga | Sequence encoding |
| | ccgggtgaccatcagctgccgggccagccaggacatcagcaagtacctga | scFv |
| | actggtatcagcagaagcccgacggcaccgtcaagctgctgatctaccac | |
| | accagccggctgcacagcggcgtgcccagccggtttagcggcagcggctc | |
| | cggcaccgactacagcctgaccatctccaacctggaacaggaagatatcg | |
| | ccacctacttttgccagcagggcaacacactgccctacacctttggcggc | |
| | ggaacaaagctggaaatcaccggcagcacctccggcagcggcaagcctgg | |
| | cagcggcgagggcagcaccaagggcgaggtgaagctgcaggaaagcggcc | |
| | ctggcctggtggcccccagccagagcctgagcgtgacctgcaccgtgagc | |
| | ggcgtgagcctgcccgactacggcgtgagctggatccggcagccccccag | |
| | gaagggcctggaatggctgggcgtgatctggggcagcgagaccacctact | |
| | acaacagcgccctgaagagccggctgaccatcatcaaggacaacagcaag | |
| | agccaggtgttcctgaagatgaacagcctgcagaccgacgacaccgccat | |
| | ctactactgcgccaagcactactactacggcggcagctacgccatggact | |
| | actggggccagggcaccagcgtgaccgtgagcagc | |
| 58 | X₁PPX₂P | Hinge |
| | X₁ is glycine, cysteine or arginine | |
| | X₂ is cysteine or threonine | |
| 59 | GSTSGSGKPGSGEGSTKG | Linker |

1. A method of treatment, the method comprising:
(1) administering to a subject having a cancer an effective amount of a kinase inhibitor that is or comprises the structure 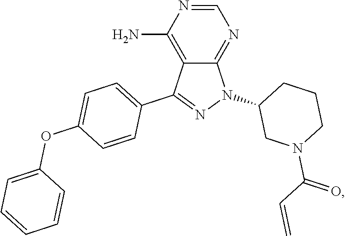 [SMILES: C=CC(=O)N1CCC[C@@H](n2nc(-c3ccc(Oc4ccccc4)cc3)c3c(N)ncnc32)C1]
or a pharmaceutically acceptable salt thereof; and
(2) administering an autologous T cell therapy to the subject, said T cell therapy comprising a dose of genetically engineered T cells expressing a chimeric antigen receptor (CAR) that specifically binds to a CD19, wherein, prior to administering the T cell therapy, a biological sample has been obtained from the subject and processed, the processing comprising genetically modifying T cells from the sample, optionally by introducing a nucleic acid molecule encoding the CAR into said T cells,
wherein the administration of the kinase inhibitor is initiated at least at or about 3 days prior to the obtaining of the sample and is carried out in a dosing regimen comprising repeat administrations of the kinase inhibitor at a dosing interval, over a period of time that extends at least to include administration on or after the day that the sample is obtained from the subject.

2. A method of treatment, the method comprising:
(1) administering to a subject having a cancer an effective amount of a kinase inhibitor that is or comprises the structure 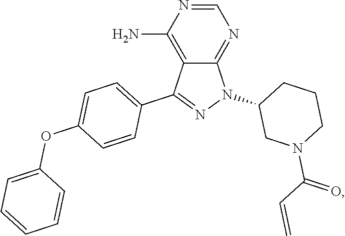 [SMILES: C=CC(=O)N1CCC[C@@H](n2nc(-c3ccc(Oc4ccccc4)cc3)c3c(N)ncnc32)C1]
or a pharmaceutically acceptable salt thereof;
(2) obtaining from the subject a biological sample and processing T cells of said sample, thereby generating a composition comprising genetically engineered T cells that express a chimeric antigen receptor (CAR) that specifically binds to a CD19; and
(3) administering to the subject an autologous T cell therapy comprising a dose of the genetically engineered T cells,
wherein the administration of the kinase inhibitor is carried out in a dosing regimen that is initiated at least at or about 3 days prior to the obtaining of the sample and that comprises repeat administrations of the inhibitor, at a dosing interval, over a period of time and extends at least to include administration of the compound on or after the day that the sample is obtained from the subject.

3. A method of treatment, the method comprising administering to a subject having a cancer an effective amount of a kinase inhibitor having the structure 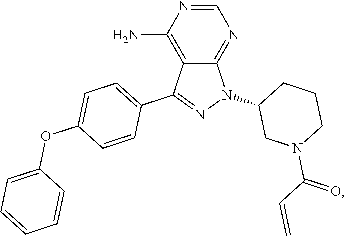 [SMILES: C=CC(=O)N1CCC[C@@H](n2nc(-c3ccc(Oc4ccccc4)cc3)c3c(N)ncnc32)C1]
or a pharmaceutically acceptable salt thereof, wherein the subject is a candidate for treatment or is to be treated with an autologous T cell therapy, said T cell therapy comprising a dose of genetically engineered T cells expressing a chimeric antigen receptor (CAR) that specifically binds to a CD19, wherein:
prior to administering the T cell therapy a biological sample has been obtained from the subject and processed, the processing comprising genetically modifying T cells from the sample, optionally by introducing a nucleic acid molecule encoding the CAR into said T cells; and
the administration of the kinase inhibitor is initiated at least at or about 3 days prior to the obtaining of the sample and is carried out in a dosing regimen comprising repeat administrations of the inhibitor at a dosing interval for a period of time that extends at least to include administration on or after the day that the sample is obtained from the subject.

4. The method of claim 3, further comprising administering to the subject the T cell therapy.

5. The method of any of claims 1, 2 and claim 4, wherein, subsequent to initiation the administration of the kinase inhibitor and prior to the administration of the T cell therapy, the subject has been preconditioned with a lymphodepleting therapy.

6. The method of any of claims 1, 2 and claim 4, further comprising, subsequent to initiating the administration of the kinase inhibitor and prior to the administration of the T cell therapy, administering a lymphodepleting therapy to the subject.

7. The method of claim 5 or claim 6, wherein the administration of the kinase inhibitor is discontinued or halted during the lymphodepleting therapy.

8. The method of any of claims 5-7, wherein the dosing regimen comprises administration of the kinase inhibitor over a period of time that extends at least to include administration up to the initiation of the lymphodepleting therapy.

9. The method of any of claims 5-7, wherein the dosing regimen comprises administration of the kinase inhibitor over a period of time that includes administration up to the initiation of the lymphodepleting therapy, followed by discontinuing or halting administration of the kinase inhibitor during the lymphodepleting therapy and then further administration of the kinase inhibitor for a period that extends for at least 15 days after initiation of administration of the T cell therapy.

10. A method of treatment, the method comprising:
(1) administering to a subject having a cancer an effective amount of a kinase inhibitor having the structure 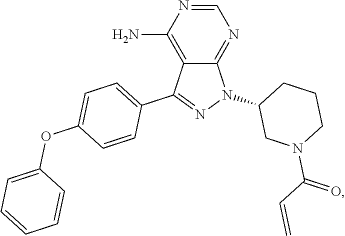 [SMILES: C=CC(=O)N1CCC[C@@H](n2nc(-c3ccc(Oc4ccccc4)cc3)c3c(N)ncnc32)C1]
or a pharmaceutically acceptable salt thereof;
(2) administering a lymphodepleting therapy to the subject; and
(3) administering an autologous T cell therapy to the subject, said T cell therapy comprising a dose of genetically engineered T cells expressing a chimeric antigen receptor (CAR) that specifically binds to a CD19, wherein, prior to administering the T cell therapy comprising biological sample has been obtained from the subject and processed, the processing comprising genetically modifying T cells from the sample, optionally by introducing a nucleic acid molecule encoding the CAR into said T cells,
wherein the administration of the kinase inhibitor is initiated at least at or about 3 days prior to obtaining the obtaining of the sample and is carried out in a dosing regimen comprising repeat administrations of the kinase inhibitor at a dosing interval over a period of time that includes administration up to the initiation of the lymphodepleting therapy, followed by discontinuing or halting administration of the kinase inhibitor during the lymphodepleting therapy, and then further administration of the kinase inhibitor for a period that extends for at least 15 days after initiation of administration of the T cell therapy.

11. The method of claim 10, wherein the method further comprises obtaining from the subject the biological sample and processing T cells of said sample, thereby generating a composition comprising the genetically engineered T cells that express the chimeric antigen receptor (CAR) that specifically binds to a CD19.

12. The method of any of claims 1-11, wherein the administration of the kinase inhibitor is initiated at least at or about 4 days, at least at or about 5 days, at least at or 6 days, at least at or about 7 days, at least at or about 14 days or more prior to the obtaining the sample from the subject.

13. The method of any of claims 1-12, wherein the administration of the kinase inhibitor is initiated at least or at or about 5 days to 7 days prior to the obtaining the sample from the subject.

14. The method of any of claims 5-13, wherein administration of the lymphodepleting therapy is completed within 7 days prior to initiation of the administration of the T cell therapy.

15. The method of any of claims 5-14, wherein administration of the lymphodepleting therapy is completed 2 to 7 days prior to initiation of the administration of the T cell therapy.

16. The method of any of claims 9-15, wherein the further administration is for a period that extends for 15 days to 29 days after initiation of administration of the T cell therapy.

17. The method of any of claims 9-16, wherein the further administration of the kinase inhibitor is for a period that extends at or about or greater than three months after initiation of administration of the T cell therapy.

18. The method of any of claims 1-17, wherein the administration of the kinase inhibitor is carried out once per day on each day it is administered during the dosing regimen.

19. The method of any of claims 1-18, wherein the effective amount comprises from or from about 140 mg to or to about 840 mg or from or from about 140 mg to or to about 560 mg per each day the kinase inhibitor is administered.

20. A method of treatment, the method comprising:
(1) administering to a subject having a cancer a kinase inhibitor, wherein the kinase inhibitor is or comprises the structure 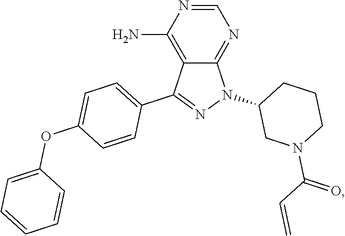 [SMILES: C=CC(=O)N1CCC[C@@H](n2nc(-c3ccc(Oc4ccccc4)cc3)c3c(N)ncnc32)C1]
or is a pharmaceutically acceptable salt thereof; and
(2) administering an autologous T cell therapy to the subject, said T cell therapy comprising a dose of genetically engineered T cells expressing a chimeric antigen receptor (CAR) that specifically binds to a CD19, wherein, prior to administering the T cell therapy a biological sample has been obtained from the subject and processed, the processing comprising genetically modifying T cells from the sample, optionally by introducing a nucleic acid molecule encoding the CAR into said T cells,
wherein the administration of the kinase inhibitor is initiated at least at or about 5 to 7 days prior to the obtaining of the sample and is carried out in a dosing regimen comprising repeat administration of the kinase inhibitor at a dosing interval over a period of time that extends at least to include administration on or after the day that the sample is obtained from the subject and further administration that extends for at or about or greater than three months after initiation of administration of the T cell therapy, wherein the kinase inhibitor is administered in an amount from or from about 140 mg to or to about 560 mg once per day each day it is administered during the dosing regimen.

21. The method of claim 20, wherein, subsequent to initiating administration of the kinase inhibitor and prior to the administration of the T cell therapy, the subject has been preconditioned with a lymphodepleting therapy.

22. The method of claim 20, further comprising, subsequent to the administration of the kinase inhibitor and prior to the administration of the T cell therapy, administering a lymphodepleting therapy to the subject.

23. The method of any of claims 20-22, wherein the administration of the lymphodepleting therapy is completed within 7 days prior to initiation of the administration of the T cell therapy.

24. The method of any of claims 20-23, wherein the administration of the lymphodepleting therapy is completed 2 to 7 days prior to initiation of the administration of the T cell therapy.

25. The method of any of claims 22-24, wherein the dosing regimen comprises discontinuing or halting administration of the kinase inhibitor during the lymphodepleting therapy.

26. A method of treatment, the method comprising:
(1) administering to a subject having a cancer a kinase inhibitor, wherein the kinase inhibitor has the structure 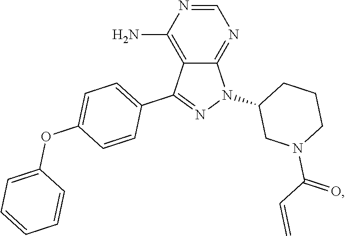 [SMILES: C=CC(=O)N1CCC[C@@H](n2nc(-c3ccc(Oc4ccccc4)cc3)c3c(N)ncnc32)C1]
or is a pharmaceutically acceptable salt thereof; and
(2) administering a lymphodepleting therapy to the subject; and
(3) administering an autologous T cell therapy to the subject within 2 to 7 days after completing the lymphodepleting therapy, said T cell therapy comprising a dose of genetically engineered T cells expressing a chimeric antigen receptor (CAR) that specifically binds to a CD19, wherein, prior to administering the T cell therapy a biological sample has been obtained from the subject and processed, the processing comprising genetically modifying T cells from the sample, optionally by introducing a nucleic acid molecule encoding the CAR into said T cells,
wherein the administration of the kinase inhibitor is initiated at least at or about 5 to 7 days prior to the obtaining of the sample and is carried out in a dosing regimen comprising repeat administration of the kinase inhibitor at a dosing interval that includes administration up to the initiation of the lymphodepleting therapy, followed by discontinuing or halting administration of the kinase inhibitor during the lymphodepleting therapy, and then further administration for a period that extends for at or greater than three months after initiation of administration of the T cell therapy, wherein the kinase inhibitor is administered in an amount from or from about 140 mg to or to about 560 mg once per day each day it is administered during the dosing regimen.

27. The method of any of claims 20-26, wherein the method further comprises obtaining from the subject the biological sample and processing T cells of said sample, thereby generating a composition comprising the genetically engineered T cells that express the chimeric antigen receptor (CAR) that specifically binds to a CD19

28. The method of any of claims 1-27, wherein the administration of the kinase inhibitor per day it is administered is from or from about 280 mg to or to about 560 mg.

29. The method of any of claims 1-28, wherein administration of the kinase inhibitor is initiated at least at or about 7 days prior to obtaining the sample from the subject.

30. The method of any of claims 1-29, wherein:
the administration of the kinase inhibitor is initiated from or from about 30 to or to about 40 days prior to initiating the administration of the T cell therapy;
the sample is obtained from the subject from or from about 23 to or to about 38 days prior to initiating the administration of the T cell therapy; and/or
the lymphodepleting therapy is completed at or about 5 to 7 days prior to initiating administration of the T cell therapy.

31. The method of any of claims 1-30, wherein:
the administration of the kinase inhibitor is initiated at or about 35 days prior to initiating the administration of the T cell therapy;
the sample is obtained from the subject from or from about 28 to or to about 32 days prior to initiating the administration of the T cell therapy; and/or
the lymphodepleting therapy is completed at or about 5 to 7 days prior to initiating administration of the T cell therapy.

32. The method of any of claims 5-31, wherein the lymphodepleting therapy comprises the administration of fludarabine and/or cyclophosphamide.

33. The method of any of claims 5-32, wherein the lymphodepleting therapy comprises administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days, or wherein the lymphodepleting therapy comprises administration of cyclophosphamide at or about 500 mg/m².

34. The method of any one of claims 5-33, wherein:
the lymphodepleting therapy comprises administration of cyclophosphamide at or about 300 mg/m² and fludarabine at or about 30 mg/m² daily for 3 days; and/or
the lymphodepleting therapy comprises administration of cyclophosphamide at or about 500 mg/m² and fludarabine at or about 30 mg/m² daily for 3 days.

35. The method of any of claims 1-34, wherein the administration of the kinase inhibitor per day it is administered is at an amount of at or about 140 mg.

36. The method of any of claims 1-34, wherein the administration of the kinase inhibitor per day it is administered is at an amount of at or about 280 mg.

37. The method of any of claims 1-34, wherein the administration of the kinase inhibitor per day it is administered is at an amount of at or about 420 mg.

38. The method of any of claims 1-34, wherein the administration of the kinase inhibitor per day it is administered is at an amount of at or about 560 mg.

39. The method of any of claims 9-38, wherein the period extends for at or about or greater than four months after the initiation of the administration of the T cell therapy or at or about or greater than five months after the initiation of the administration of the T cell therapy.

40. The method of any of claims 9-39, wherein the further administration is for a period that extends at or about or greater than six months.

41. The method of any of claims 9-40, wherein:
the further administration of the kinase inhibitor is stopped at the end of the period, if, at the end of the period, the subject exhibits a complete response (CR) following the treatment; or
the further administration of the kinase inhibitor is stopped at the end of the period if, at the end of the period, the cancer has progressed or relapsed following remission after the treatment.

42. The method of any of claims 9-41, wherein the period extends for from or from at or about three months to at or six months.

43. The method of any of claims 9-42, wherein the period extends for at or about three months after initiation of administration of the T cell therapy.

44. The method of any of claims 9-42, wherein the period extends for at or about 3 months after initiation of administration of the T cell therapy if the subject has, prior to at or about 3 months, achieved a complete response (CR) following the treatment or the cancer has progressed or relapsed following remission after the treatment.

45. The method of claim 44, wherein the period extends for at or about 3 months after initiation of administration of the T cell therapy if the subject has at 3 months achieved a complete response (CR).

46. The method of any of claims 9-42, wherein the period extends for at or about six months after initiation of administration of the T cell therapy.

47. The method of any of claims 9-42, wherein the period extends for at or about 6 months after initiation of administration of the T cell therapy if the subject has, prior to at or about 6 months, achieved a complete response (CR) following the treatment or the cancer has progressed or relapsed following remission after the treatment.

48. The method of claim 47, wherein the period extends for at or about 6 months after initiation of administration of the T cell therapy if the subject has at 6 months achieved a complete response (CR).

49. The method of any of claims 9-48, wherein the further administration is continued for the duration of the period even if the subject has achieved a complete response (CR) at a time point prior to the end of the period.

50. The method of any of claims 9-49, wherein the subject achieves a complete response (CR) at a time during the period and prior to the end of the period.

51. The method of any of claims 9-40, 42, 43, 44, 46 and 47, further comprising continuing the further administration after the end of the period, if, at the end of the period, the subject exhibits a partial response (PR) or stable disease (SD).

52. The method of any of claims 9-40, 42, 43, 44, 46, 47 and 51, wherein the further administration is continued for greater than six months if, at or about six months, the subject exhibits a partial response (PR) or stable disease (SD) after the treatment.

53. The method of claim 51 or claim 52, wherein the further administration is continued until the subject has achieved a complete response (CR) following the treatment or until the cancer has progressed or relapsed following remission after the treatment.

54. The method of any of claims 1-53, wherein the kinase inhibitor inhibits Bruton's tyrosine kinase (BTK) and/or inhibits IL2 inducible T-cell kinase (ITK).

55. The method of any of claims 1-54, wherein the kinase inhibitor inhibits ITK and the inhibitor inhibits ITK or inhibits ITK with a half-maximal inhibitory concentration (IC₅₀) of less than or less than about 1000 nM, 900 nM, 800 nM, 600 nM, 500 nM, 400 nM, 300 nM, 200 nM, 100 nM or less.

56. The method of any of claims 1-55, wherein the subject had previously been administered the kinase inhibitor prior to the administration of the kinase inhibitor in (1).

57. The method of any of claims 1-55, wherein the subject has not previously been administered the kinase inhibitor prior to the administration of the kinase inhibitor in (1).

58. The method of any of claims 1-57, wherein:
(i) the subject and/or the cancer (a) is resistant to inhibition of Bruton's tyrosine kinase (BTK) and/or (b) comprises a population of cells that are resistant to inhibition by the kinase inhibitor, optionally wherein the population of cells is or comprises a population of B cells and/or does not comprise T cells;
(ii) the subject and/or the cancer comprises a mutation in a nucleic acid encoding a BTK, optionally wherein the mutation is capable of reducing or preventing inhibition of the BTK by the kinase inhibitor, optionally wherein the mutation is C481S;
(iii) the subject and/or the cancer comprises a mutation in a nucleic acid encoding phospholipase C gamma 2 (PLCgamma2), optionally wherein the mutation results in constitutive signaling activity, optionally wherein the mutation is R665W or L845F;
(iv) at the time of the initiation of administration of the kinase inhibitor in (1), and optionally at the time of the initiation of administration of the T cell therapy, the subject has relapsed following remission after a previous treatment with, or been deemed refractory to a previous treatment with, the kinase inhibitor and/or with a BTK inhibitor therapy;
(v) at the time of the initiation of administration of the kinase inhibitor in (1), and optionally at the time of the initiation of the T cell therapy, the subject has progressed following a previous treatment with the inhibitor and/or with a BTK inhibitor therapy, optionally wherein the subject exhibited progressive disease as the best response to the previous treatment or progression after previous response to the previous treatment; and/or
(vi) at the time of the initiation of administration of the kinase inhibitor in (1), and optionally at the time of the initiation of the T cell therapy, the subject exhibited a response less than a complete response (CR) following a previous treatment for at least 6 months with the inhibitor and/or with a BTK inhibitor therapy.

59. The method of any one of claims 1-58, wherein the cancer is a B cell malignancy.

60. The method of claim 59, wherein the B cell malignancy is a lymphoma.

61. The method of claim 60, wherein the lymphoma is a non-Hodgkin lymphoma (NHL).

62. The method of claim 61, wherein the NHL comprises aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma with MYC and BCL2 and/or BCL6 rearrangements with DLBCL histology (double/triple hit).

63. The method of any one of claims 1-62, wherein the subject is or has been identified as having an Eastern Cooperative Oncology Group Performance Status (ECOG) status of less than or equal to 1.

64. The method of any of claims 1-63, wherein the kinase inhibitor is administered orally.

65. The method of any of claims 1-64, wherein the CD19 is a human CD19.

66. The method of any of claims 1-65, wherein the chimeric antigen receptor (CAR) comprises an extracellular antigen-recognition domain that specifically binds to the CD19 and an intracellular signaling domain comprising an ITAM.

67. The method of claim 66, wherein the intracellular signaling domain comprises a signaling domain of a CD3-zeta (CD3) chain, optionally a human CD3-zeta chain.

68. The method of claim 66 or claim 67, wherein the chimeric antigen receptor (CAR) further comprises a costimulatory signaling region.

69. The method of claim 68, wherein the costimulatory signaling region comprises a signaling domain of CD28 or 4-1BB, optionally human CD28 or human 4-1BB.

70. The method of claim 68 or claim 69, wherein the costimulatory domain is or comprises a signaling domain of human 4-1BB.

71. The method of any of claims 1-70, wherein:
the CAR comprises an scFv specific for the CD19; a transmembrane domain; a cytoplasmic signaling domain derived from a costimulatory molecule, which optionally is or comprises a 4-1BB, optionally human 4-1BB; and a cytoplasmic signaling domain derived from a primary signaling ITAM-containing molecule, which optionally is or comprises a CD3zeta signaling domain, optionally a human CD3zeta signaling domain; and optionally wherein the CAR further comprises a spacer between the transmembrane domain and the scFv;
the CAR comprises, in order, an scFv specific for the CD19; a transmembrane domain; a cytoplasmic signaling domain derived from a costimulatory molecule, which optionally is or comprises a 4-1BB signaling domain, optionally a human 4-1BB signaling domain; and a cytoplasmic signaling domain derived from a primary signaling ITAM-containing molecule, which optionally is a CD3zeta signaling domain, optionally human CD3zeta signaling domain; or
the CAR comprises, in order, an scFv specific for the CD19; a spacer; a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

72. The method of claim 71, wherein
the CAR comprises a spacer and the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P (SEQ ID NO:58), where X₁ is glycine, cysteine or arginine and X₂ is cysteine or threonine; and/or
the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or
the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or
the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a V_{H}, a linker, optionally comprising SEQ ID NO: 41, and a V_{L}, and/or the scFv comprises a flexible linker and/or comprises the amino acid sequence set forth as SEQ ID NO: 42.

73. The method of any of claims 1-72, wherein the dose of genetically engineered T cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

74. The method of any of claims 1-73, wherein the dose of genetically engineered T cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

75. The method of any of claims 1-74, wherein the dose of genetically engineered T cells comprises at or about 5×10⁷ total CAR-expressing T cells.

76. The method of any of claims 1-75, wherein the dose of genetically engineered T cells comprises at or about 1×10⁸ CAR-expressing cells.

77. The method of any of claims 1-76, wherein the dose of genetically engineered T cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and the second composition comprising the other of the CD4+ T cells or the CD8+ T cells.

78. The method of claim 77, wherein:
the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or
the initiation of administration of the first composition and the initiation of administration of the second composition are carried out between about 1 minute and about 1 hour apart or between about 5 minutes and about 30 minutes apart.

79. The method of claim 77 or claim 78, wherein the first composition and second composition are administered no more than 2 hours, no more than 1 hour, no more than 30 minutes, no more than 15 minutes, no more than 10 minutes or no more than 5 minutes apart.

80. The method of any of claims 77-79, wherein the first composition comprises the CD4+ T cells.

81. The method of any of claims 77-79, wherein the first composition comprises the CD8+ T cells.

82. The method of any of claims 77-81, wherein the first composition is administered prior to the second composition.

83. The method of any of claims 1-82, wherein the dose of cells is administered parenterally, optionally intravenously.

84. The method of any of claims 1-83, wherein the T cells are primary T cells obtained from the sample from the subject.

85. The method of any of claims 1-82, wherein the T cells are autologous to the subject.

86. The method of any of claims 1-85, wherein the processing comprises:
isolating T cells, optionally CD4+ and/or CD8+ T cells, from the sample obtained from the subject, thereby producing an input composition comprising primary T cells; and
introducing the nucleic acid molecule encoding the CAR into T cells of the input composition.

87. The method of claim 86, wherein the isolating comprising carrying out immunoaffinity-based selection.

88. The method of any of claims 1-87, wherein the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.

89. The method of any of claims 86-88, wherein prior to the introducing, the processing comprises incubating the input composition under stimulating conditions, said stimulating conditions comprising the presence of a stimulatory reagent capable of activating one or more intracellular signaling domains of one or more components of a TCR complex and/or one or more intracellular signaling domains of one or more costimulatory molecules, thereby generating a stimulated composition, wherein the nucleic acid molecule encoding the CAR is introduced into the stimulated composition.

90. The method of claim 89, wherein the stimulatory reagent comprises a primary agent that specifically binds to a member of a TCR complex, optionally that specifically binds to CD3.

91. The method of claim 90, wherein the stimulatory reagent further comprises a secondary agent that specifically binds to a T cell costimulatory molecule, optionally wherein the costimulatory molecule is selected from CD28, CD137 (4-1-BB), OX40, or ICOS.

92. The method of claim 90 or claim 91, wherein the primary and/or secondary agents comprise an antibody, optionally wherein the stimulatory reagent comprises incubation with an anti-CD3 antibody and an anti-CD28 antibody, or an antigen-binding fragment thereof.

93. The method of any of claims 90-92, wherein the primary agent and/or secondary agent are present on the surface of a solid support.

94. The method of claim 93, wherein the solid support is or comprises a bead, optionally wherein the bead is magnetic or superparamagnetic.

95. The method of claim 94, wherein the bead comprises a diameter of greater than or greater than about 3.5 μm but no more than about 9 μm or no more than about 8 μm or no more than about 7 μm or no more than about 6 μm or no more than about 5 μm.

96. The method of claim 94 or claim 95, wherein the bead comprises a diameter of or about 4.5 μm.

97. The method of any of claims 1-96, wherein the introducing comprises transducing cells of the stimulated composition with a viral vector comprising a polynucleotide encoding the recombinant receptor.

98. The method of claim 97, wherein the viral vector is a retroviral vector.

99. The method of claim 97 or claim 98, wherein the viral vector is a lentiviral vector or gammaretroviral vector.

100. The method of any of claims 86-99, wherein the processing further comprises after the introducing cultivating the T cells, optionally wherein the cultivating is carried out under conditions to result in the proliferation or expansion of cells to produce an output composition comprising the T cell therapy.

101. The method of claim 100, wherein subsequent to the cultivating, the method further comprises formulating cells of the output composition for cryopreservation and/or for administration of the T cell therapy to the subject, optionally wherein the formulating is in the presence of a pharmaceutically acceptable excipient.

102. The method of any of claims 1-101, wherein the subject is a human.

103. The method of any of claims 1-102, wherein:
at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or
wherein at least 60, 70, 80, 90, or 95% of subjects achieving a CR by six months remain in response, remain in CR, and/or survive or survive without progression, for greater at or greater than 3 months and/or at or greater than 6 months and/or at greater than nine months; and/or
at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or
wherein at least 60, 70, 80, 90, or 95% of subjects achieving an OR by six months remain in response or surviving for greater at or greater than 3 months and/or at or greater than 6 months.

104. The method of any of claims 60-103, wherein, at or immediately prior to the time of the administration of the dose of cells the subject has relapsed following remission after treatment with, or become refractory to, one or more prior therapies for the lymphoma, optionally the NHL, optionally one, two or three prior therapies other than another dose of cells expressing the CAR.

105. The method of any of claim 60-104, wherein, at or prior to the administration of the T cell therapy comprising the dose of cells:
the subject is or has been identified as having a double/triple hit lymphoma;
the subject is or has been identified as having a chemorefractory lymphoma, optionally a chemorefractory DLBCL; and/or
the subject has not achieved a complete response (CR) in response to a prior therapy.

106. A kit comprising one or more unit doses of a kinase inhibitor that is or comprises the structure 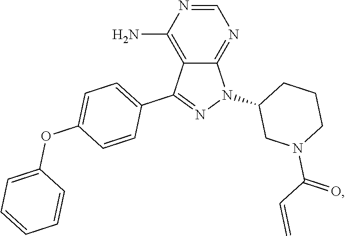 [SMILES: C=CC(=O)N1CCC[C@@H](n2nc(-c3ccc(Oc4ccccc4)cc3)c3c(N)ncnc32)C1]
or is a pharmaceutically acceptable salt thereof, and instructions for administering the one or more unit doses to a subject having a cancer that is a candidate for treatment with or who is to be treated with an autologous T cell therapy, said T cell therapy comprising a dose of genetically engineered T cells expressing a chimeric antigen receptor (CAR) that specifically binds to a CD19, and in which, prior to administration of the T cell therapy, a biological sample is obtained from the subject and processed, the processing comprising genetically modifying T cells from the sample, optionally by introducing a nucleic acid encoding the CAR into the T cells,
wherein the instructions specify initiating administration of a unit dose of the kinase inhibitor to the subject at or at least about 3 days prior to the obtaining of the sample and in a dosing regimen comprising repeat administrations of one or more unit doses at a dosing interval over a period of time that extends at least to include administration on or after the day the sample is obtained from the subject.

107. The kit of claim 106, wherein the instructions further specify administering the T cell therapy to the subject.

108. The kit of claim 106 or claim 107, wherein the instructions further specify, subsequent to initiating the administration of the kinase inhibitor and prior to the administration of the T cell therapy, administering a lymphodepleting therapy to the subject.

109. The kit of claim 108, wherein the instructions specify administration of the kinase inhibitor is to be discontinued during administration of the lymphodepleting therapy.

110. The kit of claim 108 or claim 109, wherein the instructions specify the dosing regimen comprises administration of the kinase inhibitor for a period of time that extends at least until the initiation of the lymphodepleting therapy.

111. The kit of any of claims 108-110, wherein the instructions specify the dosing regimen comprises administration of the kinase inhibitor over a period of time that includes administration up to the initiation of the lymphodepleting therapy, followed by discontinuing or halting administration of the kinase inhibitor during the lymphodepleting therapy, and then further administration of the kinase inhibitor for a period that extends for at least 15 days after initiation of administration of the T cell therapy.

112. A kit comprising one or more unit doses of a kinase inhibitor that is or comprises the structure 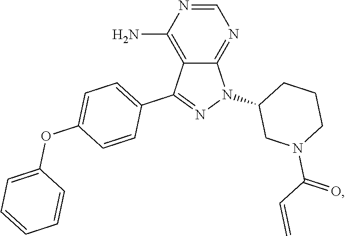 [SMILES: C=CC(=O)N1CCC[C@@H](n2nc(-c3ccc(Oc4ccccc4)cc3)c3c(N)ncnc32)C1]
or is a pharmaceutically acceptable salt thereof, and instructions for carrying out the methods of any of claims 1-105.

113. An article of manufacture comprising the kit of any of claims 106-112.
